(19) United States
(12) Patent Application Publication
(10) Pub. No.: US 2013/0296183 A1
(43) Pub. Date: Nov. 7, 2013

(54) FUNCTIONAL GENOMICS ASSAY FOR CHARACTERIZING PLURIPOTENT STEM CELL UTILITY AND SAFETY

(71) Applicants: Kevin C. Eggan, Boston, MA (US); Alexander Meissner, Cambridge, MA (US); Christoph Bock, Vienna (AT); Evangelos Kiskinis, Boston, MA (US); Griet Annie Frans Verstappen, Moltsel (BE)

(72) Inventors: Kevin C. Eggan, Boston, MA (US); Alexander Meissner, Cambridge, MA (US); Christoph Bock, Vienna (AT); Evangelos Kiskinis, Boston, MA (US); Griet Annie Frans Verstappen, Moltsel (BE)

(73) Assignee: PRESIDENT AND FELLOWS OF HARVARD COLLEGE, Cambridge, MA (US)

(21) Appl. No.: 13/822,336

(22) PCT Filed: Sep. 16, 2011
(86) PCT No.: PCT/US2011/051931

§ 371 Date: Jul. 23, 2013

Related U.S. Application Data
(60) Provisional application No. 61/384,030, filed on Sep. 17, 2010.
(60) Provisional application No. 61/429,965, filed on Jan. 5, 2011.

(51) Int. Cl. C12N 15/10

(52) U.S. Cl. CPC C12N 15/1072
USPC 506/9; 506/16

(57) ABSTRACT

The present invention generally relates set of reference data or “scorecard” for a pluripotent stem cell, and methods, systems and kits to generate a scorecard for predicting the functionality and suitability of a pluripotent stem cell line for a desired use. In some aspects, a method for generating a scorecard comprises using at least 2 stem cell assays selected from: epigenetic profiling, differentiation assay and gene expression assay to predict the functionality and suitability of a pluripotent stem cell line for a desired use. In some embodiments, the scorecard reference data can be compared with the pluripotent stem cells data to effectively and accurately predict the utility of the pluripotent stem cell for a given application, as well as any to identify specific characteristics of the pluripotent stem cell line to determine their suitability for downstream applications, such as for example, their suitability for therapeutic use, drug screening and toxicity assays, differentiation into a desired cell lineage, and the like.

### CROSS REFERENCE TO RELATED APPLICATIONS

This application claims priority under 35 U.S.C. 119(e) of U.S. Provisional Patent Application Ser. No. 61/384,030 filed on Sep. 17, 2010, and provisional application 61/429,965 filed on Jan. 5, 2011, the contents of which are incorporated herein by reference in their entirety.

### GOVERNMENT SUPPORT

This invention was made in part, with government support under NIH Roadmap Initiative on Epigenomics, Grant Number U01ES017155 awarded by National Institutes of Health. The Government of the U.S. has certain rights in the invention.

### FIELD OF THE INVENTION

The present invention relates to method for characterizing, such as characterizing by high throughput methods, stem cells, and for methods and compositions for standardizing and optimizing the selection of pluripotent cell lines for disease modeling, studying stem cell population and their use for therapeutic treatment of diseases.

### REFERENCES TO TABLES

This application includes as part of the originally filed subject matter three compact discs, labeled “Copy 1” and “Copy 2,” and “Copy 3” each disc containing eleven (11) text files. Each of the compact discs (“Copy 1”, “Copy 2” and “Copy 3”) includes eleven (11) text files for ten separate lengthy tables, which are named “002806-067741-P2_TABLE 3.txt” (9,919 KB, created Jan. 7, 2011), “002806-067741-P2_TABLE 4.txt” (19,381 KB, created Jan. 7, 2011), “002806-067741-P2_TABLE 5.txt” (10,006 KB, created Jan. 7, 2011), “002806-067741-P2_TABLE 8.txt” (98 KB, created Jan. 7, 2011), “002806-067741-P2_TABLE 10.txt” (180 KB, created Jan. 7, 2011), “002806-067741-P2_TABLE 12A.txt” (160 KB, created Jan. 7, 2011); “002806-067741-P2_TABLE 12B.txt” (160 KB, created Jan. 7, 2011); “002806-067741-P2_TABLE 12C.txt” (31 KB, created Jan. 7, 2011), 002806-067741-P2_TABLE 13A.txt (25 KB, created Jan. 7, 2011), 002806-067741-P2_TABLE 13B.txt (28 KB, created Jan. 7, 2011), 002806-067741-P2_TABLE 14.txt (10 KB, created Jan. 7, 2011). The machine format of each compact disc (“Copy 1”, “Copy 2” and “Copy 3”) is IBM-PC and the operating system of each compact disc is MS-Windows. The contents of the compact discs labeled “Copy 1” and “Copy 2” and “Copy 3” are hereby incorporated by reference herein in their entireties.

### Lengthy Tables

The specification includes eleven (11) lengthy Tables; Tables 3, Table 4, Table 5, Table 8, Table 10, Table 12A, Table 12B, Table 12C, Table 13A, Table 13B and Table 14. Lengthy Table 3 is the integrated DNA methylation and gene expression data for Ensembl genes and promoter regions (defined as −5 kb to +1 kb surrounding the Ensembl-annotated transcription start site) and is provided herein in an electronic format on a CD, as file “002806-067741-P2_TABLE 3.txt”. Lengthy Table 4 is the DNA methylation data for 35 cell lines and 31,929 Ensembl gene promoter regions, sorted in descending order of epigenetic variation among all ES cell lines (column BF) and is provided herein in an electronic format on a CD, as file “002806-067741-P2_TABLE 4.txt”. Lengthy Table 5 is the Gene expression data for 35 cell lines and 15,079 Ensembl genes, sorted in descending order of transcription variation among all ES cell lines (column BG) and is provided herein in an electronic format on a CD, as file “002806-067741-P2_TABLE 5.txt”. Lengthy Table 8 is a table of the details of the individual measurements contributing to the lineage scorecard prediction and is provided herein in an electronic format on a CD, as file “002806-067741-P2_TABLE 8.txt”. Lengthy Table 10 is a table of the Gene expression data used for construction and validation of the lineage scorecard and is provided herein in an electronic format on a CD, as file “002806-067741-P2_TABLE 10.txt”. Lengthy Tables Table 12A, 12B and 12C are tables of the list of target genes for use in the score card, or assays and methods, with Table 12A showing, genes listed in descending order of priority which have been identified based on the variability in the reference set of DNA methylation variation among human pluripotent cell lines and Table 12B showing genes listed in descending order of priority that have been identified based on the variability in the reference set of gene expression variation among human pluripotent cell lines, and Table 12C showing genes are listed in descending order of priority and have been retrieved from the literature using an statistical ranking and information retrieval scheme, where genes from Table 12A, and/or Table 12B and/or Table 12C can be used for determining the score card and is provided herein in an electronic format on a CD, as files “002806-067741-P2_TABLE 12A.txt”, “002806-067741-P2_TABLE 12B.txt” and “002806-067741-P2_TABLE 12C.txt” respectively. Lengthy Tables 13A and 13B are tables of an alternative list of target genes listed as “included genes” which can be used for DNA methylation and gene expression measurement for determining the score card and lineage scorecard and is provided herein in an electronic format on a CD, as files “002806-067741-P2_TABLE 13A.txt” and “002806-067741-P2_TABLE 13B.txt” respectively. Lengthy Tables 14 is a table of an alternative list of target genes which are subgroup of genes of Table 13A which can be used for DNA methylation and gene expression measurement for determining the score card and lineage scorecard and is provided herein in an electronic format on a CD, as files “002806-067741-P2_TABLE 14.txt” Table 3, Tables 4, Table 5, Table 8, Table 10 and Tables 12A-12C, provided herein in an electronic format on a CD, as files “002806-067741-P2_TABLE 3.txt”; “002806-067741-P2_TABLE 4.txt”; “002806-067741-P2_TABLE 5.txt”; “002806-067741-P2_TABLE 8.txt”; “002806-067741-P2_TABLE 10.txt”, “002806-067741-P2_TABLE 12A.txt”, “002806-067741-P2_TABLE 12B.txt”, “002806-067741-P2_TABLE 12C.txt”, “002806-067741-P2_TABLE 13A.txt”, “002806-067741-P2_TABLE 13B.txt” and “002806-067741-P2_TABLE 14.txt” respectively are incorporated herein by reference in their entirety. Please refer to the end of the specification for access instructions.

### BACKGROUND OF THE INVENTION

One goal of regenerative medicine is to be able to convert pluripotent cells into other cell types for tissue repair and regeneration. Human pluripotent cell lines exhibit a level of developmental plasticity that is similar to the early embryo, enabling in vitro differentiation into all three embryonic germ layers (Rossant, 2008; Thomson et al., 1998). At the same time it is possible to maintain these pluripotent cell lines for many passages in the undifferentiated state (Adewumi et al., 2007). These unique characteristics render human embryonic stem (ES) and human induced pluripotent stem (iPS) cells a promising tool for biomedical research (Colman and Dreesen, 2009). ES cell lines have already been established as a model system for dissecting the cellular basis of monogenic human diseases. For example, it has been shown that ES cells carrying the mutation causing fragile X syndrome recapitulate phenotypic aspects of this disease when differentiated in vitro (Eiges et al., 2007). Additionally, human ES-cell derived motor neurons have been used to develop an in-vitro model for familial amyotrophic lateral sclerosis (ALS) that is compatible with drug screening (Di Giorgio et al., 2008). The discovery of defined reprogramming methods (Takahashi and Yamanaka, 2006) and their use in the derivation of patient-specific iPS cell lines (Dimos et al., 2008; Park et al., 2008) has further expanded the utility of pluripotent cells for monogenic disease modeling, enabling in vitro studies of spinal muscular atrophy (Ebert et al., 2009) and familial dysautonomia (Lee et al., 2009).

Until recently, only a few human pluripotent cell lines were widely available for biomedical research. For this reason, researchers have mostly relied on these readily accessible and well characterized cell lines (e.g., Thomson, bresigen and HUES 1-17 cell lines). Additionally, funding restrictions placed on ES cell research in the United States further limited the number of cell lines that were widely used. As a result, investigators used the lines that were available to them for their application of interest and there was little need for a diagnostic that could predict how a cell line behaved in a given assay.

Embryonic stem cells are unique in the ability to maintain pluripotency over significant periods in culture, making them leading candidates for use in cell therapy. Embryonic stem (ES) cell differentiation involves epigenetic mechanisms to control lineage-specific gene expression patterns. ES cell-based therapies hold great promise for the treatment of many currently intractable heritable, traumatic, and degenerative disorders. However, these therapeutic strategies inevitably involve the introduction of human cells that have been maintained, manipulated, and/or differentiated ex vivo to provide the desired precursor cells (e.g., somatic stem cells, etc.), raising the possibility that aberrant cells (e.g., cancer cells or cells predisposed to cancer that may occur during such manipulations and differentiation protocols) may be administered along with desired pluripotent stem cells or their differentiated progeny.

However, several recent developments have greatly increased the need for a diagnostic that can predict the behavior of pluripotent human cell lines. First, the continued derivation of human ES cell lines by many labs and the lifting of funding restrictions in the U.S. has substantially increased the number of ES cell lines that investigators may choose from. Additionally, it has become clear that not all human ES cell lines are equally suited for every purpose (Osafune et al., 2008). This suggests that any new research project should perform a deliberate and informed selection of the cell lines that are most qualified for an application of interest.

The discovery of factors that reprogram somatic cells from patients into iPS cells has also lead to a further increase in the number of pluripotent cell lines available to, and used by, the research community. As investigators gather together existing cell lines, or derive new ones for their application of interest, there is little information or guidance concerning how to select cell lines that are most appropriate for use.

Future applications of human pluripotent stem cell lines will likely include the study of common diseases that arise as the result of complex interactions between a person's genotype and their environment (Colman and Dreesen, 2009). In addition, pluripotent cells will eventually serve as a renewable source of both cells and tissue for transplantation medicine (Daley, 2010). Both of these proposed applications for pluripotent stem cells will require the selection of cell lines that reliably, reproducibly, efficiently and stably differentiate into disease-relevant cell types. However, a significant amount of variation has been reported in the efficiency by which various human ES cell lines differentiate into different derivatives of the three embryonic germ layers (Di Giorgio et al., 2008; Osafune et al., 2008). Concerns regarding the functional consequences of variation between pluripotent stem cell lines have been further fueled by studies of iPS cell lines. Specifically, it has been reported that iPS cells collectively deviate from ES cells in the expression of hundreds of genes (Chin et al., 2009), in their genome-wide DNA methylation patterns (Doi et al., 2009) and in their ability to differentiate down the motor neuron lineage (Hu et al., 2010). In contrast, it has also been reported that in some contexts iPS cell lines can differentiate as efficiently as ES cells (Boland et al., 2009; Miura et al., 2009; Zhao et al., 2009) and that published gene expression signatures of iPS cells may not be reproducible (Stadtfeld et al., 2010). These discrepancies must be resolved before human ES and iPS cell lines can be widely deployed as a tool for either disease modeling or transplantation therapy. In particular, it is necessary to establish a reference of normal variation among high-quality pluripotent cell lines, in order to provide a baseline against which variation from cell-line to cell-line can be identified and to enable systematic comparisons between classes of pluripotent cells (e.g., ES vs. iPS cell lines, iPS cell lines that carry a specific mutation vs. those that do not, iPS cell lines derived by different reprogramming protocols).

Therefore, there is a need in the art for novel, effective and efficient methods for pluripotent stem cell monitoring and validation, and for determining where in the spectrum of normal variation a pluripotent stem cell lines in comparison to other pluripotent stem cells, and effective and efficient methods to determine the safety profile and differentiation propensity of a pluripotent stem cell population prior to its use, e.g., in therapeutic administration to preclude administration of aberrant cells (e.g., cancer cells or cells predisposed to cancer), or in use on disease modeling, drug development and screening and toxicity assays.

### SUMMARY OF THE INVENTION

The present invention is directed to systems and methods to rapidly and relatively inexpensively screen for stem cells for their general quality and differentiation capacity, as well as their propensity for possible malignant growth. The systems and methods of the invention allow for a high throughput screening system which allows rapid identification and selection of cells, in some instances, an automated selection of cells which are suitable for further use or specific cells for a particular utility. The present invention relates to a method of characterization of pluripotent stem cells, including induced pluripotent stem cells (iPSCs) where the natural differentiation propensity analysis is highly predictive for how a specific cell line will perform in directed differentiation regimines and paradigms.

Presently, existing methods cannot predict how a pluripotent stem cell line will behave in a given directed differentiation paradigm. The methods and systems as disclosed herein provides a far superior system for pluripotent stem cell characterization as compared to the current existing and widely used systems, such as teratoma formation which are cumbersome, time consuming and very expensive to use, thus preventing these methods from becoming useful in a large scale characterization of stem cells. For example, use of teratoma formation or analysis of reprogramming factor silencing alone is not able to predict how the cell line will perform in directed differentiation, nor can these methods identify sub-optimal stem cell lines. The present methods and systems are not only faster, less expensive and suitable for automation, they provide for robust pluripotent stem cell characterization which is significantly more sensitive in identifying suitable or unsuitable stem cells and clones than the current gold standard method (e.g. using teratoma formation), and can be used to identify optimal pluripotent stem cells as well as identification of stem cell lines which fail to differentiate appropriately (e.g., stem cells which differentiate inefficienty or are poor pluripotent stem cell performing cells). Accordingly, the methods, systems and kits as disclosed herein provide a rapid, inexpensive and quantitative apprach for characterizing pluripotent stem cell lines which is highly useful in prediciting the differentiation ability of the cell as compared to traditional methods, and can identify stem cell lines which may be unsuitable for reasons such as high predisposition to become a malignant cell line.

Thus, the methods and systems as disclosed herein enable one to forecast the differentiation efficiency of a pluripotent stem cell line being analysed. For example, the methods and systems have been demonstrated to be highly predictive for differentiation of a pluripotent stem cell line along a particular lineage, e.g., a neuronal lineage such as a motor neuron lineage. The method and systems as disclosed herein has broad utility and can be used to prospectively predict how well a given pluripotent stem cell will differentiate along any desired lineage, for example, hematopoeitoic lineage, endoderm lineage, pancreatic lineage and the like.

The disclosed methods and system is based on the development of a novel system based on the gene expression of a determined set of genes that allows, in a high throughput manner, to screen for selected stem cell characteristics. Additionally, the novel system is also based on determination of DNA methylation of a determined set of genes. The sets of genes for gene expression and DNA methylation can be any predetermined set of genes, as disclosed herein, and include for example, but are not limited to lineage marker genes, as well as oncogenes and tumor suppressor genes and the like. The methods and systems further allow one to combine the obtained data automatically enabling selection of suitable cells or clones. Specifically, the system relies on determination of functional genomics data, such as posttranslational modification, gene expression data, DNA methylation, and epigenetic modifications and differentiation markers, such that the cells deviating from a normal range of functional genomic data, including DNA methylation, epigenetic modification, posttranslational modification, and differentiation marker expression pattern can be excluded, and the cells that fall within the normal ranges can be selected for further use. Statistical analysis methods are used to automate the system. In some embodiments, the functional genomic data is DNA methylation. In alternative embodiments, the functional genomic data is any, or a combination of posttranslational modification, such as, for example, methylation, ubiquitination, phosphorylation, glycosylation, sumoylation, acetylation, S-nitrosylation or nitrosylation, citrullination or deimination, neddylation, OClcNAc, ADP-ribosylation, hydroxylation, fattenylation, ufmylation, prenylation, myristoylation, S-palmitoylation, tyrosine sulfation, formylation, and carboxylation of histone and non-histone proteins (including cananical and variants of the proteins). In some embodiments, the functional genomic data, e.g., methylation and/or posttranslational modification is determined on gene sequences, as well as small non-coding RNAs and non-covalent structural modifications of the chromatin (e.g., condensation and decondensation).

Epigenetic modification and functional genomic modifications, such as methylation differences, or are associated with, for example, malignant cell growth. The present invention provides normal ranges of methylation patterns to allow the system of the invention to screen out the cells that are outliers and thus have potential for, for example malignant growth.

Screening for a set of desired cell differentiation markers allows selection of clones that have potential to develop to a desired tissue. For example, one can screen for markers for development into mesodermal, endodermal and ectodermal lineages. If the stem cell does not fit within the predetermined parameters for a multipotent cell expressing the appropriate marker set, it can be discarded.

The long-term proliferation and differentiation potential of human pluripotent stem cells suggests that they can produce large quantities of various cell types for disease modeling and transplantation therapy. However, before embryonic stem (ES) cells or induced pluripotent stem (iPS) cells can be used with confidence in therapeutic application or disease modeling, or for use in drug screening or toxicity assays, the extent of variation between human pluripotent cell lines must be understood. To obtain a comprehensive view of such variation, the inventors subjected 31 human ES and iPS cell lines to genome-wide DNA methylation and transcription analysis as well as quantified their in-vitro differentiation propensities.

In order to firmly establish the nature and magnitude in variation that exists among pluripotent stem cell lines, the inventors performed three genome-scale assays to 19 ES cell lines, 12 iPS cell lines and 6 primary fibroblast cell lines. The three assays included DNA methylation mapping by genome-scale bisulfate sequencing (Gu et al., 2010; Meissner et al., 2008), gene expression profiling using high-throughput microarrays, and a quantitative differentiation assay that utilizes transcript counting of 500 genes in embryoid bodies.

The inventors demonstrate the use of genome-wide analyses of DNA methylation and gene transcription profiles in a large cohort of human iPS and ES cell lines, and provide a newly discovered reference of common variation between pluripotent stem cell lines. The inventors use the genome-wide analyses of DNA methylation and gene transcription to provide a “lineage scorecard” that can be used to predict the differentiation propensities and utility of any pluripotent cell line. The inventors also demonstrate that human ES cells show variation and that iPS cells exhibit variation at similar loci. The inventors were unable to detect a single locus that can accurately distinguish between human ES cells and human iPS cells. Therefore, discovery of a system relying a pattern of multiple markers is important for screening stem cells that are useful for their intended purposes.

In particular, the inventors have demonstrated methods to acquire data from a plurality of pluripotent stem cell populations which provide a reference level of the normal variation of DNA methylation levels and/or gene expression levels among a variety of different pluripotent cell lines, which can be used to predict the behavior of individual pluripotent stem cell populations, e.g., stem cell lines, and provides a platform for systematic comparison between different classes of pluripotent stem cells, (e.g., ES cells versus iPS cells, or iPS cells versus partially induced iPS cells and the like).

In some embodiments, the inventors demonstrate the utility of the methods and systems of the present invention by predicting which pluripotent stem cell lines optimally differentiate into, for example motor neurons, and by performing quantitative comparisons between ES and iPS cell lines. This comparison demonstrates that there are no specific changes in DNA methylation or transcription that can be used universally to distinguish between an iPS and ES cell line. Accordingly, the inventors demonstrate that use of datasets, herein referred to “scorecards” and bioinformatics data tools enable high-throughput characterization of human pluripotent cell lines, such as iPS cells lines and embryonic cell lines using genomic assays.

Accordingly, the inventors have discovered efficient and effective methods, systems and kits which can be used to validate pluripotent stem cell populations in order to determine variability between different pluripotent cell populations, to predict their therapeutic utility and safety profile, (e.g., determining if the pluripotent stem cell population is predisposed to continual self-renewal and has high potential malignant transformation which is important if the pluripotent stem cell is to be transplanted for therapeutic use), and also enables one to predict the pluripotent stem cell populations differentiation potential of which lineages and developmental pathways the pluripotent stem cell line will efficiently differentiate into. As such, the methods, systems and kits as disclosed herein enable one to select a pluripotent stem cell with desirable characteristics, e.g., positively select for pluripotent stem cells with similar characteristics to other pluripotent stem cells, or pluripotent stem cells which have a predisposition to optimally differentiate into a desired cell type or along a specific cell lineage, or alternatively, the methods enable one to negatively select for, e.g., identify and discard, pluripotent stem cells which undesirable characteristic, e.g., cells which have a predisposition to develop into cancer cells.

Accordingly, the present invention relates to methods, systems and kits for effective and efficient pluripotent stem cell and/or precursor cell monitoring and validation, and for identifying pluripotent stem cells which are suitable for specific applications, e.g., for novel therapeutic methods, or for differentiating along specific lineages, the methods comprising monitoring and/or validating pluripotent stem cells prior to therapeutic administration to preclude introduction of aberrant cells (e.g., to avoid administering a pluripotent stem cell line which are proposed to become cancer cells or cells which are unlikely to differentiate along a specific desired lineage).

Specifically, according to some aspects of the present invention, applicants show that pluripotent stem-cells can be monitored for at least two datasets selected from (i) identification of epigenetic silencing of specific genes by promoter methylation of specific, e.g., oncogenes, tumor suppressor genes and development genes, (ii) identification of gene expression, e.g. developmental genes and lineage marker genes, and (iii) differentiation propensity to differentiate along different lineages to allow identification of characteristics of pluripotent stem cells and to predict which pluripotent stem cell lines are likely to contribute to a stem-cell originated cancer. For example, one can select out cells which have cancer-specific promoter DNA hypermethylation, in which reversible gene repression is replaced by permanent silencing, locking the cell into a perpetual state of self-renewal and thereby predisposing the cell to subsequent malignant transformation.

In one embodiment, the present invention relates generally to methods and a plurality of assays for predicting the functionality and suitability of a pluripotent stem cell line for a desired use. In some embodiments, at least one, or at least 2 or at least three of stem cell assays are used alone or in any combination, to predict the functionality and suitability of a pluripotent stem cell line for a desired use. In some embodiments, one assay is epigenetic profiling, e.g., assessment of gene methylation of specific defined gene set to determine genes activated in the pluripotent stem cell line. In some embodiments, a second assay is a differentiation assay to determine the propensity of the pluripotent stem cell line to differentiate along specific lineages. In some embodiments, the assay is a gene expression assay, e.g., a whole genome gene expression assay to determine the gene expression pattern of cell differentiation-related genes.

In some embodiments, the epigenetic profiling is performed first and the gene expression analysis for differentiation second. In some embodiments, the gene expression analysis for differentiation related genes is performed first and the epigenetic marker profiling second. In some embodiments, one performs the second screen only for the cells that were determined to be within normal parameters using the first screen to increase efficiency and reduce cost of performing the assays.

Another aspect relates to a set of reference data, herein referred to a “scorecard” which refers to the average data or otherwise aggregated data from results of a number of different pluripotent stem cell lines from the three combined assays of the present invention. The reference data which constitutes a “scorecard” can be used by one of ordinary skill in the art to compare, for example using a computer algorithm or software, a pluripotent stem cell line of interest to normal well functioning stem cell. The comparison with the reference “scorecard” can be used to effectively and accurately predict the utility of the pluripotent stem cell for a given application, as well as any specific characteristics of the pluripotent stem cell line of interest, e.g., a ES cell or iPS cell line. Accordingly, the methods, assays and scorecards as disclosed herein can be used for identify specific characteristics of stem cells to determine their suitability for downstream applications, such as, their suitability for therapeutic use, drug screening and toxicity assays, differentiation into a desired cell lineage, and the like.

Particular embodiments provide a method for identifying, screening, selecting or enriching for preferred pluripotent stem cells comprising: identifying in the pluripotent stem cell (i) the presence or absence of genes which have hypermethylated DNA promoters, or identifying genes which have a statistically significant difference (increase or decrease) in the methylation states of specific methylation target genes as compared to the normal variation, and identifying (ii) the level of gene expression of particular target genes, e.g., developmental genes and/or lineage marker genes, and (iii) the differentiation propensity to differentiate along different lineages to identify a pluripotent stem cell line with desirable characteristics.

Additional aspects of the present invention provide methods for validating and/or monitoring a stem cell, e.g., a pluripotent, multipotent, unipotent, or somatic stem cell, or terminally differentiated cell population, e.g., but not limited to precursor cells, embryonic stem (ES) cells, somatic stem cells, cancer stem cells, progenitor cells, induced pluripotent stem (iPS) cells, partially induced pluripotent (piPS) cells, reprogrammed cells, directly reprogrammed cells etc., comprising screening or monitoring at least one of the following; DNA methylation status of target methylation genes, expression level of target genes, and propensity to differentiate into ectoderm, mesoderm and endoderm to predict if the pluripotent stem cell line is likely to undergo a malignant transformation and has the ability to differentiate along a desired or particular developmental pathway and into a specific cell lineage.

One embodiment of the present invention provides a method for validating and selecting a pluripotent stem cell line or precursor cell population for a particular indication, comprising (i) measuring the differentiation potential of a pluripotent stem cell population using a quantitative differentiation assay as disclosed herein, and (ii) selecting a pluripotent stem cell population which has a medium or high efficiency of differentiation along a desired cell lineage or into a desired cell type, (iii) measuring the DNA methylation of a set of DNA methylation target genes in the pluripotent stem cell population and performing a comparison of the DNA methylation data with a reference DNA methylation level of the same target genes; and (iv) selecting a pluripotent stem cell line which does not differ by a statistically significant amount in the methylation of the target genes as compared to the reference DNA methylation level, and optionally performing steps (v) and (vi) where step (v) comprises measuring the expression level of target genes in the pluripotent stem cell line and performing a comparison of the gene expression level data with a reference gene expression level of the same target genes; and step (vi) comprises selecting a pluripotent stem cell line which does not differ by a statistically significant amount in the level of gene expression of the target genes as compared to the reference gene expression level. In some embodiments, a pluripotent stem cell is selected based on first, the differentiation along a desired cell lineage or into a desired cell types, secondly on either the DNA methylation or expression level of genes in the pluripotent stem cell, to negatively select (e.g., discard) pluripotent stem cells with undesirable characteristics, for example, pluripotent stem cells which have aberrant (increased or decreased) expression of oncogenes and/or tumor suppressor genes. By way of example only, one can discard cells with low methylation of oncogenes or high oncogene expression, and/or discard cells which have high methylation of tumor suppressor genes or high gene expression of tumor suppressor genes. In alternative embodiments, one can discard cells which have high methylation of developmental genes and/or lineage marker genes which are normally expressed in the desired cells which the pluripotent stem cells are to be differentiated into.

One aspect of the present invention relates to a scorecard of the performance parameters of a pluripotent stem cell, the scorecard comprising: (i) a first data set comprising the DNA methylation levels for a plurality of DNA methylation target genes from at least 5 pluripotent stem cell populations; (ii) a second data set comprising the gene expression levels for a plurality of target genes from at least 5 pluripotent stem cell populations; and (iii) a third data set comprising the differentiation propensity levels for differentiation into ectoderm, mesoderm and endoderm lineages from at least 5 pluripotent stem cell populations. In some embodiments, the plurality of reference DNA methylation genes is at least about 1000 reference DNA methylation genes, or at least about 2000 reference DNA methylation genes or in some embodiments, the DNA methylation status of the whole genome. In some embodiments, the reference DNA methylation genes are any selected from the group comprising cancer gene, oncogenes, and tumor suppressor genes, lineage marker genes and developmental genes.

In some embodiments, the DNA methylation target genes are any, and in any combination of genes selected from the group consisting of: BMP4, CAT, CD14, CXCL5, DAZL, DNMT3B, GATA6, GAPDH, LEFTY2, MEG3, PAX6, S100A6, SOX2, SNAI1, TF.

In some embodiments, the first and second data set of the scorecard are connected to a data storage device, such as a data storage device which is a database located on a computer device.

In some embodiments, at least 15 pluripotent stem cell lines are used to generate the first or second or third data set for the scorecard. In some embodiments, the first, second or third data set are obtained from at least 5 or more, or at least 6, or at least 7, or at least 8, or at least 9, or at least 10, or at least 11, or at least 12, or at least 13 or at least 14, or at least 15, or at least 16, or at least 17, or at least 18, or all 19 of the following pluripotent stem cells lines selected from the group; HUES64, HUES3, HUES8, HUES53, HUES28, HUES49, HUES9, HUES48, HUES45, HUES1, HUES44, HUES6, H1, HUES62, HUES65, H7, HUES13, HUES63, HUES66.

In some embodiments, the pluripotent stem cell populations used to generate the data sets for the scorecards are mammalian pluripotent stem cell populations, such as human pluripotent stem cell populations, or induced pluripotent stem (iPS) cell populations, or embryonic stem cell populations, or adult stem cell populations, or autologous stem cell populations, or embryonic stem (ES) stem cell populations.

In some embodiments, the scorecard as disclosed herein can be compared with the DNA methylation levels, gene expression levels and differentiation propensity levels of a pluripotent stem cell population of interest, and can be used to validate and/or predict the behavior of a pluripotent stem cell population by predicting the optimal differentiation along a specific lineage and/or propensity to have undesirable characteristic, e.g., pluripotent stem cell populations which have a predisposition to develop into cancer cells. Thus, in some embodiments, the scorecard can be used in methods to select for, e.g., positive selection pluripotent stem cell population of interest with desirable characteristics (e.g., high differentiation potential along a specific lineage), and/or to negatively select cells with undesirable characteristics, e.g., cells with a predisposition to develop into cancer cells.

Another aspect of the present invention relates to a method for generating a pluripotent stem cell score card comprising: (i) measuring DNA methylation in a set of target genes in a plurality of pluripotent stem populations; (ii) measuring gene expression in a second set of target genes in the plurality of pluripotent stem cell lines; and (iii) measuring differentiation potential of the plurality of pluripotent stem cell lines. In some embodiments, the method to generate a pluripotent stem cell score card can be used to generate a scorecard comprising the values of normal variations of DNA methylation, normal variation of DNA gene expression and normal differentiation propensity from a plurality of pluripotent stem cell lines, for example, at least 5, or at least 6, or at least 7, or at least 8, or at least 9, or at least 10, or at least 15, or at least 20, or a least 30, or at least 40 or more than 40 different pluripotent stem cell populations.

Another aspect of the present invention relates to a method for selecting a pluripotent stem cell population, comprising (i) measuring the DNA methylation of a set of DNA methylation target genes in the pluripotent stem cell population and performing a comparison of the DNA methylation data with a reference DNA methylation level of the same target genes; (ii) measuring the differentiation potential of the pluripotent stem cell population and comparing the differentiation potential data with a reference differentiation potential data; and (ii) selecting a pluripotent stem cell line which does not differ by a statistically significant amount in the methylation of the target genes as compared to the reference DNA methylation level, and does not differ by a statistically significant amount in the propensity to differentiate along mesoderm, ectoderm and endoderm lineages as compared to a reference differentiation potential.

In some embodiments, the method for selecting a pluripotent stem cell population further comprises: (i) measuring the gene expression level of a second set of target genes in the pluripotent stem cell line and performing a comparison of the gene expression level data with a reference gene expression level of the same target gene; and (ii) selecting a pluripotent stem cell line which does not differ by a statistically significant amount in the gene expression level of the target genes as compared to a reference gene expression level.

One aspect of the present invention relates to a computer system for generating a quality assurance scorecard of a pluripotent stem cell, comprising: (a) at least one memory containing at least one program comprising the steps of: (i) receiving DNA methylation data of a set of DNA methylation target genes in the pluripotent stem cell line and performing a comparison of the DNA methylation data with a reference DNA methylation level of the same target genes; (ii) receiving differentiation potential data of the pluripotent stem cell line and comparing the differentiation potential data with a reference differentiation potential data; (iii) generating a quality assurance scorecard based on the comparison of the DNA methylation data as compared to reference DNA methylation parameters and comparing the differentiation propensity as compared to reference differentiation data; and (b) a processor for running said program.

In some embodiments, the program of the system further comprises a step of: (i) receiving gene expression data of a second set of target genes in the pluripotent stem cell line and comparing the expression data with a reference gene expression level of the same second set of target genes; (ii) generating a quality assurance scorecard based on the comparison of the DNA methylation data as compared to reference DNA methylation parameters, and the comparison of the differentiation propensity as compared to reference differentiation data, and the comparison of the gene expression data as compared to reference gene expression levels.

In some embodiments of all aspects of the present invention, the DNA methylation target genes have variable methylation, and in some embodiments, the DNA methylation target genes are selected from any and all combinations of cancer genes, oncogenes, tumor suppressor genes, development genes, lineage marker genes. In some embodiments, the DNA methylation target genes are selected from the group consisting of: BMP4, CAT, CD14, CXCL5, DAZL, DNMT3B, GATA6, GAPDH, LEFTY2, MEG3, PAX6, S100A6, SOX2, SNAI1, TF.

In some embodiments of all aspects of the present invention, the reference DNA methylation level is the level of normal variation of the methylation of the DNA methylation target gene in a reference pluripotent stem cell population. In some embodiments, the reference DNA methylation level, (e.g., the level of normal variation of the methylation of the DNA methylation target gene), is generated from the variation of the level of methylation for the target DNA methylation gene from a plurality of different pluripotent stem cell populations, e.g., at least 2, or at least 3, or at least 4 or at least 5, or at least 6 or at least 10 or different pluripotent stem cell populations. In some embodiments, where the level of methylation of a DNA methylation target gene of a pluripotent stem cell of interest falls outside the reference DNA methylation level, such as is increased or decreased methylation level by a statically significant amount as compared to reference DNA methylation level, it can indicate an increase or decrease in a epigenetic silencing of the target DNA methylation gene, respectively.

In some embodiments, where the DNA methylation target gene is an oncogene, a decrease in the methylation by a statistically significant level as compared to the reference DNA methylation level for that oncogene can indicate a decrease in epigenetic silencing and lack of repression of the oncogene and can indicate the pluripotent stem cell has a predisposition for malignant transformation into a cancer cell. Alternatively, in some embodiments where the DNA methylation target gene is a tumor suppressor gene, an increase in the methylation by a statistically significant level as compared to the reference DNA methylation level for that tumor suppressor gene can indicate an increase in epigenetic silencing and repression of the tumor suppressor expression and can indicate the pluripotent stem cell has a predisposition for malignant transformation into a cancer cell.

In some embodiments, where the DNA methylation target gene is a developmental gene or a lineage marker gene, an increase in the methylation by a statistically significant level as compared to the reference DNA methylation level for that developmental gene or lineage marker gene can indicate an increase in epigenetic silencing and repression of the expression of the developmental gene or lineage marker gene, and can predict that the pluripotent stem cell will have a low efficiency for differentiating along the developmental pathway in which the developmental gene is normally expressed or will have low efficiency of differentiating into a cell type which expresses the lineage marker. Conversely, in embodiments where the DNA methylation target gene is a developmental gene or a lineage marker gene, a decrease in the methylation by a statistically significant level as compared to the reference DNA methylation level for that developmental gene or lineage marker gene can indicate a decrease in epigenetic silencing and a decrease in the repression of the expression of the developmental gene or lineage marker gene, and can be used to predict that the pluripotent stem cell of interest will have a high or optimal efficiency for differentiating along the developmental pathway in which the developmental gene is normally expressed and/or will have a high efficiency of differentiating into a cell type which expresses the lineage marker.

In some embodiments, the system further comprises a report generating module for generating a stem cell scorecard report based on quality of the pluripotent stem cell population. In some embodiments, the system comprises a memory, where the memory further comprises a database. In some embodiments, the database arranges the DNA methylation gene set in a hierarchical manner, for example, where the database arranges the propensity of differentiation of the pluripotent stem cell of interest into different lineages in a hierarchical manner. In some embodiments, the database can arrange the gene expression data in a hierarchical manner. In some embodiments, the memory of the system is connected to the first computer via a network, for example, a wide area network, or a world-wide network.

In some embodiments, the scorecard report provides an indication of suitable uses or applications of the pluripotent stem cell population, or in alternative embodiments, provide an indication of uses or applications that the pluripotent stem cell line is not suitable for.

In some embodiments, the reference DNA methylation level is range of normal variation of methylation for that DNA methylation target gene in a plurality of pluripotent stem cells. In some embodiments, the reference gene expression level is a range of normal variation of gene expression level for that target gene in a plurality of pluripotent stem cells. In some embodiments, the DNA methylation target genes are the same as gene expression target genes, and in some embodiments, the DNA methylation target genes include at least one or more of the gene expression target genes, and in some embodiments, the gene expression target genes include at least one or more of the DNA methylation target genes.

Another aspect of the present invention relates to a computer readable medium comprising instructions for generating quality assurance scorecard of a pluripotent stem cell line, comprising: (i) receiving DNA methylation data of a set of DNA methylation target genes in the pluripotent stem cell line and performing a comparison of the DNA methylation data with a reference DNA methylation level of the same target genes; (ii) receiving differentiation potential data of the pluripotent stem cell line and comparing the differentiation potential data with a reference differentiation potential data; (iii) generating a quality assurance scorecard based on the comparison of the DNA methylation data as compared to reference DNA methylation parameters and comparing the differentiation propensity as compared to reference differentiation data. In some embodiments, the computer-readable medium further comprises instructions for: (i) receiving gene expression data of a second set of target genes in the pluripotent stem cell line and comparing the expression data with a reference gene expression level of the same second set of target genes; (ii) generating a quality assurance scorecard based on the comparison of the DNA methylation data as compared to reference DNA methylation parameters, and the comparison of the differentiation propensity as compared to reference differentiation data, and the comparison of the gene expression data as compared to reference gene expression levels.

Another aspect of the present invention relates to an assay for characterizing a plurality of properties of a pluripotent cell, the assay comprising at least 2 of the following: (i) a DNA methylation assay; (ii) a gene expression assay; and (iii) a differentiation assay. In some embodiments, the DNA methylation assay is a bisulfite sequencing assay, or a whole genome sequencing assay, e.g., a reduced-representation bisulfite sequencing (RRBS). In some embodiments, the gene expression assay is a microarray assay.

In some embodiments, the differentiation assay a quantitative differentiation assay, e.g., a differentiation assay which can assess the ability of the pluripotent cell to differentiate into at least one of the following lineages; mesoderm, endoderm and ectoderm, neuronal hematopoietic lineages. In some embodiments, the ability of the pluripotent cell to differentiate into at least one of the following lineages; mesoderm, endoderm and ectoderm is determined by immunostaining or FAC sorting using an antibody to at least one marker for mesoderm, endoderm and ectoderm lineages. In some embodiments, the ability of the pluripotent cell to differentiate into at least one of the following lineages; mesoderm, endoderm and ectoderm is determined by immunostaining the pluripotent stem cell after at least about 0 days in EB. In some embodiments, the ability of the pluripotent cell to differentiate into at least one of the following lineages; mesoderm, endoderm and ectoderm is determined at anywhere between 0 days in EB, or between 0-32 days in EB, e.g., at least 1 day, or at least 2 days, or at least about 3 days, or at least about 4 days, or at least about 5 days, or at least about 6 days, or at least about 7 days, or more than about 7 days in EB, e.g., between 5-7 days in EB, or between about 7-10 days in EB, or between about 10-14 days in EB, or between about 14-21 days in EB, or between about 21-32 days in EB or longer than 32 days in EB. In some embodiments, a pluripotent stem cell ability to differentiate is determined between 5-10 days EB, for example at about 7 days in EB. Examples of lineage markers for mesoderm, endoderm and ectoderm lineages are well know by persons of ordinary skill in the art, and include but are not limited to mesoderm lineage markers VEGF receptor II (KDR) or actin α-2 smooth muscle (ACTA2), ectoderm lineage markers Nestin or Tubulin β3 and endoderm lineage markers alpha-feto protein (AFP). In some embodiments, one of ordinary skill in the art can use chemical or other stimuli, e.g., growth factors etc., to increase time-to-result in terms of differentiation and to reduce signal to noise ratio and variability in determining the propensity of the pluripotent stem cell to differentiate along mesoderm, endoderm and ectoderm lineages.

In some embodiments, the assay is a high-throughput assay for assaying a plurality of different pluripotent stem cells, for example, enabling one to assess a plurality of different induced pluripotent stem cells derived from reprogramming a somatic cell obtained from the same or a different subject, e.g., a mammalian subject or a human subject.

In some embodiments, the assay as disclosed herein can be used to generate a scorecard as disclosed herein from at least one, or a plurality of pluripotent stem cell populations.

In some embodiments of all aspects as disclosed herein, the reference DNA methylation level is range of normal variation of methylation for that DNA methylation target gene in a pluripotent stem cell population.

In some embodiments of all aspects as disclosed herein, the reference gene expression level is range of normal variation of gene expression level for that target gene, in a pluripotent stem cell population.

Another aspect of the present invention relates to a kit for determining the quality of a pluripotent stem cell line, comprising: (i) reagents for measuring methylation status of a plurality of DNA methylation genes, (ii) reagents for measuring gene expression levels of a plurality of genes; and (iii) reagents for measuring the differentiation propensity of the pluripotent stem cell into ectoderm, mesoderm and endoderm lineages. In some embodiments, the kit further comprises a score card as disclosed herein. In some embodiments, the kit further comprises instructions for use.

The inventors herein have provided a clear path that investigators can navigate to proceed from patient samples, to fully reprogrammed iPS cells, to a selected and manageable set of pluripotent iPS cell lines that can be used at a reasonable scale for disease modeling. In particular, in order to firmly establish the nature and magnitude of variation that exists among pluripotent stem cell lines, three genome-scale assays were applied to 19 ES cell lines, 12 iPS cell lines and 6 primary fibroblast cell lines. These assays included DNA methylation mapping by genome-scale bisulfite sequencing (Gu et al., 2010; Meissner et al., 2008), gene expression profiling using high-throughput microarrays, and a quantitative differentiation assay that utilizes transcript counting of 500 genes in embryoid bodies.

In aggregate, the inventors have used the systems and methods as disclosed herein, to generate data from at least two of the three assays to provide at least one scorecard which comprises a reference level of normal variation of the level of DNA methylation and level of gene expression in human pluripotent cell lines. For most genes, the inventors observed little variation in terms of DNA methylation and transcription levels. However, the inventors discovered that there was a notable class of genes that exhibited either highly variable DNA methylation or transcription between the individual pluripotent cell lines. Surprisingly, the inventors demonstrate that an understanding of this variation is significant and enables one to predict the behavior of a given pluripotent stem cell line. In addition, using a quantitative differentiation assay, the inventors demonstrated that the prediction of optimal differentiation of the pluripotent stem cell into a specific lineage was correct, and also demonstrated that each pluripotent cell line had it's own specific and reproducible propensity for differentiation down a given developmental lineage. Importantly, the inventors also demonstrate that knowledge of the differentiation propensities can be used to accurately predict the efficiency at which each cell line performed in directed differentiation experiments carried out independently by Boulting and colleagues. In summary, the inventors have combined the results of these three assays (DNA methylation, gene expression profiling and quantitative differentiation assays) to produce a “lineage scorecard” that can be used by anyone to predict the utility of a particular ES cell or iPS cell line for a given application.

A “summary score card” as disclosed herein comprises a “deviation scorecard” which provides a reference of normal variation in human pluripotent cell lines and a “lineage scorecard”. In a deviation scorecardm for most of the genes analyzed, the inventors observed little variation in terms of DNA methylation and transcription levels. However, the inventors discovered that a notable subset or class of genes that exhibited either highly variable DNA methylation or transcription between the individual cell lines. Here, the inventors demonstrate that understanding this variation is significant as it can be used for predictions of the behavior of a given pluripotent stem cell-line.

For example, aspects of the present invention relate to methods and the production of two scorecards for characterizing pluripotent stem cell lines, a first scorecard which can be referred to a “deviation scorecard” or “pluripotency scorecard” is useful to provide information of how the pluripotent stem cell line of interest compares to previously established or control pluripotent stem cell lines, and can be used to identify the number or % of genes which deviate in terms of DNA methylation or gene expression as compared to a reference pluripotent stem cell line and/or a plurality of reference pluripotent stem cell lines. Such a scorecard is useful for identifying the pluripotency of the stem cell line of interest as well as to identify if the stem cell line of interest has atypical gene expression or DNA methylation of cancer genes which may predispose the stem cell line of interest to abberant proliferation and formation of cancer at a later time point. A second score card, herein referred to as a “lineage scorecard” is useful as a quantification of the differentiation potential of the pluripotent stem cell of interest, and provides information of how efficienty the pluripotent stem cell line of interest will differentiation into particular lineages of interest as compared to previously established or control pluripotent stem cell lines.

In summary, the three assays as described herein, used alone or in any combination, including the combined results of all three assays, can be used to generate a “summary scorecard” (e.g., comprising a deviation scorecard and/or a lineage scorecard) that can be used by one of ordinary skill in the art to validate a pluripotent stem cells, and predict the utility of a particular pluripotent stem cell, e.g., a ES cell or iPS cell line for a given application.

The assays as disclosed herein can be configured to be high-throughput, for example using multiplex qPCR and high-throughput sample processing to produce deviation scorecards and lineage scorecards which would enable the characterization of hundreds or thousands of ES and/or iPS cell lines at one time, for example where it is desirable to characterize 100's and 1000's stem cell lines in high-throughput centres, for example to determine stem cell lines for utility in drug screening for therapeutic use. Use of the methods and scorecards as disclosed herein allow rapid and inexpensive characterization of large numbers of stem cell lines which would be highly expensive and impractical using traditional teratoma methods of characterization. Alternatively, the assays, methods, systems and scorecards as disclosed herein can be used in an individual manner to accelerate research and be used in research to address a research question of interest, for example, the assays, methods, systems and scorecards as disclosed herein can be used to characterize a pluripotent stem cell line to identify the most suitable pluripotent stem cell line for further analysis to address the research question of interest.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1C show reference maps of human ES cell lines span a corridor of normal variation among pluripotent cell lines. FIG. 1A shows joint hierarchical clustering of 19 human ES cell lines and six primary fibroblast cell lines. DNA methylation levels were averaged across promoter regions ranging from −5 kb to +1 kb around each Ensembl-annotated transcription start site. Gene expression levels were calculated for each Ensembl gene by averaging over all associated probes on the microarray. Prior to hierarchical clustering the two datasets were separately normalized to zero mean and unit variance, Euclidean distance matrices were calculated for both DNA methylation and gene expression, and the two distance matrices were averaged. Hierarchical clustering was performed using average linkage, and the heatmaps show a representative selection of 250 genes. Lighter colors indicate higher levels of DNA methylation (red) or gene expression (green), darker colors indicate lower levels. The combined DNA methylation and gene expression data are shown in Table 3. The lists of all genes and promoter regions ordered by their levels of epigenetic and transcriptional variation are shown in Tables 4 and 5.

FIG. 1B shows a high-resolution view of the DNA methylation and gene expression measurements at four selected genes. DNA methylation patterns are shown for promoter regions ranging from −5 kb to +1 kb around Ensembl-annotated transcription start sites. Each box on the left represents a single CpG dinucleotide located within the promoter region (dark red: high methylation, light red: partial methylation, white: full methylation). The single boxes on the right visualize the normalized expression levels of each gene (dark green: high expression, light red: moderate expression, white: no expression). Measurements are shown for four representative ES cell lines and one representative fibroblast cell line. Note that the DNA methylation patterns are not drawn to scale. All high-resolution data are available as genome browser tracks via the Supplementary Website at http://scorecard.computational-epigenetics.org/.

FIG. 1C shows Boxplots of gene-specific DNA methylation (left) and gene expression (right) among 19 low-passage human ES cell lines, illustrating the concept of an epigenetic and transcriptional reference corridor. The combined data of many ES cell lines quantifies observed variation among human pluripotent cell lines and provides a reference against which single cell lines can be compared. The corridor spans a total of 31,929 promoter regions (DNA methylation) and 15,079 genes (expression); this diagram focuses on 15 selected genes that cover a wide range of different variation levels. Boxplot boxes correspond to center quartiles with the median marked by a black bar, and whiskers extend to the most extreme data point which is no more than 1.5 times the interquartile range from the box. The full ES-cell reference corridor is available from the Website http://scorecard.computational-epigenetics.org/(data not shown), which is incorporated herein in its entirety reference.

FIGS. 2A-2G show epigenetic and transcriptional variation targets specific genes and influences cellular differentiation. FIG. 2A shows the distribution of cell-line specific deviation from the ES-cell reference averaged across 19 ES cell lines, providing a gene-specific measure of susceptibility toward epigenetic and transcriptional variation. The histogram shows the number of genes (y-axis) that fall into each interval of average deviation levels (x-axis). The position of selected genes within each histogram is highlighted on top. Note that the DNA methylation histogram (left) is extremely skewed; for better representation the x-axis has been compressed five-fold for the right half of the diagram, which gives rise to a spurious peak in the center of the histogram. In the gene expression histogram (right) there is a strong peak at zero, which is due to a large number of genes exhibiting zero expression (and thus zero variation) in all ES cell lines.

FIG. 2B shows Chromosomal distribution of the 1,000 most variable genes in terms of DNA methylation (top left) or gene expression (bottom left), indicating that epigenetically but not transcriptionally variable genes are predominantly located on the human sex chromosomes X and Y. Variability was measured as the cell-line specific deviation from the ES-cell reference averaged across 19 ES cell lines. The diagram also shows the chromosomal distribution of all genes with sufficient DNA methylation (top right) or gene expression data (bottom right), underlining that the differences in genomic location of the most variable genes are not a side-effect of biased sequencing coverage.

FIG. 2C shows a comparison of the 1,000 most variable genes in terms of DNA methylation (top) and gene expression (bottom). To prevent the sex-chromosome bias from influencing this analysis, all X-linked and Y-linked genes were excluded. Significance of overlap was established using Fisher's exact test.

FIG. 2D shows the structural and functional characteristics of the 1,000 most variable genes (and gene promoters) in terms of DNA methylation (top) and gene expression (bottom). Functional annotation clustering was analyzed with the DAVID software (Huang et al., 2007), and the promoter characteristics were analyzed with the EpiGRAPH web service (Bock et al., 2009). This panel provides a summary of the results; the full results are shown in tables 3 and 5. To prevent the sex-chromosome bias from influencing this analysis, all X-linked and Y-linked genes were excluded.

FIG. 2E shows the scatterplots of DNA methylation (left, center) and gene expression (right) differences between two ES cell lines during undirected EB differentiation, indicating that DNA methylation differences of the ES-cell state (left) are maintained in 16-day EBs (center) and are negatively correlated with gene expression in the EBs (right). Those genes that were differentially methylated (threshold: 20 percentage points) between the two ES cell lines in the pluripotent state (left) are highlighted in all three diagrams (orange: hypermethylated in HUES6, blue: hypermethylated in HUES8). The location of the macrophage/granulocyte-specific marker gene CD14 is indicated by arrows, providing an example of a gene that maintains its cell-line specific differential methylation in 16-day EBs and that is upregulated only in the absence of DNA methylation at its promoter.

FIG. 2F shows the epigenetic and transcriptional differences between two ES cell lines (HUES6 and HUES8) subjected to a defined hematopoietic differentiation protocol. DNA methylation levels were measured by clonal bisulfite sequencing at day 0 and day 18 of the differentiation protocol. White beads correspond to unmethylated CpGs, and black beads correspond to methylated CpGs. Rows correspond to individual clones, and columns correspond to specific CpGs in the promoter region of CD14. Similarly, gene expression of CD14 and two additional macrophage marker genes (CD33 and CD64) was measured by qPCR in two independent experiments (shown are three technical replicates) at day 0 and day 18 of the differentiation protocol.

FIG. 2G shows cell-line specific DNA methylation and gene expression levels at four genes with a known role in hematopoiesis (TFCP2, LY6H) and neural processes (COMT, CAT). Each data point denotes the combined DNA methylation (x-axis) and gene expression (y-axis) levels of an ES cell lines (“ES”) or the corresponding 16-day embryoid body (“EB”).

FIGS. 3A-3D show genomic maps detect a trend toward higher variability in iPS cell lines but no iPS-specific defect.

FIG. 3A shows joint hierarchical clustering of 11 iPS cell lines (“hiPSx”), 19 ES cell lines (“HUESx” or “Hx”) and six primary fibroblast cell lines (“hFibx”), indicating that all iPS cell lines cluster with the ES cell lines and that there is not clear separation into subclusters among the pluripotent cell lines. Clustering was performed in the same way as in FIG. 1A. An extended version with heatmaps and MEG3 expression status is available from FIG. 9B.

FIG. 3B shows Scatterplots comparing the cell-line specific deviation of 19 ES cell lines (x-axis) with the cell-line specific deviation of 11 iPS cell lines (y-axis), in both cases measured relative to the ES-cell reference and averaged over the relevant cell lines. To prevent comparing cell lines to themselves, each ES cell line was temporarily removed from the ES-cell reference when it was scored against the reference. Selected genes are highlighted in orange, and the inset Venn diagrams visualize the overlap between the 2,000 most deviating genes averaged across all ES cell lines and across all iPS cell lines. The reprogramming factors OCT4, SOX2 and KLF4 were excluded from the analysis because transgene silencing gives rise to spurious hypermethylation among the iPS cell lines (FIG. 9C). The lists of all genes and promoter regions with their average cell-line specific deviations among ES and iPS cell lines are shown in Tables 4 and 5.

FIG. 3C shows boxplots of the cell-line specific deviation of 19 ES cell lines, 11 iPS cell lines and six primary fibroblast cell lines, measured relative to the ES-cell reference and averaged over all genes. The distribution of cell-line specific deviation among the 19 ES cell lines was normalized to zero mean and unit variance, and the two other distributions were rescaled accordingly. (This normalization does not affect the comparison between the three distributions because the same scaling parameters were used.)

FIG. 3D shows a performance table summarizing the predictive power of three previously published iPS cell signatures and three newly derived classifiers for distinguishing between ES and iPS cell lines. For comparison, the table also lists the performance of three newly derived classifiers for distinguishing between ES cell lines and fibroblasts (positive controls) and the performance of three trivial classifiers (negative controls). Shown are the prediction accuracy, sensitivity and specificity for identifying iPS cell lines (true positives, TP) among ES cell lines (true negatives, TN), while minimizing the number of cell lines that are incorrectly predicted as iPS cell lines (false positives, FP) or incorrectly predicted as ES cell lines (false negatives, FN). To increase the robustness of the results, all values were averaged over 100 randomized repetitions of the cross-validation. Minor numerical inconsistencies in the table are due to rounding all values to whole numbers. The performance estimates of the cross-validated classifiers and the published signatures should be considered test-set accuracies, which are likely to be reproducible on new data of the same type (same culture conditions, same assay, etc.).

FIGS. 4A-4B show a statistical comparison with the ES-cell reference identifies ES/iPS cell-line specific deviations.

FIG. 4A shows the distribution of DNA methylation (left) and gene expression (right) among 19 ES cell lines and 11 iPS cell lines relative to the ES-cell reference corridor, which is indicated by boxplots (see FIG. 1C for details). ES or iPS cell lines that deviate from the ES-cell reference by more than 20 percentage points and an FDR below 0.1% (DNA methylation) or by an absolute log fold-change above one and an FDR below 10% (gene expression) are highlighted by colored triangles. To prevent comparing cell lines to themselves, each ES cell line was temporarily removed from the ES-cell reference when it was scored against the reference. Full lists of differentially methylated and expressed genes are available from the Website “http://scorecard.computational-epigenetics.org/” and are available in Tables 4 and 5, as disclosed herein

FIG. 4B shows a deviation scorecard summarizing the cell-line specific number of outliers relative to the ES-cell reference, in terms of DNA methylation (left) and gene expression (right). As an additional indication of a cell line's quality, the scorecard lists the number of affected lineage marker genes, which have the potential to undermine a cell line's propensity for differentiation along certain trajectories as shown for CD14 in FIG. 2D. The table also shows the mean number of deviating genes in the 20 low-passage ES cell lines (bottom row), providing an indication of what numbers are within a range that is also observed among low-passage ES cell lines. A more comprehensive version of this scorecard that includes data for all ES cell lines and lists all affected genes is shown in Table 6. Differences with an FDR below 10% were considered significant, but only if the absolute difference exceeded 20 percentage points (DNA methylation) or the absolute log fold-change exceeded one (gene expression). When using the scorecard for cell line selection these data should be carefully reviewed for evidence of gene-specific deviations that may interfere with the application of interest.

FIGS. 5A-5D show cell-line specific differentiation propensities can be measured by a quantitative EB assay.

FIG. 5A shows a schematic outline of an assay for quantifying cell-line specific differentiation propensities. The main result of this as—say is a lineage scorecard as shown in FIGS. 5B and 5D.

FIG. 5B shows a lineage scorecard summarizing cell-line specific differentiation propensities of a set of low-passage human ES cell lines. The numbers indicate relative enrichment (positive values) or depletion (negative values) on a linear scale. They were calculated by performing moderated t-tests comparing all biological replicates for a given ES cell line to the ES-cell reference (consisting of biological replicates for all other ES cell lines), followed by a gene set enrichment analysis for sets of markers genes with relevance for the cellular lineage or germ layer of interest (Table 7). All columns are centered on zero, such that an ES cell line will exhibit differentiation propensities of zero if it differentiates just like the average of all other ES cell lines that were used to calibrate the assay. Values should be interpreted relative to each other, with higher numbers indicating higher differentiation propensities and lower values indicating lower differentiation propensities, while the absolute values have no measurement unit and no direct biological interpretation. Pictures of representative EBs are shown in FIG. 10A; immunostaining validating a subset of the predictions are shown in FIG. 10B; the list of all marker genes is available from Table 7; the gene expression data from which the scorecard was constructed are available from Table 10; and a documentation of the link between single-gene expression levels and lineage scorecard differentiation propensities is shown in Table 8.

FIG. 5C shows a two-dimensional multidimensional scaling map of the transcriptional similarity of ES and iPS cell lines, ES-derived and iPS-derived EBs, and primary fibroblast cell lines. Gene expression of 500 lineage marker genes was measured using the nCounter system, and the normalized data were projected onto a plane such that the distance of the points to each other represents their distance in the 500-dimensional space of gene expression levels. Each point corresponds to a single biological replicate, and the projection was performed using multidimensional scaling. Two iPS cell lines were significantly impaired in their ability to form normal EBs (hiPS 15b, hiPS 29e, highlighted by an arrow and labeled as “impaired EBs”), and one iPS cell line completely failed to from normal EBs (hiPS 27e, highlighted by an arrow and labeled “failed EBs”), maintaining a gene expression profile that is reminiscent of pluripotent cells even after 16-day EB differentiation. All biological replicates of these three cell lines are highlighted by arrows, and all three cell lines also exhibit significantly reduced differentiation propensities according to the lineage scorecard (FIG. 5D).

FIG. 5D shows a Lineage scorecard summarizing cell-line specific differentiation propensities of a set of human iPS cell lines. The scorecard was derived as described for FIG. 5B and normalized against the ES-cell reference. The scores were calculated across all biological replicates that were available fore each cell line. Pictures of representative EBs are shown in FIG. 10C. A FACS analysis validating specific aspects of the lineage scorecard is shown in FIG. 10D.

FIGS. 6A-6C shows the lineage scorecard predicts cell-line specific differences of motor neuron differentiation.

FIG. 6A shows an outline of a procedure for measuring cell-line specific differences in the efficiency of making motor neurons in vitro. 13 iPS cell lines (see Table 1) were subjected to a 32-day neural differentiation protocol, and the differentiation efficiencies were quantified by automated counting of cells that stain positive for the motor neuron markers ISL1 and HB9 (Boulting et al., co-submitted). All experiments were performed at least in biological triplicate.

FIG. 6B shows the correlation between the lineage scorecard estimate for neural lineage differentiation and the cell-line specific efficiency of making motor neurons in vitro (r_{p, Pearson's correlation coefficient; r}ₛ, Spearman's correlation coefficient). Motor neuron efficiencies were measured by the percentage of ISL1-positive (left) and HB9-positive cells (right) at the end point of a 32-day neural differentiation protocol. Further details including biological replicates and standard errors are shown in Table 9.

FIG. 6C shows the correlation between the lineage scorecard estimates for the three germ layers and the cell-line specific efficiency of making motor neurons in vitro (r_{p, Pearson's correlation coefficient; r}ₛ, Spearman's correlation coefficient). Motor neuron efficiencies were measured by the percentage of ISL1-positive cells at the end point of a 32-day neural differentiation protocol. A similar comparison with the percentage of HB9-positive cells is shown in FIG. 11A. Further details including biological replicates and standard errors are shown in Table 9.

FIGS. 7A-7E shows that small modifications of the scorecard enable high-throughput characterization of human iPS cell lines.

FIG. 7A shows a summary of one embodiment of the scorecard for quantifying ES/iPS cell line quality and utility along multiple dimensions. This table combines data from FIG. 4B and FIG. 5D, providing an overview of (i) gene-specific DNA methylation deviations from the ES-cell reference, (ii) up- or downregulated genes relative to the ES-cell reference, and (iii) quantitative differentiation propensities for the three germ layers.

FIG. 7B shows the pairwise correlations between the different dimensions of the scorecard, indicating that the number of genes exhibiting epigenetic and transcriptional deviation as well as the estimates of differentiation propensity provide complementary—rather than redundant—information about ES/iPS cell line quality and utility.

FIG. 7C shows the simulation of the scorecard performance with reduced genomic coverage of the DNA methylation assay. Based on the data of all 19 ES cell lines (or random subsets of size 10, 5 and 1), all genes were ranked according to the average deviation from the ES-cell reference. Next, the top-1%, 5%, 10%, up to 90% most ES-cell variable genes were selected and evaluated for the percentage of iPS cell-line specific deviations that would have been detected if only these genes were monitored for deviations. These data indicate that it is possible to detect 90% of iPS cell-line specific deviations by focusing on the 20% most susceptible promoter regions. FIG. 12 shows that a similar focus on the most transcriptionally variable genes leads to a much stronger reduction in the ability to detect cell-line specific deviations in gene expression than it does for DNA methylation.

FIG. 7D shows the simulation of the scorecard performance without EB differentiation. Gene expression profiles were obtained for ES and iPS cell lines using the nCounter system and processed in the same way as the gene expression pro files from the 16-day EBs, giving rise to a lineage scorecard that is exclusively based on gene expression profiles of ES/iPS cell lines maintained under normal growth conditions. The scatterplots visualize the correlation between lineage scorecard estimates calculated from 16-day EBs (x-axis) and lineage scorecard estimates calculated from the pluripotent state (y-axis), indicating good agreement between the two but a substantially reduced dynamic range in the latter.

FIG. 7E shows a schematic of an outline of a workflow for high-throughput characterization of human pluripotent cell lines. Cell line characterization is performed in an iterative fashion, starting with the—arguably most informative—quantitative differentiation assay and performing additional characterizations only on those cell lines that the lineage scorecard identifies as useful for the application of interest. Note that not every cell line is equally suited for all applications. The data from the current study clearly indicate the ES-grade iPS cell lines exist.

FIG. 8A-8D. FIG. 8A shows representative images and immunostaining of ES cell lines included in the current study.

FIG. 8B shows the genomic coverage of DNA methylation data obtained by RRBS (summary). Pie charts illustrating the RRBS coverage at gene promoters, CpG islands and putative enhancers. Coverage is measured as the number of individual observations (i.e. high-quality sequencing reads) at CpGs within each region of a given type. Data are shown for a representative human ES cell line (H1).

FIG. 8C shows the genomic coverage of DNA methylation data obtained by RRBS (specific locus). UCSC Genome Browser screenshot illustrating RRBS coverage at the SNAI1 gene locus. The promoter region of SNAI1 (violet) exhibits the highest density of CpGs (black) and also the highest RRBS coverage (blue). Additional RRBS coverage is centered on a downstream CpG island (green) and an upstream regulatory element (orange). Most CpG-rich regions are unmethylated (light blue), while CpG-poor regions tend to be methylated (dark blue). Each blue dot corresponds to a single CpG that is covered by RRBS. Some epigenetic variation can be seen between H1 and H7, but overall the promoter region is unmethylated in all shown ES cell lines.

FIG. 8D shows a global comparison of promoter DNA methylation across 19 different ES cell lines. Pairwise scatterplots comparing mean promoter DNA methylation levels across 19 ES cell lines. High similarity was observed for all pairwise comparisons. However, there were two types of differences between pairs of ES cell lines that are visible from this diagram: (i) Small but dense point clouds located in the bottom left close to the X or Y axis: These are X-chromosome associated differences which distinguish female ES cell lines with widespread X-inactivation from male ES cell lines. (ii) Off-diagonal points scattered throughout the diagram: Most of these differences are located on the autosomes and constitute epigenetic differences between the ES cell lines.

FIG. 9A-9D. FIG. 9A shows a global comparison of promoter DNA methylation in 11 iPS cell lines and 6 primary fibroblast cell lines. Pairwise scatterplots comparing mean promoter DNA methylation levels across 11 iPS cell lines and 6 primary fibroblast cell lines. High similarity was observed among the iPS cell lines, while substantial differences distinguish the iPS cell lines from the fibroblast cell lines.

FIG. 9B shows an example of results from analysis of the joint clustering of DNA methylation and gene expression data. Joint hierarchical clustering and heatmaps of human ES cell lines, iPS cell lines and fibroblasts. The clustering was performed as described in the legend of FIG. 1. In the “MEG3” column the expression status of the MEG3 non-coding RNA is indicated: “+” stands for MEG3 being expressed in the respective cell line (MEG3 expression level ≧1) and “-” indicates that MEG3 is not expressed (MEG3 expression level <1).

FIG. 9C shows that spurious hypermethylation in the coding region of KLF4 due to transgene silencing. UCSC Genome Browser screenshot illustrating how transgene silencing gives rise to spurious hypermethylation at the endogenous loci of the reprogramming factors. Due to the way in which RRBS reads are aligned to the genome, most viral transgene reads are placed in the endogenous loci of OCT4, SOX2 and KLF4. This phenomenon is illustrated for KLF4: In ES cells the KLF4 gene is largely unmethylated (green), while it appears partially methylated in iPS cells, but only at those exons that are part of the transgene (red), never at introns that are not part of the transgene (blue). Furthermore, incomplete transgene silencing in hiPS 27e (yellow) is correlated with substantially lower DNA methylation levels in transgenic KLF4.

FIG. 9D shows that MEG3 expression is not a strong predictor of epigenetic or transcriptional deviation from the ES-cell reference. Boxplots of the cell-line specific deviation from the ES-cell reference averaged across all genes, for the following cell lines: (i) those ES cell lines in which the MEG3 non-coding RNA was expressed (see FIG. 9B), (ii) those cell lines in which MEG3 was not expressed (HUES1, HUES3, HUES13, HUES44, HUES45, HUES53, HUES66, H1 and H7) and (iii) six primary fibroblast cell lines.

FIG. 10A-10D shows the scorecard enables quick and comprehensive characterization of human pluripotent cell lines.

FIG. 10A shows pairwise correlation coefficients and scatterplots comparing DNA methylation between biological replicates of three ES cell lines (HUES1, passage 28 and 29; HUES8, passage 29 and 30; H1, passage 37 and 38). In addition, the DNA methylation comparison includes two biological replicates of H1 that were grown at the University of Wisconsin (passage 25) and at Cellular Dynamics (passage 32), respectively. High similarity was observed for all pairwise comparisons. However, two types of differences between pairs of ES cell lines are visible from these diagrams: (i) Small but dense point clouds located in the bottom left close to the x-axis or y-axis (DNA methylation only). These points correspond to X-chromosome associated differences which distinguish female ES cell lines with widespread X-inactivation from male ES cell lines. (ii) Off diagonal points scattered throughout the diagram. Most of these differences are located on the autosomes and constitute epigenetic or transcriptional differences between the ES cell lines.

FIG. 10B shows pairwise correlation coefficients and scatterplots comparing gene expression between biological replicates of three ES cell lines (HUES1, passage 28 and 29; HUES8, passage 29 and 30; H1, passage 37 and 38).

FIG. 10C shows an illustration of the minimum threshold for DNA methylation differences in heterogeneous cell populations. Even small DNA methylation differences between cell lines can be highly statistically significant if the variation is low. However, this does not always imply biological significance. Therefore, and in addition to a statistical significance threshold of 10% false-discovery rate (FDR), the DNA methylation difference between two cell lines (or between one cell line and the ES-cell reference) is required to exceed 20 percentage points to be considered relevant. Taking into account that most cell lines exhibit some degree of heterogeneity, there are several ways in which a cell line can deviate by more than 20 percentage points from the ES-cell reference: (i) all cells exhibit DNA methylation levels that are increased (decreased) by 20 percentage points; (ii) a subset of 20% of all cells exhibit DNA methylation levels that are increased (decreased) by 100 percentage points, while the remaining 80% do not show any difference; (iii) any combination as shown in the figure.

FIG. 10D shows a schematic illustration of the similarity between ES and iPS cell lines in the epigenetic and transcriptional space. The density plot on the left depicts the variation observed among human ES cells. The two crosses indicate the (hypothetical) average of all ES and iPS cell lines, which this study approximated by profiling 20 human ES cell lines and 12 human iPS cell lines. The scatterplot on the right simulates the distribution of a large number of human iPS cell lines, taking into account their moderately increased variation (FIG. 3C) as well as the observation that a minority of iPS cell lines were indistinguishable from ES cell lines (FIG. 3D). Gaussians were used to simulate the ES-cell and iPS-cell distribution in silico.

FIGS. 11A-11B show outlines of the algorithms for calculating derivation scorecard based on genome-wide DNA methylation and/or gene expression data, and the lineage scorecard based on marker gene expression in differentiating EBs. FIG. 11A shows the outline of the algorithm for calculating the deviation scorecard based on genome-wide DNA methylation and/or gene expression data. FIG. 11B shows the outline of the algorithm for calculating the lineage scorecard based on marker gene expression in differentiating EBs.

FIGS. 12A-12E. FIG. 12A shows examples of representative images of ES-cell derived EBs. Images of 16-day embryoid bodies derived from low-passage human ES cell lines, which were used to establish the reference dataset of the lineage scorecard.

FIG. 12B shows images of immunostaining for selected lineage marker genes. Validation of selected lineage scorecard estimates by immunostaining, indicating good qualitative agreement between the lineage scorecard's differentiation propensities, mRNA levels, and protein staining for five marker genes. Undirected EB differentiation was performed on four representative ES cell lines. After two days, the EBs were plated onto matrigel and allowed to differentiate for another five days. After seven days of EB differentiation, immunostaining were performed for marker genes of the three germ layers. The figure shows representative pictures of the undifferentiated ES cells, the EBs at day 7 and the immunostaining The gene expression levels were obtained for 16-day EBs using the nCounter system (Table 10).

FIG. 12C shows images of iPS cell lines and derived EBs. Images of iPS cell lines and derived EBs for the lineage scorecard.

FIG. 12D shows FACS analysis for the endoderm marker gene AFP. Comparison between the number of AFP-positive cells determined by FACS and the mRNA expression levels in 16-day EBs for hiPS 17 and hiPS 27e.

FIG. 12E shows the mean lineage scorecard values for four ES cell lines (HUES1, HUES8, H1, H9) that were differentiated under conditions that favored ectoderm differentiation (blue) and mesoderm differentiation (red).

FIGS. 13A-13C show the correlation between motor neuron efficiency (HB9+ cells) and lineage scorecard propensities for the germ layers.

FIG. 13A shows a scatterplot showing the correlation between lineage scorecard estimates of cell-line specific differentiation propensities into ectoderm differentiation and the efficiency of directed differentiation into motor neurons.

FIG. 13B shows a scatterplot showing the correlation between lineage scorecard estimates of cell-line specific differentiation propensities into mesoderm differentiation and the efficiency of directed differentiation into motor neurons.

FIG. 13C shows a scatterplot showing the correlation between lineage scorecard estimates of cell-line specific differentiation propensities into endoderm differentiation and the efficiency of directed differentiation into motor neurons. For each cell line the motor neuron efficiency was measured by automatic counting of the percentage of HB9-positive cells at the end point of a 32-day motor neuron differentiation protocol. HB9 is a highly specific marker of motor neuron that is not expressed in most other neural cell types.

FIG. 14A shows the scorecard (like FIG. 7C) performance with reduced coverage (gene expression) of the most transcriptionally variable genes leads to a much stronger reduction in the ability to detect cell-line specific deviations in gene expression than it does for DNA methylation. Saturation chart showing the number of iPS cell-line specific deviations relative to the ES-cell reference that would have been detected when focused only on the top-X percent genes that exhibit the highest mean absolute deviation from the ES-cell reference among the ES cell lines.

FIG. 14B shows a saturation plot estimating the scorecard performance for DNA methylation assays with reduced genomic coverage. FIG. 14C shows a saturation plot estimating the scorecard performance for gene expression assays with reduced genomic coverage. FIGS. 14B and 14C saturation plots are based on the data of all 20 ES cell lines (or random subsets of size 10, 5 and 1), all genes were ranked according to the average deviation from the ES-cell reference. Next, the top 1%, 5%, 10%, up to 90% most ES-cell variable genes were selected and the percentage of iPS cell-line specific deviations was calculated that would have been detected if only these genes were monitored for deviations.

FIG. 15 shows some of the currently used method for quality assessment of human pluripotent cell lines. All cheap- and simple assays lack specificity, and the most stringent assays are unavailable for humans. Although, teratomas are considered the gold standard for humans, teratomas are labor intensive and costly, impose high animal testing burden, and are highly dependent on qualified pathologists' assessment thus difficult to quantify.

FIG. 16 shows one embodiment where histone methylation profiling was performed using the ChIP-seq approach for different histone methylation marks. Using this embodiment of ChIP-seq method, there was good qualitative agreement among all ES/iPS cells is seen, the ChIP-seq method results in different quantitation and requires a large number of cells. Accordingly, one can used alternative methods for determining DNA methylation.

FIG. 17 shows a schematic representation of selecting iPS cell line having abnormal DNA methylated gene(s). DNA methylation mapping in many ES cell lines using bisulfite DNA methylation sequencing is used to establish normal variations. DNA methylation levels of different genes in a cell of interest is than compared to the normal DNA methylation levels for those genes, and genes with methylation levels falling outside the normal range are considered outliers.

FIG. 18 shows one example showing the number of genes with increased or decreased methylation levels in a variety of different ES and iPS cell lines used in this study.

FIG. 19A-19B shows aVenn diagram of the number of hypermethylated (FIG. 19A) and hypomethylated (FIG. 19B) genes in ES, iPS and fibroblast cells.

FIG. 19A shows one embodiment where 116 genes that were hypermethylated in both ES and iPS cells, of which, 11 were hypermethylated in both ES cells and fibroblasts, and 65 were hypermethylated in both iPS cells and fibroblasts. In this example of this embodiment, only 6 genes were hypermethylated in all 3 types of cells.

FIG. 19B shows one embodiment where there were also 116 genes that were hypomethylated in both ES and iPS cells; and 83 were hypermethylated in both ES cells and fibroblasts, and 217 were hypermethylated in both iPS cells and fibroblasts. In this example of this embodiment, only 58 genes were hypermethylated in all 3 types of cells.

FIG. 20 shows one embodiment of the score card showing the number of genes having increased or decreased methylation as compared to the normal variation methylation levels and number of cancer genes having increased or decreased methylation levels as compared to normal variation methylation reference levels in a variety of different ES and iPS cells. Pluripotent cell lines with low number of hypermethylated and/or hypomethylated cancer genes were designated as epigenetically “safe” ES or iPS cells, and cells with higher number of hypermethylated and/or hypomethylated cancer genes were designated as epigenetic outliers, and potentially unsafe for use in therapeutic and/or other applications.

FIG. 21 shows a schematic of generating a lineage scorecard, summarizing cell-line differentiation assay to determine differentiation bias or propensity of a set of human iPS lines. In this embodiment, a scorecard was derived using a 16-day embryoid body (EB) differentiation protocol, however, shorter differentiation protocols can be used, e.g., any duration from EB0 (EB day 0) to EB32 (EB day 21) or greater. The gene expression profiling of 500 “lineage gene expression genes” was used to quantify the propensity of the pluripotent stem cell line to differentiate along different cell types and lineages, and bioinformatic analysis was used to determine enriched vs. depleted gene sets and to compare with a plurality of other pluripotent cell lines (e.g., ES and iPS cell lines) to produce a lineage scorecard.

FIG. 22A shows experimental validation of lineage scorecard in the directed differentiation of human iPS lines into motor neurons. All iPS cell lines were differentiated into motor neurons. FIG. 22B shows an embodiment of a lineage scorecard indicating differentiation efficiency into motor neurons, which was measured by staining for Islet 1 (2-3 independent repetitions with >60,000 cell). Transgene expression was assayed by qPCR. Such a lineage scorecard was generated by gene expression profiling of 500 “lineage gene expression genes” to quantify the propensity of the pluripotent stem cell line to differentiate along different cell types and lineages, and bioinformatic analysis was used to determine enriched vs. depleted gene sets and to compare with a plurality of other pluripotent cell lines (e.g., ES and iPS cell lines) to produce a lineage scorecard.

FIG. 23 shows a flow chart of an embodiment of instructions for a computer program for producing a deviation scorecard for a pluripotent stem cell line of interest. The data is inputted into a computer comprising a processor and associated memory or storage device, and a gene mapping module, a reference comparison module, a normalization module a relevance filter module a gene set module and a scorecard display module to display the deviation scorecard.

FIG. 24 shows a flow chart of one embodiment of instructions for a computer program for producing a lineage scorecard for a pluripotent stem cell line of interest. While the data obtained for the generation of the deviation scorecard (e.g., DNA methylation data and/or gene expression data for the pluripotent stem cell line of interest) can be used, in this embodiment, input data is gene expression data of the pluripotent stem cell line of interest. The data is inputed into a computer comprising a processor and associated memory and/or storage device, and an assay normalization module. A sample normalization module, a reference comparison module, a gene set module, an enrichment analysis module and a scorecard display module to display the lineage scorecard.

FIG. 25 shows a simplified block diagram of an embodiment of the present invention which relates to a high-throughput system for characterizing a pluripotent stem cell of interest and producing a deviation and/or lineage scorecard. The determination module can be any apparatus or machine for measuring gene expression and/or DNA methylation.

FIG. 26 shows a simplified block diagram of an embodiment of the present invention which enables the data from the DNA methylation assay and gene expression assays to be configured to be processed by a computer system at any location and accessible through a used interface, where the data for each pluripotent stem cell is stored in a database.

FIG. 27 shows an exemplary block diagram of a computer system that can be configured to execute the instructions outlined in FIGS. 23 and 24.

### DETAILED DESCRIPTION OF THE INVENTION

The present invention generally relates to a reference data set or “scorecard” for a pluripotent stem cell, and methods, systems and kits to generate a scorecard for predicting the functionality and suitability of a pluripotent stem cell line for a desired use. The “scorecard” provides a reference value range for at least one normal posttranslational modification, such as methylation, in stem cells, and optionally a reference value range for normal expression pattern for differentiation-related genes in stem cells, and optionally further a normal range of lineage-specific markers, such as neural stem cell, hematopoietic stem cell, pancreatic stem cell and other more limited stem cell markers. In some aspects, the scorecard comprises at least two reference data sets selected from a posttranslational modification reference set, such as DNA methylation reference set, a differentiation propensity reference set and a gene expression data set. In some embodiments, the scorecard further provides guidelines to determine if a pluripotent stem cell of interest falls within normal parameters of normal pluripotent stem cell variation. Such guidelines are preferably in a computer executable format.

In some embodiments, the scorecard comprises at least two reference data sets selected from a epigenetic or posttranslational modification, such as DNA methylation reference set, a differentiation propensity reference set and a gene expression data set compiled from the data of 19 different ES cell lines set forth in this specification. In alternative embodiments, the scorecard is a scorecard compiled from the data of a pluripotent stem cell with desirable characteristics, for example, a pluripotent stem cell with differentiation propensity to differentiate into endoderm lineages, such as pancreatic lineages and the like, such as ectoderm or mesoderm differentiation markers.

Another aspect of the present invention relates to a method for generating a scorecard comprises using at least 2 stem cell assays selected from: epigenetic profiling, differentiation assay and gene expression assay to predict the functionality and suitability of a pluripotent stem cell line for a desired use. In some embodiments, the scorecard reference data can be compared with the pluripotent stem cells data to effectively and accurately predict the utility of the pluripotent stem cell for a given application, as well as any to identify specific characteristics of the pluripotent stem cell line to determine their suitability for downstream applications, such as for example, their suitability for therapeutic use, drug screening and toxicity assays, differentiation into a desired cell lineage, and the like.

In some embodiments, the DNA methylation reference set relates to the level of methylation of a first set of reference genes, where the DNA methylation reference genes can be cancer genes, and/or developmental genes, and are disclosed in Tables 12A. In some embodiments, the genes used in a first set of reference DNA methylation genes are at least about 200, or at least about 300, or at least about 400, or at least about 500, or at least about 600, or at least about 800, or at least about 1000, or at least about 1500, or at least about 2000, or at least about 3000, or at least about 4000, or at least about 5000 genes, in any combination, selected from the list of genes in Table 12A and/or Table 12C and/or Tables 13A, 13B or Table 14. In some embodiments, the genes are any combination of sets of genes selected with numbers 1-200, or numbers 1-500, or numbers 1-1000 of the genes listed in any of Tables 12A, Table 12C, Table 13A, Table 13B or Table 14.

Accordingly, one aspect of the present invention relates to methods and a plurality of assays for predicting the functionality and suitability of a pluripotent stem cell line for a desired use. In some embodiments, at least one, or at least 2 or at least three of stem cell assays can be used alone or in any combination, to predict the functionality and suitability of a pluripotent stem cell line for a desired use. In some embodiment, a first assay is epigenetic profiling, e.g., assessment of gene methylation of specific defined gene set to determine genes activated in the pluripotent stem cell line. In some embodiments, a second assay is a differentiation assay to determine the propensity of the pluripotent stem cell line to differentiate along specific lineages. In some embodiments, the assay is a gene expression assay, e.g., a whole genome gene expression assay to determine the Another aspect relates to a set of reference data, herein referred to a “scorecard” which is the average data from results of a number of different pluripotent stem cell lines from the three combined assays of the present invention, providing reference data which constitutes a “scorecard” that can be used by one of ordinary skill in the art to compare with their pluripotent stem cell line of interest, where the comparison with the reference “scorecard” can be used to effectively and accurately predict the utility of the pluripotent stem cell for a given application, as well as any specific characteristics of the pluripotent stem cell line of interest, e.g., a ES cell or iPS cell line. Accordingly, the methods, assays and scorecards as disclosed herein can be used for identify specific characteristics of stem cells to determine their suitability for downstream applications, such as for example, their suitability for therapeutic use, drug screening and toxicity assays, differentiation into a desired cell lineage, and the like.

In some embodiments, the assays as disclosed herein can be used to characterize and determine the quality of a variety of a pluripotent stem cell line, such as for example, but not limited to embryonic stem cells, autologous adult stem cells, iPS cell, and other pluripotent stem cell lines, such as reprogrammed cells, direct reprogrammed cells or partially reprogrammed cells. In some embodiments, a stem cell line is a human stem cell line. In some embodiments, a pluripotent stem cell line is a genetically modified pluripotent stem cell line. In some embodiments, where the pluripotent stem cell line is for therapeutic use or for transplantation into a subject, a pluripotent stem cell line is an autologous pluripotent stem cell line, e.g., derived from a subject to which a population of stem cells will be transplanted back into, and in alternative embodiments, a pluripotent stem cell line is an allogenic pluripotent stem cell line.

### DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The term “scorecard” as disclosed herein refers to a listing of a summary of the DNA methylation and/or gene expression differences of selected genes in one or more pluripotent stem cell lines of interest as compared to a reference pluripotent stem cell line, and functions as record of the pluripontent stem cell's predicted performance, for example, differentation ability and/or pluripotency capacity and/or predispostion to become cancerous cell line. A scorecard can exist in any form, for example, in a database, a written form, an electronic form and the like, and can be electronically or digitally recorded and stored in annotated databases. In some embodiments, a scorecard can be a graphical representation of a prediction of the pluripotent stem cell capabilities (e.g., differentiation capabilities, pluripotency etc.) as compared to a reference pluripotent cell line or plurality of lines. Accordingly, the scorecards as disclosed herein serve as an indicator or listing of the characteristics and potential of a pluripotent stem cell line and can be used to assist in fast and efficient selection of a particular pluripotent stem cell line for a particular use and/or to reach a specific objective.

The term “reprogramming” as used herein refers to a process that alters or reverses the differentiation state of a differentiated cell (e.g. a somatic cell). Stated another way, reprogramming refers to a process of driving the differentiation of a cell backwards to a more undifferentiated or more primitive type of cell. Complete reprogramming involves complete reversal of at least some of the heritable patterns of nucleic acid modification (e.g., methylation), chromatin condensation, epigenetic changes, genomic imprinting, etc., that occur during cellular differentiation as a zygote develops into an adult. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent, although the compositions and methods of the invention may also be of use for such purposes.

The term “stable reprogrammed cell” as used herein refers to a cell which is produced from the partial or incomplete reprogramming of a differentiated cell (e.g. a somatic cell). A stable reprogrammed cell is used interchangeably herein with “piPSC”. A stable reprogrammed cell has not undergone complete reprogramming and thus has not had global remodeling of the epigenome of the cell. A stable reprogrammed cell is a pluripotent stem cell and can be further reprogrammed to an iPSC, as that term is defined herein, or alternatively can be differentiated along different lineages. In some embodiments, a partially reprogrammed cell expresses markers from all three embryonic germ layers (i.e. all three layers of endoderm, mesoderm or ectoderm layers). In mouse, markers of endoderm germ cells include, Gata4, FoxA2, PDX1, Nodal, Sox7 and Sox17. In mouse, markers of mesoderm germ cells include, Brachycury, GSC, LEF1, Mox1 and Tie1. In mouse, markers of ectoderm germ cells include cripto1, EN1, GFAP, Islet 1, LIM1 and Nestin. In some embodiments, a partially reprogrammed cell is an undifferentiated cell. Markers for human endoderm germ cells, ectoderm germ cells and mesoderm germ cells are disclosed herein in Table 7, and for example, markers for ectoderm germ cells include, but are not limited to, NCAM1, EN1, FGFR2, GATA2, GATA3, HAND1, MNX1, NEFL, NES, NOG, OTX2, PAX3, PAX6, PAX7, SNAI2, SOX10, SOX9, TDGF1, APOE, PDGFRA, MCAM, FUT4, NGFR, ITGB1, CD44, ITGA4, ITGA6, ICAM1, THY1, FAS, ABCG2, CRABP2, MAP2, CDH2, NES, NEUROG3, NOG, NOTCH1, SOX2, SYP, MAPT, TH. Markes for human endoderm germ cells include, but are not limited to, APOE, CDX2, FOXA2, GATA4, GATA6, GCG, ISL1, NKX2-5, PAX6, PDX1, SLC2A2, SST, ITGB1, CD44, ITGA6, THY1, CDX2, GATA4, HNF1A, HNF1B, CDH2, NEUROG3, CTNNB1, SYP, and markers for mesoderm germ cells include, but are not limited to, CD34, DLL1, HHEX, INHBA, LEF1, SRF, T, TWIST1, ADIPOQ, MME, KIT, ITGAL, ITGAM, ITGAX, TNFRSF1A, ANPEP, SDC1, CDH5, MCAM, FUT4, NGFR, ITGB1, PECAM1, CDH1, CDH2, CD36, CD4, CD44, ITGA4, ITGA6, ITGAV, ICAM1, NCAM1, ITGB3, CEACAM1, THY1, ABCG2, KDR, GATA3, GATA4, MYOD1, MYOG, NES, NOTCH1, SPI1, STAT3.

The term “induced pluripotent stem cell” or “iPSC” or “iPS cell” refers to a cell derived from a complete reversion or reprogramming of the differentiation state of a differentiated cell (e.g. a somatic cell). As used herein, an iPSC is fully reprogrammed and is a cell which has undergone complete epigenetic reprogramming. As used herein, an iPSC is a cell which cannot be further reprogrammed (e.g., an iPSC cell is terminally reprogrammed).

The term “remodeling of the epigenome” refers to chemical modifications of the genome which do not change the genomic sequence or a gene's sequence of base pairs in the cell, but alter the expression.

The term “global remodeling of the epigenome” refers to where chemical modifications of the genome have occurred where there is no memory of prior gene expression from the differentiated cell from which the reprogrammed cell or iPSC was derived.

The term “incomplete remodeling of the epigenome” refers to where chemical modifications of the genome have occurred where there is memory of prior gene expression from the differentiated cell from which the stable reprogrammed cell or piPSC was derived.

The term “epigenetic reprogramming” as used herein refers to the alteration of the pattern of gene expression in a cell via chemical modifications that do not change the genomic sequence or a gene's sequence of base pairs in the cell.

The term “epigenetic” as used herein refers to “upon the genome”. Chemical modifications of DNA that do not alter the gene's sequence, but impact gene expression and may also be inherited. Epigenetic modification can also include, in some instances posttranslational modifications or “PTM”, which are changes to DNA which to not alter the genes DNA or nucleic acid sequence, and are important, for example, in imprinting and cellular reprogramming. Post-translational modifications include, for example, DNA methylation, ubiquitination, phosphorylation, glycosylation, sumoylation, acetylation, S-nitrosylation or nitrosylation, citrullination or deimination, neddylation, OClcNAc, ADP-ribosylation, hydroxylation, fattenylation, ufmylation, prenylation, myristoylation, S-palmitoylation, tyrosine sulfation, formylation, and carboxylation.

The term “methylation” as used herein, refers to the covalent attachment of a methyl group at the C5-position of the nucleotide base cytosine within the CpG dinucleotides of gene regulatory region. The term “methylation state” or “methylation status” refers to the presence or absence of 5-methyl-cytosine (“5-mCyt”) at one or a plurality of CpG dinucleotides within a DNA sequence. As used herein, the terms “methylation status” and “methylation state” are used interchangeably. A methylation site is a sequence of contiguous linked nucleotides that is recognized and methylated by a sequence-specific methylase. A methylase is an enzyme that methylates (i.e., covalently attaches a methyl group) one or more nucleotides at a methylation site.

The term “methylation level” refers to the amount of methylation present on the DNA sequence of a target DNA methylation gene, e.g., in all genomic regions, and some non-genomic regions. In some embodiments, the methylation level is determined in a promoter region of a target gene.

As used here, the term “CpG islands” are short DNA sequences rich in CpG dinucleotide and can be found in the 5′ region of about one-half of all human genes. The term “CpG site” refers to the CpG dinucleotide within the CpG islands. CpG islands are typically, but not always, between about 0.2 to about 1 kb in length.

The terms “gene profile” as used herein is intended to refer to the gene expression level of a gene, or a set of genes, in a pluripotent stem cell sample. In one embodiment of the invention the term “gene profile” refers to a gene or a set of genes listed in Table 12B and/or 12C or to any selection of the genes of Table 12B or Table 12C, Table 13A, Table 13B or Table 14, which are described herein.

The terms “differential expression” in the context of the present invention means the gene is up-regulated or down-regulated in comparison to its normal variation of expression in a pluripotent stem cell. Statistical methods for calculating differential expression of genes are discussed elsewhere herein.

By “genes of Table 12B” is used interchangeably herein with “gene listed in Table 12B” and refers to the gene products of genes listed under “Gene name” in Table 12B. By “gene product” is meant any product of transcription or translation of the genes, whether produced by natural or artificial means. In some embodiments of the invention, the genes referred to herein are those listed in Table 12A and 12B and 12C as defined in the column 2, “Gene name”. The genes are also listed in Tables 12A, Table 12C, Table 13A, Table 13B or Table 14.

The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. In some embodiments, a pluripotent cell is an undifferentiated cell.

The term “pluripotency” or a “pluripotent state” as used herein refers to a cell with the ability to differentiate into all three embryonic germ layers: endoderm (gut tissue), mesoderm (including blood, muscle, and vessels), and ectoderm (such as skin and nerve), and typically has the potential to divide in vitro for a long period of time, e.g., greater than one year or more than 30 passages.

The term “multipotent” when used in reference to a “multipotent cell” refers to a cell that is able to differentiate into some but not all of the cells derived from all three germ layers. Thus, a multipotent cell is a partially differentiated cell. Multipotent cells are well known in the art, and examples of multipotent cells include adult stem cells, such as for example, hematopoietic stem cells and neural stem cells. Multipotent means a stem cell may form many types of cells in a given lineage, but not cells of other lineages. For example, a multipotent blood stem cell can form the many different types of blood cells (red, white, platelets, etc . . . ), but it cannot form neurons.

The term “multipotency” refers to a cell with the degree of developmental versatility that is less than totipotent and pluripotent.

The term “totipotency” refers to a cell with the degree of differentiation describing a capacity to make all of the cells in the adult body as well as the extra-embryonic tissues including the placenta. The fertilized egg (zygote) is totipotent as are the early cleaved cells (blastomeres)

The term “differentiated cell” is meant any primary cell that is not, in its native form, pluripotent as that term is defined herein. The term a “differentiated cell” also encompasses cells that are partially differentiated, such as multipotent cells, or cells that are stable non-pluripotent partially reprogrammed cells. It should be noted that placing many primary cells in culture can lead to some loss of fully differentiated characteristics. Thus, simply culturing such cells are included in the term differentiated cells and does not render these cells non-differentiated cells (e.g. undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture. In some embodiments, the term “differentiated cell” also refers to a cell of a more specialized cell type derived from a cell of a less specialized cell type (e.g., from an undifferentiated cell or a reprogrammed cell) where the cell has undergone a cellular differentiation process.

As used herein, the term “somatic cell” refers to any cell other than a germ cell, a cell present in or obtained from a pre-implantation embryo, or a cell resulting from proliferation of such a cell in vitro. Stated another way, a somatic cell refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for reprogramming a differentiated cell can be performed both in vivo and in vitro (where in vivo is practiced when an differentiated cell is present within a subject, and where in vitro is practiced using isolated differentiated cell maintained in culture). In some embodiments, where a differentiated cell or population of differentiated cells are cultured in vitro, the differentiated cell can be cultured in an organotypic slice culture, such as described in, e.g., meneghel-Rozzo et al., (2004), Cell Tissue Res, 316(3); 295-303, which is incorporated herein in its entirety by reference.

As used herein, the term “adult cell” refers to a cell found throughout the body after embryonic development.

In the context of cell ontogeny, the term “differentiate”, or “differentiating” is a relative term meaning a “differentiated cell” is a cell that has progressed further down the developmental pathway than its precursor cell. Thus in some embodiments, a reprogrammed cell as this term is defined herein, can differentiate to lineage-restricted precursor cells (such as a mesodermal stem cell), which in turn can differentiate into other types of precursor cells further down the pathway (such as an tissue specific precursor, for example, a cardiomyocyte precursor), and then to an end-stage differentiated cell, which plays a characteristic role in a certain tissue type, and may or may not retain the capacity to proliferate further.

The term “embryonic stem cell” is used to refer to the pluripotent stem cells of the inner cell mass of the embryonic blastocyst (see U.S. Pat. Nos. 5,843,780, 6,200,806, which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “phenotype” refers to one or a number of total biological characteristics that define the cell or organism under a particular set of environmental conditions and factors, regardless of the actual genotype.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The term “exogenous” refers to a substance present in a cell other than its native source. The terms “exogenous” when used herein refers to a nucleic acid (e.g. a nucleic acid encoding a sox2 transcription factor) or a protein (e.g., a sox2 polypeptide) that has been introduced by a process involving the hand of man into a biological system such as a cell or organism in which it is not normally found or in which it is found in lower amounts. A substance (e.g. a nucleic acid encoding a sox2 transcription factor, or a protein, e.g., a sox2 polypeptide) will be considered exogenous if it is introduced into a cell or an ancestor of the cell that inherits the substance. In contrast, the term “endogenous” refers to a substance that is native to the biological system or cell (e.g. differentiated cell).

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from. In some embodiments, the isolated population is an isolated population of reprogrammed cells which is a substantially pure population of reprogrammed cells as compared to a heterogeneous population of cells comprising reprogrammed cells and cells from which the reprogrammed cells were derived.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of reprogrammed cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not reprogrammed cells or their progeny as defined by the terms herein. In some embodiments, the present invention encompasses methods to expand a population of reprogrammed cells, wherein the expanded population of reprogrammed cells is a substantially pure population of reprogrammed cells.

As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, and refers to a process of a cell making more copies of itself (e.g. duplication) of the cell. In some embodiments, reprogrammed cells are capable of renewal of themselves by dividing into the same undifferentiated cells (e.g. pluripotent or non-specialized cell type) over long periods, and/or many months to years. In some instances, proliferation refers to the expansion of reprogrammed cells by the repeated division of single cells into two identical daughter cells.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

The term “lineages” as used herein describes a cell with a common ancestry or cells with a common developmental fate. By way of an example only, a cell that is of endoderm origin or is “endodermal lineage” this means the cell was derived from an endodermal cell and can differentiate along the endodermal lineage restricted pathways, such as one or more developmental lineage pathways which give rise to definitive endoderm cells, which in turn can differentiate into liver cells, thymus, pancreas, lung and intestine.

The terms “decrease”, “reduced”, “reduction”, “decrease” or “inhibit” are all used herein generally to mean a decrease by a statistically significant amount. However, for avoidance of doubt, ““reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2 SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, the term “DNA” is defined as deoxyribonucleic acid.

The term “differentiation” as used herein refers to the cellular development of a cell from a primitive stage towards a more mature (i.e. less primitive) cell.

The term “directed differentiation” as used herein refers to forcing differentiation of a cell from an undifferentiated (e.g. more primitive cell) to a more mature cell type (i.e. less primitive cell) via genetic and/or environmental manipulation. In some embodiments, a reprogrammed cell as disclosed herein is subject to directed differentiation into specific cell types, such as neuronal cell types, muscle cell types and the like.

The term “functional assay” as used herein is a test which assesses the properties of a cell, such as a cell's gene expression or developmental state by evaluating its growth or ability to live under certain circumstances. In some embodiments, a reprogrammed cell can be identified by a functional assay to determine the reprogrammed cell is a pluripotent state as disclosed herein.

The term “disease modeling” as used herein refers to the use of laboratory cell culture or animal research to obtain new information about human disease or illness. In some embodiments, a reprogrammed cell produced by the methods as disclosed herein can be used in disease modeling experiments.

The term “drug screening” as used herein refers to the use of cells and tissues in the laboratory to identify drugs with a specific function. In some embodiments, the present invention provides drug screening methods of differentiated cells to identify compounds or drugs which reprogram a differentiated cell to a reprogrammed cell (e.g. a reprogrammed cell which is in a pluripotent state or a reprogrammed cell which is a stable intermediate, partially reprogrammed cell, as disclosed herein). In some embodiments, the present invention provides drug screening methods of stable intermediate partially reprogrammed cells to identify compounds or drugs which reprogramming differentiated cells into fully reprogrammed cells (e.g. reprogrammed cells which are in a pluripotent state). In alternative embodiments, the present invention provides drug screening on reprogrammed cells (e.g. human reprogrammed cells) to identify compounds or drugs useful as therapies for diseases or illnesses (e.g. human diseases or illnesses).

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, e.g., “selective conditions”. To ensure an effective selection, a population of cells can be maintained for a under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (whether partial or total), whether detectable or undetectable. In some embodiments, treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. In some embodiments, the term “treatment” can also mean prolonging survival as compared to expected survival if not receiving treatment. Those in need of treatment include those already diagnosed with a disease or condition, as well as those likely to develop a disease or condition due to genetic susceptibility or other factors which contribute to the disease or condition, such as a non-limiting example, weight, diet and health of a subject are factors which may contribute to a subject likely to develop diabetes mellitus. Those in need of treatment also include subjects in need of medical or surgical attention, care, or management. The subject is usually ill or injured, or at an increased risk of becoming ill relative to an average member of the population and in need of such attention, care, or management.

As used herein, the terms “administering,” “introducing” and “transplanting” are used interchangeably in the context of the placement of reprogrammed cells as disclosed herein, or their differentiated progeny into a subject, by a method or route which results in at least partial localization of the reprogrammed cells, or their differentiated progeny at a desired site. The reprogrammed cells, or their differentiated progeny can be administered directly to a tissue of interest, or alternatively be administered by any appropriate route which results in delivery to a desired location in the subject where at least a portion of the reprogrammed cells or their progeny or components of the cells remain viable. The period of viability of the reprogrammed cells after administration to a subject can be as short as a few hours, e.g. twenty-four hours, to a few days, to as long as several years.

The term “transplantation” as used herein refers to introduction of new cells (e.g. reprogrammed cells), tissues (such as differentiated cells produced from reprogrammed cells), or organs into a host (i.e. transplant recipient or transplant subject)

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip.

The term “software” is used interchangeably herein with “program” and refers to prescribed rules to operate a computer. Examples of software include: software; code segments; instructions; computer programs; and programmed logic.

The term a “computer system” may refer to a system having a computer, where the computer comprises a computer-readable medium embodying software to operate the computer.

The term “proteomics” may refer to the study of the expression, structure, and function of proteins within cells, including the way they work and interact with each other, providing different information than genomic analysis of gene expression.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following, including the Examples, but the scope of the invention should not be limited thereto.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### In General

One aspect of the present invention relate to methods, systems and assays for the production of two scorecards for characterizing pluripotent stem cell lines, a first scorecard which can be referred to a “deviation scorecard” or “pluripotency scorecard” which is useful to provide information of how the pluripotent stem cell line of interest compares to previously established or control pluripotent stem cell lines, and can be used to identify the number or % of genes which deviate in terms of DNA methylation or gene expression as compared to a reference pluripotent stem cell line and/or a plurality of reference pluripotent stem cell lines. Such a scorecard is useful for identifying the pluripotency of the stem cell line of interest as well as to identify if the stem cell line of interest has atypical gene expression or DNA methylation of cancer genes which may predispose the stem cell line of interest to abberant proliferation and formation of cancer at a later time point. A second score card, herein referred to as a “lineage scorecard” which is useful as a quantification of the differentiation potential of the pluripotent stem cell of interest, and provides information of how efficienty the pluripotent stem cell line of interest will differentiation into particular lineages of interest as compared to previously established or control pluripotent stem cell lines. A “summary scorecard” can comprise a deviation scorecard and lineage scorecard of one or more pluripotent stem cell lines of interest.

Accordingly, further aspects of the present invention provide a method for validating and/or monitoring a pluripotent stem cell population, comprising generating a score card of a pluripotent stem cell line, by monitoring at least two datasets selected from (i) identification of epigenetic silencing of specific genes by promoter methylation of specific, e.g., oncogenes, tumor suppressor genes and development genes, (ii) identification of gene expression, e.g. developmental genes and lineage marker genes, and (iii) differentiation propensity to differentiate along different lineages to allow identification of characteristics of pluripotent stem cells and to predict which pluripotent stem cell lines are likely to contribute to a stem-cell originated cancer.

In some embodiments, for example, one can determine the differentiation propensity for a given cell line (using differentially modified methylation and/or differentially gene expression of lineage marker genes), followed by determination of quality of determining changes in DNA methylation of target genes (e.g., some or a combination of genes listed in any of Tables 12A and/or Table 12C, Table 13A, Table 13B or Table 14) and/or determining changes in gene expression levels of target genes (e.g., some or a combination of genes listed in any of Tables 12B and/or Table 12C, or selected from Table 13A, Table 13B or Table 14) as compared to a reference or “standard” pluripotent stem cell line.

As discussed herein, the scorecard as comprises several components: (i) identification of DNA methylation gene outliers in a pluripotent cell as compared to the normal variation of DNA methylation for the target genes in reference pluripotent cell lines, (ii) identification of gene expression outliers in a pluripotent cell line as compared to the normal variation of DNA expression level for the target genes in reference pluripotent cell lines, (iii) prediction of cellular differentiation bias based on the DNA methylation and/or gene expression data from (i) and (ii), and/or gene expression/DNA methylation data from pluripotent cell lines that have been induced to differentiate.

The present invention has substantial utility for determining the quality and utility for various types of pluripotent stem cells and precursor cells (e.g., ES cell, somatic stem cells, hematopoietic stem cells, leukemic stem cells, skin stem cells, intestinal stem cells, gonadal stem cells, brain stem cells, muscle stem cells (muscle myoblasts, etc.), mammary stem cells, neural stem cells (e.g., cerebellar granule neuron progenitors, etc.), etc), and for various stem cell or precursor cells (e.g., such as those described in Table 1 of Sparmann & Lohuizen, Nature 6, 2006 (Nature Reviews Cancer, November 2006), incorporated herein by reference), as well as in vitro and in vivo derived stem cells, such as induced pluripotent stem cells (iPSC) as well as terminally differentiated cells.

In some aspects of the invention, the invention relates to generating a scorecard of a pluripotent stem cell line, for validating and monitoring and to serve as a general quality control of the pluripotent stem cell line, by monitoring at least two datasets selected from (i) identification of epigenetic silencing of specific genes by promoter methylation of specific, e.g., oncogenes, tumor suppressor genes and development genes, (ii) identification of gene expression, e.g. developmental genes and lineage marker genes, and (iii) differentiation propensity to differentiate along different lineages to allow identification of characteristics of pluripotent stem cells and to predict which pluripotent stem cell lines are likely to contribute to a stem-cell originated cancer.

In some embodiments, the present invention provides a method for selecting a pluripotent stem cell line, comprising' (i) measuring epigenetic modification of a set of target genes in the pluripotent stem cell line by contacting at least one pluripotent stem cell with an agent that differentially binds to an epigenetic modification in the DNA, and performing a comparison of the epigenetic modification data with a reference epigenetic modification data of the same target genes; (ii) measuring differentiation potential of the pluripotent stem cell line by undirected or directed differentiation of the pluripotent stem cell and labeling the transcripts to allow detection of the level of gene expression of a plurality of lineage marker genes; and comparing the differentiation potential data with a reference differentiation potential data; and (iii) selecting a pluripotent stem cell line which does not differ by a statistically significant amount in the epigenetic modification of DNA of the target genes as compared to the reference epigenetic modification level, and does not differ by a statistically significant amount in the propensity to differentiate along mesoderm, ectoderm and endoderm lineages as compared to a reference differentiation potential; or discarding a pluripotent stem cell line which differs by a statistically significant amount in the in the epigenetic modification of the target genes as compared to the reference epigenetic modification level, and differs by a statistically significant amount in the propensity to differentiate along mesoderm, ectoderm and endoderm lineages as compared to a reference differentiation potential.

In some embodiments, the epigenetic modification comprises measuring epigenetic modification in a set of target genes in the pluripotent stem cell line, for example, epigenetic modification can be measured by any one of the following selected from the group consisting of: enrichment-based methods (e.g. MeDIP, MBD-seq and MethylCap), bisulfite sequencing and bisulfite-based methods (e.g. RRBS, bisulfite sequencing, Infinium, GoldenGate, COBRA, MSP, MethyLight) and restriction-digestion methods (e.g., MRE-seq), or differential-conversion, differential restriction, differential weight of the DNA methylated target gene of the pluripotent stem cell as compared to the reference DNA methylation data of the same target genes.

In some embodiments, the method further comprises (iv) measuring the gene expression of a second set of target genes in the pluripotent stem cell line and performing a comparison of the gene expression data with a reference gene expression level of the same target genes; and (v) selecting a pluripotent stem cell line which does not differ by a statistically significant amount in the level of gene expression of the target genes as compared to the reference gene expression level; or discarding a pluripotent stem cell line which differs by a statistically significant amount in the expression level of the target genes as compared to the reference gene expression level.

In some embodiments, the reference DNA methylation level is a range of normal variation of methylation for that DNA methylation target gene, and can be in some instances, an average and optionally plus or minus a standard variation of DNA methylation for that DNA methylation target gene, wherein the average is calculated from DNA methylation of that target gene in a plurality of pluripotent stem cell lines, e.g., at least 5 or more pluripotent stem lines.

In some embodiments, the reference gene expression level is range of normal variation of for that target gene, and in some embodiments, it an average of expression level for that target gene, wherein the average is calculated from expression level of that target gene in a plurality of pluripotent stem cell lines, for example, at least 5 or more different pluripotent stem cell lines.

In some embodiments, gene expression is determined by a microarray assay, such as a quantitative differentiation assay.

In some embodiments, the reference differentiation potential is the ability to differentiate into a lineage selected from the group consisting of mesoderm, endoderm, ectoderm, neuronal, hematopoietic lineages, and any combinations thereof, where the reference differentiation potential data is generated from a plurality of pluripotent stem cell lines, for example, at least 5 different pluripotent stem cell lines. In some embodiments, the differentiation potential of a test pluripotent stem cell and/or a reference pluripotent stem cell is determined by allowing the pluripotent stem cell to differentiate (either directed differentiation or spontaneous differentiation for a predefine period of time) and the difference in DNA methylation and/or gene expression is determined.

In some embodiments of all aspects of the present invention, DNA methylation target genes and/or the reference DNA methylation target genes are selected from the group consisting of cancer genes, oncogenes, tumor suppressor genes, developmental genes, lineage marker genes, and any combinations thereof, and include DNA methylation target genes and/or the reference DNA methylation target genes are selected from the group listed in Table 12A, or selected from Table 13A, Table 13B or Table 14, and any combinations thereof. In some embodiments, oncogenes genes are selected from c-Sis, epidermal growth factor receptor, platelet-derived growth factor receptor, vascular endothelial growth factor receptor, HER2/new, Src family of tyrosine kinases, Syk-Zap-70 family of tyrosine kinases, BTK family of tyrosine kinases, Raf kinase, cyclin-dependent kinases, Ras protein, and myc gene. In some embodiments, tumor suppressor genes are selected from TP53, PTEN, APC, CD95, ST5, ST7 and ST14 gene. In some embodiments, developmental genes are selected from any combination of genes listed in Table 7. In some embodiments, lineage marker genes are selected from VEGF receptor II (KDR), actin α-2 smooth muscle (ACTA2), Nestin, Tublin P3, alpha-feto protein (AFP), syndecan-4, CD64IFcyRI, Oct-4, beta-HCG, beta-LH, oct-3, Brachyury T, Fgf-5, nodal, GATA-4, flk-1, Nkx-2.5, EKLF, and Msx3. In some embodiments, DNA methylation target genes and/or the reference DNA methylation target genes are selected from the group consisting of BMP4, CAT, CD14, CXCL5, DAZL, DNMT3B, GATA6, GAPDH, LEFTY2, MEG3, PAX6, S100A6, SOX2, SNAI1, TF, and any combinations thereof. In some embodiments, DNA methylation of least about 200 target genes selected from any combination of genes in the list in Table 12A, or selected from Table 13A, Table 13B or Table 14, are measured in the pluripotent cell line, and compared to the reference DNA methylation level of the same set of at least 200 target genes, or can be at least about 200 target genes selected from any combination of genes in the list in Table 12A, or selected from Table 13A-13B or Table 14 are selected from any combination of genes of Numbers 1-500 listed in Table 12A, or selected from Table 13A, Table 13B or Table 14, or can be at least about 200 target genes are selected from Numbers 1-200 listed in Table 12A, or selected from Table 13A, Table 13B or Table 14. In some embodiments, DNA methylation of least about 500 target genes selected from any combination of genes in the list in Table 12A are measured in the pluripotent cell line, and compared to the reference DNA methylation level of the same set of at least 500 target genes. In some embodiments, the DNA methylation of least about 500 target genes selected from any combination of genes in the list in Table 12A, or selected from Table 13A, Table 13B or Table 14 are selected from any combination of genes of Numbers 1-1000 listed in Table 12A, or selected from Table 13A, Table 13B or Table 14.

In some embodiments of all aspects of the present invention, gene expression target genes and/or the reference gene expression target genes are selected from the group listed in Table 12B, or selected from Table 13A, Table 13B or Table 14, and any combinations thereof, such as, for example, at least about 200 or at least about 500 target genes are selected from Numbers 1-500 listed in Table 12A, or at least about 1000 target genes selected from any combination of genes in the list in Table 12A, or selected from Table 13A, Table 13B or Table 14, or at least about 1000 target genes are selected from Numbers 1-2000 listed in, or selected from Table 13A, Table 13B or Table 14A.

In some embodiments, a number of DNA methylation genes in the pluripotent stem cell line has a statistically significant difference in methylation relative to the reference genes is 10, 9, 8, 7, 6, 5, 4, 3, 2, 1, or 0. In some embodiments, a number of genes in the pluripotent stem cell line having a statistically significant difference in gene expression level relative to the reference genes is 10, 9, 8, 7, 6, 5, 4, 3, 2, 1, or 0.

In some embodiments, a pluripotent stem cell is a mammalian pluripotent stem cell, such as a human pluripotent stem cell.

Another aspect of the present invention relates to the use of a pluripotent stem cell for screening a compound for biological activity. For example, such an embodiment comprises (i) optionally causing or permitting the pluripotent stem cell to differentiate along a specific lineage; (ii) contacting the cell with a test compound; and (iii) determining any effect of the compound on the cell.

In some embodiments, a compound is selected from the group consisting of small organic molecule, small inorganic molecule, polysaccharides, peptides, proteins, nucleic acids, an extract made from biological materials such as bacteria, plants, fungi, animal cells, animal tissues, and any combinations thereof, and can be used at a concentration in the range of about 0.01 nM to about 1000 mM. In some embodiments, screen is a high-throughput screening method. In some embodiments, a biological activity is elicitation of a stimulatory, inhibitory, regulatory, toxic, electrical stimuli or lethal response in a biological assay. In some embodiments, a biological activity is selected from the group consisting of modulation of an enzyme activity, inactivation of a receptor, stimulation of a receptor, modulation of the expression level of one or more genes, modulation of cell proliferation, modulation of cell division, modulation of cell morphology, and any combinations thereof. In some embodiments, specific lineage is genotypic or phenotypic of a disease, for example a genotypic or phenotypic of an organ, tissue, or a part thereof.

Another aspect of the present invention relates to the use of a pluripotent stem cell validated and characterized using the methods and scorecards as disclosed herein for treatment of a subject by administering to a subject a pluripotent stem cell, for example a treatment of a mammalian subject, e.g., a mouse or rodent animal model or a human subject, such as for regenerative medicine and cell replacement/enhancement therapy. In some embodiments, a subject suffers from or is diagnosed with a disease or conditions selected from the group consisting of cancer, diabetes, cardiac failure, muscle damage, Celiac Disease, neurological disorder, neurodegenerative disorder, lysosomal storage disease, and any combinations thereof. In some embodiments, the pluripotent stem cell is administered locally, or alternatively, administration is transplantation of the pluripotent stem cell into the subject.

In some embodiments, the a pluripotent stem cell is differentiated before administering the pluripotent stem cell, or differentiated progeny thereof to the subject, for example, differentiated along a lineage selected from the group consisting of mesoderm, endoderm, ectoderm, neuronal, hematopoietic lineages, and any combinations thereof, or differentiated into an insulin producing cell (pancreatic cell, beta-cell, etc.), neuronal cell, muscle cell, skin cell, cardiac muscle cell, hepatocyte, blood cell, adaptive immunity cell, innate immunity cell and the like.

Another aspect of the present invention relates to a kit comprising a pluripotent stem cell selected by using the methods, assays and scorecards as disclosed herein. The kit can further comprise instructions for use.

Another aspect of the present invention relates to an assay for characterizing a plurality of properties of a pluripotent cell, the assay comprising at least 2 of the following: (i) a DNA methylation assay; (ii) a gene expression assay; and (iii) a differentiation assay. In some embodiments, the assay can be in the form of a kit. In some embodiments, the assay is performed by an investigator or by a service provider. In some embodiments, the assay provides a report in the format of a scorecard to validate and/or characterize a pluripotent stem cell line according to the methods as disclosed herein.

In some embodiments, the assays comprises a DNA methylation assay which is a bisulfite sequencing assay, or a whole genome bisulfite sequencing assay, or can be any DNA methylation assay selected from the group consisting of: enrichment-based methods (e.g. MeDIP, MBD-seq and MethylCap), bisulfite sequencing and bisulfite-based methods (e.g. RRBS, bisulfite sequencing, Infinium, GoldenGate, COBRA, MSP, MethyLight) and restriction-digestion methods (e.g., MRE-seq).

In some embodiments, the assays comprises a gene expression assay which is a microarray assay, e.g., a quantitative differentiation assay. In some embodiments, the assays comprises a differentiation assay which assess the ability of the pluripotent cell to differentiate into at least one of the following lineages: mesoderm, endoderm, ectoderm, neuronal, or hematopoietic lineages, where the ability of the pluripotent cell to differentiate into particular lineages is determined by DNA methylation assays, and/or gene expression assays as disclosed herein, or alternatively, immunostaining or FAC sorting using an antibody to at least one marker for mesoderm, endoderm and ectoderm lineages. In some embodiments, the ability of the pluripotent cell to differentiate into specific lineages is determined after at least about 0 days, for example between about 0-3 days, or about 3-7 days, or about 7-10 days or about 10-14 days or more than 14 days of culturing the EB.

In some embodiments, the differentiation assay assesses the ability of the pluripotent cell to differentiate along mesoderm lineage is determined by positive immunostaining for VEGF receptor II (KDR) or actin α-2 smooth muscle (ACTA2), or can assess the ability of the pluripotent cell to differentiate along ectoderm lineage is determined by positive immunostaining for Nestin or Tubulin β3, or can assess the ability of the pluripotent cell to differentiate along endoderm lineage is determined by positive immunostaining for alpha-feto protein (AFP).

In some embodiments, the assay is a high-throughput assay for assaying a plurality of different pluripotent stem cells, including a plurality of different induced pluripotent stem cells from a subject, such as a human or other mammalian subject.

Another aspect of the present invention relates to the use of the assay as disclosed herein to generate a scorecard from at least one or a plurality of pluripotent stem cell lines.

Another aspect of the present invention relates to a method for generating a pluripotent stem cell scorecard comprising: (i) measuring DNA methylation in a first set of target genes in a plurality of pluripotent stem cell lines; (ii) measuring gene expression in a second set of target genes in the plurality of pluripotent stem cell lines; and (iii) measuring differentiation potential of the plurality of pluripotent stem cell lines. In some embodiment, the method further comprises (iv) calculating an average methylation level for each target gene in the first set of target genes; and (v) calculating an average gene expression level for each target gene in the second set of target genes.

Another aspect of the present invention relates to a scorecard of the performance parameters of a pluripotent stem cell, the scorecard comprising: (i) a first data set comprising the DNA methylation levels for a plurality of DNA methylation target genes from a plurality of pluripotent stem cell lines; (ii) a second data set comprising the gene expression levels for a plurality of gene expression target genes from a plurality of pluripotent stem cell lines; and (iii) a third data set comprising the differentiation propensity levels for differentiation into ectoderm, mesoderm and endoderm lineages from a plurality of pluripotent stem cell lines.

In some embodiments, the scorecard is derived from measuring the DNA methylation levels at least about 500, at least about 1000, at least about 1500, or at least about 200 reference DNA methylation genes, such as any DNA methylation genes from any combination of genes listed in Table 12A or 12C, or selected from Table 13A, Table 13B or Table 14.

In some embodiments, the scorecard is derived from measuring the gene expression levels at least about 500, at least about 1000, at least about 1500, or at least about 200 reference DNA methylation genes, such as any DNA methylation genes from any combination of genes listed in Table 12B or 12C, or selected from Table 13A, Table 13B or Table 14.

In some embodiments, at least the first and/or the second data set are connected to a data storage device, for example, a data storage device which is a database located on a computer device.

In some embodiments, a score card as disclosed herein is determined from a plurality of stem cell lines is at least 5, at least 10, at least 15, or at least 20 pluripotent stem cell lines. In some embodiments, a score card as disclosed herein is determined from one stem cell lines, where each assay is run in triplicate or more. In some embodiments, where a “reference scorecard” is desired, a plurality of stem cell lines for generating a score card comprises at least one pluripotent stem cell line selected from the group consisting of HUES64, HUES3, HUES8, HUES53, HUES28, HUES49, HUES9, HUES48, HUES45, HUES1, HUES44, HUES6, H1, HUES62, HUES65, H7, HUES13, HUES63, HUES66, and any combinations thereof.

In some embodiments, stem cell lines for generating a score card are mammalian pluripotent stem cell lines, e.g., human pluripotent stem cell line, including embryonic stem cells and/or induced pluripotent stem (iPS) cell lines, and/or adult stem cells, or somatic stem cells, or autologous stem cells.

Another aspect of the present invention relates to the use of the scorecard as disclosed herein to distinguish an induced pluripotent stem cell from an embryonic stem cell line.

Another aspect of the present invention relates to a kit for carrying out a method as disclosed herein, where the kit comprises: (i) reagents for measuring DNA methylation status; and (ii) reagents for measuring differentiation propensity of a pluripotent stem cell.

Another aspect of the present invention relates to a computer system for generating a quality assurance scorecard of a pluripotent stem cell, comprising: (i) at least one memory containing at least one program comprising the steps of: (a) receiving DNA methylation data of a set of DNA methylation target genes in the pluripotent stem cell line and performing a comparison of the DNA methylation data with a reference DNA methylation level of the same target genes; (b) receiving differentiation potential data of the pluripotent stem cell line and comparing the differentiation potential data with a reference differentiation potential data; (c) generating a quality assurance scorecard based on the comparison of the DNA methylation data as compared to reference DNA methylation parameters and comparing the differentiation propensity as compared to reference differentiation data; and (ii) a processor for running said program. In some embodiments, the program of the system further comprises (d) receiving gene expression data of a second set of target genes in the pluripotent stem cell line and comparing the expression data with a reference gene expression level of the same second set of target genes; (e) generating a quality assurance scorecard based on the comparison of the DNA methylation data as compared to reference DNA methylation parameters, and the comparison of the differentiation propensity as compared to reference differentiation data, and the comparison of the gene expression data as compared to reference gene expression levels. In some embodiments, the system further comprises a report generating module which generates a stem cell scorecard report based on quality of the pluripotent stem cell line. In some embodiments, the system comprises a memory, wherein the memory comprises a database. In some embodiments, the database arranges the DNA methylation gene set in a hierarchical manner, e.g., the DNA methylated genes ordered in the order of Table 12A or 12B, or selected from Table 13A, Table 13B or Table 14, and the gene expression genes ordered in the order of Table 12B or Table 12C. In some embodiments, a database arranges the propensity to differentiation into different lineages in a hierarchical manner. In some embodiments, the memory is connected to the first computer via a network, e.g., a local network (LAN) or a wide area network, such as the internet, where access to the network is via a secure site or via password access.

In some embodiments, the system as disclosed herein provides a scorecard which provides an indication of suitable uses, utility or applications of the pluripotent stem cell line tested.

Another aspect of the present invention relates to a computer readable medium comprising instructions for generating a quality assurance scorecard of a pluripotent stem cell line, comprising: (i) receiving DNA methylation data of a set of DNA methylation target genes in the pluripotent stem cell line and performing a comparison of the DNA methylation data with a reference DNA methylation level of the same target genes; (ii) receiving differentiation potential data of the pluripotent stem cell line and comparing the differentiation potential data with a reference differentiation potential data; and (iii) generating a quality assurance scorecard based on the comparison of the DNA methylation data as compared to reference DNA methylation parameters and comparing the differentiation propensity as compared to reference differentiation data. In some embodiments, the computer-readable medium further comprises instructions for: (iv) receiving gene expression data of a second set of target genes in the pluripotent stem cell line and comparing the expression data with a reference gene expression level of the same second set of target genes; and (v) generating a quality assurance scorecard based on the comparison of the DNA methylation data as compared to reference DNA methylation parameters, and the comparison of the differentiation propensity as compared to reference differentiation data, and the comparison of the gene expression data as compared to reference gene expression levels.

Another aspect of the present invention relates to a kit for determining the quality of a pluripotent stem cell line, comprising at least two of the following: (i) reagents for measuring methylation status of a plurality of DNA methylation genes, (ii) reagents for measuring gene expression levels of a plurality of genes; and (iii) reagents for measuring the differentiation propensity of the pluripotent stem cell into ectoderm, mesoderm and endoderm lineages.

### Scorecard

One aspect of the present invention relates to a scorecard of the performance parameters of a pluripotent stem cell, the scorecard comprising: (i) a first data set comprising the DNA methylation levels for a plurality of DNA methylation target genes from at least 5 pluripotent stem cell populations; (ii) a second data set comprising the gene expression levels for a plurality of gene expression target genes from at least 5 pluripotent stem cell populations; and (iii) a third data set comprising the differentiation propensity levels for differentiation into ectoderm, mesoderm and endoderm lineages from at least 5 pluripotent stem cell populations. In some embodiments, the plurality of reference DNA methylation genes is at least about 1000 reference DNA methylation genes, or at least about 2000 reference DNA methylation genes or in some embodiments, the DNA methylation status of the whole genome. In some embodiments, the reference DNA methylation genes are any selected from the group comprising cancer gene, oncogenes, and tumor suppressor genes, lineage marker genes and developmental genes.

In some embodiments, the DNA methylation target genes are any, and in any combination of genes selected from the group consisting of: BMP4, CAT, CD14, CXCL5, DAZL, DNMT3B, GATA6, GAPDH, LEFTY2, MEG3, PAX6, S100A6, SOX2, SNAI1, TF. In some embodiments, the DNA methylation target genes is any combination of genes selected from Table 12A or Table 12C, or selected from Table 13A, Table 13B or Table 14. In some embodiments, DNA methylation is determined in promoter regions of the target genes listed in Tables 12A and Table 12C, however the present invention encompasses determining the DNA methylation in all genomic regions (as well as non-genomic regions), including the promoter regions of the genes listed in Table 13A, Table 13B or Table 14. In some embodiments, DNA methylation is determined in any genomic region, or a specific type of genomic region, such as promoters, enhancers, insulator elements, CpG islands, CpG island shores, etc. Additionally, the DNA methylation can be determined in non-coding genes, as well as non-coding transcripts e.g., natural antisense transcripts (NATs), microRNA (miRNAs) genes and all other types of nucleic acid and/or RNA transcripts. In some embodiments, one can also use DNA methylation data to directly derive regions that are highly variable, and DNA sequence data to predict genomic regions that are susceptible to epigenetic alterations. Furthermore, in some embodiments one can use prior knowledge of genes and genomic regions that are involved in cancer, normal and abnormal development and diseases as candidates. In some embodiments, DNA methylation target genes are at least about 200, or at least about 300, or at least about 400, or at least about 500, or at least about 600, or at least about 800, or at least about 1000, or at least about 1500, or at least about 2000, or at least about 3000, or at least about 4000, or at least about 5000 genes, in any combination, selected from the list of genes in Table 12A and/or Table 12C, or selected from Table 13A, Table 13B or Table 14. In some embodiments, the genes are any combination of sets of genes selected with numbers 1-200, or numbers 1-500, or numbers 1-1000 of the genes listed in Table 12A or Table 12C, or selected from Table 13A, Table 13B or Table 14.

In some embodiments, a first and a second data set of the scorecard are connected to a data storage device, such as a data storage device which is a database located on a computer device.

In some embodiments, at least 15 pluripotent stem cells lines are used to generate the first or second or third data set for the scorecard. In some embodiments, the first, second or third data set are obtained from at least 5 or more, or at least 6, or at least 7, or at least 8, or at least 9, or at least 10, or at least 11, or at least 12, or at least 13 or at least 14, or at least 15, or at least 16, or at least 17, or at least 18, or all 19 of the following pluripotent stem cells lines selected from the group; HUES64, HUES3, HUES8, HUES53, HUES28, HUES49, HUES9, HUES48, HUES45, HUES1, HUES44, HUES6, H1, HUES62, HUES65, H7, HUES13, HUES63, HUES66.

In some embodiments, the pluripotent stem cell populations used to generate the data sets for the scorecards are mammalian pluripotent stem cell populations, such as human pluripotent stem cell populations, or induced pluripotent stem (iPS) cell populations, or embryonic stem cell populations, or adult stem cell populations, or autologous stem cell populations, or embryonic stem (ES) stem cell populations.

In some embodiments, the scorecard as disclosed herein can be compared with the DNA methylation levels, gene expression levels and differentiation propensity levels of a pluripotent stem cell population of interest, and can be used to validate and/or predict the behavior of a pluripotent stem cell population by predicting the optimal differentiation along a specific lineage and/or propensity to have undesirable characteristic, e.g., pluripotent stem cell populations which have a predisposition to develop into cancer cells. Thus, in some embodiments, the scorecard can be used in methods to select for, e.g., positive selection pluripotent stem cell population of interest with desirable characteristics (e.g., high differentiation potential along a specific lineage), and/or to negatively select, e.g., identify and discard, cells with undesirable characteristics, e.g., cells with a predisposition to develop into cancer cells.

In some embodiments, a pluripotent stem cell line which has a DNA methylation level of a target gene which is statistically significant (FDR<5%) and/or an absolute difference of >20% points of level of DNA methylation as compared to the normal variation of DNA methylation for that gene (e.g., the normal reference value) in a pluripotent stem cell would be considered an epigenetic outlier DNA methylation gene. A pluripotent stem cell which has numerous, e.g., at least about 5, or at least about 6, or at least about 7, or at least about 8, or at least about 5-10, or at least about 10-15, or at least about 10-50, or at least about 50-100, or at least about 100-150, or at least about 150-200 or more than 200 total epigenetic outlier DNA methylation genes as compared to a reference pluripotent stem cell will be considered an outlier pluripotent stem cell. Accordingly, such a pluripotent stem cell can be used to negatively select, e.g., isolate and discard the cells with undesirable characteristics.

In some embodiments, a pluripotent stem cell line which has a DNA methylation level of a target cancer gene which is statistically significant (FDR<5%) and/or an absolute difference of >20% points of level of DNA methylation as compared to the normal variation of DNA methylation for that target cancer gene (e.g., the normal reference DNA methylation level for a cancer gene) in a pluripotent stem cell would be considered an epigenetic outlier DNA methylation cancer gene. A pluripotent stem cell which has numerous, e.g., at least about 5, or at least about 6, or at least about 7, or at least about 8, or at least about 5-10, or at least about 10-15, or at least about 10-50, more than 50 total epigenetic outlier DNA methylation cancer genes as compared to a reference pluripotent stem cell will be considered an outlier pluripotent stem cell. Accordingly, such a pluripotent stem cell can be used to negatively select, e.g., isolate and discard the cells with undesirable characteristics, such as an increase or decrease in DNA methylation of a cancer gene.

In some embodiments, a pluripotent stem cell line which has a gene expression level of a target gene which is statistically significant (FDR<10%) and/or an absolute difference of >1 log-2 fold change of level of gene expression as compared to the normal variation of gene expression for that gene (e.g., the normal reference value) in a pluripotent stem cell would be considered a gene expression outlier gene. A pluripotent stem cell which has numerous, e.g., at least about 5, or at least about 6, or at least about 7, or at least about 8, or at least about 5-10, or at least about 10-15, or at least about 10-50, or at least about 50-100 or more total outlier gene expression genes as compared to a reference pluripotent stem cell will be considered an outlier pluripotent stem cell. Accordingly, such a pluripotent stem cell can be used to negatively select, e.g., isolate and discard the cells with undesirable characteristics.

In some embodiments, a pluripotent stem cell line which has a gene expression level of a lineage gene which is statistically significant (FDR<5%) and/or an absolute difference of >1 log-2 fold change of level of lineage gene expression as compared to the normal variation of gene expression for that lineage gene (e.g., the normal reference value) in a pluripotent stem cell would be considered a differentiation outlier gene. A pluripotent stem cell which has numerous, e.g., at least about 5, or at least about 6, or at least about 7, or at least about 8, or at least about 5-10, or at least about 10-15, or at least about 10-50, or at least about 50-100 or more total outlier lineage gene expression genes as compared to a reference pluripotent stem cell will be considered an outlier pluripotent stem cell, which may not differentiate along the same lineages as a reference pluripotent stem cell line. Accordingly, such a pluripotent stem cell can be used to negatively select, e.g., isolate and discard the cells with undesirable characteristics, e.g., cells which may not differentiate along particular lineages.

### Method for Generating a Scorecard of a Preferred Pluripotent Stem Cell

Another aspect of the present invention relates to a method for generating a pluripotent stem cell score card comprising: (i) measuring DNA methylation in a set of target genes in a plurality of pluripotent stem populations; (ii) measuring gene expression in a second set of target genes in the plurality of pluripotent stem cell lines; and (iii) measuring differentiation potential of the plurality of pluripotent stem cell lines. In some embodiments, the method to generate a pluripotent stem cell score card can be used to generate a scorecard comprising the values of normal variations of DNA methylation, normal variation of DNA gene expression and normal differentiation propensity from a plurality of pluripotent stem cell lines, for example, at least 5, or at least 6, or at least 7, or at least 8, or at least 9, or at least 10, or at least 15, or at least 20, or a least 30, or at least 40 or more than 40 different pluripotent stem cell populations.

### Assays

Another aspect of the present invention relates to an assay for characterizing a plurality of properties of a pluripotent cell, the assay comprising at least 2 of the following: (i) a DNA methylation assay; (ii) a gene expression assay; and (iii) a differentiation assay.

In some embodiments, the DNA methylation assay is a bisulfite sequencing assay, or a whole genome sequencing assay, e.g., a reduced-representation bisulfite sequencing (RRBS). In some embodiments, a DNA methylation assay is enrichment-based DNA methylation assay (e.g. MeDIP) or restriction-enzyme base DNA methylation assay (e.g. CHARM or HELP), or other means of DNA methylation assays as disclosed herein and in the Examples. In some embodiments, DNA methylation assay the DNA methylation assay is an Illumina Methylation Assay. In some embodiments, the gene expression assay is a microarray assay.

In some embodiments, the differentiation propensity assay a quantitative differentiation assay, e.g., a differentiation assay which can assess the ability of the pluripotent cell to differentiate into at least one of the following lineages; mesoderm, endoderm and ectoderm, neuronal hematopoietic lineages. In some embodiments, the ability of the pluripotent cell to differentiate into at least one of the following lineages; mesoderm, endoderm and ectoderm is determined by gene expression profiling on embryoid bodies (EBs) in combination with a bioinformatic algorithm to assess differentiation propensity, where the level of gene expression of lineage genes, as disclosed in Table 7 herein is determined, and a statistically significant difference (FDR<5%) change in level of gene expression, and/or a >1 log-2 fold change in the level of gene expression of a lineage marker gene will indicate a propensity to differentiate along a different lineage as compared to a reference pluripotent stem cell line. In alternative embodiments, the ability of the pluripotent cell to differentiate into at least one of the following lineages; mesoderm, endoderm and ectoderm is determined by immunostaining or FAC sorting using an antibody to at least one marker for mesoderm, endoderm and ectoderm lineages. In some embodiments, the ability of the pluripotent cell to differentiate into at least one of the following lineages; mesoderm, endoderm and ectoderm is determined by immunostaining the pluripotent stem cell after at least about 7 days in EB. Examples of lineage markers for mesoderm, endoderm and ectoderm lineages are well know by persons of ordinary skill in the art, and include but are not limited to mesoderm lineage markers VEGF receptor II (KDR) or actin α-2 smooth muscle (ACTA2), ectoderm lineage markers Nestin or Tubulin β3 and endoderm lineage markers alpha-feto protein (AFP).

In some embodiments, the assay is a high-throughput assay for assaying a plurality of different pluripotent stem cells, for example, enabling one to assess a plurality of different induced pluripotent stem cells derived from reprogramming a somatic cell obtained from the same or a different subject, e.g., a mammalian subject or a human subject.

In some embodiments, the assay as disclosed herein can be used to generate a scorecard as disclosed herein from at least one, or a plurality of pluripotent stem cell populations.

### Epigenetic Mapping

While not wishing to be bound by theory, epigenetic events play a significant role in the expression of genes, and are important in development and progression of cancer. Epigenetic changes such as DNA methylation act to regulate gene expression in normal mammalian development. Promoter hypermethylation also plays a major role in cancer through transcriptional silencing of critical growth regulators such as tumor suppressor genes. Loss of function of genes, such as tumor suppressor genes can occur through epigenetic changes such as DNA methylation. The term “epigenetics” refers to heritable changes in gene expression that do not result from alterations in the gene nucleotide sequence. For example, when DNA is methylated in the promoter region of genes, where transcription is initiated, genes are inactivated and silenced. Epigenetic modification includes for example, without limitation, DNA methylation, posttranslational modification of chromatin, small non-coding RNA's, and non-covalent structural modifications to chromatin, such as condensation and decondensation of chromatin. In some instances, epigenetic modification can also be in the form of posttranslational modification (PTM) of proteins, including, DNA methylation, ubiquitination, phosphorylation, glycosylation, sumoylation, acetylation, S-nitrosylation or nitrosylation, citrullination or deimination, neddylation, OClcNAc, ADP-ribosylation, hydroxylation, fattenylation, ufmylation, prenylation, myristoylation, S-palmitoylation, tyrosine sulfation, formylation, and carboxylation.

In some embodiments of the methods, systems and kits of the present invention, the level of epigenetic modification is determined in a pluripotent stem cell line of interest. In some embodiments, the epigenetic modification is DNA methylation. In some embodiments, methylation of a DNA methylation target genes is determined. Accordingly, in some embodiments a DNA methylation target gene is any gene where is desirable to determine the repression (e.g., epigenetic silencing) of the expression of the gene. In some embodiments, the DNA methylation target gene is a cancer gene, e.g., an oncogene or a tumor suppressor gene. In some embodiments, the DNA methylation target gene is a developmental gene, and in some embodiments, the DNA methylation target gene is a lineage marker gene.

In some embodiments, the DNA methylation is determined or measured any gene selected from the group of BMP4, CAT, CD14, CXCL5, DAZL, DNMT3B, GATA6, GAPDH, LEFTY2, MEG3, PAX6, S100A6, SOX2, SNAI1, TF. In some embodiments, the DNA methylation is a gene with variable DNA methylation levels, such as DAZL, LEFTY2, CXCL5, MEG3, S100A6, CAT, TF, CD14. In some embodiments, the DNA methylation is a gene which has low DNA methylation variability, such as: PAX6, DNMT3B, GATA6, GAPDH, SOX2, SNAI1, BMP4.

In some embodiments, the DNA methylation is determined or measured in a set of reference DNA methylation target genes, where the DNA methylation reference genes can be cancer genes, and/or developmental genes, and are disclosed in Tables 12A. In some embodiments, the genes used in a first set of reference DNA methylation genes are at least about 200, or at least about 300, or at least about 400, or at least about 500, or at least about 600, or at least about 800, or at least about 1000, or at least about 1500, or at least about 2000, or at least about 3000, or at least about 4000, or at least about 5000 genes, in any combination, selected from the list of genes in Table 12A and/or Table 12C, or selected from Table 13A, Table 13B or Table 14. In some embodiments, the genes are any combination of sets of genes selected with numbers 1-200, or numbers 1-500, or numbers 1-1000 of the genes listed in Table 12A or Table 12C, or selected from Table 13A, Table 13B or Table 14.

In some embodiments, the DNA methylation is measured in at least 50 genes, or at least 100 genes, in any combination of the following 140 gene set: PON3; CD14; PEG3AS; CRCT1, LCE5A; HIST1; H2BB; HIST1; H3C, CRCT1, LCE5A, PTK2B, TF, CAT, SLC38A11, ZNF528, CALCB, ERAS, INGX, TMPRSS12, ZNF248, ZNF876P, SLC17A3, TDRD5, LCE3A, ASB3, GPR75, ZNF354C, PEG3AS, KAAG1, PCDHA2, HPDL, ZNF737, AGBL2, COMT, TXNRD2, SLC30A8, H2AFZP1, CTSF, ZNF833, S100A5, S100A6, PRDM9, CYP2E1, ZNF177, CR1L, ZNF572, MOS, FAM70A, GPS, PAPOLB, ZDHHC15, HSF5, CDX4, GOLGA8B, KLF8; ARMCX5; CBLN4, POU3F4, LYNX1, DENND2D, CYP2E1, ZNF562, PPYR1, KLHL34, ZNF562, TMLHE, CCDC11, GYG2P, TCEAL2, ZNF454, ZNF667, TRIM4, FAM24B, ZNF397OS, PAQR6, DENND2D, LYNX1, BHMT2, DMGDH, PF4, LTF, NAP1L6, ALOX15B, CES1, PPP1R13L, COMT, TXNRD2, LYNX1, DNAJC15, ARMCX1, TRPM2, GOLGA8A, ZPBP, ZNF630, BHMT2, DMGDH, SLC7A3, SLFN13, PLEK2, DYNLT3, SLC2A14, SPATS1, SLCO1A2, TCEAL6, SLC2A14, TAF9B, KIAA1210, CNTD2, PLD6, CFLAR, PHF8, TBPL2, RWDD2B, DEFB124, REM1, TCEAL6, CD14, BCL2L10, ZNF630, DCDC2, CRYGD, ZNF440, RFPL2, MYCL2, TRPM2, MEG3, TEKT4, FAM104B, EDNRB, OSGIN1, NKAP, NROB1, SPIN3, NDUFA1, RNF113A, ZNF726, ZNF502 and C3orf62.

As the function(s) of many genes are now known, one can assign putative effects to the differential expression and/or DNA methylation of cancer genes, such as increased or decreased cancer risk, differences in the ability to differentiate into specific cell types and lineages, resistance against drugs and the general usefulness for disease modeling, drug screening and regenerative therapies.

Cancer cells contain extensive aberrant epigenetic alterations, including promoter CpG island DNA hypermethylation and associated alterations in histone modifications and chromatin structure. Aberrant epigenetic silencing of tumor-suppressor genes in cancer involves changes in gene expression, chromatin structure, histone modifications and cytosine-5 DNA methylation.

Accordingly, in some embodiments, the DNA methylation target genes include cancer genes, e.g., oncogenes and tumor suppressor genes, and developmental genes, as well as lineage marker genes. For instance, where the presence of hypermethylation of a promoter of an oncogene is detected, it would indicate that epigenetic silencing has occurred and that the oncogene is repressed or permanently silenced, and may be a desirable characteristic. However, a decreased level of methylation would indicate the absence of epigenetic silencing and that the oncogene could be expressed, which may indicate that the pluripotent stem cell is predisposed to self-renewal and high potential for malignant transformation. Similarly, where the cancer gene is a tumor suppressor gene, the presence of hypermethylation promoter or a statistically significant high level of methylation as compared to the normal variation of methylation for that tumor suppressor gene, it would indicate epigenetic silencing and that the expression of the tumor suppressor is permanently repressed, indicating that the pluripotent stem cell is predisposed to continual self-renewal and high potential malignant transformation. Accordingly, the methylation status of oncogenes and/or tumor suppressor genes can be used to predict if a pluripotent stem cell is predisposed to continual self-renewal and high potential malignant transformation. Furthermore, in some embodiments the DNA methylation level is measured and determined in a set of cancer genes, e.g., oncogenes and tumor suppressor genes enables one to predict if the pluripotent stem cell predisposed to continual self-renewal and high potential malignant transformation.

In alternative embodiments, the DNA methylation level is measured and determined in a set of lineage-specific (e.g., lineage marker genes) or developmental-specific genes, which enables one to predict if the pluripotent stem cell can differentiate along specific developmental pathways or into a cell type which expresses the lineage marker.

Importantly, in the differentiation propensity assay and methods as disclosed herein, the DNA methylation level in a set of lineage-specific (e.g., lineage marker genes) or developmental-specific genes is determined after a pluripotent stem cell line has been cultured and allowed to spontaneously differentiate for a pre-defined period of time, where the results from a DNA methylation assay of a set of lineage marker genes enables one to predict the lineage differentiation bias of the pluripotent stem cell line. In some embodiments of the differentiation propensity assay, a DNA methylation assay of a set of lineage marker genes is performed on the pluripotent stem cell line after directed differentiation along a particular lineage.

In instances where the methylation target gene is a developmental gene or a lineage marker gene, the presence of hypermethylation of a gene promoter, or a statistically significant high level of DNA methylation as compared to the normal variation of DNA methylation for that developmental gene or lineage marker gene indicates epigenetic silencing and that the expression of the developmental gene or lineage marker is permanently repressed, indicating that the pluripotent stem cell is predisposed not to express the developmental gene and/or lineage marker and therefore is predicted not to differentiate along the developmental pathway the developmental gene or differentiate into a cell type which expresses the lineage marker. In alternative situations, where the methylation level of developmental gene or a lineage marker gene in the pluripotent stem cell is within the normal variation for the level of methylation for that gene can be used to predict that a pluripotent stem cell will be able to proceed to differentiate along the developmental pathway the developmental gene or differentiate into a cell type which expresses the lineage marker. Accordingly, the methylation status of developmental genes and/or lineage markers can be used to predict if a pluripotent stem cell can differentiate along specific developmental pathways or into a cell type which expresses the lineage marker.

While the measurement of DNA methylation as described above focuses mostly on the effect of single genes, in some embodiments, the scorecard measures the DNA methylation in a combination of data for multiple genes, e.g., multiple genes in “cancer gene” sets, or multiple genes in “lineage marker gene” sets, for example, to predict a cell line's quality (e.g., likely to develop into a cancerous line) and utility (e.g., likely to differentiate, or not, along specific lineages of interest). Accordingly, one can select specific sets of DNA methylation target genes to develop a “customized scorecard” for sensitive and accurate characterization of a pluripotent stem cell line to identify particular desired or undesirable characteristics. This is one of the key advantages of use of the scorecard as disclosed herein to determine the quality and utility of a particular pluripotent stem cell line.

In some embodiments of the present invention, the DNA methylation status is identified in PRC2 genes, as well as other transcription factors of the Dlx, Irx, Lhx and Pax gene families (which are involved in neurogenesis, hematopoiesis and axial patterning), or the Fox, Sox, Gata and Tbx families (which are involved in developmental processes)).

As discussed herein, in some embodiments a pluripotent stem cell line which has a DNA methylation level of a target gene which is statistically significant (FDR<5%) and/or an absolute difference of >20 percentage points of level of DNA methylation as compared to the normal variation of DNA methylation for that gene (e.g., the normal reference value) in a pluripotent stem cell would be considered an epigenetic outlier DNA methylation gene. A pluripotent stem cell which has numerous, e.g., at least about 5, or at least about 6, or at least about 7, or at least about 8, or at least about 5-10, or at least about 10-15, or at least about 10-50, or at least about 50-100, or at least about 100-150, or at least about 150-200 or more than 200 total epigenetic outlier DNA methylation genes as compared to a reference pluripotent stem cell will be considered an outlier pluripotent stem cell. Accordingly, such a pluripotent stem cell can be used to negatively select, e.g., isolate and discard the cells with undesirable characteristics.

In some embodiments, a pluripotent stem cell line which has a DNA methylation level of a target cancer gene which is statistically significant (FDR<5%) and/or an absolute difference of >20% points of level of DNA methylation as compared to the normal variation of DNA methylation for that target cancer gene (e.g., the normal reference DNA methylation level for a cancer gene) in a pluripotent stem cell would be considered an epigenetic outlier DNA methylation cancer gene. A pluripotent stem cell which has numerous, e.g., at least about 5, or at least about 6, or at least about 7, or at least about 8, or at least about 5-10, or at least about 10-15, or at least about 10-50, more than 50 total epigenetic outlier DNA methylation cancer genes as compared to a reference pluripotent stem cell will be considered an outlier pluripotent stem cell. Accordingly, such a pluripotent stem cell can be used to negatively select, e.g., isolate and discard the cells with undesirable characteristics, such as an increase or decrease in DNA methylation of a cancer gene.

### DNA Methylation Methods and Assays

One can use any method to measure DNA methylation which is commonly known to persons of ordinary skill in the art, including, but not limited to, enrichment-based methods (e.g. MeDIP, MBD-seq and MethylCap), bisulfite-based methods (e.g. RRBS, bisulfite sequencing, Infinium, GoldenGate, COBRA, MSP, MethyLight) and restriction-digestion methods (e.g., MRE-seq). In one embodiment, a method for epigenetic profiling and epigenetic mapping is whole genome epigenetic mapping. One can use any method for epigenetic mapping of a pluripotent stem cell line known to one of ordinary skill in the art, and includes, for example reduced-representation bisulfite sequencing (RRBS), as well as methods disclosed in U.S. Patent Application US2010/0172880, which is incorporated herein in its entirety by reference. Other DNA methylation assays are disclosed in U.S. Application US2008/0213789 and US2010/0075331 and in U.S. Pat. Nos. 6,960,434 and 7,425,415, which are incorporated herein in their entirety by reference. Method for measuring DNA methylation of pluripotent stem cells is also described in “Genome-wide mapping of DNA methylation: a quantitative technology comparison” by Bock et al., which is incorporated herein in its entirety by reference, where the inventors evaluated a variety of DNA methylation methods (MeDIP-seq: methylated DNA immunoprecipitation, MethylCap-seq: methylated DNA capture by affinity purification, RRBS: reduced representation bisulfite sequencing, and the Infinium HumanMethylation assay) produce accurate DNA methylation data of pluripotent stem cells.

In some embodiments, the DNA methylation assays are species-specific, so the use of mouse embryonic fibroblasts as a feeder layer for human pluripotent stem cells will not interfere with the epigenetic analysis.

Several methods have been developed to enable DNA methylation profiling on a genomic scale. Most of these methods combine DNA analysis by microarrays or high-throughput sequencing with one of four ways of translating DNA methylation patterns into DNA sequence information or library enrichment: (i) Methylated DNA immunoprecipitation (MeDIP) uses an antibody that is specific for 5-methyl-cytosine to retrieve methylated fragments from sonicated DNA11, (ii) Methylated DNA capture by affinity purification (MethylCap) employs a methyl-binding domain protein to obtain DNA fractions with similar methylation levels. (iii) Bisulfite-based methods utilize a chemical reaction that selectively converts unmethylated (but not methylated) cytosines into uracils, thus introducing methylation-specific single-nucleotide polymorphisms into the DNA sequence. (iv) Methylation-specific digestion uses prokaryotic restriction enzymes to fractionate DNA in a methylation-specific way.

Four popular methods, with a special emphasis on their practical utility for biomedical research and biomarker development were assessed previously by the inventors, which included MeDIP-seq, MethylCap-seq, RRBS and the Infinium HumanMethylation assay, (see “Genome-wide mapping of DNA methylation: a quantitative technology comparison” by Bock et al.). These methods are useful in the methods, systems and assays of the present invention, based on the following considerations: (i) All four methods are relatively easy to set up because detailed protocols have been published and/or commercial kits are available. (ii) RRBS has an advantage over other genome-wide bisulfite sequencing because its per-sample cost are comparable to the other methods and realistic for large sample sizes. (iii) The Infinium HumanMethylation assay is useful in the methods, systems and assays as disclosed herein because of its wide use and easy integration with existing genotyping pipelines; and is also a microarray-based method. In some embodiments, other DNA methylation methods that utilize microarrays and or Methylation-specific digestion can be used in the methods, systems and assays as disclosed herein, as these have been benchmarked previously. The methods for performing these assays and the analysis of the date is disclosed herein in the Examples, in the Methods section under the subtitle “Other DNA methylation mapping methods”.

A large number of different epigenetic profiling technologies have been developed (e.g., Laird, P. W. Hum Mol Genet. 14, R65-R76, 2005; Laird, P. W. Nat Rev Cancer 3, 253-66, 2003; Squazzo, S. L. et al. Genome Res 16, 890-900, 2006; and Lieb, J. D. et al. Cytogenet Genome Res 114, 1-15, 2006, all incorporated by reference herein). These can be divided broadly into chromatin interrogation techniques, which rely primarily on chromatin immunoprecipitation with antibodies directed against specific chromatin components or histone modifications, and DNA methylation analysis techniques. Chromatin immunoprecipitation can be combined with hybridization to high-density genome tiling microarrays (ChIP-Chip) to obtain comprehensive genomic data. However, chromatin immunoprecipitation is not able to detect epigenetic abnormalities in a small percentage of cells, whereas DNA methylation analysis has been successfully applied to the highly sensitive detection of tumor-derived free DNA in the bloodstream of cancer patients (Laird, P. W. Nat Rev Cancer 3, 253-66, 2003). Preferably, a sensitive, accurate, fluorescence-based methylation-specific PCR assay (e.g., METHYLIGHT™) is used, which can detect abnormally methylated molecules in a 10,000-fold excess of unmethylated molecules (Eads, C A. et al., Nucleic Acids Res 28, E32, 2000), or an even more sensitive variation of METHYLIGHT™ that allows detection of a single abnormally methylated DNA molecule in a very large volume or excess of unmethylated molecules. In particular aspects, METHYLIGHT™ analyses are performed as previously described by the present applicants {e.g., Weisenberger, D J. et al. Nat Genet. 38:787-793, 2006; Weisenberger et al., Nucleic Acids Res 33:6823-6836, 2005; Siegmund et al., Bioinformatics 25, 25, 2004; Eads et al., Nucleic Acids Res 28, E32, 2000; Virmani et al., Cancer Epidemiol Biomarkers Prey 11:291-297, 2002; Uhlmann et al., Int J Cancer 106:52-9, 2003; Ehrlich et al., Oncogene 25:2636-2645, 2006; Eads et al., Cancer Res 61:3410-3418, 2001; Ehrlich et al., Oncogene 21; 6694-6702, 2002; Marjoram et al., BMC Bioinformatics 7, 361, 2006; Eads et al., Cancer Res 60:5021-5026, 2000; Marchevsky et al., /Mol Diagn 6:28-36, 2004; Sarter et al., Hum Genet. 117:402-403, 2005; Trinh et al., Methods 25:456-462, 2001; Ogino et al., Gut 55:1000-1006, 2006; Ogino et al., J Mol Diagn 8:209-217, 2006, and Woodson, K. et al. Cancer Epidemiol Biomarkers Prey 14:1219-1223, 2005).

High-throughput Illumina platforms, for example, can be used to screen PRC2 targets (or other targets) for aberrant DNA methylation in a large collection of human ES cell DNA samples (or other derivative and/or precursor cell populations), and then METHYLIGHT™ and METHYLIGHT™ variations can be used to sensitively detect abnormal DNA methylation at a limited number of loci {e.g., in a particular number of cell lines during cell culture and differentiation).

Illumina DNA Methylation Profiling. Illumina, Inc. (San Diego) has recently developed a flexible DNA methylation analysis technology based on their GOLDENGATE™ platform, which can interrogate 1,536 different loci for 96 different samples on a single plate (Bibikova, M. et al. Genome Res 16:383-393, 2006). Recently, Illumina reported that this platform can be used to identify unique epigenetic signatures in human embryonic stem cells (Bibikova, M. et al. Genome Res 16:1075-83, 200)). Therefore, Illumina analysis platforms are preferably used. High-throughput Illumina platforms, for example, can be used to screen PRC2 targets (or other targets) for aberrant DNA methylation in a large collection of human ES cell DNA samples (or other derivative and/or precursor cell populations), and then MethyLight and MethyLight variations can be used to sensitively detect abnormal DNA methylation at a limited number of loci {e.g., in a particular number of cell lines during cell culture and differentiation).

There is extensive experience in the analysis and clustering of DNA methylation data, and in DNA methylation marker selection that can be preferably used (e.g., Weisenberger, D J. et al. Nat Genet. 38:787-793, 2006; Siegmund et al., Bioinformatics 25, 25, 2004; Virmani et al. Cancer Epidemiol Biomarkers Prey 11:291-297, 2002; Marjoram et al., Bioinformatics 7, 361, 2006); Siegmund et al., Cancer Epidemiol Biomarkers Prey 15:567-572, 2006); and Siegmun & Laird, Methods 27:170-178, 2002, all incorporated herein by reference). For example, stepwise strategies {e.g., Weisenberger et al., Nat Genet 38:787-793, 2006, incorporated herein) are used as taught by the methods exemplified herein to provide DNA methylation markers that are targets for oncogenic epigenetic silencing in ES cells.

By way of example only, a methylation assay can be conducted by a service provider, e.g. epigenomics (Berlin) and other service providers. Briefly, after quality control was performed on the samples, genomic DNA is treated with sodium bisulphite. PCR primers were designed for the regions of interest in the specified genes. The selected genes of interest, e.g., DNA methylation target genes, such as those listed in Table 12A and/or Table 12C, or any gene selected from Table 13A, Table 13B or Table 14 are assessed. For example, if one DNA methylation target gene to be assessed is POU5F1 (annotated OCT4 orthologous human gene) and NANOG genes: POU5F1 gene (reference sequence: NM.sub.-002701) AMP1000122 located at the 59 UTR of the annotated Ensembl transcript POUF1_HUMAN (ENST00000259915), 150 bp upstream of the TSS. NANOG gene (reference sequence: NM.sub.-024865) AMP1000123 located at the 59 UTR of the annotated Ensembl transcript NANOG_HUMAN (ENST00000229307), 25 by upstream of the TSS. The following bisulphite primers can be used for PCR and for sequencing: POU5F1 5′-ATGGTGTTTGTGGAAGGGG-AA-3′ (SEQ ID NO: 1) and 5′-TCCAAACAACTAAAATATACAAAACCT-3′ (SEQ ID NO: 2); NANOG 5′-TAATATGAGGTAATTAGTTTAGTTTAGT-3′ (SEQ ID NO: 3) and 5′-TAATTTCAAACTCTAACTTCAAATAAT-3′ (SEQ ID NO: 4).

### Gene Expression Profiling

In some embodiments, the assays, systems and methods comprise a quantitative gene profiling assay, such as a microarray or the like. Any method for determining gene expression levels commonly known to persons of ordinary skill in the art are encompassed for use in the methods, systems and assays as disclosed herein, and include Affymetrix microarray methods, and other methods to measure DNA or transcript expression. In some embodiments, gene expression is measured using cDNA and RNA sequencing, imaging-based methods such as NanoString and a wide range of methods that use PCR as well as qPCR. Normalization for these methods has been widely described. The inventors have used the gcRMA algorithm for normalizing Affymetrix microarray data.

In some embodiments, the gene expression level is measured in a set of gene expression target genes, where the gene expression target genes can be cancer genes, and/or developmental genes, and are disclosed in Tables 12B. In some embodiments, the which are measured in the methods, systems and assays of the invention are a set of gene expression target genes are at least about 200, or at least about 300, or at least about 400, or at least about 500, or at least about 600, or at least about 800, or at least about 1000, or at least about 1500, or at least about 2000, or at least about 3000, or at least about 4000, or at least about 5000 genes, in any combination, selected from the list of genes in Table 12B and/or Table 12C, or selected from the list of genes listed in Table 13A, Table 13B or Table 14. In some embodiments, the genes are any combination of sets of genes selected with numbers 1-200, or numbers 1-500, or numbers 1-1000 of the genes listed in Table 12B or Table 12C, or selected from the list of genes listed in Table 13A, Table 13B or Table 14.

In some embodiments, the DNA methylation is measured in at least 50 genes, or at least 100 genes, in any combination of the following 134 gene set: PON3, CD14, PEG3AS, CRCT1, LCE5A, HIST1, H2BB, HIST1, H3C, CRCT1, LCE5A, PTK2B, TF, CAT, SLC38A11, ZNF528, CALCB, ERAS, INGX, TMPRSS12, ZNF248, ZNF876P, SLC17A3, TDRD5, LCE3A, ASB3, GPR75, ZNF354C, PEG3AS, KAAG1, PCDHA2, HPDL, ZNF737, AGBL2, COMT, TXNRD2, SLC30A8, H2AFZP1, CTSF, ZNF833, S100A5, S100A6, PRDM9, CYP2E1, ZNF177, CR1L, ZNF572, MOS, FAM70A, GPS, PAPOLB, ZDHHC15, HSF5, CDX4, GOLGA8B, KLF8, ARMCX5, CBLN4, POU3F4, LYNX1, DENND2D, CYP2E1, ZNF562, PPYR1, KLHL34, ZNF562, TMLHE, CCDC11, GYG2P, TCEAL2, ZNF454, TRIM4, FAM24B, ZNF397OS, PAQR6, DENND2D, LYNX1, BHMT2, DMGDH, PF4, LTF, NAP1L6, ALOX15B, CES1, PPP1R13L, COMT, TXNRD2, LYNX1, DNAJC15, ARMCX1, TRPM2, GOLGA8A, ZPBP, ZNF630, BHMT2, DMGDH, SLC7A3, SLFN13, PLEK2, DYNLT3, SLC2A14, SPATS1, SLCO1A2, TCEAL6, SLC2A14, TAF9B, KIAA1210, CNTD2, PLD6, CFLAR, PHF8, TBPL2, RWDD2B, DEFB124, REM1, TCEAL6, BCL2L10, ZNF630, DCDC2, CRYGD, ZNF440, RFPL2, MYCL2, TRPM2, MEG3, TEKT4, FAM104B, EDNRB, OSGIN1, NKAP, NROB1, SPIN3, SPIN3, NDUFA1, RNF113A, ZNF726.

In alternative embodiments, gene expression is measured and determined in a set of lineage-specific (e.g., lineage marker genes) or developmental-specific genes, which enables one to predict if the pluripotent stem cell can differentiate along specific developmental pathways or into a cell type which expresses the lineage marker.

Importantly, in the differentiation propensity assay and methods as disclosed herein, the level of gene expression of a set of lineage-specific (e.g., lineage marker genes) or developmental-specific genes is determined after a pluripotent stem cell line has been cultured and allowed to spontaneously differentiate for a pre-defined period of time, where the results from a gene expression assay of a set of lineage marker genes enables one to predict the lineage differentiation bias of the pluripotent stem cell line. In some embodiments of the differentiation propensity assay, a gene expression assay of a set of lineage marker genes is performed on the pluripotent stem cell line after directed differentiation along a particular lineage.

In instances where the gene expression target gene is a developmental gene or a lineage marker gene, a high level of expression, and/or a statistically significant high level of DNA methylation as compared to the normal variation of level of gene expression for that developmental gene or lineage marker gene indicates that the expression of the developmental gene or lineage marker is increased and indicates that the pluripotent stem cell is predisposed to differentiate along the developmental pathway the developmental gene or differentiate into a cell type which expresses the lineage marker. Similarly, in situations where the gene expression level of developmental gene or a lineage marker gene in the pluripotent stem cell is within the normal variation for the level of gene expression for that gene, the information can be used to predict that a pluripotent stem cell will be able to proceed to differentiate along the developmental pathway the developmental gene or differentiate into a cell type which expresses the lineage marker. Accordingly, the gene expression level of developmental genes and/or lineage markers can be used to predict if a pluripotent stem cell can differentiate along specific developmental pathways or into a cell type which expresses the lineage marker.

While the measurement of gene expression as described above focuses mostly on the effect of single genes, in some embodiments, the scorecard measures the gene expression of a combination of gene expression target genes (e.g., any combination of genes listed in Tables 12A and/or 12C), e.g., multiple genes in “cancer gene” sets, or multiple genes in “lineage marker gene” sets, for example, to predict a cell line's quality (e.g., likely to develop into a cancerous line) and utility (e.g., likely to differentiate, or not, along specific lineages of interest). Accordingly, one can select specific sets of gene expression target genes to develop a “customized scorecard” for sensitive and accurate characterization of a pluripotent stem cell line to identify particular desired or undesirable characteristics. This is one of the key advantages of use of the scorecard as disclosed herein to determine the quality and utility of a particular pluripotent stem cell line.

As discussed herein, in some embodiments a pluripotent stem cell line which has a gene expression level of a target gene which is statistically significant (FDR<10%) and/or an absolute difference of >1 log-2 fold change of level of gene expression as compared to the normal variation of gene expression for that gene (e.g., the normal reference value) in a pluripotent stem cell would be considered a gene expression outlier gene. A pluripotent stem cell which has numerous, e.g., at least about 5, or at least about 6, or at least about 7, or at least about 8, or at least about 5-10, or at least about 10-15, or at least about 10-50, or at least about 50-100 or more total outlier gene expression genes as compared to a reference pluripotent stem cell will be considered an outlier pluripotent stem cell. Accordingly, such a pluripotent stem cell can be used to negatively select, e.g., isolate and discard the cells with undesirable characteristics.

Gene Expression Assays

In some embodiments, gene expression is determined on any gene level, for example, the expression of non-coding genes, as well as non-coding transcripts e.g., natural antisense transcripts (NATs), microRNA (miRNAs) genes and all other types of nucleic acid and/or RNA transcripts that are normally or abnormally present in pluripotent and differentiated cells.

In some embodiments, where the level of gene expression measured is the level of gene transcript expression measured, protein expression gene transcript expression can be measured at the level of messenger RNA (mRNA). In some embodiments, detection uses nucleic acid or nucleic acid analogues, for example, but not limited to, nucleic acid analogous comprise DNA, RNA, PNA, pseudo-complementary DNA (pcDNA), locked nucleic acid and variants and homologues thereof. In some embodiments, gene transcript expression can be assessed by reverse-transcription polymerase-chain reaction (RT-PCR) or quantitative RT-PCR by methods commonly known by persons of ordinary skill in the art.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

In an alternative embodiment, a gene expression target gene can be determined by reverse-transcription (RT) PCR and by quantitative RT-PCR (QRT-PCR) or real-time PCR methods. Methods of RT-PCR and QRT-PCR are well known in the art, and are described in more detail below.

Real time PCR is an amplification technique that can be used to determine levels of mRNA expression. (See, e.g., Gibson et al., Genome Research 6:995-1001, 1996; Heid et al., Genome Research 6:986-994, 1996). Real-time PCR evaluates the level of PCR product accumulation during amplification. This technique permits quantitative evaluation of mRNA levels in multiple samples. For mRNA levels, mRNA is extracted from a biological sample, e.g. a tumor and normal tissue, and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes can be obtained commercially from, for example, Perkin Elmer/Applied Biosystems (Foster City, Calif.). To quantitate the amount of the specific nucleic acid of interest in a sample, a standard curve is generated using a control. Standard curves can be generated using the Ct values determined in the real-time PCR, which are related to the initial concentration of the nucleic acid of interest used in the assay. Standard dilutions ranging from 10-106 copies of the gene of interest are generally sufficient. In addition, a standard curve is generated for the control sequence. This permits standardization of initial content of the nucleic acid of interest in a tissue sample to the amount of control for comparison purposes.

Methods of real-time quantitative PCR using TaqMan° probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

The TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq®, results in the cleavage of the TaqMan probe. This cleavage separates the 5′ fluorescent dye and the 3′ quenching agent, thereby resulting in an increase in fluorescence as a function of amplification (see, for example, at the world-wide web site: “perkin-elmer-dot-com”).

In another embodiment, detection of RNA transcripts can be achieved by Northern blotting, wherein a preparation of RNA is run on a denaturing agarose gel, and transferred to a suitable support, such as activated cellulose, nitrocellulose or glass or nylon membranes. Labeled (e.g., radiolabeled) cDNA or RNA is then hybridized to the preparation, washed and analyzed by methods such as autoradiography.

Detection of RNA transcripts can further be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap lipase chain reaction (RT-AGLCR) as described by R. L. Marshall, et al., PCR Methods and Applications 4: 80-84 (1994). One suitable method for detecting enzyme mRNA transcripts is described in reference Pabic et. al. Hepatology, 37(5): 1056-1066, 2003, which is herein incorporated by reference in its entirety.

Other known amplification methods which can be utilized herein include but are not limited to the so-called “NASBA” or “35R” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO 9322461.

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

Alternatively, mRNA expression can be detected on a DNA array, chip or a microarray. In such an embodiment, probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the present invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayyem et al. U.S. Pat. No. 5,952,172 and by Kelley, S. O. et al. (1999) Nucleic Acids Res. 27:4830-4837.

Oligonucleotides corresponding to gene expression target gene are immobilized on a chip which is then hybridized with labeled nucleic acids of a test sample obtained from a patient. A positive hybridization signal is obtained with a sample containing a gene expression target gene mRNA transcript. Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

Microarrays

A microarray is an array of discrete regions, typically nucleic acids, which are separate from one another and are typically arrayed at a density of between, about 100/cm.sup.2 to 1000/cm.sup.2, but can be arrayed at greater densities such as 10000/cm.sup.2. The principle of a microarray experiment, is that mRNA from a given cell line or tissue is used to generate a labeled sample typically labeled cDNA, termed the ‘target’, which is hybridized in parallel to a large number of, nucleic acid sequences, typically DNA sequences, immobilized on a solid surface in an ordered array.

Tens of thousands of transcript species can be detected and quantified simultaneously. Although many different microarray systems have been developed the most commonly used systems today can be divided into two groups, according to the arrayed material: complementary DNA (cDNA) and oligonucleotide microarrays. The arrayed material has generally been termed the probe since it is equivalent to the probe used in a northern blot analysis. Probes for cDNA arrays are usually products of the polymerase chain reaction (PCR) generated from cDNA libraries or clone collections, using either vector-specific or gene-specific primers, and are printed onto glass slides or nylon membranes as spots at defined locations. Spots are typically 10-300 μm in size and are spaced about the same distance apart. Using this technique, arrays consisting of more than 30,000 cDNAs can be fitted onto the surface of a conventional microscope slide. For oligonucleotide arrays, short 20-25 mers are synthesized in situ, either by photolithography onto silicon wafers (high-density-oligonucleotide arrays from Affymetrix or by ink-jet technology (developed by Rosetta Inpharmatics, and licensed to Agilent Technologies).

Alternatively, presynthesized oligonucleotides can be printed onto glass slides. Methods based on synthetic oligonucleotides offer the advantage that because sequence information alone is sufficient to generate the DNA to be arrayed, no time-consuming handling of cDNA resources is required. Also, probes can be designed to represent the most unique part of a given transcript, making the detection of closely related genes or splice variants possible. Although short oligonucleotides may result in less specific hybridization and reduced sensitivity, the arraying of presynthesized longer oligonucleotides (50-100 mers) has recently been developed to counteract these disadvantages.

Thus in performing a microarray to ascertain the level of gene expression of target gene expression genes in pluripotent stem cells, the following steps can be performed: obtain mRNA from the sample comprising pluripotent stem cells and prepare nucleic acids targets, contact the array under conditions, typically as suggested by the manufactures of the microarray (suitably stringent hybridization conditions such as 3×SSC, 0.1% SDS, at 50 degrees C.) to bind corresponding probes on the array, wash if necessary to remove unbound nucleic acid targets and analyze the results.

It will be appreciated that the mRNA may be enriched for sequences of interest such as those present in a gene profile as described herein by methods known in the art, such as primer specific cDNA synthesis. The population may be further amplified, for example, by using PCR technology. The targets or probes are labeled to permit detection of the hybridization of the target molecule to the microarray. Suitable labels include isotopic or fluorescent labels which can be incorporated into the probe.

The Affymetrix HG-U133.Plus 2.0 gene chips can be used and hybridized, washed and scanned according to the standard Affymetrix protocols. Some RNAs can be replicated on arrays, making 96 the total number of available hybridizations for subsequent analysis.

To monitor mRNA levels, for example, mRNA is extracted from the sample comprising pluripotent stem cells to be tested, reverse transcribed, and fluorescent-labeled cDNA probes are generated. The microarrays capable of hybridizing to gene expression target cDNA's are then probed with the labeled cDNA probes, the slides scanned and fluorescence intensity measured. This intensity correlates with the hybridization intensity and expression levels.

Methods of “quantitative” amplification are well known to those of skill in the art. For example, quantitative PCR involves simultaneously co-amplifying a known quantity of a control sequence using the same primers. This provides an internal standard that can be used to calibrate the PCR reaction. Detailed protocols for quantitative PCR are provided, for example, in Innis et al. (1990) PCR Protocols, A Guide to Methods and Applications, Academic Press, Inc. N.Y.

Although the same procedures and hardware described by Affymetrix could be employed in connection with the present invention, other alternatives are also available. Many reviews have been written detailing methods for making microarrays and for carrying out assays (see, e.g., Bowtell, Nature Genetics Suppl. 27:25-32 (1999); Constantine, et al, Life Sci. News 7:11-13 (1998); Ramsay, Nature Biotechnol. 16:40-44 (1998)). In addition, patents have issued describing techniques for producing microarray plates, slides and related instruments (U.S. Pat. No. 6,902,702; U.S. Pat. No. 6,594,432; U.S. Pat. No. 5,622,826, which are incorporated herein in their entirety by reference) and for carrying out assays (U.S. Pat. No. 6,902,900; U.S. Pat. No. 6,759,197 which are incorporated herein in their entirety by reference). The two main techniques for making plates or slides involve either polylithographic methods (see U.S. Pat. No. 5,445,934; U.S. Pat. No. 5,744,305 which are incorporated herein in their entirety by reference) or robotic spotting methods (U.S. Pat. No. 5,807,522 which are incorporated herein in their entirety by reference). Other procedures may involve inkjet printing or capillary spotting (see, e.g., WO 98/29736 or WO 00/01859 which are incorporated herein in their entirety by reference).

The substrate used for microarray plates or slides can be any material capable of binding to and immobilizing oligonucleotides including plastic, metals such a platinum and glass. A preferred substrate is glass coated with a material that promotes oligonucleotide binding such as polylysine (see Chena, et al, Science 270:467-470 (1995)). Many schemes for covalently attaching oligonucleotides have been described and are suitable for use in connection with the present invention (see, e.g., U.S. Pat. No. 6,594,432 which is incorporated herein in its entirety by reference). The immobilized oligonucleotides should be, at a minimum, 20 bases in length and should have a sequence exactly corresponding to a segment in the gene targeted for hybridization.

### Differentiation Propensity Assay

As disclosed herein, the methods, systems and assays as disclosed herein to generate a score card can optionally include a differentiation propensity assay. In some embodiments for example, a DNA methylation assay and gene expression assay can be performed after a differentiation propensity assay. In some embodiments, a differentiation propensity assay can be omitted if one is interested in determining the quality (e.g., safety) of a pluripotent stem cell line in which the user already knows differentiates along a desired cell lineage.

In general, the differentiation propensity assay allows a pluripotent stem cell line to spontaneously differentiate along different lineages for a pre-defined period of time, and then the nucleic acid material from the differentiated cells is collected and used as starting material for a DNA methylation assay and/or gene expression assay, as discussed herein. In alternative embodiments, the differentiation propensity assay also encompasses direct differentiation of a pluripotent stem cell line along a specific lineage (e.g., neuronal lineage, pancreatic lineage, cardiac lineage etc) for a pre-defined period of time, after which and then the nucleic acid material from the differentiated cells is collected and used as starting material for a DNA methylation assay and/or a gene expression assay. In some embodiments, the differentiation propensity assay encompasses spontaneous or direct differentiation of a pluripotent stem cell line for at least 0 days, or for about 1 day, or about 2 days, or about 3 days, or about 4 days, or about 5 days, or about 6 days, or about 7 days, or about 8 days, or about 8-10 days, or about 10-12 days, or about 12-14 days, or about 14-16 days, or about 16-20 days, or more than 20 days, before the differentiated cells are processed in DNA methylation assay and/or gene expression assay, as disclosed herein.

In the differentiation propensity assay, the DNA methylation assay and/or gene expression assay is performed on measuring the DNA methylation and gene expression, respectively, on a variety of lineage marker genes, and/or developmental genes as disclosed herein. In some embodiments, DNA methylation and/or gene expression is measured in a plurality of lineage marker genes, and/or developmental genes listed in Table 7.

As discussed herein, in some embodiments a pluripotent stem cell line which has a gene expression level of a lineage gene which is statistically significant (FDR<5%) and/or an absolute difference of >1 log-2 fold change of level of lineage gene expression as compared to the normal variation of gene expression for that lineage gene (e.g., the normal reference value) in a pluripotent stem cell would be considered a differentiation outlier gene. A pluripotent stem cell which has numerous, e.g., at least about 5, or at least about 6, or at least about 7, or at least about 8, or at least about 5-10, or at least about 10-15, or at least about 10-50, or at least about 50-100 or more total outlier lineage gene expression genes as compared to a reference pluripotent stem cell will be considered an outlier pluripotent stem cell, which may not differentiate along the same lineages as a reference pluripotent stem cell line. Accordingly, such a pluripotent stem cell can be used to negatively select, e.g., isolate and discard the cells with undesirable characteristics, e.g., cells which may not differentiate along particular lineages.

In some embodiments, pluripotent stem cells which are being cultured for spontaneous differentiation for use in the methods of the present invention, for example, can be monitored daily for morphology and medium exchange. Additional analysis and validation is optionally performed for stem cell markers on a routine basis, including Alkaline Phosphatase every 5 passages, OCT4, NANOG, TRA-160, TRA-181, SEAA-4, CD30 and Karyotype by G-banding every 10-15 passages, which will identify if the pluripotent stem cells have differentiated away from pluripotent stem cells.

In additional aspects, the pluripotent stem cells are cultured in conditions and under different differentiation protocols and analyzed for their tendency to predispose pluripotent stem cells to the acquisition of aberrant epigenetic alterations. For example, undirected differentiation by maintenance in suboptimal culture conditions, such as the cultivation to high density for four to seven weeks without replacement of a feeder layer is analyzed as an exemplary condition having such a tendency. For this or other culture conditions and/or protocols, DNA samples are, for example, taken at regular intervals from parallel differentiation cultures to investigate progression of abnormal epigenetic alterations. Likewise, directed differentiation protocols, such as differentiation to neural lineages 32′33 can be analyzed for their tendency to predispose ES cells to the acquisition of aberrant epigenetic alterations, pancreatic lineages (Segev et al., J. Stem Cells 22:265-274, 2004; and Xu, X. et al. Cloning Stem Cells 8:96-107, 2006, incorporated by reference herein) and/or cardiomyocytes (Yoon, B. S. et al. Differentiation 74:149-159, 2006; and Beqqali et al., Stem Cells 24:1956-1967, 2006, incorporated by reference herein).

In some embodiments, a pluripotent stem cell line is directed to be differentiated along one or more different lineages. In some embodiments, the differentaion of the pluripotent stem cell line can be assessed by DNA methylation and/or gene expression assay as disclosed herein. In alternative embodiments, the differentaion of the pluripotent stem cell line can be assessed by immunostaining and immunoassays commonly known by persons of ordinary skill in the art. Exemplary immunoassays include, enzyme linked immunoabsorbant assay (ELISA), radioimmunoassay (RIA), Immunoradiometric assay (IRMA), Western blotting, immunocytochemistry or immunohistochemistry, each of which are described in more detail below. Immunoassays such as ELISA or RIA, which can be extremely rapid, are more generally preferred. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

Immunoassays: The most common enzyme immunoassay is the “Enzyme-Linked Immunosorbent Assay (ELISA).” ELISA is a technique for detecting and measuring the concentration of an antigen using a labeled (e.g. enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904. In a “sandwich ELISA”, an antibody (e.g. anti-enzyme) is linked to a solid phase (i.e. a microtiter plate) and exposed to a biological sample containing antigen (e.g. enzyme). The solid phase is then washed to remove unbound antigen. A labeled antibody (e.g. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured.

In a “competitive ELISA”, antibody is incubated with a sample containing antigen (i.e. enzyme). The antigen-antibody mixture is then contacted with a solid phase (e.g. a microtiter plate) that is coated with antigen (i.e., enzyme). The more antigen present in the sample, the less free antibody that will be available to bind to the solid phase. A labeled (e.g., enzyme linked) secondary antibody is then added to the solid phase to determine the amount of primary antibody bound to the solid phase.

In an “immunohistochemistry assay” a section of tissue is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or beta-galactosidase), or chemical methods (e.g., DAB/Substrate chromagen). The sample is then analyzed microscopically, most preferably by light microscopy of a sample stained with a stain that is detected in the visible spectrum, using any of a variety of such staining methods and reagents known to those skilled in the art.

Alternatively, “Radioimmunoassays” can be employed. A radioimmunoassay is a technique for detecting and measuring the concentration of an antigen using a labeled (e.g. radioactively or fluorescently labeled) form of the antigen. Examples of radioactive labels for antigens include 3H, 14C, and 125I. The concentration of antigen enzyme in a biological sample is measured by having the antigen in the biological sample compete with the labeled (e.g. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (e.g., covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

An “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

Other techniques can be used to detect the level of lineage markers expressed by differentiated pluripotent stem cell populations can be performed according to a practitioner's preference. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Detectably labeled antibodies or protein binding molecules can then be used to assess the level of an expressed lineage markers, where the intensity of the signal from the detectable label corresponds to the amount of the expressed lineage marker. Levels of the amount of the expressed lineage marker present can also be quantified, for example by densitometry.

In one embodiment, the level expressed lineage marker in a biological sample can be determined by mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference. In particular embodiments, these methodologies can be combined with the machines, computer systems and media to produce an automated system for determining the level of expressed lineage marker expressed in a pluripotent stem cell population and analysis to produce a printable report which identifies, for example, the level of level of protein expression in a biological sample.

Pluripotent Stem Cells for Use in Generating a Scorecard or for Determining Functionality by Comparison with a Scorecard.

The methods, kits, systems and scorecards as disclosed herein can be used to validate and monitor any pluripotent stem cell, from any species, e.g. a mammalian species, such as a human.

Generally, a pluripotent stem cell for use in the methods, assays, systems, kits and to generate scorecards can be obtained or derived from any available source. Accordingly, a pluripotent cell can be obtained or derived from a vertebrate or invertebrate. In some embodiments, the pluripotent stem cell is mammalian pluripotent stem cell. In all aspects as disclosed herein, pluripotent stem cells for use in the methods, assays and to generate scorecards or to compare with an existing scorecard as disclosed herein can be any pluripotent stem cell. For example, a pluripotent stem cell can be obtained or derived from a vertebrate or a invertebrate. In some embodiments of the aspects of the invention the pluripotent stem cell is mammalian pluripotent stem cell.

In some embodiments of the aspects of the invention, the pluripotent stem cell is primate or rodent pluripotent stem cell. In some embodiments of the aspects of the invention, the pluripotent stem cell is selected from the group consisting of chimpanzee, cynomologous monkey, spider monkey, macaques (e.g. Rhesus monkey), mouse, rat, woodchuck, ferret, rabbit, hamster, cow, horse, pig, deer, bison, buffalo, feline (e.g., domestic cat), canine (e.g. dog, fox and wolf), avian (e.g. chicken, emu, and ostrich), and fish (e.g., trout, catfish and salmon) pluripotent stem cell.

In some embodiments of the aspects of the invention, the pluripotent stem cell is a human pluripotent stem cell. In some embodiments, the pluripotent stem cell is a human stem cell line known to one of ordinary skill in the art. In some embodiments, the pluripotent stem cell is an induced pluripotent stem (iPS) cell, or a stably reprogrammed cell which is an intermediate pluripotent stem cell and can be further reprogrammed into an iPS cell, e.g., partial induced pluripotent stem cells (also referred to as “piPS cells”). In some embodiments, the pluripotent stem cell, iPSC or piPSC is a genetically modified pluripotent stem cell.

In some embodiments, the pluripotent state of a pluripotent stem cell used in the present invention can be confirmed by various methods. For example, the cells can be tested for the presence or absence of characteristic ES cell markers. In the case of human ES cells, examples of such markers are identified supra, and include SSEA-4, SSEA-3, TRA-1-60, TRA-1-81 and OCT 4, and are known in the art.

Also, pluripotency can be confirmed by injecting the cells into a suitable animal, e.g., a SCID mouse, and observing the production of differentiated cells and tissues. Still another method of confirming pluripotency is using the subject pluripotent cells to generate chimeric animals and observing the contribution of the introduced cells to different cell types. Methods for producing chimeric animals are well known in the art and are described in U.S. Pat. No. 6,642,433, which is incorporated by reference herein.

Yet another method of confirming pluripotency is to observe ES cell differentiation into embryoid bodies and other differentiated cell types when cultured under conditions that favor differentiation (e.g., removal of fibroblast feeder layers). This method has been utilized and it has been confirmed that the subject pluripotent cells give rise to embryoid bodies and different differentiated cell types in tissue culture.

The resultant pluripotent cells and cell lines, preferably human pluripotent cells and cell lines, which are derived from DNA of entirely female original, have numerous therapeutic and diagnostic applications. Such pluripotent cells may be used for cell transplantation therapies or gene therapy (if genetically modified) in the treatment of numerous disease conditions.

In this regard, it is known that some mouse embryonic stem (ES) cells have a propensity of differentiating into some cell types at a greater efficiency as compared to other cell types. Similarly, human pluripotent (ES) cells possess similar selective differentiation capacity. Accordingly, the present invention can be used to identify and select a pluripotent stem cell with desired characteristics and differentiation propensity for the desired use of the pluripotent stem cell. For example, where the pluripotent cell line has been screened according to the methods of the invention, a pluripotent stem cell can be selected due to its increased efficiency of differentiating along a particular cell line, (as well as other desirable characteristics such as epigenetic silencing of oncogenes, low methylation of tumor suppressor genes and/or particular developmental genes) and can be induced to differentiate to obtain the desired cell types according to known methods. For example, a human pluripotent stem cell, e.g., a ES cell or iPS cell can be induced to differentiate into hematopoietic stem cells, muscle cells, cardiac muscle cells, liver cells, islet cells, retinal cells, cartilage cells, epithelial cells, urinary tract cells, etc., by culturing such cells in differentiation medium and under conditions which provide for cell differentiation, according to methods known to persons of ordinary skill in the art. Medium and methods which result in the differentiation of ES cells are known in the art as are suitable culturing conditions.

In some embodiments, a pluripotent stem cell is an induced pluripotent stem cell (e.g., an iPS cell) or a stable partially reprogrammed cell, e.g., piPSC. In some embodiments, the stable reprogrammed cells as disclosed herein can be produced from the incomplete reprogramming of a somatic cell. In some embodiments, the somatic cell is a human cell, and can be a diseased somatic cell, e.g., obtained from a subject with a pathology, or from a subject with a genetic predisposition to have, or be at risk of a disease or disorder.

One can use any method for reprogramming a somatic cell to an iPS cell or an piPS cell, for example, as disclosed in International patent applications; WO2007/069666; WO2008/118820; WO2008/124133; WO2008/151058; WO2009/006997; and U.S. Patent Applications US2010/0062533; US2009/0227032; US2009/0068742; US2009/0047263; US2010/0015705; US2009/0081784; US2008/0233610; U.S. Pat. No. 7,615,374; U.S. patent application Ser. No. 12/595,041, EP2145000, CA2683056, AU8236629, Ser. No. 12/602,184, EP2164951, CA2688539, US2010/0105100; US2009/0324559, US2009/0304646, US2009/0299763, US2009/0191159, the contents of which are incorporated herein in their entirety by reference. In some embodiments, an iPS cell for use in the methods, assays and to generate scorecards or to compare with an existing scorecard as disclosed herein can be produced by any method known in the art for reprogramming a cell, for example virally-induced or chemically induced generation of reprogrammed cells, as disclosed in EP1970446, US2009/0047263, US2009/0068742, and 2009/0227032, which are incorporated herein in their entirety by reference.

In some embodiments, an iPS cell for use in the methods, assays and to generate scorecards or to compare with an existing scorecard as disclosed herein can be produced from the incomplete reprogramming of a somatic cell by chemical reprogramming, such as by the methods as disclosed in WO2010/033906, the contents of which is incorporated herein in its entirety by reference. In alternative embodiments, the stable reprogrammed cells disclosed herein can be produced from the incomplete reprogramming of a somatic cell by non-viral means, such as by the methods as disclose in WO2010/048567 the contents of which is incorporated herein in its entirety by reference.

Other pluripotent stem cells for use in the methods, assays and to generate scorecards or to compare with an existing scorecard as disclosed herein can be any pluripotent stem cell known to persons of ordinary skill in the art. Exemplary stem cells include embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Descriptions of stem cells, including method for isolating and culturing them, may be found in, among other places, Embryonic Stem Cells, Methods and Protocols, Turksen, ed., Humana Press, 2002; Weisman et al., Annu. Rev. Cell. Dev. Biol. 17:387 403; Pittinger et al., Science, 284:143 47, 1999; Animal Cell Culture, Masters, ed., Oxford University Press, 2000; Jackson et al., PNAS 96(25):14482 86, 1999; Zuk et al., Tissue Engineering, 7:211 228, 2001 (“Zuk et al.”); Atala et al., particularly Chapters 33 41; and U.S. Pat. Nos. 5,559,022, 5,672,346 and 5,827,735. Descriptions of stromal cells, including methods for isolating them, may be found in, among other places, Prockop, Science, 276:7174, 1997; Theise et al., Hepatology, 31:235 40, 2000; Current Protocols in Cell Biology, Bonifacino et al., eds., John Wiley & Sons, 2000 (including updates through March, 2002); and U.S. Pat. No. 4,963,489. The skilled artisan will understand that the stem cells and/or stromal cells selected for inclusion in a transplant with mixed SVF cells or SVF-matrix construct (e.g. for encapsulating a tissue or cell transplant according to the constructs and methods as disclosed herein) are typically appropriate for the intended use of that construct.

Additional pluripotent stem cells for use in the methods, assays and to generate scorecards or to compare with an existing scorecard as disclosed herein can be any cells derived from any kind of tissue (for example embryonic tissue such as fetal or pre-fetal tissue, or adult tissue), which stem cells have the characteristic of being capable under appropriate conditions of producing progeny of different cell types that are derivatives of all of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, an embryo has not been destroyed in obtaining a pluripotent stem cell for use in the methods, assays, systems and to generate scorecards or to compare with an existing scorecard as disclosed herein.

In another embodiment, the stem cells, e.g., adult or embryonic stem cells can be isolated from tissue including solid tissues (the exception to solid tissue is whole blood, including blood, plasma and bone marrow) which were previously unidentified in the literature as sources of stem cells. In some embodiments, the tissue is heart or cardiac tissue. In other embodiments, the tissue is for example but not limited to, umbilical cord blood, placenta, bone marrow, or chondral villi.

Stem cells of interest for use in the methods, assays, systems and to generate scorecards or to compare with an existing scorecard as disclosed herein also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.). In some embodiments, the pluripotent stem cells may be obtained from any mammalian species, e.g. human, equine, bovine, porcine, canine, feline, rodent, e.g. mice, rats, hamster, primate, etc. In some embodiments, where the pluripotent stem cell is a human pluripotent stem cell, an embryo has not been destroyed in obtaining a pluripotent stem cell for use in the methods, assays, systems and to generate scorecards or to compare with an existing scorecard as disclosed herein.

By way of background only, an ES cell is considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-I-60, TRA-1-81, and alkaline phosphatase. The globo-series glycolipid GL7, which carries the SSEA-4 epitope, is formed by the addition of sialic acid to the globo-series glycolipid Gb5, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-L Methods for proliferating hES cells in the undifferentiated form are described in WO 99/20741, WO 01/51616, and WO 03/020920, which are incorporated herein in their entirety by reference.

In some embodiments, a pluripotent stem cell for use in the methods, assays, systems and to generate scorecards or to compare with an existing scorecard as disclosed herein is a human umbilical cord blood cell. Human umbilical cord blood cells (HUCBC) have recently been recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e. acute lymphoid leukemia, acute myeloid leukemia, chronic myeloid leukemia, myelodysplastic syndrome, and nueroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985 J. Immunol. 134:1493-1497).

Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985 J. Immunol. 134:1493-1497 Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096). One source of cells is the hematopoietic micro-environment, such as the circulating peripheral blood, preferably from the mononuclear fraction of peripheral blood, umbilical cord blood, bone marrow, fetal liver, or yolk sac of a mammal. In some embodiments, pluripotent stem cells, especially neural stem cells, may also be derived from the central nervous system, including the meninges.

### Computer Systems

One aspect of the present invention relates to a computerized system for processing the assay data and generating a measure or rating of one or more target cells, such as one or more quality assurance scorecards of a pluripotent stem cell. The computer system can include: (a) at least one memory containing at least one computer program adapted to control the operation of the computer system to implement a method that includes: (i) receiving DNA methylation data e.g., the level of methylation of a set of DNA methylation target genes in the pluripotent stem cell line of interest and performing a comparison of the DNA methylation data with a reference DNA methylation level of the same target genes in a control pluripotent stem cell line or a plurality of reference pluripotent stem cell lines; (ii) receiving differentiation potential data of the pluripotent stem cell line and comparing the differentiation potential data with a reference differentiation potential data; (iii) generating a deviation scorecard based on the comparison of the DNA methylation data as compared to reference DNA methylation data parameters and generating a lineage scorecard based on comparing the differentiation propensity of the stem cell line of interest as compared to reference differentiation data; and (b) at least one processor for executing the computer program.

In some embodiments, the computer system can include: (a) at least one memory containing at least one computer program adapted to control the operation of the computer system to implement a method that includes: (i) receiving DNA methylation data, e.g., the level of methylation of a set of DNA methylation target genes in the pluripotent stem cell line of interest and performing a comparison with the DNA methylation data, (e.g., the level of DNA methylation) of the same DNA methylation target genes in a control pluripotent stem cell line or a plurality of reference pluripotent stem cell lines; (ii) receiving the gene expression data, e.g., level of gene expression of a set of lineage marker genes in a pluripotent stem cell line of interest and performing a comparison of the gene expression data (e.g., gene expression level) of the same lineage marker genes in a control pluripotent stem cell line or a plurality of reference pluripotent stem cell lines, (iii) generating a deviation scorecard based on the comparison of the DNA methylation data as compared to reference DNA methylation parameters and generating a lineage scorecard based on the comparison of the level of gene expression of lineage marker genes in the pluripotent stem cell of interest as compared to reference level of gene expression of lineage markers for the genes; and (b) at least one processor for executing the computer program.

In some embodiments, the computer program is adapted to control the operation of the computer system to implement a method that further includes: (i) receiving gene expression data (e.g., gene expression levels) of a second set of target genes in the pluripotent stem cell line of interest and comparing the gene expression data (e.g., gene expression levels) with a reference gene expression data (e.g., gene expression levels of the same second set of target genes in a control pluripotent stem cell line or a plurality of pluripotent stem cell lines); (ii) generating a derivation scorecard based on the comparison of the gene expression data (e.g., gene expression levels) as compared to reference gene expression data (e.g., reference gene expression levels in reference pluripotent stem cell line(s)).

Another aspect of the present invention relates to a computer readable medium comprising instructions, such as computer programs and software, for controlling a computer system to process assay data and generate one or more quality assurance scorecards of a pluripotent stem cell line, comprising: (i) receiving DNA methylation data, e.g., the level of methylation of a set of DNA methylation target genes in the pluripotent stem cell line of interest and performing a comparison with the DNA methylation data, (e.g., the level of DNA methylation) of the same DNA methylation target genes in a control pluripotent stem cell line or a plurality of reference pluripotent stem cell lines; (ii) receiving the gene expression data, e.g., level of gene expression of a set of lineage marker genes in a pluripotent stem cell line of interest and performing a comparison of the gene expression data (e.g., gene expression level) of the same lineage marker genes in a control pluripotent stem cell line or a plurality of reference pluripotent stem cell lines, (iii) generating a deviation scorecard based on the comparison of the DNA methylation data as compared to reference DNA methylation parameters and generating a lineage scorecard based on the comparison of the level of gene expression of lineage marker genes in the pluripotent stem cell of interest as compared to reference level of gene expression of lineage markers for the genes. In some embodiments, the computer-readable medium further comprises instructions for: (i) receiving gene expression data (e.g., gene expression levels) of a second set of target genes in the pluripotent stem cell line of interest and comparing the gene expression data (e.g., gene expression levels) with a reference gene expression data (e.g., reference gene expression levels) of the same second set of target genes in a control pluripotent stem cell line or a plurality of pluripotent stem cell lines); (ii) generating a derivation scorecard based on the comparison of the gene expression data (e.g., gene expression levels) as compared to reference gene expression data (e.g., reference gene expression levels in reference pluripotent stem cell line(s)).

The computer system can include one or more general or special purpose processors and associated memory, including volatile and non-volatile memory devices. The computer system memory can store software or computer programs for controlling the operation of the computer system to make a special purpose system according to the invention or to implement a system to perform the methods according to the invention. The computer system can include an Intel or AMD x86 based single or multi-core central processing unit (CPU), an ARM processor or similar computer processor for processing the data. The CPU or microprocessor can be any conventional general purpose single- or multi-chip microprocessor such as an Intel Pentium processor, an Intel 8051 processor, a RISC or MISS processor, a Power PC processor, or an ALPHA processor. In addition, the microprocessor may be any conventional or special purpose microprocessor such as a digital signal processor or a graphics processor. The microprocessor typically has conventional address lines, conventional data lines, and one or more conventional control lines. As described below, the software according to the invention can be executed on dedicated system or on a general purpose computer having a DOS, CPM, Windows, Unix, Linix or other operating system. The system can include non-volatile memory, such as disk memory and solid state memory for storing computer programs, software and data and volatile memory, such as high speed ram for executing programs and software.

Computer-readable physical storage media useful in various embodiments of the invention can include any physical computer-readable storage medium, e.g., solid state memory (such as flash memory), magnetic and optical computer-readable storage media and devices, and memory that uses other persistent storage technologies. In some embodiments, a computer readable media can be any tangible media that allows computer programs and data to be accessed by a computer. Computer readable media can include volatile and nonvolatile, removable and non-removable tangible media implemented in any method or technology capable of storing information such as computer readable instructions, program modules, programs, data, data structures, and database information. In some embodiments of the invention, computer readable media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store information and which can read by a computer including and any suitable combination of the foregoing.

The present invention can be implemented on a stand-alone computer or as part of a networked computer system. In a stand-alone computer, all the software and data can reside on local memory devices, for example an optical disk or flash memory device can be used to store the computer software for implementing the invention as well as the data. In alternative embodiments, the software or the data or both can be accessed through a network connection to remote devices. In one networked computer system embodiment, the invention use a client-server environment over a public network, such as the internet or a private network to connect to data and resources stored in remote and/or centrally located locations. In this embodiment, a server including a web server can provide access, either open access, pay as you go or subscription based access to the information provided according to the invention. In a client server environment, a client computer executing a client software or program, such as a web browser, connects to the server over a network. The client software or web browser provides a user interface for a user of the invention to input data and information and receive access to data and information. The client software can be viewed on a local computer display or other output device and can allow the user to input information, such as by using a computer keyboard, mouse or other input device. The server executes one or more computer programs that enable the client software to input data, process data according to the invention and output data to the user, as well as provide access to local and remote computer resources. For example, the user interface can include a graphical user interface comprising an access element, such as a text box, that permits entry of data from the assay, e.g., the DNA methylation data levels or DNA gene expression levels of target genes of a reference pluripotent stem cell population and/or pluripotent stem cell population of interest, as well as a display element that can provide a graphical read out of the results of a comparison with a score card, or data sets transmitted to or made available by a processor following execution of the instructions encoded on a computer-readable medium.

Embodiments of the invention also provide for systems (and computer readable medium for causing computer systems) to perform a method for determining quality assurance of a pluripotent stem cell population according to the methods as disclosed herein.

In some embodiments of the invention, the computer system software can include one or more functional modules, which can be defined by computer executable instructions recorded on computer readable media and which cause a computer to perform a method according to the invention, when executed. The modules can be segregated by function for the sake of clarity, however, it should be understood that the modules need not correspond to discreet blocks of code and the described functions can be carried out by the execution of various software code portions stored on various media and executed at various times. Furthermore, it should be appreciated that the modules can perform other functions, thus the modules are not limited to having any particular function or set of functions. In some embodiments, functional modules for producing a deviation score card are, for example, but are not limited to, a storage module, a gene mapping module, a reference comparison module, a normalization module, a relevance filter module, a gene set module, and a scorecard display module to display the deviation scorecard. Functional modules for producing a lineage scorecard are, for example, but are not limited to, a storage device, an assay normalization module, a sample normalization module, a reference comparison module, a gene set module, an enrichment analysis module, and a scorecard display module to display the lineage scorecard. The functional modules can be executed using one or multiple computers, and by using one or multiple computer networks.

The information embodied on one or more computer-readable media can include data, computer software or programs, and program instructions, that, as a result of being executed by a computer, transform the computer to special purpose machine and can cause the computer to perform one or more of the functions described herein. Such instructions can be originally written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied can reside on one or more of the components of a computer system or a network of computer systems according to the invention.

In some embodiments, a computer-readable media can be transportable such that the instructions stored thereon can be loaded onto any computer resource to implement the aspects of the present invention discussed herein. In addition, it should be appreciated that the instructions stored on computer readable media are not limited to instructions embodied as part of an application program running on a host computer. Rather, the instructions may be embodied as any type of computer code (e.g., object code, software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

In some embodiments, a system as disclosed herein, can receive gene expression level data from an automated gene expression analysis system, e.g., an automated protein expression analysis including but not limited Mass Spectrometry systems including MALDI-TOF, or Matrix Assisted Laser Desorption Ionization-Time of Flight systems; SELDI-TOF-MS ProteinChip array profiling systems, e.g. Machines with Ciphergen Protein Biology System II™ software; systems for analyzing gene expression data (see for example U.S. 2003/0194711); systems for array based expression analysis, for example HT array systems and cartridge array systems available from Affymetrix (Santa Clara, Calif. 95051) AutoLoader, Complete GeneChip® Instrument System, Fluidics Station 450, Hybridization Oven 645, QC Toolbox Software Kit, Scanner 3000 7G, Scanner 3000 7G plus Targeted Genotyping System, Scanner 3000 7G Whole-Genome Association System, GeneTitan™ Instrument, GeneChip® Array Station, HT Array; an automated ELISA system (e.g. DSX® or DS2® form Dynax, Chantilly, Va. or the ENEASYSTEM III®, Triturus®, The Mago® Plus); Densitometers (e.g. X-Rite-508-Spectro Densitometer®, The HYRYS™ 2 densitometer); automated Fluorescence insitu hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, Becton Dickinson); radio isotope analyzers (e.g. scintillation counters).

In some embodiments of the present invention, the reference data can be electronically or digitally recorded, annotated and retrieved from databases including, but not limited to GenBank (NCBI) protein and DNA databases such as genome, ESTs, SNPS, Traces, Celara, Ventor Reads, Watson reads, HGTS, etc.; Swiss Institute of Bioinformatics databases, such as ENZYME, PROSITE, SWISS-2DPAGE, Swiss-Prot and TrEMBL databases; the Melanie software package or the ExPASy WWW server, etc., the SWISS-MODEL, Swiss-Shop and other network-based computational tools; the Comprehensive Microbial Resource database (The institute of Genomic Research). The resulting information can be stored in a relational data base that may be employed to determine homologies between the reference data or genes or proteins within and among genomes.

In some embodiments, the gene expression levels of target genes in a pluripotent stem cell can be received from a memory, a storage device, or a database. The memory, storage device or database can be directly connected to the computer system retrieving the data, or connected to the computer through a wired or wireless connection technology and retrieved from a remote device or system over the wired or wireless connection. Further, the memory, storage device or database, can be located remotely from the computer system from which it is retrieved.

Examples of suitable connection technologies for use with the present invention include, for example parallel interfaces (e.g., PATA), serial interfaces (e.g., SATA, USB, Firewire), local area networks (LAN), wide area networks (WAN), Internet, Intranet, and Extranet, and wireless (e.g., Blue Tooth, Zigbee, WiFi, WiMAX, 3G, 4G) communication technologies

Storage devices are also commonly referred to in the art as “computer-readable physical storage media” which is useful in various embodiments, and can include any physical computer-readable storage medium, e.g., magnetic and optical computer-readable storage media, among others. Carrier waves and other signal-based storage or transmission media are not included within the scope of storage devices or physical computer-readable storage media encompassed by the term and useful according to the invention. The storage device is adapted or configured for having recorded thereon cytokine level information. Such information can be provided in digital form that can be transmitted and read electronically, e.g., via the Internet, on diskette, via USB (universal serial bus) or via any other suitable mode of communication.

As used herein, “stored” refers to a process for recording information, e.g., data, programs and instructions, on the storage device, that can be read back at a later time. Those skilled in the art can readily adopt any of the presently known methods for recording information on known media to contribute to a reference scorecard data, e.g., the level of DNA methylation, and/or gene expression level, and/or differentiation propensity data of a pluripotent stem cell as disclosed in the methods herein.

A variety of software programs and formats can be used to store the scorecard data and information on the storage device. Any number of data processor structuring formats (e.g., text file or database) can be employed to obtain or create a medium having recorded scorecard thereon.

In one embodiment, the reference scorecard data can be electronically or digitally recorded and annotated from databases including, but not limited to protein expression databases commonly known in the art, such as Yale Protein Expression Database (YPED), as well as GenBank (NCBI) protein and DNA databases such as genome, ESTs, SNPS, Traces, Celara, Ventor Reads, Watson reads, HGTS, and the like; Swiss Institute of Bioinformatics databases, such as ENZYME, PROSITE, SWISS-2DPAGE, Swiss-Prot and TrEMBL databases; the Melanie software package or the ExPASy WWW server, and the like; the SWISS-MODEL, Swiss-Shop and other network-based computational tools; the Comprehensive Microbial Resource database (available from The Institute of Genomic Research). The resulting information of the level of DNA methylation, and/or Gene expression level, and/or differentiation propensity data of a pluripotent stem cell line can be stored in a relational database that may be employed to determine differences as compared to different pluripotent stem cell populations, or compared to reference DNA methylation levels, reference Gene expression levels and reference propensity differentiation data between different pluripotent stem cell populations, e.g., ES cells, and iPS cells and piPS cells, and somatic stem cells, or among pluripotent stem cells of the same type (e.g., iPS cells) from different genomes, species and different populations of individuals.

In some embodiment, the system has a processor for running one or more programs, e.g., where the programs can include an operating system (e.g., UNIX, Windows), a relational database management system, an application program, and a World Wide Web server program. The application program can be a World Wide Web application that includes the executable code necessary for generation of database language statements (e.g., Structured Query Language (SQL) statements). The executables can include embedded SQL statements. In addition, the World Wide Web application can include a configuration file which contains pointers and addresses to the various software entities that provide the World Wide Web server functions as well as the various external and internal databases which can be accessed to service user requests. The Configuration file can also direct requests for server resources to the appropriate hardware devices, as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

In one embodiment, the system as disclosed herein can be used to compare DNA methylation data (e.g., DNA methylation profiles or levels of DNA methylation of a plurality of DNA methylation target genes) and/or Gene expression profiles (e.g., gene expression profiles or levels of gene expression of a plurality of gene expression target genes). For example, the system can receive onto its memory gene expression profiles or data of the test pluripotent stem cell line and compare it with one or more stored gene expression profiles (e.g. the normal variation of gene expression in one or more reference pluripotent stem cell lines), or compare with one or more gene expression profiles from the pluripotent stem cell line previously analyzed at an earlier timepoint. In some embodiments, gene expression profiles are obtained using Affymetrix Microarray Suite software version 5.0 (MAS 5.0) (available from Affymetrix, Santa Clara, Calif.) to analyze the relative abundance of a gene or genes on the basis of the intensity of the signal from probe sets, and the MAS 5.0 data files can be transferred into a database and analyzed with Microsoft Excel and GeneSpring 6.0 software (available from Agilent Technologies, Santa Clara, Calif.). In some embodiments, a comparison algorithm of MAS 5.0 software can be used to obtain a comprehensive overview of how many transcripts are detected in given samples and allows a comparative analysis of 2 or more microarray data sets.

In some embodiments of this aspect and all other aspects of the present invention, the system can compare the data in a “comparison module” which can use a variety of available software programs and formats for the comparison operative to compare sequence information determined in the determination module to reference data. In one embodiment, the comparison module is configured to use pattern recognition techniques to compare sequence information from one or more entries to one or more reference data patterns. The comparison module may be configured using existing commercially-available or freely-available software for comparing patterns, and may be optimized for particular data comparisons that are conducted. The comparison module can also provide computer readable information related to the sequence information that can include, for example, detection of the presence or absence of a CpG methylation sites in DNA sequences; determination of the level of methylation, determination of the concentration of a sequence in the sample (e.g. amino acid sequence/protein expression levels, or nucleotide (RNA or DNA) expression levels), or determination of a Gene expression profile.

In some embodiments of the invention, system comprises comparison software which is used to determine whether the DNA methylation data for a pluripotent stem cell of interest, or the gene expression level data for a pluripotent stem cell of interests falls outside a reference DNA methylation level (e.g., normal variation of DNA methylation) or reference gene expression level as disclosed herein, e.g., outside the normal variation of gene expression levels for the target genes) for a plurality of pluripotent stem cells. In one embodiment, where the DNA methylation level for a pluripotent stem cell of interest expression is higher by a statically significantly amount above reference DNA methylation levels it indicates likelihood of epigenetic silencing and repression of the DNA methylation target gene. In instances where the DNA methylation target gene is a tumor suppressor gene, it will indicate that the pluripotent stem cell has a predisposition to become a cancer cell. In instances where the DNA methylation target gene is a developmental gene and/or a lineage marker gene, the software can be configured to indicate or signal that the pluripotent stem cell line will have low efficiency of differentiation or not differentiate along that particular developmental pathway or not differentiate into a cell that expresses the lineage marker gene.

Similarly, where the gene expression level for a pluripotent stem cell of interest expression is higher by a statically significantly amount above a reference gene expression level for that gene, it indicates likelihood of expression of the target gene, and if the DNA target gene is a developmental or lineage specific marker, the software can be configured to signal (or otherwise indicate) the likelihood of optimal differentiation along that cell lineage. In instances where the DNA methylation target gene is an oncogene, the software can be configured to signal that the pluripotent stem cell line of interest will likely have a predisposition to become a cancer cell or have uncontrolled proliferation.

By providing DNA methylation data and/or gene expression level data in computer-readable form, one can use the DNA methylation data and/or gene expression level data for a pluripotent stem cell to compare with reference DNA methylation levels and reference gene expression levels of other pluripotent stem cells within the storage device. For example, search programs can be used to identify relevant reference data (i.e. reference DNA methylation levels of a target gene) that match the DNA methylation level of a same target gene for the pluripotent stem cell of interest. The comparison made in computer-readable form provides computer readable content which can be processed by a variety of means. The content can be retrieved from the comparison module, the retrieved content.

In some embodiments, the comparison module provides computer readable comparison result that can be processed in computer readable form by predefined criteria, or criteria defined by a user, to provide a report which comprises content based in part on the comparison result that may be stored and output as requested by a user using a display module. In some embodiments, a display module enables display of a content based in part on the comparison result for the user, wherein the content is a report indicative of the results of the comparison of the pluripotent stem cell of interest with a scorecard, or the utility of the pluripotent stem cell, e.g., methylation status of particular cancer (e.g., oncogene and tumor suppressor genes) and methylation status of specific developmental and/or lineage marker genes.

In some embodiments, the display module enables display of a report or content based in part on the comparison result for the end user, wherein the content is a report indicative of the results of the comparison of the pluripotent stem cell of interest with a scorecard, or the utility of the pluripotent stem cell, e.g., methylation status of particular cancer (e.g., oncogene and tumor suppressor genes) and methylation status of specific developmental and/or lineage marker genes.

In some embodiments of this aspect and all other aspects of the present invention, the comparison module, or any other module of the invention, can include an operating system (e.g., UNIX, Windows) on which runs a relational database management system, a World Wide Web application, and a World Wide Web server. World Wide Web application can includes the executable code necessary for generation of database language statements [e.g., Standard Query Language (SQL) statements]. The executables canl include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using an HTML interface provided by Web browsers and Web servers. In other embodiments of the invention, other interfaces, such as HTTP, FTP, SSH and VPN based interfaces can be used to connect to the Internet databases.

In some embodiments of this aspect and all other aspects of the present invention, a computer-readable media can be transportable such that the instructions stored thereon, such as computer programs and software, can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a processor to implement aspects of the present invention. The computer executable instructions can be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The computer instructions can be implemented in software, firmware or hardware and include any type of programmed step undertaken by modules of the information processing system. The computer system can be connected to a local area network (LAN) or a wide area network (WAN). One example of the local area network can be a corporate computing network, including access to the Internet, to which computers and computing devices comprising the data processing system are connected. In one embodiment, the LAN uses the industry standard Transmission Control Protocol/Internet Protocol (TCP/IP) network protocols for communication. Transmission Control Protocol Transmission Control Protocol (TCP) can be used as a transport layer protocol to provide a reliable, connection-oriented, transport layer link among computer systems. The network layer provides services to the transport layer. Using a two-way handshaking scheme, TCP provides the mechanism for establishing, maintaining, and terminating logical connections among computer systems. TCP transport layer uses IP as its network layer protocol. Additionally, TCP provides protocol ports to distinguish multiple programs executing on a single device by including the destination and source port number with each message. TCP performs functions such as transmission of byte streams, data flow definitions, data acknowledgments, lost or corrupt data re-transmissions, and multiplexing multiple connections through a single network connection. Finally, TCP is responsible for encapsulating information into a datagram structure. In alternative embodiments, the LAN can conform to other network standards, including, but not limited to, the International Standards Organization's Open Systems Interconnection, IBM's SNA, Novell's Netware, and Banyan VINES.

In some embodiments, the computer system as described herein can include any type of electronically connected group of computers including, for instance, the following networks: Internet, Intranet, Local Area Networks (LAN) or Wide Area Networks (WAN). In addition, the connectivity to the network may be, for example, remote modem, Ethernet (IEEE 802.3), Token Ring (IEEE 802.5), Fiber Distributed Datalink Interface (FDDI) or Asynchronous Transfer Mode (ATM). The computing devices can be desktop devices, servers, portable computers, hand-held computing devices, smart phones, set-top devices, or any other desired type or configuration. As used herein, a network includes one or more of the following, including a public internet, a private internet, a secure internet, a private network, a public network, a value-added network, an intranet, an extranet and combinations of the foregoing.

In one embodiment of the invention, the computer system can comprise a pattern comparison software can be used to determine whether the patterns of DNA methylation levels or gene expression levels in a pluripotent stem cell line of interest are indicative of that cell line being an outlier and predictive of a stem cell line functioning outside the normal characteristics of reference pluripotent stem cell lines, or the likelihood of the pluripotent stem cell line having a low efficiency of differentiating along a particular cell line of interest or possessing cancer like properties, e.g., predisposition for uncontrolled proliferation. In this embodiment, the pattern comparison software can compare at least some of the data (e.g., DNA methylation levels and/or gene expression levels) of the pluripotent stem cell of interest with predefined patterns of DNA methylation levels and gene expression levels (of DNA methylation target genes, and/or gene expression target genes and/or lineage marker target genes) of reference pluripotent stem cell lines to determine how closely they match. The matching can be evaluated and reported in portions or degrees indicating the extent to which all or some of the pattern matches.

In some embodiments of this aspect and all other aspects of the present invention, a comparison module provides computer readable data that can be processed in computer readable form by predefined criteria, or criteria defined by a user, to provide a retrieved content that may be stored and output as requested by a user using a display module.

Display Module

In accordance with some embodiments of the invention, the computerized system can include or be operatively connected to a display module, such as computer monitor, touch screen or video display system. The display module allows user instructions to be presented to the user of the system, to view inputs to the system and for the system to display the results to the user as part of a user interface. Optionally, the computerized system can include or be operative connected to a printing device for producing printed copies of information output by the system.

In some embodiments, the results can be displayed on a display module or printed in a report, e.g., a scorecard report to indicate the quality and/or utility of the pluripotent stem cell of interest, e.g., utility for a particular therapeutic use based on low risk of likelihood of developing into a cancer cell, and/or utility for a particular purpose based on likelihood of differentiating along a certain cell line lineage based on the data from the DNA methylation and/or Gene expression of developmental genes and lineage specific markers, and differentiation propensity data.

In some embodiments, the scorecard report is a hard copy printed from a printer. In alternative embodiments, the computerized system can use light or sound to report the scorecard, e.g., to indicate the quality and utility of a pluripotent stem cell line of interest. For example, in all aspects of the invention, the scorecard produced by the methods, assays, systems and present in the kits as disclosed herein can comprise a report which is color coded to signal or indicate the quality of the pluripotent stem cell of interest as compared to one or more reference pluripotent stem cell lines (e.g., the standard human ES cell lines and iPS cells as tested herein), or compared another “gold” standard pluripotent stem cell line of the investigators choice.

For example, a red color or other predefined signal can indicate that the pluripotent stem cell line is an outlier pluripotent stem cell line, and has one or more genes where the level of DNA methylation and or level of gene expression vary by a stastistically significant amount as compared to levels in one or more reference pluripotent stem cell lines, thus signalling that the pluripotent stem cell line has different characteristics to the reference pluripotent stem cell lines, e.g., may have a predisposition to differentiate into a cancer cell line and/or low efficiency to differentiate into a particular cell lineage. In another embodiment, a yellow or orange color or other predefined signal can indicate that the pluripotent stem cell line may have one genes where the level of DNA methylation and or level of gene expression varies by a stastistically significant amount as compared to levels in one or more reference pluripotent stem cell lines, thus signalling that the pluripotent stem cell line has slightly different characteristic to the reference pluripotent stem cell line(s), but that difference may not be important to the function, e.g., the pluripotent stem cell line of interest is still of the characteristic quality to be used, and does not have a predisposition to differentiate into a cancer cell line etc. In another embodiment, a green color or other predefined signal can indicate that the pluripotent stem cell line is of high quality and the level of DNA methylation and or level of gene expression of the majority of genes does not vary by a stastistically significant amount as compared to levels in one or more reference pluripotent stem cell lines, thus signalling that the pluripotent stem cell line is of high quality and likely to have similar characteristic to the reference pluripotent stem cell line(s). In some embodiments, a “heat map” or gradient color scheme can be used in the report, e.g., scorecard report to signal the quality of the pluripotent stem cell line, for example, where the gradient is a red to yellow to green gradient, where a red signal will signal an inferior and/or poor quality, and a yellow signal will indicate a good quality and a green signal will indicate a high quality pluripotent stem cell of interest as compared to one or more reference pluripotent stem cell line(s). Colors between red and yellow and yellow and green will signal the characteristics of the pluripotent stem cell line with respect to a red-yellow-green scale. Other color schemes and gradient schemes in the report are also encompassed.

In some embodiments, the report, e.g., scorecard can display the total %, and/or absolute total number of genes which differentiate in the DNA methylation levels as compared to the normal variation of DNA methylation. Similarly, the report, e.g., scorecard can display the total %, and/or absolute total number of genes which have a differential gene expression levels as compared to the normal variation of gene expression. As an illustrative example only, the score card can indicate that the test pluripotent stem cell has 21% genes and/or 1057 of the genes assessed differentially methylated, and also indicate that the normal variation (e.g., in a plurality of reference pluripotent stem cell lines) for differentially methylated genes is 14.6-15.7% and/or 731-785 genes. Note, this example is based on DNA methylation analysis of about 5000 genes, e.g., as shown in Table 12A.

In some embodiments, the report, e.g., scorecard, can display the normalized values of the test pluripotent stem cell line, which are normalized to a reference pluripotent stem cell line (e.g., a selected “gold” standard line of the investigators choice) or the normal variation in reference pluripotent stem cell lines. Accordingly, a scorecard can display the % difference, and/or the change in absolute number of genes with altered DNA methylation levels as compared to the normal variation of DNA methylation. Similarly, the report, e.g., the scorecard can display the % difference, and/or the change in absolute number of genes which are differentially expressed as compared to the normal variation of gene expression levels. As an illustrative example only, the score card can indicate that the test pluripotent stem cell has a 34% increase, and/or an increase of 272 genes which are differentially methylated as compared to the normal variation of differentially methylated genes (e.g., in a plurality of reference pluripotent stem cell lines).

In some embodiments, the report, e.g., scorecard can subdivide the DNA methylated gene results and the gene expression results into cancer genes and/or developmental genes, e.g., the scorecard can display the % (total %, or % change), and/or absolute number (total number or change in number) of cancer genes, and/or lineage marker genes which have different DNA methylation levels as compared to the normal variation of DNA methylation levels, as well as display the % (total %, or % change), and/or absolute number (total number or change in number) of cancer genes, and/or lineage marker genes which are differentially expressed as compared to the normal variation level of gene expression.

In some embodiments, the report can be color-coded, for instance, if the % or absolute number of differentially DNA methylated genes or differentially expressed genes is above a certain pre-defined threshold level, the color of the % value or absolute number value can be a bright color (e.g., red), or otherwise marked (e.g. by a *) or highlighted for easy identification that this value indicates that the pluripotent stem cell line may have some undesirable characteristics and may be of questionable quality (e.g. likelihood of predisposed to form cancers) and/or have restricted utility.

In some embodiments, the scorecard can also display the reference values (either in % or absolute numbers) of the normal number of differentially methylated genes in a reference pluripotent stem cell line, which can be used to compare with the values from the pluripotent stem cell line tested. Similarly, in some embodiments the scorecard can also display the reference values (either in % or absolute numbers) of the normal number of differentially expressed genes in a reference pluripotent stem cell line, which can be used to compare with the values from the pluripotent stem cell line tested.

In an alternative embodiment, the report, e.g., scorecard can display the % or relative differentiation propensities to differentiate along specific lineages, e.g., neuronal, endoderm, ectoderm, mesoderm, pancreatic, cardiac lineages etc.

In some embodiments, the report, e.g., scorecard can also present text, either verbally or written, giving a recommendation of which applications and/or utility the pluripotent cell line is appropriate for, and/or which applications and/or utility the pluripotent cell line is not appropriate for.

In some embodiments of this aspect and all other aspects of the present invention, the report data, e.g., scorecard from the comparison module can be displayed on a computer monitor as one or more pages of the printed report, e.g., scorecard. In one embodiment of the invention, a page of the retrieved content can be displayed through printable media. The display module can be any device or system adapted for display of computer readable information to a user. The display module can include speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc

In some embodiments of the present invention, a World Wide Web browser can be used to provide a user interface to allow the user to interact with the system to input information, construct requests and to display retrieved content. In addition, the various functional modules of the system can be adapted to use a web browser to provide a user interface. Using a Web browser, a user can construct requests for retrieving data from data sources, such as data bases and interact with the comparison module to perform comparisons and pattern matching. The user can point to and click on user interface elements such as buttons, pull down menus, scroll bars, etc. conventionally employed in graphical user interfaces to interact with the system and cause the system to perform the methods of the invention. The requests formulated with the user's Web browser can be transmitted over a network to a Web application that can process or format the request to produce a query of one or more database that can be employed to provide the pertinent information related to the DNA methylation levels and gene expression levels, the retrieved content, process this information and output the results, e.g. at least one of any of the following: (i) display of an indication of the presence or absence (% and/or absolute numbers) of DNA methylation target genes with a variation of DNA methylation level as compared to the reference DNA methylation levels (e.g., of reference pluripotent stem cell line(s)); (ii) display of the presence or absence (% and/or absolute numbers) of gene expression target genes with a variation of gene expression level as compared to the reference gene expression levels (e.g., of reference pluripotent stem cell line(s)) (iii) display of the presence or absence (% and/or absolute numbers) of lineage marker target genes with a variation of gene expression level as compared to the reference lineage marker gene expression levels (e.g., of reference pluripotent stem cell line(s)). In one embodiment, DNA methylation level or gene expression level or gene expression level of lineage marker genes of one or more reference pluripotent stem cell lines can also displayed.

While, the assays, methods, systems, and kits described herein reference DNA methylation, it is to be understood that other epigenetic markers can be also used in the assays, methods, systems, and kits of the invention. For example, one can use patterns and levels of histone modifications or post-translational modifications in place of or in addition to DNA methylation and/or gene expression levels. Patterns of post-translational changes in certain polypeptides are known to correlate with certain diseases, such as Alzheimer's disease and cancer. See for example Table 3 in Int. Pat. App. Pub. No. WO/2010/044892. As used herein, the term “post-translational modification” or “PTM” refers to a reaction wherein a chemical moiety is covalently added to a protein. Many proteins can be post-translationaly modified through the covalent addition of a chemical moiety (also referred to herein as a “modifying moiety”) after the initial synthesis (i.e., translation) of the polypeptide chain. Such chemical moieties usually are added by an enzyme to an amino acid side chain or to the carboxyl or amino terminal end of the polypeptide chain, and may be cleaved off by another enzyme. Single or multiple chemical moieties, either the same or different chemical moieties, can be added to a single protein molecule. PTM of a protein can alter its biological function, such as its enzyme activity, its binding to or activation of other proteins, or its turnover, and is important in cell signaling events, development of an organism, and disease. Examples of PTM include, but are not limited to, ubiquitination, phosphorylation, glycosylation, sumoylation, acetylation, S-nitrosylation or nitrosylation, citrullination or deimination, neddylation, OClcNAc, ADP-ribosylation, methylation, hydroxylation, fattenylation, ufmylation, prenylation, myristoylation, S-palmitoylation, tyrosine sulfation, formylation, and carboxylation. Assays for determining and mapping post-translational modifications are well known to the skilled artisan. See for example, U.S. Pat. Nos. 6,465,199 and 6,495,664; and U.S. Pat. App. Publ. No. 2006/0078998, 2006/0210978 and 2008/007025, content of all of which is herein incorporated by reference.

### Kits

Another aspect of the present invention relates to a kit for determining the quality of a pluripotent stem cell line, comprising: (i) reagents for measuring methylation status of a plurality of DNA methylation genes, (ii) reagents for measuring gene expression levels of a plurality of Gene expression genes; and (iii) reagents for measuring the differentiation propensity of the pluripotent stem cell into ectoderm, mesoderm and endoderm lineages. In some embodiments, the kit further comprises a score card as disclosed herein. In some embodiments, the kit further comprises instructions for use.

In one aspect the invention provides a kit comprising a scorecard. In some embodiments, a kit further comprises the reagents for reprogramming a somatic cell or differentiated cell into an induced pluripotent stem cell (iPSC) and also comprises the reagents for quality-assessing the generated iPS cell lines. Examples of reagents used to reprogram a somatic cell into an induced pluripotent stem (iPS) cell are well known to persons of ordinary skill in the art, and include those as discussed herein, for example, but not limited to the methods and kits for reprogramming a somatic cell to an iPS cell or an piPS cell, as disclosed in International patent applications; WO2007/069666; WO2008/118820; WO2008/124133; WO2008/151058; WO2009/006997; and U.S. Patent Applications US2010/0062533; US2009/0227032; US2009/0068742; US2009/0047263; US2010/0015705; US2009/0081784; US2008/0233610; U.S. Pat. No. 7,615,374; U.S. patent application Ser. No. 12/595,041, EP2145000, CA2683056, AU8236629, Ser. No. 12/602,184, EP2164951, CA2688539, US2010/0105100; US2009/0324559, US2009/0304646, US2009/0299763, US2009/0191159, the contents of which are incorporated herein in their entirety by reference. In some embodiments, the kit comprises the reagents for virally-induced or chemically induced generation of reprogrammed cells e.g., iPS cells, as disclosed in EP1970446, US2009/0047263, US2009/0068742, and 2009/0227032, which are incorporated herein in their entirety by reference.

In some embodiments, a kit as disclosed herein also comprises at least one reagent for selecting a desired pluripotent stem cell line among many cell lines, e.g., reagents to select one or more appropriate pluripotent stem cell line for the intended use of the cell line. Such agents are well known in the art, and include without limitation, labeled antibodies to select for cell-specific lineage markers and the like. In some embodiments, the labeled antibodies are fluorescently labeled, or labeled with magnetic beads and the like. In some embodiments, a kit as disclosed herein can further comprise at least one or more reagents for profiling and annotating an existing ES cell and/or iPS cell bank in high throughput, etc. according to the methods as disclosed herein.

In one aspect the invention provide a kit comprising a pluripotent stem cell selected by an assay, method, or system of the invention. In addition to the above mentioned component(s), the kit can also include informational material. The informational material can be descriptive, instructional, marketing or other material that relates to the methods described herein and/or the use of the components for the assays, methods and systems described herein. For example, the informational material may describe methods for selecting a pluripotent stem cell, for characterizing a plurality of properties of a pluripotent cell, or generating a scorecard according to the invention. Without limitations, if a kit includes material suitable for administering to a subject, the kit can optionally include a delivery device.

In some embodiments, the methods, systems, kits and devices as disclosed herein can be performed by a service provider, for example, where an investigator can have one or more samples (e.g., an array of samples) each sample comprising a pluripotent stem cell line, or a different population of pluripotent stem cells, for assessment using the methods, kits and systems as disclosed herein in a diagnostic laboratory operated by the service provider. In such an embodiment, after performing the assays, methods and systems of the invention as disclosed, the service provider can performs the analysis and provide the investigator a report, e.g., a score card, of the characteristics of each pluripotent stem cell line analyzed. In alternative embodiments, the service provider can provide the investigator with the raw data of the assays and leave the analysis to be performed by the investigator. In some embodiments, the report is communicated or sent to the investigator via electronic means, e.g., uploaded on a secure web-site, or sent via e-mail or other electronic communication means. In some embodiments, the investigator can send the samples to the service provider via any means, e.g., via mail, express mail, etc., or alternatively, the service provider can provide a service to collect the samples from the investigator and transport them to the diagnostic laboratories of the service provider. In some embodiments, the investigator can deposit the samples to be analyzed at the location of the service provider diagnostic laboratories. In alternative embodiments, the service provider provides a stop-by service, where the service provider send personnel to the laboratories of the investigator and also provides the kits, apparatus, and reagents for performing the assays, methods and systems of the invention as disclosed herein of the investigators pluripotent stem cell lines in the investigators laboratories, and analyses the result and provides a report to the investigator of the characteristics of each pluripotent stem cell line, or a plurality of pluripotent stem cell line analyzed.

Example Workflow of a High-Throughput Sample Processing to Produce a Deviation or Lineage Scorecard

As an exemplary example, but by no way a limitation, a scorecard workflow is illustrated by the following case study: A large company (or foundation) plans to establish a stem cell bank providing HLA-matched iPS cell lines for X % of the US population, which requires 10,000 iPS cell lines. All cell lines will be commercially available, and to make the resource most valuable to researchers and companies, it is planned to publish scorecard characterizations for each cell line. To facilitate automatization, all iPS cell lines are grown in 96-well plates or 384-well plates. Most sample processing is robotized, and all cell lines are barcoded and tracked by a central LIMS. The scorecard characterization is performed as follows:

(1) Deviation Scorecard/Confirmation of Pluripotency:

A researcher loads a liquid-handling robot as follows: (i) one 96-well plate with one iPS cell line per well; (ii) 96-well RNA extraction kit, (iii) custom qPCR plates (96-well or 384-well) with pre-spotted primers for 96 marker genes and controls.

(2) A robot performs RNA extraction of the entire plate and pipettes the RNA from each well into separate qPCR plates (when using 96-well qPCR plates) or into ¼ of a plate (when using 384-well qPCR plates). Reverse transcription is performed in the same plate, and barcoded Ct tables are transferred to the LIMS.

(3) Lineage Scorecard/Quantification of Differentiation Potential:

Starting from a 96-well plate with one iPS cell line per well, a researcher will harvest the cells from each well and plate them into three new 96-well plates, giving rise to three biological replicates for embryoid body (EB) differentiation. Differentiation-inducing medium is added and the plates are left in the incubator for N days without media changes.

(4) After a defined period of time (e.g. n days) of EB differentiation, the plates are loaded into a liquid-handling robot and qPCR analysis is performed as described in steps 1 and 2, with the only exception that custom qPCR plates with differentiation-specific marker genes are used.

(5) Upon completion of the experiments, the researcher loads the unprocessed Ct values into a custom scorecard software. This software imports the output data format from any of the common qPCR machines, performs relative normalization using a number of house-keeping genes and calculates the scorecard prediction.

(6) Gene Set Selection.

As disclosed herein, the scorecard comprises two independent but complementary parts: (i) the deviation scorecard, and (ii) the lineage scorecard. In some embodiments, the assay for generation of data for the deviation scorecard can consist of a single 96-well qPCR plate (or in some embodiments, four samples on a 384-well qPCR plate) with the most relevant genes for determining whether or not a given cell line classifies as pluripotent. In some embodiments, the assay for generation of data for the lineage scorecard can consist of two 96-well plates (or in some embodiments, two samples on a 384-well qPCR plate) with the most relevant genes for quantifying the differentiation propensities of a given cell line.

In some embodiments, the optimal gene selection for both assays for both scorecards using a multiplex qPCR assay can be further validated and optimized. Furthermore, in some embodiments, one may perform the deviation assay prior to the lineage scorecard assay to determine the pluripotent state of the stem cell line of interest, and possibly obviating the need for EB differentiation assay for the lineage scorecard assay. Accordingly, in some embodiments, a validation phase can be performed which uses a single 384-well qPCR plate designed for both the deviation scorecard assay and the lineage scorecard assay. In some embodiments, multiple plates are used for the assay of each cell line, which includes plates for each biological stem cell line of interest replicate, plates for stem cell line in its pluripotent state and one for the stem cell line in its EB state. In some embodiments, genes to be included in such a 384-well qPCR plate (“tech-dev plate”) can be selected using the following gene set selection:

1. Normalization: Each plate contains six normalization genes in technical duplicate, three positive controls and one negative control.

2. Supported cell types/lineages: Lineage marker genes can be selected which are the same as the NanoString-based prototype for the qPCR-based scorecard (ectoderm, mesoderm and endoderm germ layers as well as the neural and hematopoietic lineages, or any selection of genes listed in Table 7 or 13A and 13B and Table 14). In addition, in some embodiments, a lineage marker genes can comprise additional categories of gene sets, including but not limited to: pluripotent cell signature, epidermis, mesenchymal stem cells, bone, cartilage, fat, muscle, blood vessel, heart, lymphoid cells, myeloid cells, liver, pancreas, epithelium, motor neurons, monocytes-macrophages (see Tables 13A and 13B and Table 14).

3. Additional features: In some embodiments, a qPCR plate for deviation and lineage scorecard assays can also comprise (i) qPCR primers for the four reprogramming viruses commonly used for reprogramming somatic cells to iPSC (e.g. primers to any of the reprogramming genes Sox2, Oct4, c-myc, Klf4 etc) as well as (ii) a five-gene signature for male-female classification in order to detect potential sample mix-ups (see Table 14); and (iii) a one-gene signature for detecting extensive apoptosis. In some embodiments, a qPCR plate for deviation and lineage scorecard assays can also comprise a subset of the most transcriptionally and/or epigenetically variable genes in ES and iPS cell lines that the inventors have identified herein.

Validation: In some embodments, one can validate a qPCR plate for assays for producing data for a deviation scorecard and a lineage scorecard. Validation can be performed in three phases. During an initial validation phase, one will assess the qPCR plate to determine if it provides similar accuracy and predictive power as the NanoString assay. A second biological validation phase can be performed which will assess and confirm the predictiveness of the qPCR-based scorecard for many more pluripotent stem cell lines and propensity to differentatin into a variety of different lineages of interest. A final assay validation can be performed which will optimize the qPCR plate for technical consistency with all earlier data. More specifically, in some embodiments, a validation phases will be conducted as follows:

1. Technical qPCR assay validation. One can directly compare the results from a NanoString-based scorecard with a qPCR-based scorecard, comparing the accuracy, sensitivity and robustness of each gene between the NanoString and qPCR platform. Furthermore, one can also confirm that the qPCR-based scorecard is able to predict cell-line specific differences in the efficiency of directed motor neuron differentiation.

2. Biological qPCR assay validation and extension of scope. The inventors have extensively validated the lineage scorecard for predicting motor neuron differentiation using an EB-based protocol. One can perform similar validation of the lineage scorecard for hematopoietic differentiation using a similar EB-based protocol. Accordingly, one can validate the lineage scorecard predictability using several different additional differentiation protocols to quantitatively determine the efficiencies of differentiation into various different lineages. Furthermore, one can validate the qPCR assays using at least about 100 or more pluripotent stem cell lines, for example, selected from but not limited to, human pluripotent cell lines, partially reprogrammed cell lines, embryonic cancer cell lines etc., in order to calibrate the deviation scorecard. Such validation can be used optimize and redesign qPCR-based scorecard assay will be for large-scale production and tailored to a particular stem cell line or lineage preference.

3. Technical validation. In some embodiments further validation may be desired to validate software and assay handling of a qPCR assay, for example, stability of the plates, easy of reading the output from the qPCR plates and the like. Such validation and optimization is commonly know by persons of ordinary skill in the art.

### Uses of the Scorecards.

In some embodiments, the methods, systems, kits and scorecards as disclosed herein can be used in a variety of ways clinically and in research applications. For instance, methods, systems, kits and scorecards as disclosed herein are useful for identifying epigenetic and functional genomic changes in pluripotent stem cell lines in response to a drug, or for selecting a plurality of pluripotent stem cell lines to have the same properties to be used in a drug screen, which is useful to ensure the quality of the drug screen and ensure that any potential hits are the effect of the drug rather than due to variations in the different pluripotent stem cells. In some embodiments, methods, systems, kits and scorecards as disclosed herein are useful for identifying and selecting a pluripotent stem cell line which would be suitable for therapeutic use, e.g., stem cell therapy or other regenerative medicine, to ensure that the implanted stem cell line does not have a predisposition to differentiate into cancer cells. Similarly, the methods, systems, kits and scorecards as disclosed herein are useful for characterizing and validating an iPSC generated from a mammal, e.g., a human, to ensure that the iPSC possess qualities, and can be compared to other pluripotent stem cells.

In some embodiments, the methods, systems, kits and scorecards as disclosed herein can be used in clinics to determine clinical safety and utility of a particular pluripotent stem cell line.

In some embodiments, the methods, systems, kits and scorecards as disclosed herein can be used as a quality control to monitor the characteristics of pluripotent stem cells over different passages and/or before and after cryopreservation procedures, for example, to ensure that no significant epigenetic or functional genomic changes has occurred over time (e.g., over passages and after cryopreservation). For example, the methods, systems, kits and scorecards as disclosed herein can be used to characterize all stem cells in stem cell bank, to catalogue each stem cell line which is placed in the bank, and to ensure that the stem cells have the same properties after thawing as they did prior to cryopreservation.

In some embodiments, the raw data (e.g., DNA methylation and/or gene expression data) and/or scorecard data for each pluripotent stem cell line can be stored in a centralized database, where the data and/or scorecard can be used to select a pluripotent stem cell line for a particular use or utility. Accordingly, one aspect of the present invention relates to a database comprising at least one of: the DNA methylation data, gene expression data, and scorecard for a plurality of pluripotent stem cell lines, and in some embodiments, the database comprises the DNA methylation data, gene expression data, and/or scorecard for a plurality of pluripotent stem cell lines in a stem cell bank.

In some embodiments, the methods, systems, kits and scorecards as disclosed herein can be used in research to monitor functional genomic changes as a pluripotent stem cell differentiates into different lineages. In some embodiments, the methods, systems, kits and scorecards as disclosed herein can be used to monitor and determine the characteristics of pluripotent stem cells from particular diseases, e.g., one can monitor pluripotent stem cells from subjects with genetic defects or particular genetic polymorphisms, and/or having a particular disease, e.g., one can determine the monitor and determine the functional genomic differences between an iPSC cell derived from a subject with a neurodegenerative disease, such as ALS, as compared to a normal iPSC cell from a healthy subject, such a health sibling. Similarly, one can determine if iPS cell are comparable in functional genomics and differentiation propensity as compared to ES cells or other pluripotent stem cell. Additionally, the methods, systems, kits and scorecards as disclosed herein can fully characterize the pluripotency of a stem cell line without the need for teratoma assays and/or generation of chimera mice, therefore significantly increasing the high-throughput ability of characterizing pluripotent stem cell lines.

In some embodiments, the scorecard can be included in an “all-included” kit for making and validating patient-specific iPS-cell lines. For example, in such an embodiment, the kit can comprise (i) a sample collection device, e.g., needle or tube as required for collecting patient somatic or differentiated cells, and in some embodiments, a patient consent form, (ii) reagents for reprogramming the patients collected somatic or differentiated cell into an iPS cell, e.g., where the kit comprises any number or combination of reprogramming factors, such as virus/DNA/RNA/protein as described herein, and ES-cell media), and (iii), the assays for generating a scorecard as disclosed herein, e.g., reagents for performing at DNA methylation assay, reagents for performing a gene expression assay, and reagents for performing the verification of the iPS cell line differentiation potential). In some embodiments, the kit can comprise one or more reference pluripotent stem cell lines, which can be used as a positive control (or a negative control, e.g., where the pluripotent stem cell line has been identified with an undesirable characteristic) as a quality control for the kit. In some embodiments, the kit can also comprise a scorecard of a reference pluripotent stem cell to be used, for example, for comparison purposes for with the patient iPS cell being assessed. In some embodiments, the “all-included” kit can be used for utility prediction of the patient iPS cell line based on the results from the quality control (e.g., as determined by the bioinformatic determination as disclosed herein). In some embodiments, an “all-included” kit can also additionally comprise the materials, reagents and protocols for directed differentiation of the newly generated patent iPS cell line into a particular cell type of interest (e.g., cardiomyocytes, beta cells, hepatocytes, hair follicle stem cells, cartilage, hematopoietic cells, and the like).

In some embodiments, the scorecard, methods, kits and assays as disclosed herein can be used to provide a service, such as a “cell-to-quality assured pluripotent stem cell line” service, which can be carried out, for example, in a directly in a clinic, or in a clinical diagnostics lab, or as a mail-in service carried out by a dedicated facility. For example, such a service would operate in that an investigator, or a patient sends in somatic cells (e.g., differentiated cells) into the service provider, whereby the service provider generates iPS cell lines from the somatic cells, using commonly known methods as disclosed herein, and the service provider performs the methods and assays as disclosed herein on the generated pluripotent iPS cell lines, for example, the service provider will perform (i) the differentiation propensity assay, (ii) the DNA methylation assay and optionally, (iii) gene expression assay, and subsequently perform the analysis to generate a scorecard for each individual iPS cell analyzed. The service provider can also optionally suggest the suitability of one or more selected iPS cell lines for a particular use, e.g., the service provider can suggest “iPS cell line 1” which was identified to have a high efficiency of differentiating along motor neuron differentiation pathways would be suitable for neuronal differentiation, or similarly the service provider can suggest “iPS cell line 2” which was identified to have a high efficiency of differentiating along hepatic lineages would be suitable for differentiation into liver cells for use in liver cell regenerative medicine. Similarly, the service provider can suggest “iPS cell line 6” which was identified to outlier DNA methylated genes, and/or outlier gene expression levels of specific genes, e.g., outlier DNA methylation or gene expression of cancer genes, may not be suitable for therapeutic uses in regenerative medicine due to a risk of potential cancer formation. In some embodiment, the service provider can not make a recommendation, but rather provide a report of the scorecard for each iPS cell line generated and analyzed by the service provider. In some embodiments, the service provider returns the iPS cell lines to the investigator, or patient with a copy of the report scorecard.

In some embodiments, the scorecard, methods, kits and assays as disclosed herein can be used in creating a database, and where such a database would be useful in organizing and cataloguing a pluripotent stem cell repository, e.g., a central repository (e.g., a tissue and/or cell bank) containing a large number of quality-controlled and utility-predicted pluripotent cell lines, such that one can use a database comprising the data of each scorecard for each pluripotent stem cell line in the bank to specifically select a particular pluripotent stem cell line for the investigators intended use. For example, a user of the database can click a “suggest best cell line for my application” button on the website linked to the database, and obtain information and the identity a number useful cell lines for the investigators particular use. In some embodiments, the use of such a database can be easily extended such that a user can upload microarray data (e.g., DNA methylation data and/or gene expression data) for a particular cell type of interest, this microarray data can be run through the scorecard algorithm and the results compared with the database scorecard results for the pluripotent stem cell bank. In a simple analogy, the database could function similar to Google's “search for similar sites”, whereby the database could be used as an efficient way to select useful cell lines for novel and/or mixed tissue types, or to identify pluripotent stem cell lines in a cell bank that may have potential to differentiate into a desired differentiated stem cell line.

In some embodiments, the scorecard, methods, kits and assays as disclosed herein can be used for identification and selection of a desired pluripotent stem cell line for mass production, for example use of the methods, assays and scorecards as disclosed herein to identify and characterize and validate the quality of pluripotent stem cell lines that grow well and/or efficiently in large quantities, e.g., large batch cultures or in bioreactors, and selection of pluripotent stem cell lines that can be differentiated efficiently in bulk cultures into a specific cell type.

In another embodiment, the scorecard, methods, kits and assays as disclosed herein can be used for selection of a pluripotent stem cell line based on properties of pluripotent robustness, for example, the methods, assays and scorecards as disclosed herein can be used to identify pluripotent stem cell lines which are easy to culture in vitro (e.g., require little attention, and/or do not readily spontaneously differentiate, and/or maintain the pluripotency properties). For example, in some embodiments, a pluripotent stem cell line can be assessed using the methods, assays and scorecards prior to culturing, and then at different timepoints during and after culturing, and in different culture conditions and media conditions to identify one or more pluripotent stem cell lines which maintain their initial qualities in short- and long-term culture conditions.

In another embodiment, the scorecard, methods, kits and assays as disclosed herein can be used for selection of a pluripotent stem cell line for drug responsiveness, for example, a pluripotent stem cell line can be assessed using the methods, assays and scorecards as disclosed herein to prior to, during, and after contacting with a drug or other agent or stimuli (e.g., electric stimuli for cardiac pluripotent progenitors) to generate a drug metabolism and/or pharmacogenomics signature of the pluripotent stem cell line, for example which can be used to identify pluripotent stem cell lines which can be particularly useful for drug screening and drug discovery, including, for example drug toxicity assays.

In another embodiment, the scorecard, methods, kits and assays as disclosed herein can be used for selection of a pluripotent stem cell line based on its safety profile, for example, a pluripotent stem cell line can be assessed using the methods, assays and scorecards as disclosed herein to identify its likelihood to transduce into a cancer cell or likelihood of metastasis or differentiate into a particular cell type, or likelihood to dedifferentiate, which is very useful in validating the safety of a pluripotent stem cell line or its differentiated progeny in clinical applications, such as cell replacement therapy and regenerative medicine.

In another embodiment, the scorecard, methods, kits and assays as disclosed herein can be used for selection of a pluripotent stem cell line for efficacy. For example, one can use a scorecard predictions of a particular pluripotent stem cell line to predict whether, and/or how well differentiated cells derived from the pluripotent cell line will continue to differentiate along a particular desired cell lineage, and/or if they will proliferate once implanted into a subject, e.g., a human patient or in an animal model (e.g., a rat or mouse disease model etc.). More generally, in some embodiments the scorecard can be used to predict not only the behavior of a pluripotent cell line, but also from differentiated cells that are directly or indirectly derived from the pluripotent cell line.

In another embodiment, the scorecard, methods, kits and assays as disclosed herein can be used for selection of a pluripotent stem cell line which has the same or very similar characteristics of a pluripotent stem cell in vivo (e.g., to select pluripotent stem cell which are a truthful representation of the cell in an in vivo environment). For example, a pluripotent stem cell line can be assessed using the methods, assays and scorecards as disclosed herein to identify a pluripotent stem cell line suitable for disease modeling, as it is important to use pluripotent stem cell lines that closely resemble their corresponding cells in vivo. Accordingly, one of ordinary skill in the art can easy use the scorecard as disclosed herein to predict which pluripotent cell lines resemble their corresponding cells in vivo, e.g. by comparing the properties (listed on the scorecard) of the pluripotent stem cell line with corresponding cells harvested from a subject (e.g. an animal model, or disease model such as a rodent disease model), to minimize deviations from a reference population of clean ES cell lines as compared to how the cell behaves in vivo.

In another embodiment, the scorecard, methods, kits and assays as disclosed herein can be used for selection and/or quality control, and/or validation of a pluripotent stem cell line in different or new states of pluripotency or multipotency, for example to provide information of pluripotent stem cell lines which are useful for differentiating and making cell types in vitro but do not fall under the usual definition of human ES cell lines (e.g., human ground-state ES cell and partially reprogrammed cell lines, e.g., partially induced pluripotent stem (piPS) cells, which are capable of being reprogrammed further to a pluripotent stem cell).

It has been shown that continued in vitro culture and passaging improves the quality of iPS cell lines (see Polo et al., Nat. Biotechnol. 2010 August; 28(8):848-55, and Nat Rev Mol Cell Biol. 2010 September; 11(9):601, and Nat Rev Genet. 2010 September; 11(9): 593). On the other hand, continued passaging is expensive. Accordingly, in some embodiments, the scorecard, methods, kits and assays as disclosed herein can be used for measuring how much passaging is sufficient for improving the quality of the pluripotent stem cell line.

In further embodiments, the scorecard, methods, kits and assays as disclosed herein can be used in a variety of different research and clinical uses to characterize and monitor and validate pluripotent stem cells, for example, typical application includes in areas such as, but not limited to, (i) labs and/or companies interested in disease mechanisms (e.g., using the kits or services as disclosed herein to reduce the complexity of generating iPS cell lines, as well as differentiated cells for disease modeling and small-scale drug screening, (ii) labs and/or companies trying to identify small molecules and/or biologicals for a disease given target (e.g., using the kits and/or services as disclose herein to enable the production of large numbers of highly standardized cells for drug screening), (iii) clinical and pre-clinical research groups for quality control and validating pluripotent stem cell lines where they are interested in producing cells for implantation into humans or animals (e.g., using a kit and/or service as disclosed herein to enables quality control at a level of accuracy that will be sufficient for regulatory approval, e.g., FDA approval), (iv) tissue banks that desire to give their customers information, including advice, and data about the performance and quality and utility of the pluripotent stem cell lines on offer (e.g., using a kit and/or service as disclosed herein which provides unbiased assessment of the quality and/or utility of a large number of pluripotent cell lines, for example in a cheap, high throughput manner, for example, ultimately running the assays on 100,000s of pluripotent stem cell lines to cover the whole population of cell lines stored in the cell bank), (v) private consumers who desire to generate, and optionally, bank at least one or more pluripotent cell lines, e.g., iPS cell lines (or piPS cell lines) generated from their somatic differentiated cells, either for themselves and/or their children or other offspring, for example, as a type of health insurance policy for future regenerative medicine purposes.

### Therapeutic Uses

Various disease and disorders have been suggested as potential targets for stem cell therapy, such as cancer, diabetes, cardiac failure, muscle damage, Celiac Disease, neurological disorder, neurodegenerative disorder, and lysosomal storage diseases, as well as, any of the following diseases, ALS, Parkinson, monogenetic diseases and Mendelian diseases, ageing, general wear and tear of the human body, rheumatic arthritis and other inflammatory diseases, birth defects, etc. Accordingly, the assays, methods, systems and kits of the invention can be used to select pluripotent stem cells for administering to a subject for treatment.

Therefore, in one aspect the invention provide for a method of treatment, prevention, or amelioration of disease or disorder in a subject, the method comprising administering to the subject a pluripotent stem cell, (e.g., pluripotent cells, differentiated cells derived from pluripotent cells, and differentiated cells obtained by other methods that involve reprogramming (e.g. transdifferentiation)) wherein the pluripotent stem cell is selected by an assay, kit, method, or system of the invention. Without limitation, the pluripotent stem cell can be treated for differentiation along a specific lineage before administration to a subject.

Routes of administration suitable for the methods of the invention include both local and systemic administration. Generally, local administration results in of the cells being delivered to a specific location as compared to the entire body of the subject, whereas, systemic administration results in delivery of the cells to essentially the entire body of the subject. Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. One method of local administration is by intramuscular injection.

One preferred method of administration is transplantation of such a pluripotent cell, or differentiated progeny derived from the pluripotent stem cell, in a subject. The term “transplantation” includes, e.g., autotransplantation (removal and transfer of cell(s) from one location on a patient to the same or another location on the same patient), allotransplantation (transplantation between members of the same species), and xenotransplantation (transplantations between members of different species). Skilled artisan is well aware of methods for implanting or transplantation of cells for treatment of various disease, which are amenable to the present invention.

For administration to a subject, the pluripotent stem cells can be provided in pharmaceutically acceptable compositions. These pharmaceutically acceptable compositions comprise one or more of the pluripotent cells, formulated together with one or more pharmaceutically acceptable carriers (additives) and/or diluents. As described in detail below, the pharmaceutical compositions of the present invention can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, cells can be implanted into a subject or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In the context of administering a pluripotent stem cell, the term “administering” also include transplantation of such a cell in a subject. As used herein, the term “transplantation” refers to the process of implanting or transferring at least one cell to a subject. The term “transplantation” includes, e.g., autotransplantation (removal and transfer of cell(s) from one location on a patient to the same or another location on the same patient), allotransplantation (transplantation between members of the same species), and xenotransplantation (transplantations between members of different species).

The pluripotent stem cell can be administrated to a subject in combination with a pharmaceutically active agent. As used herein, the term “pharmaceutically active agent” refers to an agent which, when released in vivo, possesses the desired biological activity, for example, therapeutic, diagnostic and/or prophylactic properties in vivo. It is understood that the term includes stabilized and/or extended release-formulated pharmaceutically active agents. Exemplary pharmaceutically active agents include, but are not limited to, those found in Harrison's Principles of Internal Medicine, 13^{th} Edition, Eds. T. R. Harrison et al. McGraw-Hill N.Y., NY; Physicians Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with autoimmune disease or inflammation. In addition, the methods and compositions described herein can be used to treat domesticated animals and/or pets.

A subject can be one who has been previously diagnosed with or identified as suffering from or having a disorder characterized with a disease for which a stem cell based therapy would be useful.

A subject can be one who is not currently being treated with a stem cell based therapy.

In some embodiments of the aspects described herein, the method further comprising selecting a subject with a disease that would benefit from a stem cell based therapy.

As used herein, the term “neurodegenerative disease or disorder” comprises a disease or a state characterized by a central nervous system (CNS) degeneration or alteration, especially at the level of the neurons such as Alzheimer's disease, Parkinson's disease, Huntington's disease, amyotrophic lateral sclerosis, epilepsy and muscular dystrophy. It further comprises neuro-inflammatory and demyelinating states or diseases such as leukoencephalopathies, and leukodystrophies. Exemplary, neurodegenerative disorders include, but are not limited to, AIDS dementia complex, Adrenoleukodystrophy, Alexander disease, Alpers' disease, Alzheimer's disease, Amyotrophic lateral sclerosis, Ataxia telangiectasia, Batten disease, Bovine spongiform encephalopathy, Canavan disease, Corticobasal degeneration, Creutzfeldt-Jakob disease, Dementia with Lewy bodies, Fatal familial insomnia, Frontotemporal lobar degeneration, Huntington's disease, Infantile Refsum disease, Kennedy's disease, Krabbe disease, Lyme disease, Machado-Joseph disease, Multiple sclerosis, Multiple system atrophy, Neuroacanthocytosis, Niemann-Pick disease, Parkinson's disease, Pick's disease, Primary lateral sclerosis, Progressive supranuclear palsy, Refsum disease, Sandhoff disease, Diffuse myelinoclastic sclerosis, Spinocerebellar ataxia, Subacute combined degeneration of spinal cord, Tabes dorsalis, Tay-Sachs disease, Toxic encephalopathy, and Transmissible spongiform encephalopathy.

As used herein, the term “cancer” includes a malignancy characterized by deregulated or uncontrolled cell growth, for instance carcinomas, sarcomas, leukemias, and lymphomas. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

The term “carcinoma” includes malignancies of epithelial or endocrine tissues, including respiratory system carcinomas, gastrointestinal system carcinomas, genitourinary system carcinomas, testicular carcinomas, breast carcinomas, prostate carcinomas, endocrine system carcinomas, melanomas, choriocarcinoma, and carcinomas of the cervix, lung, head and neck, colon, and ovary. The term “carcinoma” also includes carcinosarcomas, which include malignant tumors composed of carcinomatous and sarcomatous tissues. An “adenocarcinoma” refers to a carcinoma derived from glandular tissue or a tumor in which the tumor cells form recognizable glandular structures.

The term “sarcoma” includes malignant tumors of mesodermal connective tissue, e.g., tumors of bone, fat, and cartilage.

The terms “leukemia” and “lymphoma” include malignancies of the hematopoietic cells of the bone marrow. Leukemias tend to proliferate as single cells, whereas lymphomas tend to proliferate as solid tumor masses. Examples of leukemias include acute myeloid leukemia (AML), acute promyelocytic leukemia, chronic myelogenous leukemia, mixed-lineage leukemia, acute monoblastic leukemia, acute lymphoblastic leukemia, acute non-lymphoblastic leukemia, blastic mantle cell leukemia, myelodyplastic syndrome, T cell leukemia, B cell leukemia, and chronic lymphocytic leukemia. Examples of lymphomas include Hodgkin's disease, non-Hodgkin's lymphoma, B cell lymphoma, epitheliotropic lymphoma, composite lymphoma, anaplastic large cell lymphoma, gastric and non-gastric mucosa-associated lymphoid tissue lymphoma, lymphoproliferative disease, T cell lymphoma, Burkitt's lymphoma, mantle cell lymphoma, diffuse large cell lymphoma, lymphoplasmacytoid lymphoma, and multiple myeloma.

For example, the pluripotent cells selected by the assays, kits, methods, and systems of the invention can be used to treat many kinds of cancers, such as oligodendroglioma, astrocytoma, glioblastomamultiforme, cervical carcinoma, endometriod carcinoma, endometrium serous carcenoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, colon adenocarcinoma, esophagus adenocarcinoma, hepatocellular carcinoma, mouth cancer, gall bladder adenocarcinoma, pancreatic adenocarcinoma, small intestine adenocarcinoma, stomach diffuse adenocarcinoma, prostate (hormone-refract), prostate (untreated), kidney chromophobic carcinoma, kidney clear cell carcinoma, kidney oncocytoma, kidney papillary carcinoma, testis non-seminomatous cancer, testis seminoma, urinary bladder transitional carcinoma, lung adenocarcinoma, lung large cell cancer, lung small cell cancer, lung squamous cell carcinoma, Hodgkin lymphoma, MALT lymphoma, non-hodgkins lymphoma (NHL) diffuse large B, NHL, thymoma, skin malignant melanoma, skin basalioma, skin squamous cell cancer, skin merkel zell cancer, skin benign nevus, lipoma, and liposarcoma abnormal cell growth.

### Drug Screening

The methods, assays, systems and kits of the invention can be used to develop in vitro assays based on well defined human cells. Existing assays for drug screening/testing and toxicology studies have several shortcomings because they are of animal origin, immortalized cell lines, or derived from cadavers. Because these alternatives often poorly reflect the physiology of normal human cells, stem-cell derived assays (e.g., homogeneous populations of heart and liver cells) could be established in the future and may play an important role for these purposes. For example, the methods, assays, systems, and kits of the invention can be used to identify and/or validate pluripotent stem cells that can differentiate along a lineage which is phenotypic of a disease. In addition to, or alternatively, the methods, assays, systems, and kits of the invention can be used to identify and/or validate pluripotent stem cells that can differentiate into an organ, and/or tissue lineage, or a part thereof. Such identified pluripotent cells then can be used for screening a test compound.

Furthermore, the flurry of new information now available on the molecular and cellular level related to human diseases (e.g., microarray data) makes it crucial to develop and test hypotheses about pathogenetic interrelations. The experimental access to specific cell types from all developmental stages and even from blastocysts deemed to harbor pathology based on pre-implantation genetic diagnosis may be useful in modeling and understanding aspects of human disease. Thus, such cell lines would also be valuable for the testing of drugs.

Accordingly, the invention provides a method for screening a test compound for biological activity, the method comprising: (a) obtaining a pluripotent stem cell, wherein the pluripotent cell is identified and validated for differentiation along a specific lineage; (b) optionally causing or permitting the pluripotent stem cell to differentiate to the specific lineage; (c) contacting the cell with a test compound; and (d) determining any effect of the compound on the cell. The effect on the cell can be one that is directly observable or indirectly by use of reporter molecules.

As used herein, the term “biological activity” or “bioactivity” refers to the ability of a test compound to affect a biological sample. Biological activity can include, without limitation, elicitation of a stimulatory, inhibitory, regulatory, toxic or lethal response in a biological assay. For example, a biological activity can refer to the ability of a compound to modulate the effect of an enzyme, block a receptor, stimulate a receptor, modulate the expression level of one or more genes, modulate cell proliferation, modulate cell division, modulate cell morphology, or a combination thereof. In some instances, a biological activity can refer to the ability of a test compound to produce a toxic effect in a biological sample.

As discussed above, the specific lineage can be a lineage which is phenotypic and/or genotypic of a disease. Alternatively, the specific lineage can be lineage which is phenotypic and/or genotypic of an organ and/or tissue or a part thereof.

As used herein, the term “test compound” refers to the collection of compounds that are to be screened for their ability to have an effect on the cell. Test compounds may include a wide variety of different compounds, including chemical compounds, mixtures of chemical compounds, e.g., polysaccharides, small organic or inorganic molecules (e.g. molecules having a molecular weight less than 2000 Daltons, less than 1000 Daltons, less than 1500 Dalton, less than 1000 Daltons, or less than 500 Daltons), biological macromolecules, e.g., peptides, proteins, peptide analogs, and analogs and derivatives thereof, peptidomimetics, nucleic acids, nucleic acid analogs and derivatives, an extract made from biological materials such as bacteria, plants, fungi, or animal cells or tissues, naturally occurring or synthetic compositions.

Depending upon the particular embodiment being practiced, the test compounds may be provided free in solution, or may be attached to a carrier, or a solid support, e.g., beads. A number of suitable solid supports may be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds may be screened individually, or in groups. Group screening is particularly useful where hit rates for effective test compounds are expected to be low such that one would not expect more than one positive result for a given group.

A number of small molecule libraries are known in the art and commercially available. These small molecule libraries can be screened for inflammasome inhibition using the screening methods described herein. For example, libraries from Vitas-M Lab and Biomol International, Inc. Chemical compound libraries such as those from of 10,000 compounds and 86,000 compounds from NIH Roadmap, Molecular Libraries Screening Centers Network (MLSCN) can be screened. A comprehensive list of compound libraries can be found at http://www.broad.harvard.edu/chembio/platform/screening/compound_libraries/index.htm. A chemical library or compound library is a collection of stored chemicals usually used ultimately in high-throughput screening or industrial manufacture. The chemical library can consist in simple terms of a series of stored chemicals. Each chemical has associated information stored in some kind of database with information such as the chemical structure, purity, quantity, and physiochemical characteristics of the compound.

Without limitation, the compounds can be tested at any concentration that can exert an effect on the cells relative to a control over an appropriate time period. In some embodiments, compounds are testes at concentration in the range of about 0.01 nM to about 1000 mM, about 0.1 nM to about 500 μM, about 0.1 μM to about 20 μM, about 0.1 μM to about 10 μM, or about 0.1 μM to about 5 μM.

The compound screening assay may be used in a high through-put screen. High through-put screening is a process in which libraries of compounds are tested for a given activity. High through-put screening seeks to screen large numbers of compounds rapidly and in parallel. For example, using microtiter plates and automated assay equipment, a pharmaceutical company may perform as many as 100,000 assays per day in parallel.

The compound screening assays of the invention may involve more than one measurement of the observable reporter function. Multiple measurements may allow for following the biological activity over incubation time with the test compound. In one embodiment, the reporter function is measured at a plurality of times to allow monitoring of the effects of the test compound at different incubation times.

The screening assay may be followed by a subsequent assay to further identify whether the identified test compound has properties desirable for the intended use. For example, the screening assay may be followed by a second assay selected from the group consisting of measurement of any of: bioavailability, toxicity, or pharmacokinetics, but is not limited to these methods.

### Algorithm and Methods of Bioinformatic Analysis for Producing a Score Card of a Pluripotent Stem Cell Line.

As discussed herein, the scorecard as comprises several components: (i) use of a DNA methylation assay to identify epigenetic modifications, e.g., DNA methylation gene outliers in a pluripotent cell as compared to the normal epigenetic variation, e.g., normal variation of DNA methylation for a set of target genes in reference pluripotent cell lines, (ii) use of a gene expression assay to identify genes where the gene expression level is an outlier in a pluripotent cell line as compared to the normal variation of DNA expression level for a set of target genes in reference pluripotent cell lines, (iii) use of a differentiation assay to predict a cellular differentiation bias using epigenetic modifications, (e.g., DNA methylation) and/or gene expression data from (i) and (ii), and/or gene expression/DNA methylation data from pluripotent cell lines that have been induced to differentiate, e.g., directed differentiation.

Each of these three applications or assays requires different bioinformatic methods in order to obtain a practically useful indication of a pluripotent cell line's quality and utility.

In some embodiments and discussed herein, any DNA methylation method can be used, for example, DNA methylation analysis can be performed by a number of methods, including, but not limited to, enrichment-based methods (e.g. MeDIP, MBD-seq and MethylCap), bisulfite-based methods (e.g. RRBS, bisulfite sequencing, Infinium, GoldenGate, COBRA, MSP, MethyLight) and restriction-digestion methods (e.g., MRE-seq). Each of these DNA methylation methods requires specific bioinformatic methods for data preprocessing and normalization in order to make the data useful for the scorecard analysis. These include, for example, correction for GC and CpG bias, bisulfite-specific alignment to the genomic DNA sequence etc.

Once the DNA methylation data are appropriately normalized, one identifies any genes and/or genomic regions that exhibit altered DNA methylation levels that may foster, or interfere with, an intended uses of the pluripotent cell line or its progeny. In some embodiments, the inventors have developed a statistical algorithm that identifies such genomic regions by comparing the DNA methylation profile of the pluripotent cell line of interest to one or more reference pluripotent stem cell lines, e.g., a previously characterized good, or alternatively, a previously characterized bad) pluripotent cell line. Technically, this is performed by applying a statistical test (e.g. t-test, Fisher's exact test, ANOVA) to each of a given set of candidate loci. To improve the robustness, one can use thresholds on the false discovery rate and the absolute DNA methylation difference between the cell line and the reference pluripotent stem cell line, and take the variability of the reference pluripotent stem cell line into account.

As disclosed in the Examples, a scorecard as disclosed herein summarizes if one or more pluripotent stem cell line of interest deviates from the ES cell reference cell line. As used herein, a ES cell reference line can be any number of ES cells of interest. In alternative embodiments, a ES cell reference line can constitute the DNA methylation and gene expression normal ranges for a number of iPSC and/or ES cells, for example, at least about 10- or at least about 20 low passage ES cell lines as used herein in the Examples.

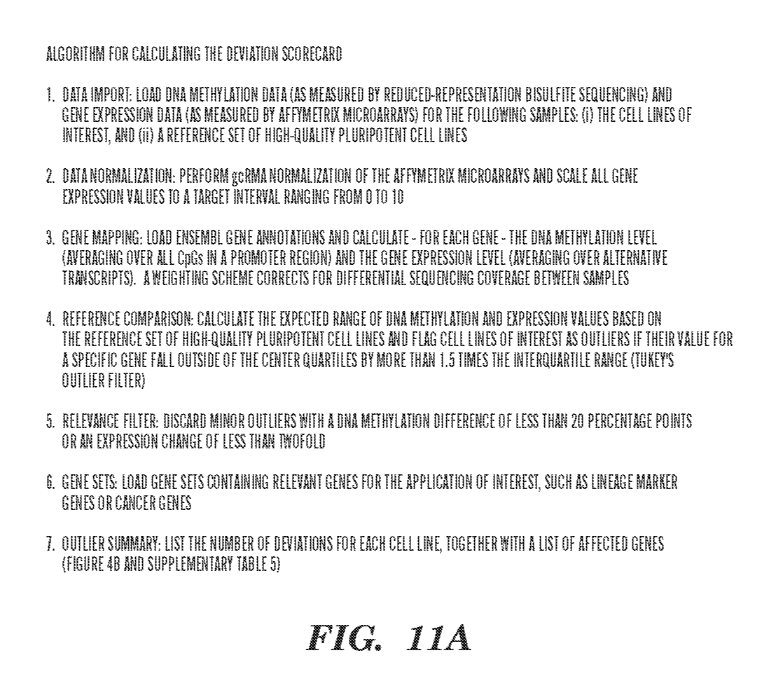

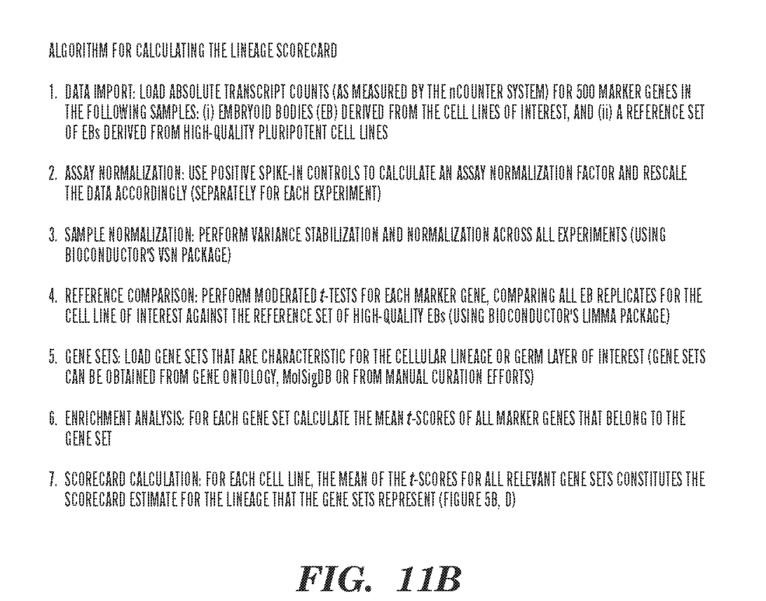

The algorithm for calculating the deviation scorecard (outlined in FIG. 11A) is the same for DNA methylation and gene expression data, with the only exception that the microarray data require an additional normalization step.

In some embodiments, the algorithum for determining a gene expression or DNA methylation scorecard includes the following steps:

(i) Data Import:

Import gene expression and/or DNA methylation data from the pluripotent stem cell of interest and at least one, or at least about 10 or more reference pluripotent stem cell lines which are used as high quality reference pluripotent stem cell control lines. In some embodiments, the gene expression data is microarray data, and in some embodiments, the DNA methylation data is whole-genome DNA methylation, or RRBS (reduced-representation bisulfite sequencing).

(ii) Optional Step of Data Normalization (Required for Gene Expression Only):

Perform normalization of the gene expression data, such as gcRMA normalization of microarray data and scale all gene expression values to a target interval range from 0 to 10. In some embodiments, the target interval reference range is normalized to 0 to 100, or from 0 to 1000 or 0 to about 500, or any preferred target interval range.

(iii) Gene Mapping:

Perform gene mapping to determine the DNA methylation level (averaging over all CpGs in a promoter region) and the gene expression levels (averaging over alternative transcripts) for each gene. In some embodiments, Ensembl gene annotations are useful to match the DNA methylation level and the gene expression levels for each gene. In some embodiments, a weighting scheme corrects for differential sequencing coverage between samples. Stated another way, a “reference corridor” or the “reference DNA methylation levels” or the “reference Gene expression levels” provide a range of values of the expected levels or range of DNA methylation and gene expression transcript levels for any gene in reference high-quality ES cell.

(iv) Reference Comparison:

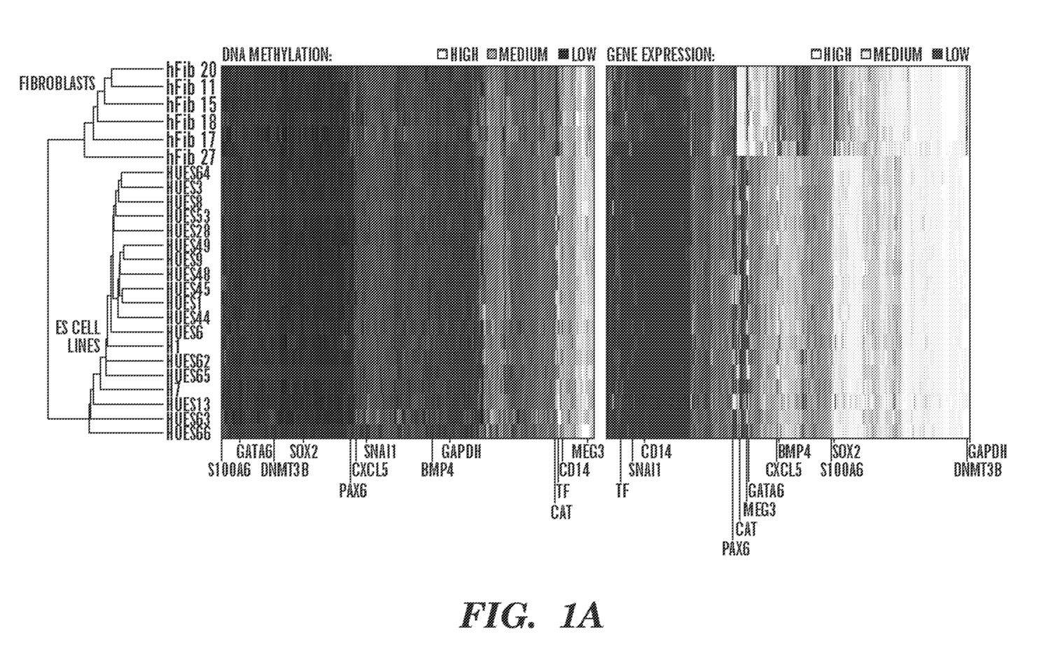

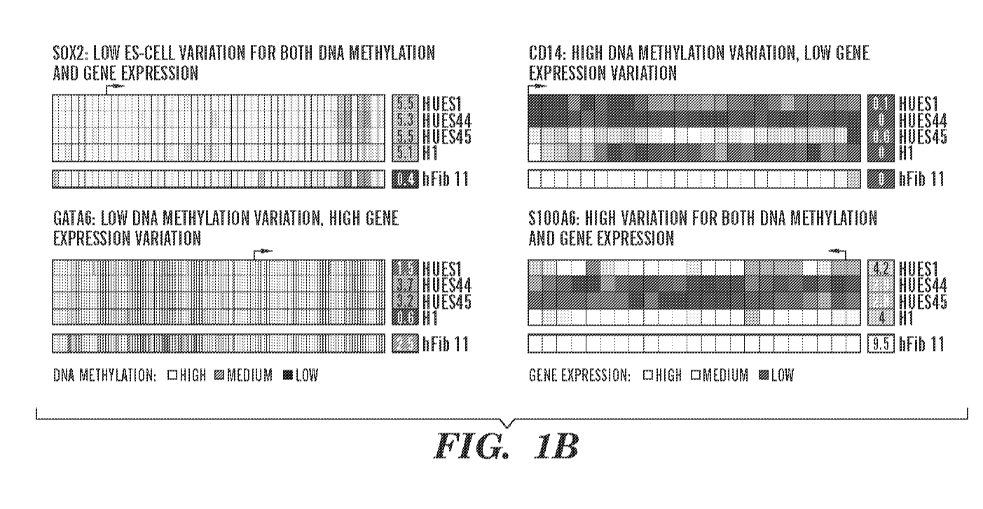

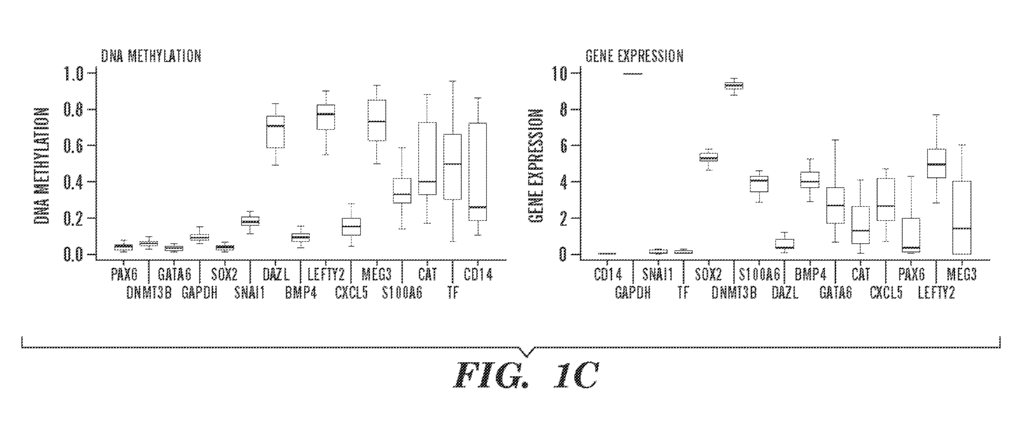

Compare the normalized DNA methylation values and the normalized gene expression values for each gene with the normalized DNA methylation values and normalized gene expression values for the reference pluripotent stem cell lines. Identify the pluripotent stem cell lines as “outlier” cell lines if their value for DNA methylation or gene expression falls outside the center quartiles by more than about 1.2-times or more than 1.5-times the interquartile range (for example, using Tukey's outlier filter). Stated another way, if the DNA methylation levels or gene expression levels fall outside a “reference corridor” or outside the “reference DNA methylation range” or the “reference Gene expression range (see FIG. 1C as an exemplary example), then the pluripotent stem cell line is considered an “outlier” stem cell line.

(v) Relevance Fitler:

Apply a relevance filter identify pluripotent stem cells identified as “outlier” stem cell lines which have a DNA methylation difference of greater than about 15% or about 20 percentage points (20%) or an expression change of at least about 1.5-fold or about at least 2-fold, and disregard the pluripotent stem cell outlier stem cell lines from use or further analysis.

(vi) Gene Sets:

Load gene sets containing relevant genes for the application of interest, such as genes lists in Table 12A, 12B, 12C, 13A, 13B and 14, and lineage marker genes (e.g., genes listed in Tables 7, 13A-13B and Table 14) and cancer genes (e.g., such as those listed in Table 6A and 6B).

(v) Report Summary:

List the number of deviations for each pluripotent stem cell line of interest. For example, the report can provide the % of deviations from the norm, or the absolute number of deviations from the norm, and optionally, the name of the affected gene(s) (see for example 4B, and Table 6A, 6B, 9A).

In some embodiments, a deviation scorecard is based on non-parametric outlier detection using Tukey's outlier filter (Tukey, 1977). All genes for which the DNA methylation or gene expression value of the cell line of interest fall outside of the center quartiles by more than 1.5 times the interquartile range are considered suspected outliers and flagged as such.

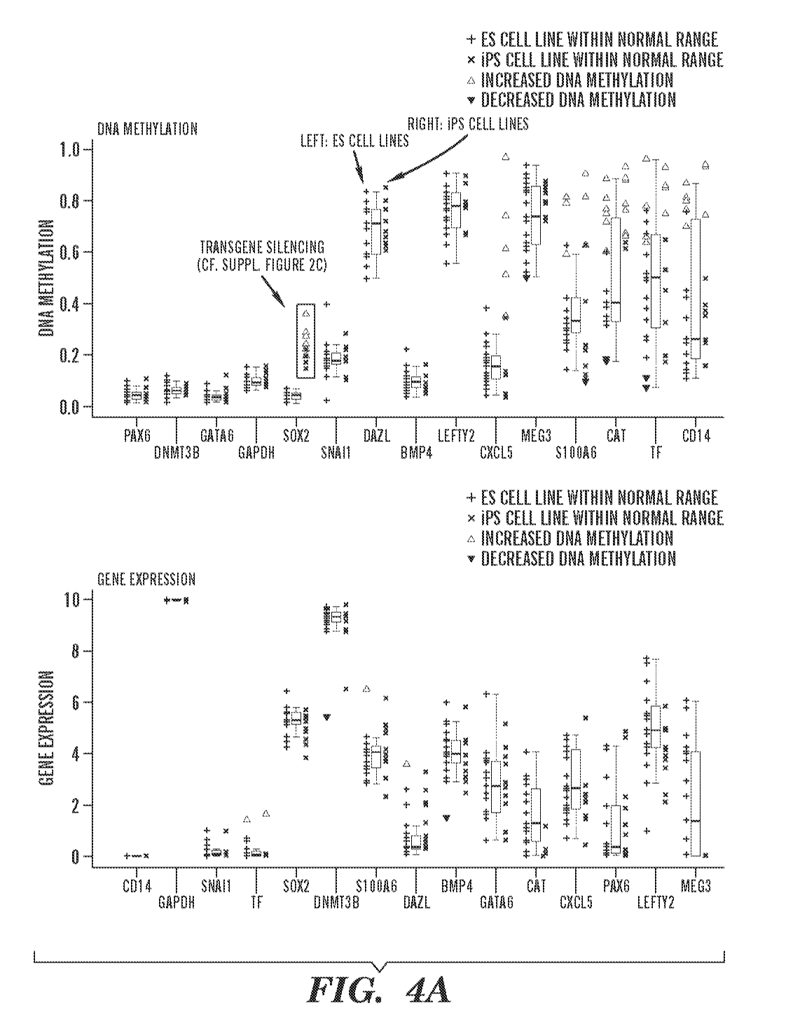

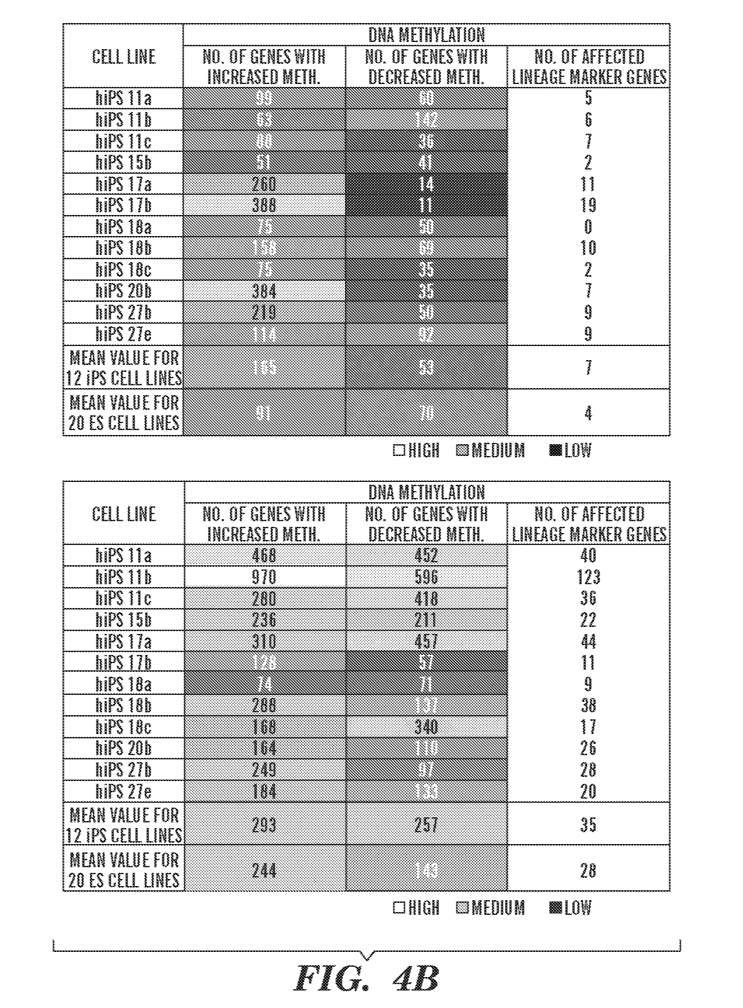

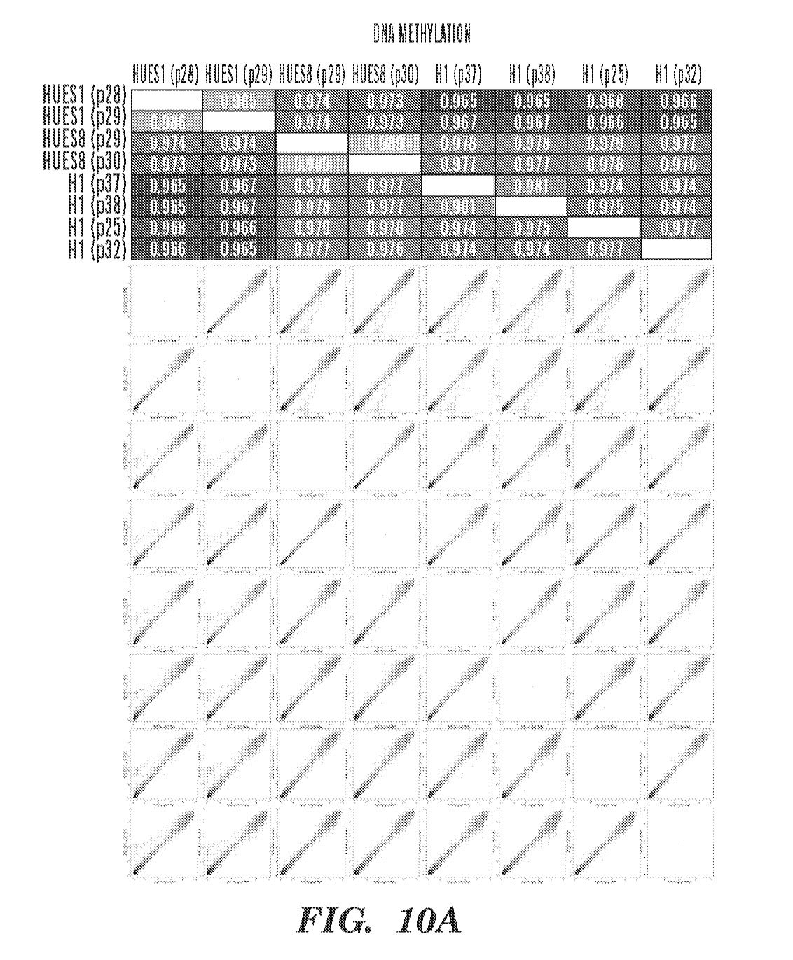

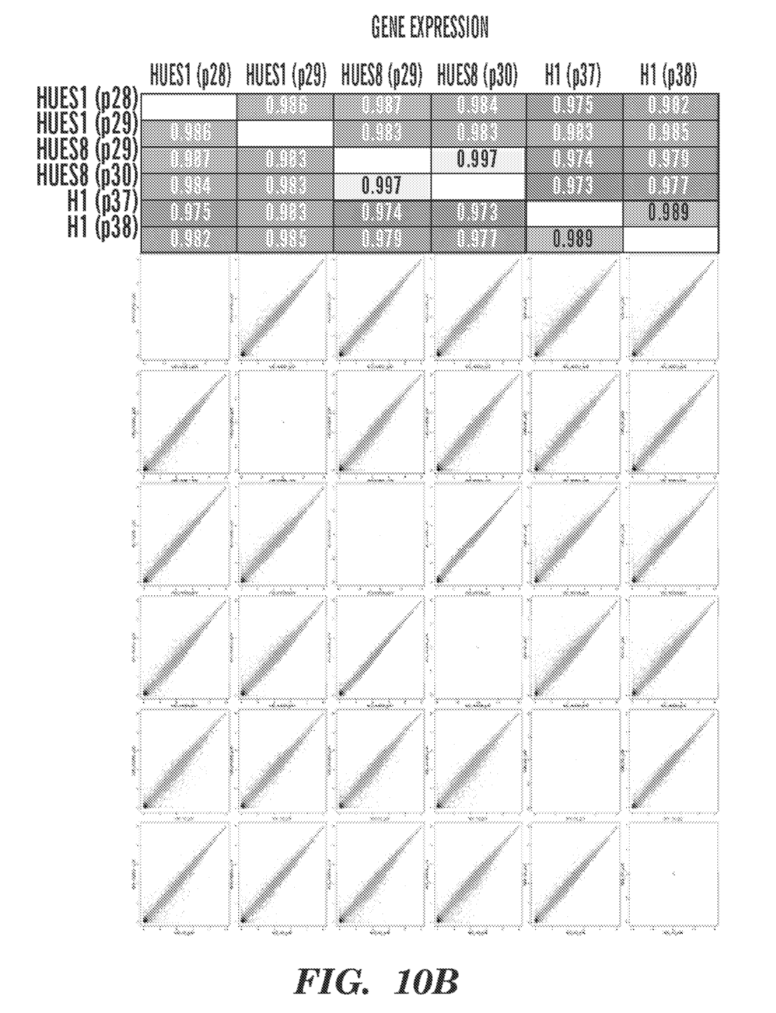

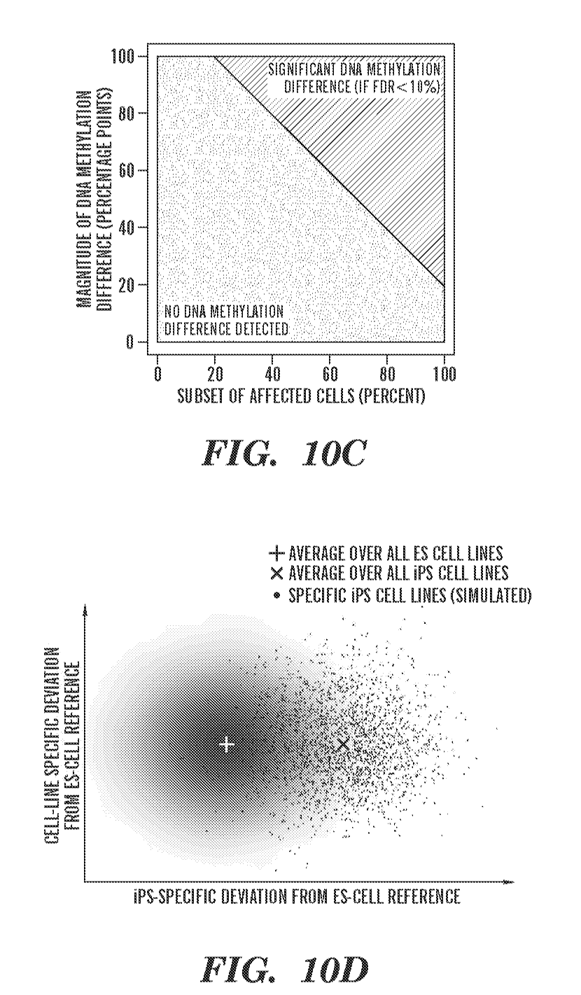

Next, the magnitude of the change is considered and only genes for which the deviation from the ES cell reference is sufficiently large to be considered biologically meaningful are ultimately reported as outliers. For the current study, the inventors used thresholds of at least 20 percentage points for DNA methylation and at least twofold for gene expression, consistent with prior work (Bock et al., 2010) and further justified in FIG. 10C. To account for the fact that deviations may be more or less concerning depending on which genes are affected, in some embodiments, one can assemble multiple lists of genes, e.g., two or more lists of genes which need to be monitored particularly closely for DNA methylation defects, namely lineage marker genes and cancer genes. Deviations at these genes are specifically highlighted in the extended version of the deviation scorecard (Table 12A, Table 12B and Table 12C). Finally, in some embodiments, one can also use alternative strategies for identifying or flagging outlier pluripotent stem cell lines, including, for example, parametric approachs based on moderated t-tests. In some embodiments, Tukey's outlier filter can be used for identifying outlier pluripotent stem cell lines, which has the additional advantage that it can be intuitively visualized by “reference corridor” boxplots (see FIGS. 1C and 4A).

Lineage Scorecard Calculation

A lineage scorecard as disclosed herein quantifies the differentiation propensity of a cell line of interest relative to one or more reference pluripotent stem cell lines, e.g., high quality and/or low-passage pluripotent stem cell lines, such as the reference values for the 19 low-passage ES cell lines as used herein in the Examples. The algorithm for calculating the lineage scorecard (outlined in FIG. 11B) uses a combination of moderated t-tests (Smyth, 2004) and gene set enrichment analysis performed on t-scores (Nam and Kim, 2008; Subramanian et al., 2005).

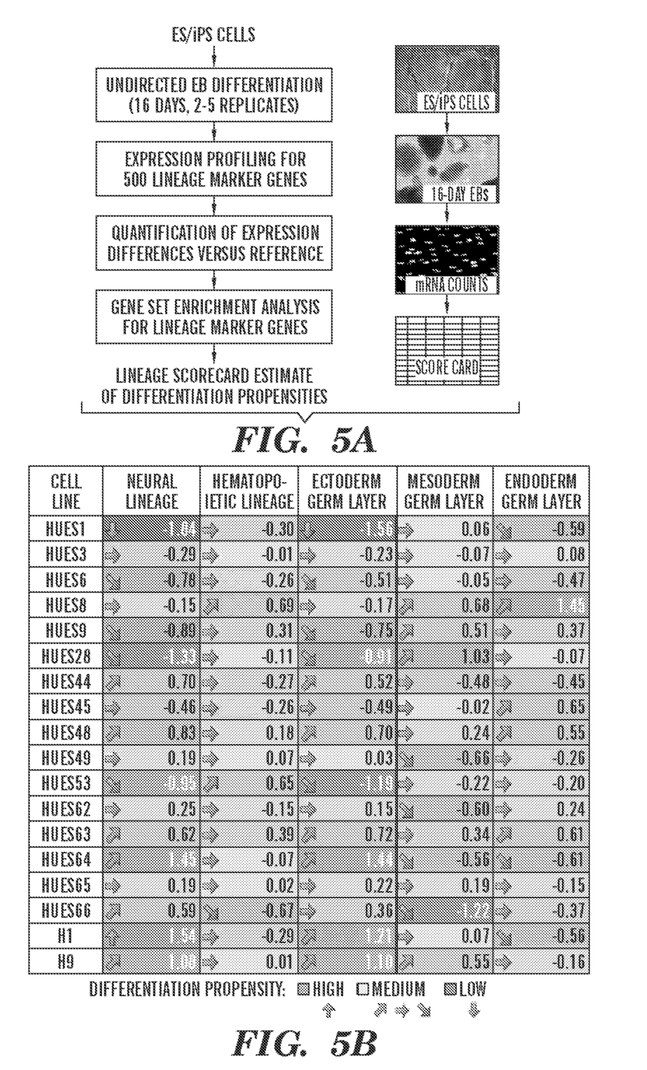

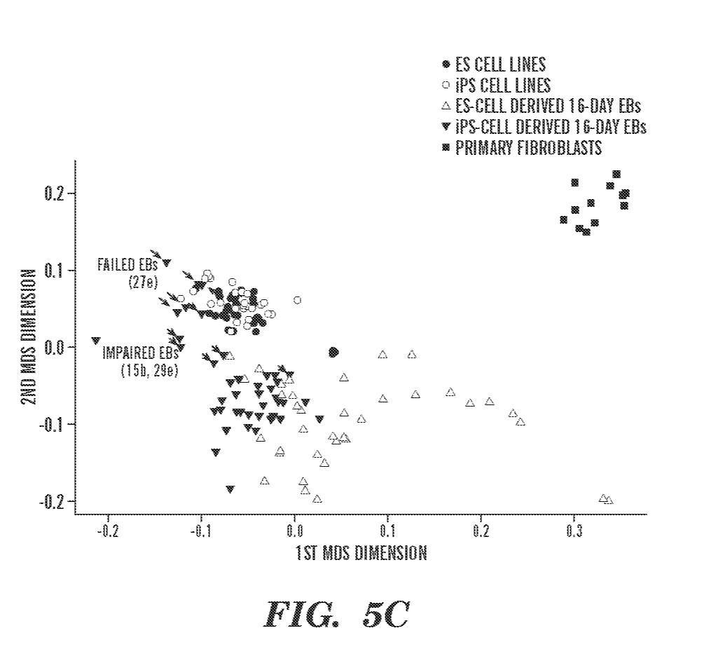

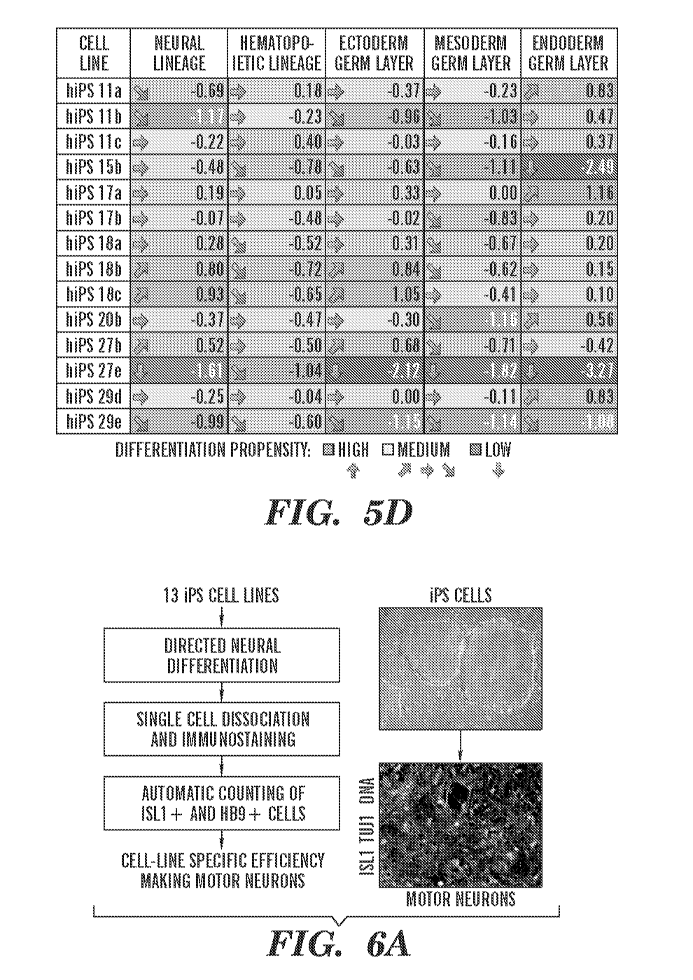

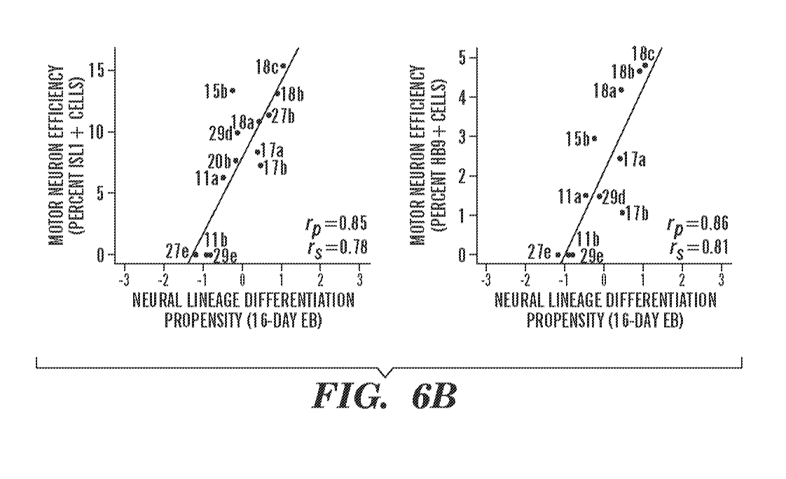

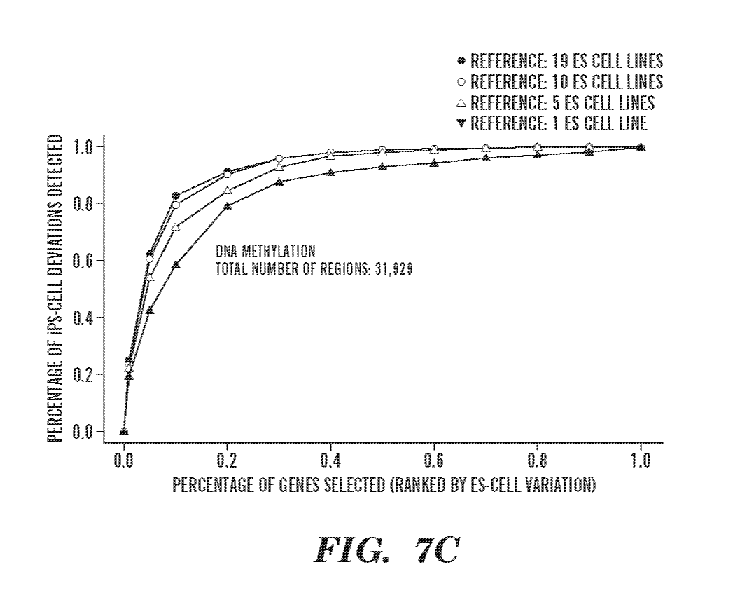

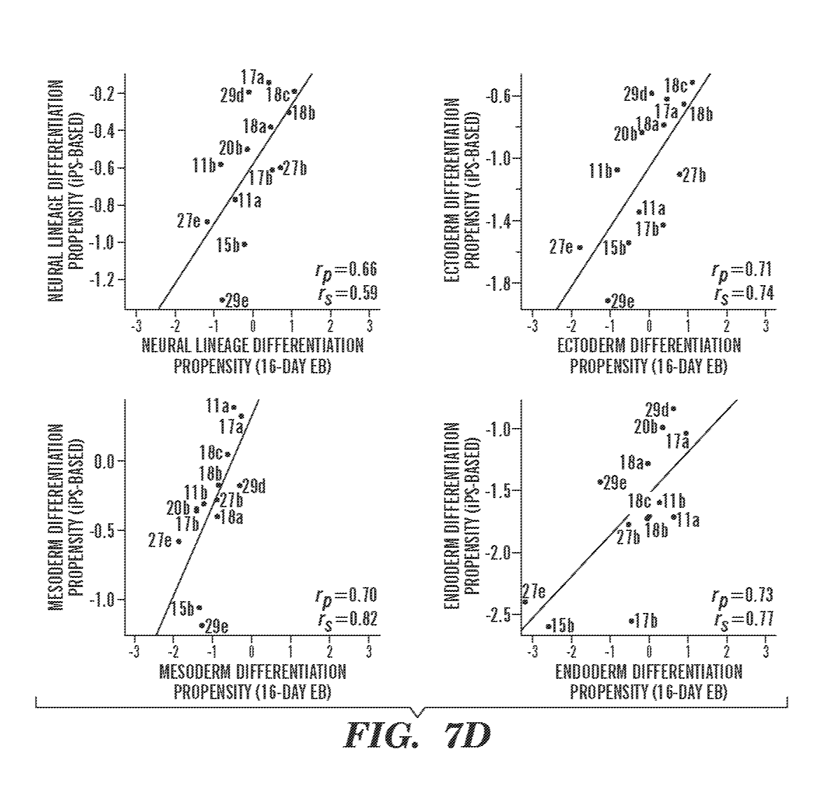

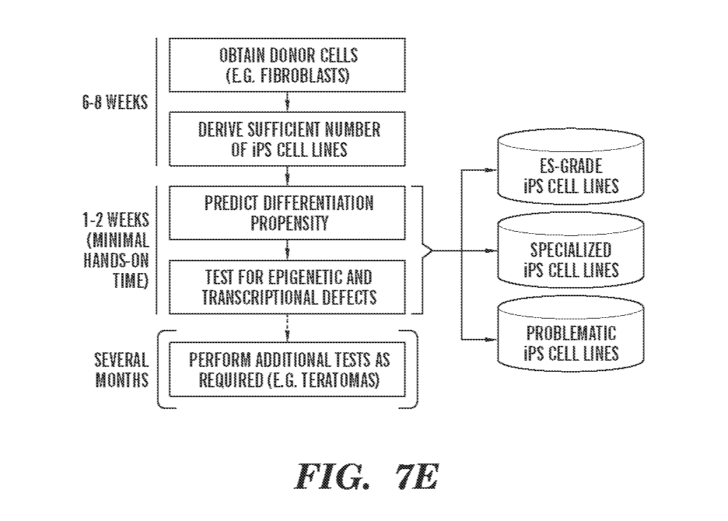

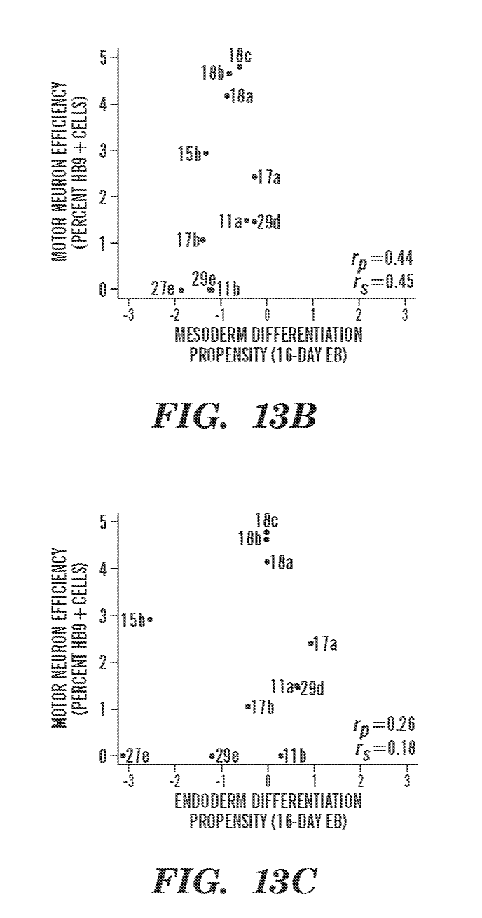

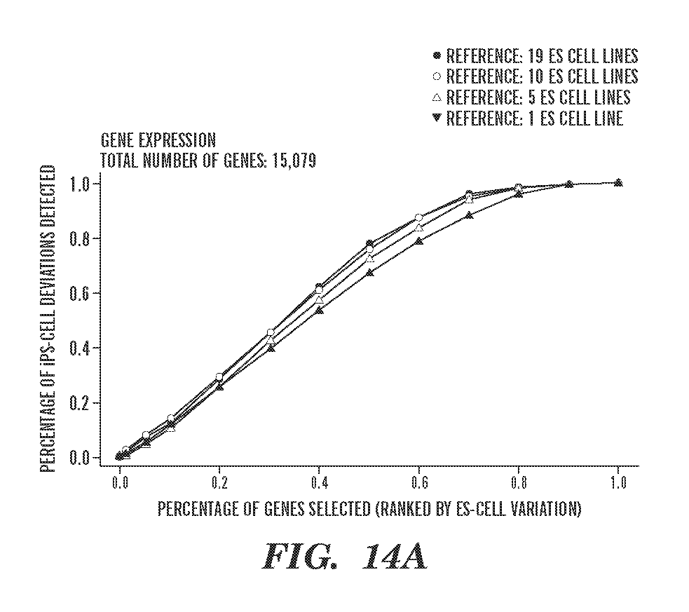

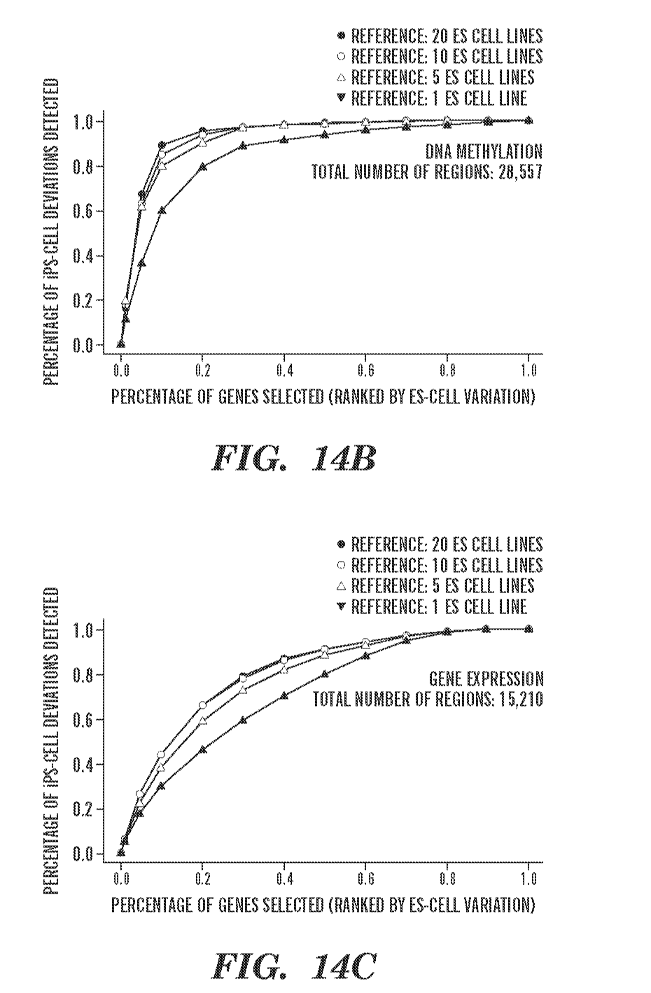

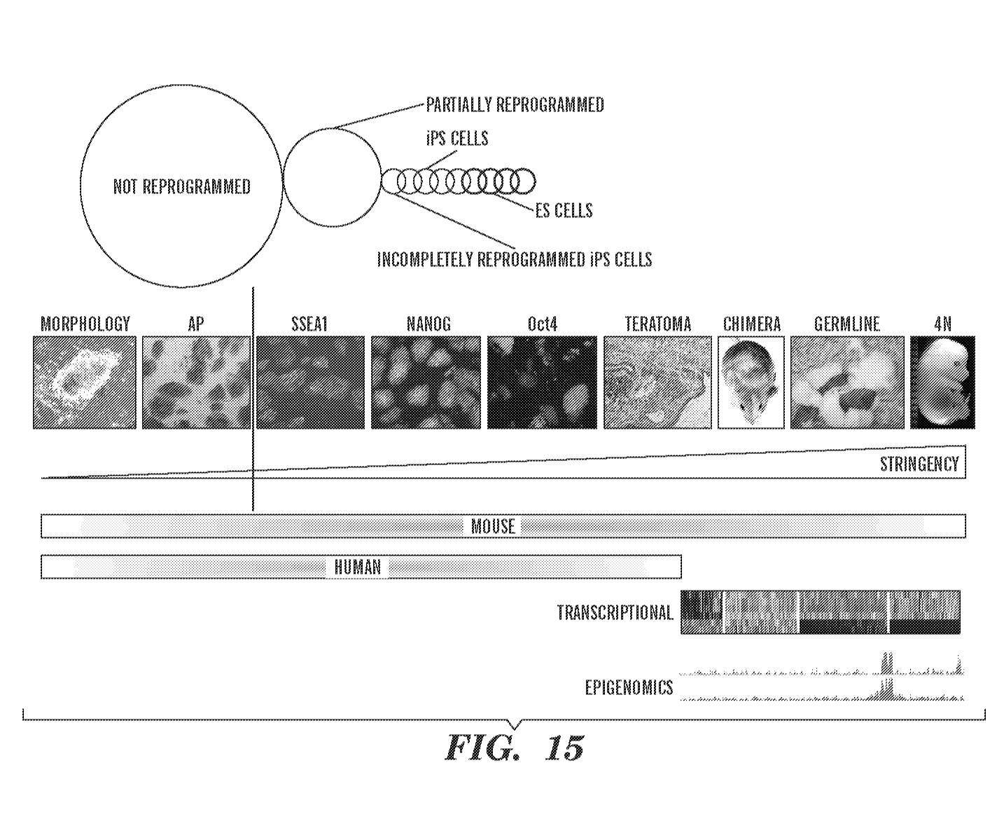

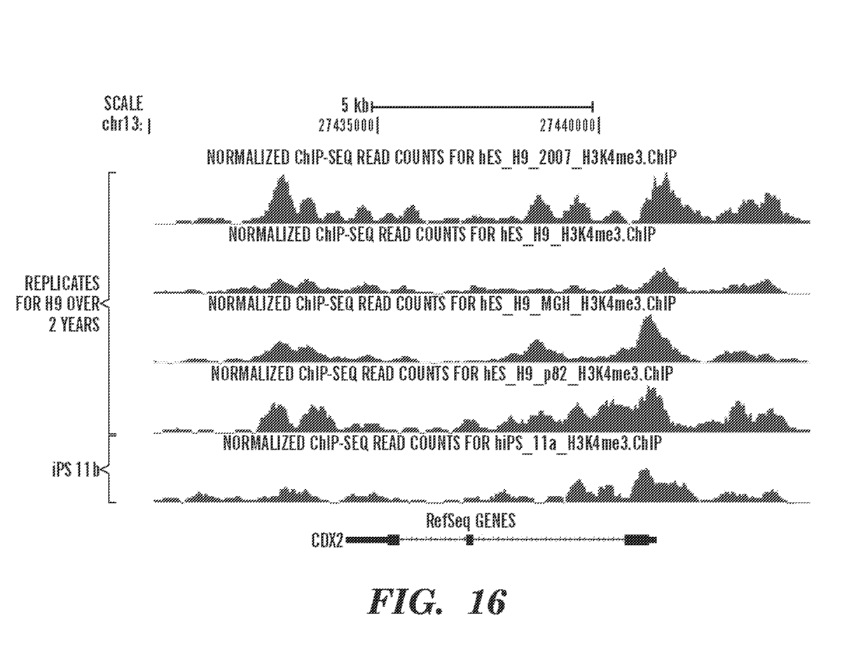

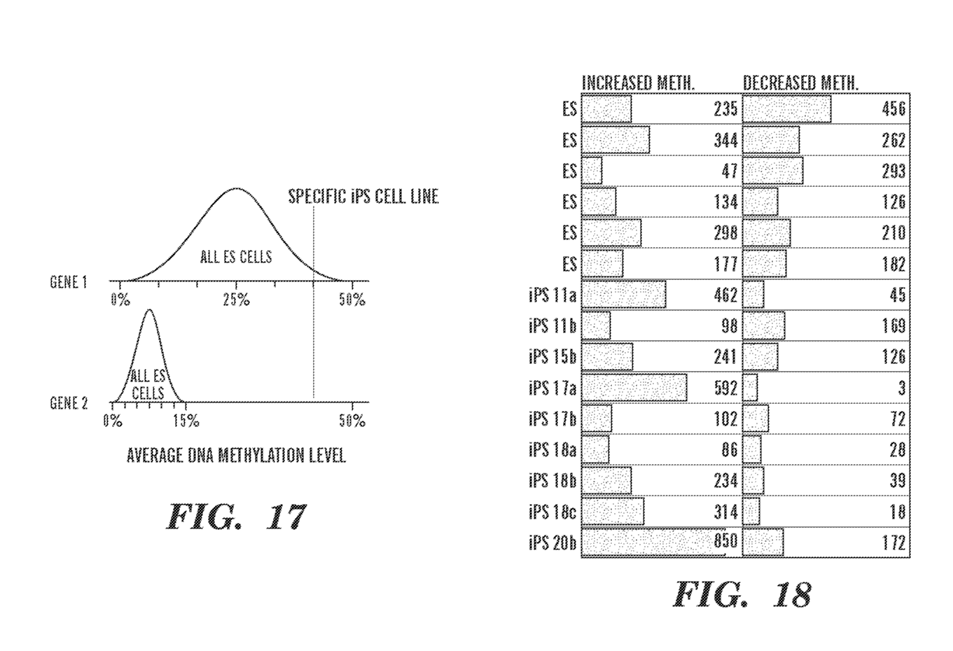

To provide a biological basis for quantifying lineage-specific differentiation propensities, the inventors created several sets of marker genes for each of the three germ layers (ectoderm, mesoderm, endoderm) as well as for the neural and hematopoietic lineages (see FIGS. 7 and 13A). Next, Bioconductor's limma package was used to perform moderated t-tests comparing the gene expression in the EBs obtained for the cell line of interest to the EBs obtained for the ES cell reference, and the mean t-scores were calculated across all genes that contribute to a relevant gene set. High mean t-scores indicate increased expression of the gene set's genes in the tested EBs and are considered indicative of a high differentiation propensity for the corresponding lineage. In contrast, low mean t-scores indicate decreased expression of relevant genes and are considered indicative of a low differentiation propensity for the corresponding lineage. To increase the robustness of the analysis, the mean t-scores were averaged over all gene sets assigned to a given lineage. The lineage scorecard diagrams (FIGS. 5B and 5D) list these “means of gene-set mean tscores” as quantitative indicators of cell-line specific differentiation propensities. The lineage scorecard analyses and validations were performed using custom R scripts (http://www.r-project.org/).

As demonstrated herein in the Examples section, specific cell differentiation efficiencies can be used as a reliable and robust test for predicting the differentiation potential of a pluripotent stem line into a particular cell lineage. For example, as demonstrated herein in the Examples, motor neuron differentiation efficiencies that were experimentally derived by Boulting et al. provided a genuine test set for determining the predictive power of the lineage scorecard: The bioinformatic algorithms of the lineage scorecard had already been finalized before the first comparisons between the two datasets were made, and no aspects of the scorecard were retrospectively optimized to improve the fit.

The algorithm for calculating the lineage scorecard (outlined in FIG. 11B) includes the following steps:

(i) Data Import:

Import gene expression and/or DNA methylation data of at least 200, or at least about 300, or at least about 400, or at least about 500 or more marker genes from (i) embroid bodies (EBs) of the pluripotent stem cell of interest, and (ii) at least one, or at least about 5, or at least about 10 or more embroid bodies (EBs) from reference pluripotent stem cell lines (e.g., pluripotent stem cell lines which are used as high quality reference pluripotent stem cell control cell lines). In some embodiments, the gene expression data is microarray data, and in some embodiments, the DNA methylation data is whole-genome DNA methylation, or RRBS (reduced-representation bisulfite sequencing).

(ii) Optional Step of Assay Normalization:

Use positive spike-in controls to calculate an assay normalization factor and rescale the data accordingly. In some embodiments the spike-in normalization is needed for each experiment or replicate experiment.

(iii) Sample Normalization:

Perform variance stabilization and normalization across all experiments. In some embodiments, variance stabilization and normalization can be performed by readily available software by one of ordinary skill in the art, such as Bioconductors VSN package).

(iv) Reference Comparison:

Compare the normalized DNA methylation values and the normalized gene expression values for each lineage marker gene (e.g., listed in Tables 7, 13A-13B and 14) of EBs from each pluripotent stem cell line of interest with the normalized DNA methylation values and normalized gene expression values for the same lineage marker genes the EBs of the reference pluripotent stem cell lines. In some embodiments, statistical analysis is used for the comparison, for example use of moderated t-test for each marker gene to compare the EB replicates of pluripotent stem cell lines of interest with the reference set of values obtained for the reference high-quality EBs. In some embodiments, any statistical package can be used, for example, using Bioconductor's limma package or the like.

(v) Gene Sets:

Load linaeage marker gene sets containing relevant genes that are characteristic for the cellular lineage or germ layer of interest. Any gene list can be used and can be readily compiled by one of ordinary skill in the art using Gene Ontology, MolSigDB or from manual curation efforts). Examples of such gene lists are disclosed in Tables 7, 13A, 13B and Table 14 herein.

(Vi) Enrichment Analysis:

For each gene set (where DNA methylation and/or gene transcript expression levels are determined), calculate the mean t-scores of all marker genes that belong to each set.

(vii) Lineage Scorecard Report:

For each pluripotent stem cell line of interest, list the mean of the t-scores for all the relevant gene sets, to provide a scorecard estimate for the lineage that the pluripotent stem cell will differentiate into (See FIGS. 5A and 5B for example).

Bioinformatic Analysis and Data Access

In addition to method-specific data normalization and the calculation of the scorecard (described above), bioinformatic analyses of the data set can be conducted as follows:

(i) Hierarchical Clustering.

Hierarchical clustering can be performed as disclosed herein in the Examples section (see FIGS. 1, 3, 8 and 9) of the DNA methylation levels (e.g., of the coverage-weighted average over all CpGs in the promoter regions of Ensembl-annotated transcripts) as well as gene expression levels (e.g., for each Ensembl gene by averaging over all associated probes on the microarray). Prior to hierarchical clustering, one can separately normalize each of the two datasets separately to zero mean and unit variance in order to give equal weight to both datasets. The heatmaps shown in FIGS. 1, 3, 8 and 9 are representative selection of 250 genes.

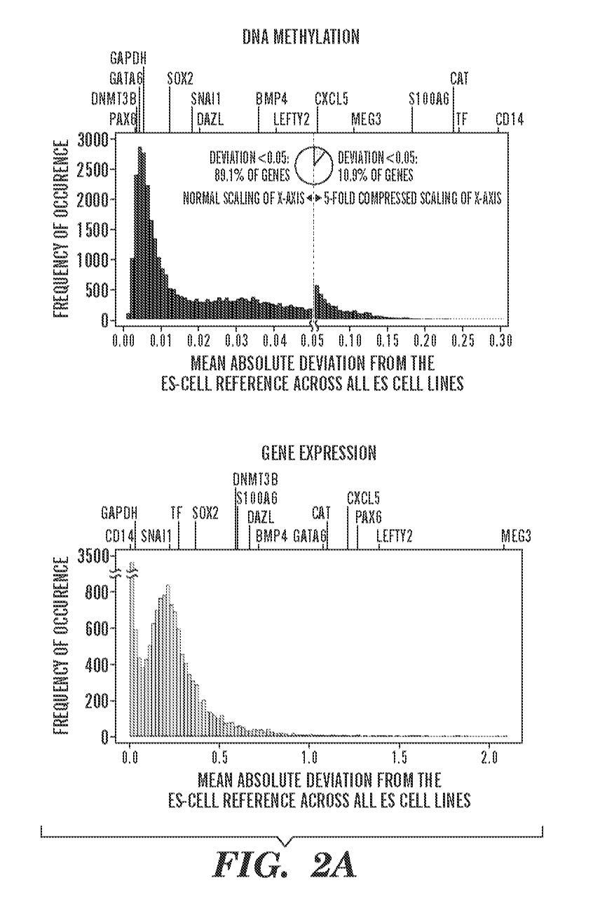

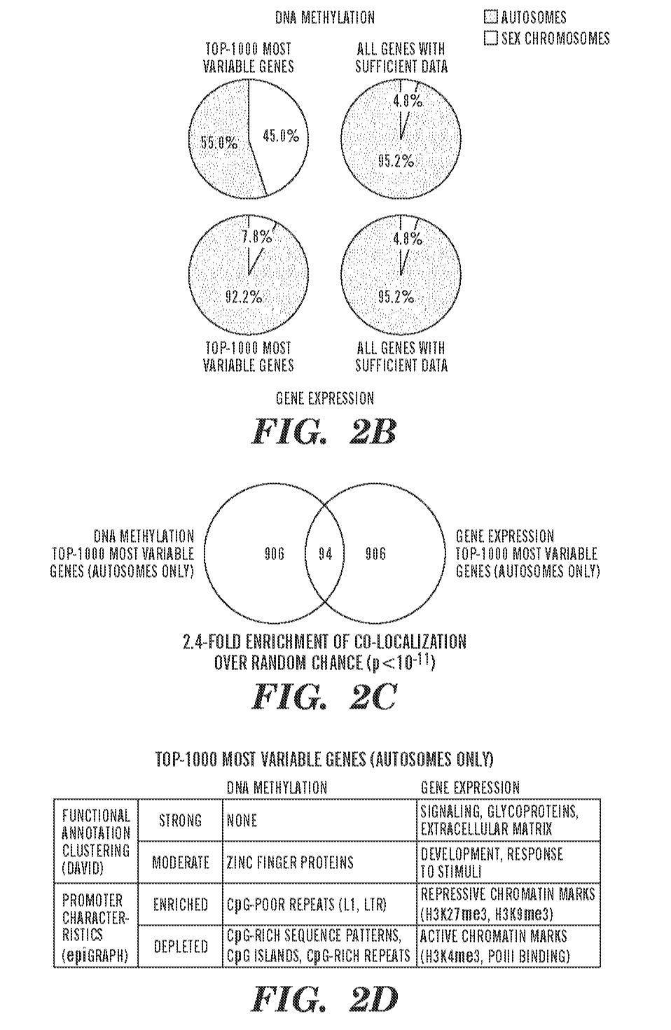

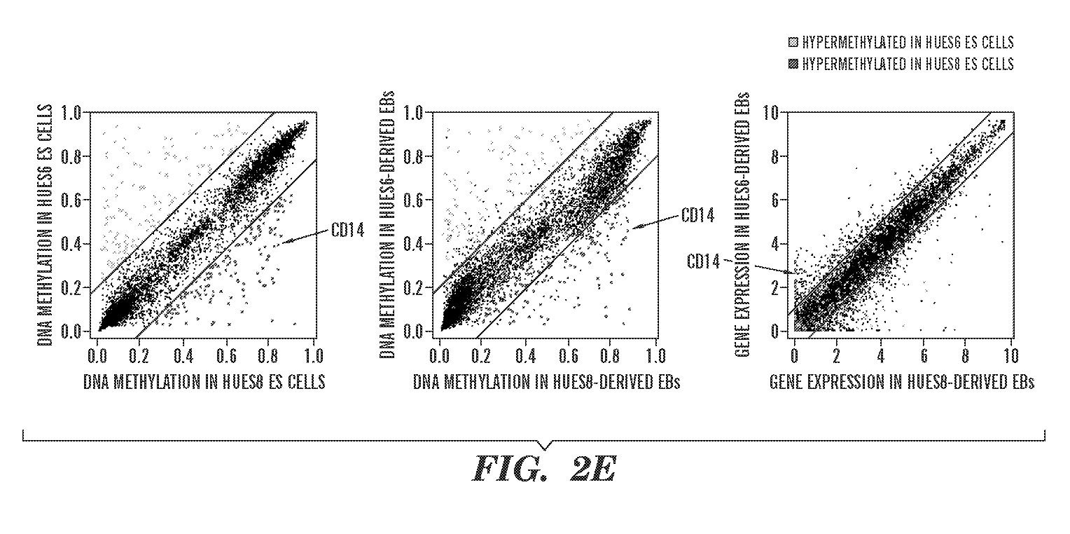

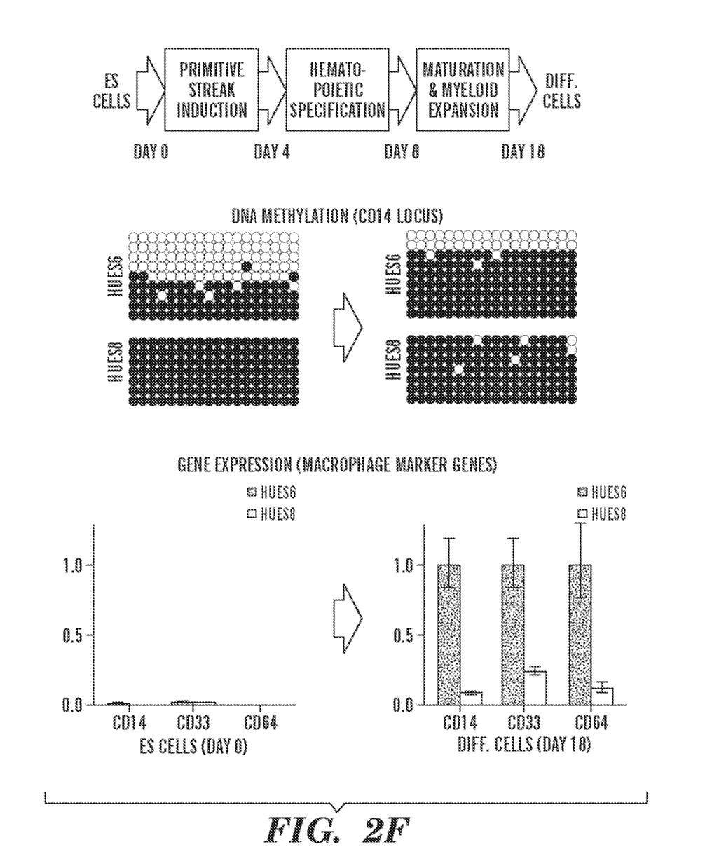

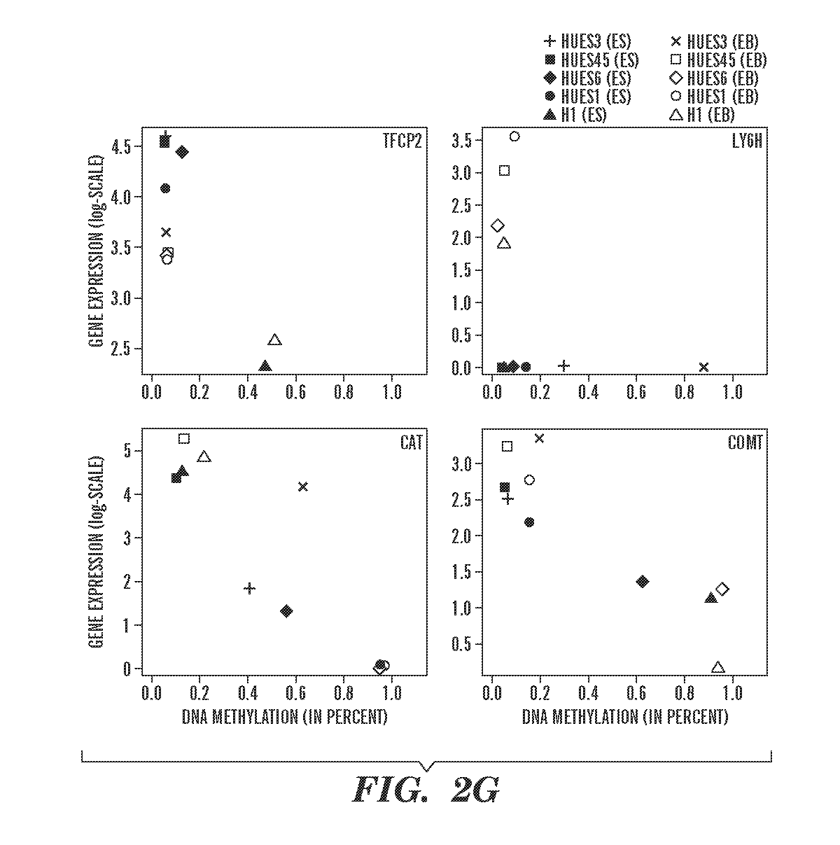

(ii) Annotation Clustering and Promoter Characteristics (FIG. 2D).

One can identify common characteristics among the most variable genes using commonly available software packages, such as, for example, DAVID (Huang et al., 2007) and EpiGRAPH (Bock et al., 2009) with default parameters and based on Ensembl gene annotations (promoters were defined as the −5 kb to +1 kb sequence window surrounding the transcription start site).

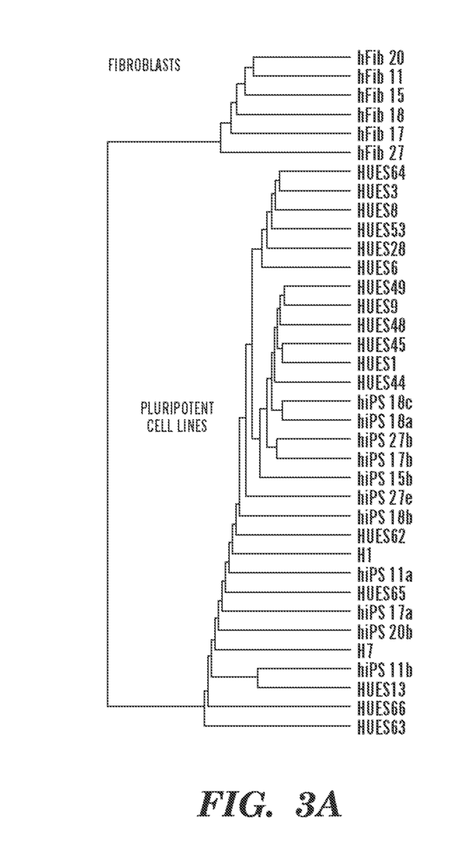

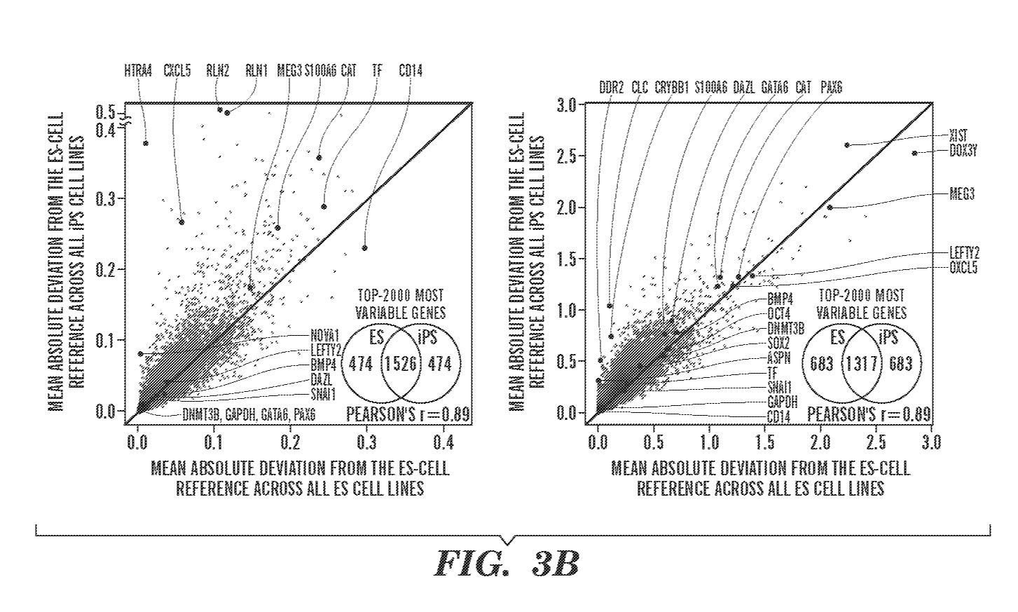

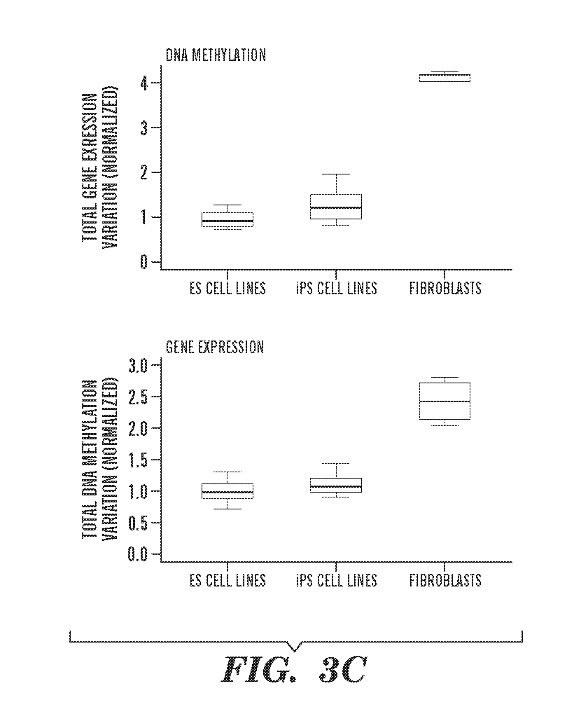

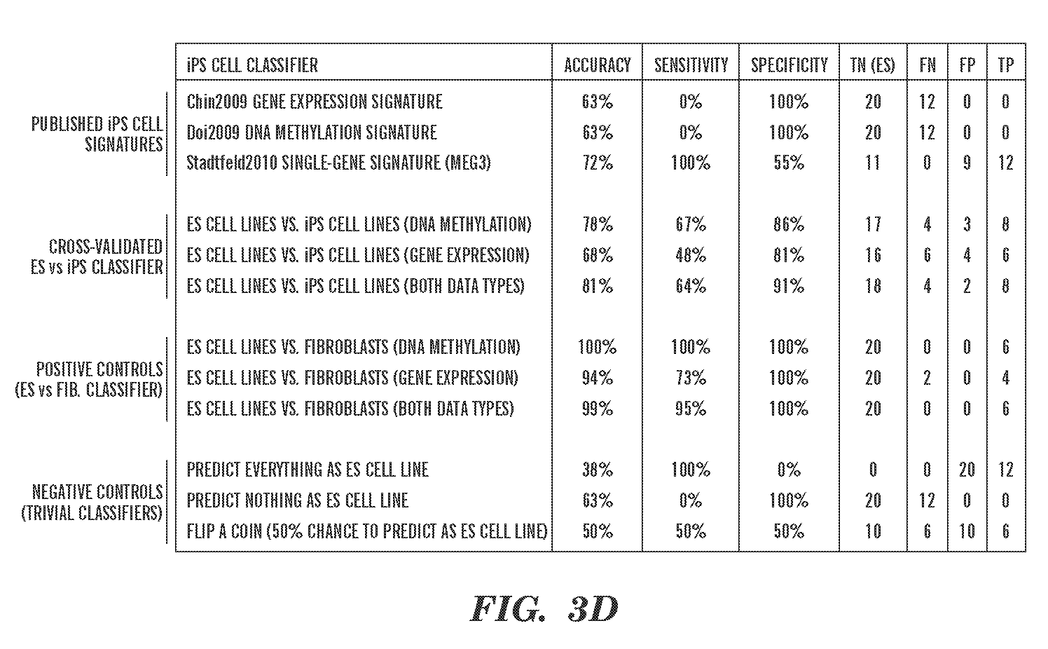

(iii) Classification of ES vs. iPS Cell Lines (FIG. 3D).

One can easily validate ES and iPS gene signatures using the mean DNA methylation or expression level over all genes in a given signature. Logistic regression can be used to select a discriminatory threshold, and the predictiveness of each signature can be evaluated by leave-one-out cross-validation. To derive new classifiers, support vector machines can be trained on the DNA methylation data, the gene expression data, or the combination of both datasets. As disclosed herein in the Examples section, one can perform each classification on 7500 randomly selected attributes, which is a maximum number of attributes that were easily, and computationally feasible for analysis in a single analysis. In some embodiments, the predictiveness of all classifiers can be evaluated by leave-one-out cross-validation, and averaging the performance over 100 classifications with random attribute sets (as shown in FIG. 3D). In some embodiments, a supervised or unsupervised feature selection could be used to increase the prediction accuracy. In some embodiments, predictions can be performed using readily available software, for example using the Weka software (Frank et al., 2004)

(iv) Linear Models of Epigenetic Memory.

One can also generate linear models of DNA methylation and/or gene expression levels. For example, as disclosed herein, two alternative linear models can be constructed for both DNA methylation and gene expression. One model can be used to regress the iPS-cell specific mean DNA methylation (or gene expression) levels of each gene on the ES-cell specific mean DNA methylation (or gene expression) levels. A second model regresses the iPS-cell specific mean DNA methylation (or gene expression) levels of each gene on the ES-cell specific and the fibroblast-specific mean DNA methylation (or gene expression) levels.

Identification of Differentially Methylated Regions (DMR)

One can identify differentially methylated genomic regions, e.g., differentially methylated genes using commonly known methods, such as a classical peak detection (as discussed in Bock, C. et al., Bioinformatics 24, 1 (2008) and (Park, P. J., Nat. Rev. Genet. 10, 669 (2009) which are incorporated herein in their entirety by reference). However, classical peak detection may not be well-suited for differentially methylated regions (DMR) identification because of the high number of spurious hits encountered when borderline peaks are detected in one sample but not in the other (C. Bock, unpublished observation).

Instead, in some embodiments, one can identify differentially methylated regions using a statistical test to compare two samples directly with each other. For a given genomic region with RRBS data, one can count the number of methylated vs. unmethylated CpGs in both samples and perform Fisher's exact test to obtain a p-value that is indicative of the likelihood of the region being a DMR. Similarly, for MeDIP and MethylCap one can count the numbers of reads that align inside the region for both samples and use Fisher's exact test to contrast these values with the total numbers of reads that align elsewhere in the genome. For example, if one is measuring methylation using an Infinium assay, one can use a paired-samples t-test to compare the two samples' β-values of all Infinium probes inside the region. These tests are performed on a large number of genomic regions in parallel (e.g., on all CpG islands), and the p-values are corrected for multiple testing using the q-value method (Storey, et al., PNAS 100, 9440 (2003)). Genomic regions with a q-value of less than 0.1 are flagged as hypermethylated or hypomethylated (depending on the directionality of the difference), but only if the absolute DNA methylation difference exceeds 20% (for RRBS and Infinium) or if there is at least a twofold difference in the read number (for MeDIP and MethylCap). These thresholds were chosen by the inventors by their practical utility in a number of comparisons between different cell types and have no further justification. In some embodiments, one can also mark genomic regions with insufficient sequencing coverage, but do not exclude them from differentially methylated region (DMR) analysis. In some embodiments, if methylation is measured using MeDIP and MethylCap assays, it is recommended to have at least ten reads per 10 million total reads for the sample with higher read coverage, and if methylation is measured using RRBS, it is recommended to have a minimum of five CpGs with at least five reads each in both samples.

In some embodiments, this statistical approach to differentially methylated region (DMR) identification requires one to define a set, or a series of sets of genomic regions on which the analysis is being performed. For example, one can select a set, or series of set of genes listed in Tables 12A and/or 12C. In some embodiments, one can pursue a two-way strategy to maximize the chances of finding interesting DMRs in the pluripotent stem cell. In some embodiments, once a set or series of sets of genomic regions are selected, one can further focus the analysis specifically on CpG islands and gene promoters, which are prime candidates for epigenetic regulation. This approach is useful as it provides increased statistical power for regions with well-known functional roles because the relatively low number of CpG islands and gene promoters reduces the burden of multiple-testing correction compared to the genome-wide case. In an alternative embodiment, one can use a 1-kilobase (or other pre-determined genomic size) tiling of the genome to detect DMRs that are located outside of any candidate regions. In some embodiments and to cast an even wider net, one can also collect a comprehensive set of 13 types of genomic regions, which includes not only CpG islands and gene promoters, but also CpG island shores (Irizarry, R. A. et al., Nat. Genet. 41, 178 (2009)), enhancers (Heintzman, N. D. et al., Nature 459, 108 (2009)), evolutionary conserved regions and other types of genomic regions. In some embodiments, the differentially methylated region (DMR) data for all of these region sets can be calculated using a set of Python and R scripts and are available online (world wide web at: “//meth-benchmark.computational-epigenetics.org/”).

Candidate loci for determination of epigenetic modifications, e.g., different levels of DNA methylation can comprise all genomic regions, or a specific type of genomic regions, such as promoters, enhancers, insulator elements, CpG islands, CpG island shores, etc. In some embodiments, one can also use DNA methylation data to directly derive regions that are highly variable, and DNA sequence data to predict genomic regions that are susceptible to epigenetic alterations. Furthermore, in some embodiments one can use prior knowledge of genes and genomic regions that are involved in cancer, normal and abnormal development and diseases as candidates.

Furthermore, one of ordinary skill in the art can use any one of, or a combination of text mining, information retrieval, statistical learning and ranking methods for prioritizing genes and genomic regions based on publicly available information and all kinds of functional genomics datasets. The inventors used these methods to define gene sets, networks and pathways.

In some embodiments, as an alternative, or on addition to DNA methylation, one can assess other epigenetic modifications, such as, but not limited to histone modifications. DNA methylation and other epigenetic modifications are highly correlated, such that it is immediately obvious that information that can be obtained from DNA methylation data can also be obtained from other epigenetic modifications such as histone methylation and acetylation, etc.

Gene expression analysis can also be performed by a number of methods, which are more widely used than methods for DNA methylation analysis. Typical example include, but are not limited to, gene expression microarrays, cDNA and RNA sequencing, imaging-based methods such as NanoString and a wide range of methods that use PCR as well as qPCR. Normalization for these methods has been widely described. Herein, the inventors have used gcRMA algorithm for normalizing Affymetrix microarray data.

In some embodiments one can use NanoString data, and the inventors herein have systematically evaluated multiple algorithms based on this data. Based on these results, the inventors discovered that the VSN algorithm was most suitable for normalizing NanoString data.

In some embodiments, gene expression is determined on any gene level, for example, the expression of non-coding genes, microRNA genes and all other types of RNA transcripts that are normally or abnormally present in pluripotent and differentiated cells.

Once the gene expression data are normalized, genes of relevance for cell line quality and utility are identified using standard methods for detecting differential gene expression between samples and/or groups of samples. Examples include t-test and its variants, non-parametric alternatives of the t-test, and ANOVA. The inventors in the Examples herein used the limma package, which implements a moderated t statistic.

Given that the function(s) of many genes are now known, it is possible to assign putative effects to the differential expression and/or DNA methylation, such as increased or decreased cancer risk, differences in the ability to differentiate into specific cell types and lineages, resistance against drugs and the general usefulness for disease modeling, drug screening and regenerative therapies.

While the DNA methylation and the gene expression assay as described above focus mostly on the effect of single genes, in some embodiments, the lineage scorecard uses the combination of data for multiple genes to predict a cell line's quality and utility. This is the most critical and bioinformatically complex step for the creation of a lineage scorecard.

The information from multiple genes is currently aggregated by mean and standard deviation calculations, however, by using statistical learning methods such as support vector machines, linear and logistic regression, hierarchical models, Bayesian algorithms and the like the effect of aggregration can be reduced. Any mathematical function that takes multiple measurements of candidate genes or genomic regions for gene expression and/or DNA methylation into account to produce a numeric or categorical value that describes an aspect of pluripotent cell quality and utility could be considered a predictor and an element of the scorecard as disclosed herein.

Importantly, these mathematical functions will in many cases take prior biological knowledge into account. In particular, the inventors have curated a substantial number of gene sets from the literature, from public databases and from functional genomics data to inform these predictors. In one embodiment of the scorecard, one can use DNA methylation and/or gene expression data from either the pluripotent cell or its differentiating progeny to assign differential methylation/expression scores to each gene and genomic region, and then use the resulting t-scores to perform a (parametric or non-parametric) gene set enrichment analysis for sets of genes that represent the three germ layers as well as other interesting cell types, cellular pathways and networks, as well as other functionally or otherwise defined sets of genes.

While the bioinformatic methods described above were applied in the Examples herein, they can also be applied directly to DNA methylation, gene expression and other epigenetic and functional genomic data of pluripotent cells, and it is also possible to induce the pluripotent cell lines to differentiate such that certain aspects of their quality and utility become more evident. This can be performed using a wide range of perturbations, from simple growth factor withdrawal and physical manipulation (as used herein for undirected embryoid body differentiation) over a wide range of chemical, peptide and protein treatments (often in combination) to the plating on dedicated surfaces and the induced expression of specific genes.

One can analyze the gene expression data using a variety of methods, for example, as disclosed in Han et al., Nucleic acid research, 2006; 34(2): e8, “Comparison of algorithms for the analysis of Affymetrix microarray data as evaluated by co-expression of genes in known operons”, and in the book entitled “Methods in microarray normalization” Edited By Phillip Stafford, Drug Discovery Series/10, published by CRC Press (which are incorporated herein in its entirety by reference). The cgRMA algorithm (GC [GC content} robust multichip analysis (RMA)) uses both the quantile normalization and medium polish summarization methods of the RMA algorithm. A stochastic modes is used to describe the observed PM and MM probe signals for each probe pair on an array. In particular, the models is:

PM_{μi}=0ₙᵢ+Sₙᵢ

NMₙᵢ=0ₙᵢ+N₂ₙᵢ

Where 0ₙᵢ represents the optical noise, N₁ and N₂ represents nonspecific binding, and Sₙⱼ is a quantity proportion to the RNA expression in the sample. In addition, the model assumes O follows a normal distribution N(μ0, σ²₀) and that log₂ (N₁ₙᵢ) and log₂ (N₂ₙᵢ) follow a bivariate-normal distribution with equal variances σ²_{N} and correlation 0.7, constant across probe pairs. The means of the distribution for the nonspecific binding terms are dependent on the probe sequence. The optical noise and nonspecific binding terms are assumed to be independent.

The method by which gcRNA includes information about the probe sequence is to compare an affinity based on the sum of position-dependent base affinities. In particular, the affinity of a probe is given by:

$$A = {\sum }_{k = 1}^{25} {\sum }_{b ∈ ( A , C , G , T )} μ b ( k ) 1 {β}_{k} = j$$

where the μ_{b}(k) are modeled as spline functions with 5 degrees of freedom. In practice, μb(k) for a single microarray (e.g., U113A microarray chips) are either estimated using the observed data for all chips in an experiment or based on some hard-coded estimates from a specific NSB experiment carried out by the creators of gcRMA. This means for the N₁ and N₂ random variables in the gcRMA model are modeled using a smooth function h of the probe affinities.

The optical noise parameters μₒ, σ²ₒ are estimated like this: The variability due to optical noise is so much smaller than the variability due to the nonspecific binding and thus effectively constant. For simplicity this is set to 0. The mean values are estimated using the lowest PM or MM probe intensities on the array, with a correlation factor to avoid negatives. Next, all probe intensities are correlated by subtracting this constant μₒ. To estimate h(Aₙᵢ) a loess curve fit to a scatterplot relating the corrected log(MM) intensities to all the MM probe affinities. The negative residuals from this loess plot are used to estimate σ²_{N} Finally, the background adjustment procedure for gcRMA is to compute the expected value of S given the observed PM, MM and model parameters. Note, that although gcRMA uses the medium polish summarization of RMA, the PLM summarization approach should not be used in its place if one wants to carry out quality assessment, although the expression estimates generated in this way are otherwise satisfactory.

In some embodiments, one can also use other methods for gene expression normalization, for example, using MAS5.0 algorithm (Microarray suite 5.0), RMA algorithm (robust multichip analysis), which are explained in detail in the “method for microarray normalization” edited by Phillip Stafford.

Statistical Methods

Methods for statistical clustering and software for the same are discussed below. For example, one parameter used in quantifying the differential expression of genes is the fold change, which is a metric for comparing a gene's mRNA-expression level between two distinct experimental conditions. Its arithmetic definition differs between investigators. However, the greater the fold change the more likely that the differential expression of the relevant genes will be adequately separated, rendering it easier to decide which category a patient falls into.

The fold change for an upregulated gene may be, for example, at least 1.4, at least 1.5, at least 1.6, at least 1.7, at least 1.8, at least 1.9 or at least 2.0 or more log-2 change. In one embodiment, in which the expression level is measured using PCR, the fold change is at least 2.0.

The fold change for a down-regulated gene may be 0.6 or less than 0.6, for example it may be 0.5 or less than 0.5, 0.4 or less than 0.4, 0.3 or less than 0.3, 0.2 or less than 0.2 or may be 0.1 or less than 0.1 log-2 change. Accordingly, a fold change of 0.1 indicates that the expression of a gene is down-regulated 10 times. A fold change of 2.0 indicates that the expression of a gene is upregulated 2 times.

For example: If the fold change of a gene expression target gene in a pluripotent stem cell is=2.0 (as compared to the normal variation of gene expression of that gene), it indicates that the gene is an “outlier” gene. Similarly, if the fold change of a gene expression target gene in a pluripotent stem cell is=0.5 (as compared to the normal variation of gene expression of that gene) of a gene=0.5, it indicates that the gene is an outlier gene. The higher number of gene expression genes in the test pluripotent stem cell line which are “outlier” genes indicates that the pluripotent stem cell line may have undesirable characteristics, e.g., quality and/or unsuitable for particular utilities. For example, if the test pluripotent stem cell has at least about 50, or at least about 100 or more than 100 outlier gene expression genes, the pluripotent stem cell line is identified as being an outlier pluripotent stem cell line and has different, potentially undesirable, characteristics as compared to a standard pluripotent stem cell line, for instance, it may be of poor quality (e.g., high propensity to transducer into a cancerous cell lineage), and/or low efficiency to differentiate along a particular lineage.

Another parameter also used to quantify differential expression is the “p” value. It is thought that the lower the p value the more differentially expressed the gene is likely to be, indicates that the gene is an outlier gene as compared to the normal variation of gene expression in a pluripotent stem cell. P values may for example include 0.1 or less, such as 0.05 or less, in particular 0.01 or less. P values as used herein include corrected “P” values and/or also uncorrected “P” values.

The present invention may be defined in any of the following numbered paragraphs:
1. A method for selecting a pluripotent stem cell line, comprising
   a. measuring DNA methylation of a set of target genes in the pluripotent stem cell line, and performing a comparison of the DNA methylation data with a reference DNA methylation data of the same target genes;
   b. measuring differentiation potential of the pluripotent stem cell line by undirected or directed differentiation of the pluripotent stem cell by measuring the gene expression and/or DNA methylation of a plurality of lineage marker genes; and comparing the gene expression and/or DNA methylation differentiation with a reference gene expression and/or DNA methylation differentiation of the same lineage marker genes; and
   c. selecting a pluripotent stem cell line which does not differ by a statistically significant amount in the DNA methylation of the target genes as compared to the reference DNA methylation level, and does not differ by a statistically significant amount in the propensity to differentiate along mesoderm, ectoderm and endoderm lineages as compared to a reference differentiation potential; or discarding a pluripotent stem cell line which differs by a statistically significant amount in the in the DNA methylation of the target genes as compared to the reference DNA methylation level, and differs by a statistically significant amount in the propensity to differentiate along mesoderm, ectoderm and endoderm lineages as compared to a reference differentiation potential.
2. The method of paragraph 1, wherein the DNA methylation is measured by contacting at least one pluripotent stem cell with an agent that differently binds an epigenetic modification in the DNA.
3. The method of paragraph 2, wherein the DNA methylation can be measured by contacting the at least one pluripotent stem cell with an agent that differentially binds to methylated and unmethylated DNA, and performing a comparison of the DNA methylation data with a reference DNA methylation data of the same target genes.
4. The method of paragraph 2, wherein the DNA methylation can be measured by any one of the following selected from the group consisting of: enrichment-based methods (e.g. MeDIP, MBD-seq and MethylCap), bisulfite sequencing and bisulfite-based methods (e.g. RRBS, bisulfite sequencing, Infinium, GoldenGate, COBRA, MSP, MethyLight) and restriction-digestion methods (e.g., MRE-seq), or differential-conversion, differential restriction, differential weight of the DNA methylated target gene of the pluripotent stem cell as compared to the reference DNA methylation data of the same target genes.
5. The method of any of paragraphs 1 to 4, further comprising:
   a. measuring the gene expression of a second set of target genes in the pluripotent stem cell line and performing a comparison of the gene expression data with a reference gene expression level of the same target genes; and
   b. selecting a pluripotent stem cell line which does not differ by a statistically significant amount in the level of gene expression of the target genes as compared to the reference gene expression level; or discarding a pluripotent stem cell line which differs by a statistically significant amount in the expression level of the target genes as compared to the reference gene expression level.
6. The method of any of paragraphs 1-5, wherein the reference DNA methylation level is a range of normal variation of methylation for that DNA methylation target gene.
7. The method of any of paragraphs 1-6, wherein the reference DNA methylation level is an average and optionally plus or minus a standard variation of DNA methylation for that DNA methylation target gene, wherein the average is calculated from DNA methylation of that target gene in a plurality of pluripotent stem cell lines.
8. The method of paragraph 7, wherein the plurality of pluripotent stem cell lines is at least 5 or more pluripotent stem lines.
9. The method of any of paragraphs 1-8, wherein DNA methylation for the pluripotent cell line and/or the reference is determined by a bisulfite assay.
10. The method of any of paragraphs 1-9, wherein DNA methylation for the pluripotent cell line and/or the reference is determined by a whole-genome bisulfite assay.
11. The method of any of paragraphs 1-10, wherein DNA methylation for the pluripotent cell line and/or the reference is determined by the reduced-representation bisulfite sequencing (RBBS) assay.
12. The method paragraph 5, wherein the reference gene expression level is range of normal variation of for that target gene.
13. The method of any of paragraphs 5-12, wherein the reference gene expression level is an average of expression level for that target gene, wherein the average is calculated from expression level of that target gene in a plurality of pluripotent stem cell lines.
14. The method of paragraph 13, wherein the plurality of pluripotent stem cell lines is at least 5 or more different pluripotent stem cell lines.
15. The method of any of paragraphs 5-14, wherein the gene expression of the pluripotent cell line and/or reference is determined by a microarray assay.
16. The method of any of paragraphs 1-15, wherein the differentiation potential of the pluripotent cell line is determined by a quantitative differentiation assay.
17. The method of any of paragraphs 1-16, wherein the reference differentiation potential is the ability to differentiate into a lineage selected from the group consisting of mesoderm, endoderm, ectoderm, neuronal, hematopoietic lineages, and any combinations thereof.
18. The method of any of paragraphs 1-17, wherein the reference differentiation potential data is generated from a plurality of pluripotent stem cell lines.
19. The method of paragraph 18, wherein the plurality of pluripotent stem cell lines is at least 5 different pluripotent stem cell lines.
20. The method of any of paragraphs 1-19, wherein the pluripotent cell line DNA methylation target genes and/or the reference DNA methylation target genes are selected from the group consisting of cancer genes, oncogenes, tumor suppressor genes, developmental genes, lineage marker genes, and any combinations thereof.
21. The method of any of paragraphs 1-19, wherein the pluripotent cell line DNA methylation target genes and/or the reference DNA methylation target genes are selected from the group listed in Table 12A or Table 13A or Table 14, and any combinations thereof.
22. The method of paragraph 20, wherein the oncogenes genes are selected from c-Sis, epidermal growth factor receptor, platelet-derived growth factor receptor, vascular endothelial growth factor receptor, HER2/new, Src family of tyrosine kinases, Syk-Zap-70 family of tyrosine kinases, BTK family of tyrosine kinases, Raf kinase, cyclin-dependent kinases, Ras protein, and myc gene.
23. The method of paragraph 20, wherein the tumor suppressor genes are selected from TP53, PTEN, APC, CD95, ST5, ST7 and ST14 gene.
24. The method of paragraph 20, wherein the developmental genes are selected from any combination of genes listed in Table 7 or Table 13A or Table 14.
25. The method of paragraph 20, wherein the lineage marker genes are selected from VEGF receptor II (KDR), actin α-2 smooth muscle (ACTA2), Nestin, Tublin P3, alpha-feto protein (AFP), syndecan-4, CD64IFcyRI, Oct-4, beta-HCG, beta-LH, oct-3, Brachyury T, Fgf-5, nodal, GATA-4, flk-1, Nkx-2.5, EKLF, and Msx3.
26. The method of paragraph any of paragraphs 1-25, wherein the pluripotent cell line DNA methylation target genes and/or the reference DNA methylation target genes are selected from the group consisting of BMP4, CAT, CD14, CXCL5, DAZL, DNMT3B, GATA6, GAPDH, LEFTY2, MEG3, PAX6, S100A6, SOX2, SNAI1, TF, and any combinations thereof.
27. The method of any of paragraphs 1-26, wherein the statistical difference is a difference of at least 1, at least 2, or at least 3 standard deviations from the reference level.
28. The method of any of paragraphs 1-27, wherein the pluripotent cell line gene expression target genes and/or the reference gene expression target genes are selected from the group listed in Table 12B or Table 13A or Table 14, and any combinations thereof.
29. The method of any of paragraphs 1-28, wherein the DNA methylation of least about 200 target genes selected from any combination of genes in the list in Table 12A or Table 13A or Table 14 are measured in the pluripotent cell line, and compared to the reference DNA methylation level of the same set of at least 200 target genes.
30. The method of any of paragraphs 1-29, wherein the DNA methylation of least about 200 target genes selected from any combination of genes in the list in Table 12A or Table 13A or Table 14 are selected from any combination of genes of Numbers 1-500 listed in Table 12A or Table 13A or Table 14.
31. The method of any of paragraphs 1-30, wherein the DNA methylation of least about 200 target genes are selected from Numbers 1-200 listed in Table 12A or Table 13A or Table 14.
32. The method of any of paragraphs 1-31, wherein the DNA methylation of least about 500 target genes selected from any combination of genes in the list in Table 12A or Table 13A or Table 14 are measured in the pluripotent cell line, and compared to the reference DNA methylation level of the same set of at least 500 target genes.
33. The method of any of paragraphs 1-32, wherein the DNA methylation of least about 500 target genes selected from any combination of genes in the list in Table 12A or Table 13A or Table 14 are selected from any combination of genes of Numbers 1-1000 listed in Table 12A or Table 13A or Table 14.
34. The method of any of paragraphs 1-33, wherein the DNA methylation of least about 500 target genes are selected from Numbers 1-500 listed in Table 12A or Table 13A or Table 14.
35. The method of any of paragraphs 1-29, wherein the DNA methylation of least about 1000 target genes selected from any combination of genes in the list in Table 12A or Table 13A or Table 14 are measured in the pluripotent cell line, and compared to the reference DNA methylation level of the same set of at least 1000 target genes.
36. The method of any of paragraphs 1-35, wherein the DNA methylation of least about 1000 target genes are selected from Numbers 1-2000 listed in Table 12A or Table 13A or Table 14.
37. The method of any of paragraphs 1-36, wherein the gene expression of least about 200 target genes selected from any combination of genes in the list in Table 12B or Table 13A or Table 14 are measured in the pluripotent cell line, and compared to the reference gene expression level of the same set of at least 200 target genes.
38. The method of any of paragraphs 1-37, wherein the gene expression of least about 200 target genes are selected from Numbers 1-500 listed in Table 12B or Table 13A or Table 14.
39. The method of any of paragraphs 1-38, wherein the gene expression of least about 500 target genes selected from any combination of genes in the list in Table 12B or Table 13A or Table 14 are measured in the pluripotent cell line, and compared to the reference gene expression level of the same set of at least 500 target genes.
40. The method of any of paragraphs 1-39, wherein the gene expression of least about 500 target genes are selected from Numbers 1-1000 listed in Table 12B or Table 13A or Table 14.
41. The method of any of paragraphs 1-40, wherein the gene expression of least about 1000 target genes selected from any combination of genes in the list in Table 12B or Tables 13A or Table 14 are measured in the pluripotent cell line, and compared to the reference gene expression level of the same set of at least 1000 target genes.
42. The method of any of paragraphs 1-41, wherein the gene expression of least about 1000 target genes are selected from Numbers 1-2000 listed in Table 12B or Tables 13A or Table 14.
43. The method of any of paragraphs 1-42, wherein number of DNA methylation genes in the pluripotent stem cell line having a statistically significant difference in methylation relative to the reference genes is 10, 9, 8, 7, 6, 5, 4, 3, 2, 1, or 0.
44. The method of any of paragraphs 1-43, wherein number of genes in the pluripotent stem cell line having a statistically significant difference in gene expression level relative to the reference genes is 10, 9, 8, 7, 6, 5, 4, 3, 2, 1, or 0.
45. The method of any of paragraphs 1-44, wherein the pluripotent stem cell is a mammalian pluripotent stem cell.
46. The method of any of paragraphs 1-45, wherein the pluripotent stem cell is human pluripotent stem cell.
47. Use of a pluripotent stem cell for screening a compound for biological activity, wherein the pluripotent cell is selected by a method of any of paragraphs 1-46.
48. The use of paragraph 47, wherein the screening comprises the steps of
   (i) optionally causing or permitting the pluripotent stem cell to differentiate along a specific lineage;
   (ii) contacting the cell with a test compound; and
   (iii) determining any effect of the compound on the cell.
49. The use of any of paragraphs 47-48, wherein the test compound is selected from the group consisting of small organic molecule, small inorganic molecule, polysaccharides, peptides, proteins, nucleic acids, an extract made from biological materials such as bacteria, plants, fungi, animal cells, animal tissues, and any combinations thereof.
50. The use of any of paragraphs 47-49, wherein the test compound is tested at concentration in the range of about 0.01 nM to about 1000 mM.
51. The use of any of paragraphs 47-50, wherein the method is a high-throughput screening method.
52. The use of any of paragraphs 47-51, wherein the biological activity is elicitation of a stimulatory, inhibitory, regulatory, toxic or lethal response in a biological assay.
53. The use of any of paragraphs 47-52, wherein the biological activity is selected from the group consisting of modulation of an enzyme activity, inactivation of a receptor, stimulation of a receptor, modulation of the expression level of one or more genes, modulation of cell proliferation, modulation of cell division, modulation of cell morphology, and any combinations thereof.
54. The use of any of paragraphs 47-53, wherein the specific lineage is genotypic or phenotypic of a disease.
55. The use of any of paragraphs 47-54, wherein the specific lineage is genotypic or phenotypic of an organ, tissue, or a part thereof.
56. Use of a pluripotent stem cell for treatment of a subject by administering to a subject a pluripotent stem cell, wherein the pluripotent stem cell is selected by a method of any of paragraphs 1-46.
57. The use of paragraph 56, wherein the subject is mammal.
58. The use of any of paragraphs 56-57, wherein the subject is mouse.
59. The use of any of paragraphs 56-57, wherein the subject is human.
60. The use of any of paragraphs 56-59, wherein the subject suffers from or is diagnosed with a disease or conditions selected from the group consisting of cancer, diabetes, cardiac failure, muscle damage, Celiac Disease, neurological disorder, neurodegenerative disorder, lysosomal storage disease, and any combinations thereof.
61. The use of any of paragraphs 56-60, wherein said administration is local.
62. The use of any of paragraphs 56-61, wherein said administration is transplantation of the pluripotent stem cell into the subject.
63. The use of any of paragraphs 56-62, further comprising differentiating the pluripotent stem cell before administering the pluripotent stem cell, or differentiated progeny thereof to the subject.
64. The use of paragraph 63, wherein the pluripotent stem cell is differentiated along a lineage selected from the group consisting of mesoderm, endoderm, ectoderm, neuronal, hematopoietic lineages, and any combinations thereof.
65. The use of any of paragraphs 63-64, wherein the pluripotent stem cell is differentiated into an insulin producing cell (pancreatic cell, beta-cell, etc.), neuronal cell, muscle cell, skin cell, cardiac muscle cell, hepatocyte, blood cell, adaptive immunity cell, innate immunity cell and the like.
66. A kit comprising a pluripotent stem cell selected by a method of any of paragraphs 1-26.
67. The kit of paragraph 66, further comprising instructions for use.
68. The kit of any of paragraphs 66-67, wherein the pluripotent stem cell is useful for a use of any of paragraphs 47-55.
69. The kit of any of paragraphs 66-67, wherein the pluripotent stem cell is useful for use of any of paragraphs 56-65.
70. An assay for characterizing a plurality of properties of a pluripotent cell, the assay comprising at least 2 of the following:
   a. a DNA methylation assay;
   b. a gene expression assay; and
   c. a differentiation assay.
71. The assay of paragraph 70, wherein the DNA methylation assay is a bisulfite sequencing assay.
72. The assay of any of paragraphs 70-71, wherein DNA methylation assay is a whole genome bisulfite sequencing assay.
73. The assay of any of paragraphs 70-72, wherein DNA methylation assay is selected from the group consisting of: enrichment-based methods (e.g. MeDIP, MBD-seq and MethylCap), bisulfide sequencing and bisulfite-based methods (e.g. RRBS, bisulfite sequencing, Infinium, GoldenGate, COBRA, MSP, MethyLight) and restriction-digestion methods (e.g., MRE-seq).
74. The assay of any of paragraphs 70-73, wherein the gene expression assay is a microarray assay.
75. The assay of any of paragraphs 70-74, wherein the differentiation assay is a quantitative differentiation assay.
76. The assay of any of paragraphs 70-75, wherein the differentiation assay assess the ability of the pluripotent cell to differentiate into at least one of the following lineages: mesoderm, endoderm, ectoderm, neuronal, or hematopoietic lineages.
77. The assay of any of paragraphs 70-76, wherein the ability of the pluripotent cell to differentiate into at least one of the following lineages: mesoderm, endoderm and ectoderm is determined by immunostaining or FAC sorting using an antibody to at least one marker for mesoderm, endoderm and ectoderm lineages.
78. The assay of any of paragraphs 70-77, wherein the ability of the pluripotent cell to differentiate into at least one of the following lineages: mesoderm, endoderm and ectoderm is determined by immunostaining the pluripotent stem cell after at least about 7 days in EB.
79. The assay of any of paragraphs 70-78, wherein the ability of the pluripotent cell to differentiate along mesoderm lineage is determined by positive immunostaining for VEGF receptor II (KDR) or actin α-2 smooth muscle (ACTA2).
80. The assay of any of paragraphs 70-79, wherein the ability of the pluripotent cell to differentiate along ectoderm lineage is determined by positive immunostaining for Nestin or Tubulin β3.
81. The assay of any of paragraphs 70-80, wherein the ability of the pluripotent cell to differentiate along endoderm lineage is determined by positive immunostaining for alpha-feto protein (AFP).
82. The assay of any of paragraphs 70-81, wherein the assay is a high-throughput assay for assaying a plurality of different pluripotent stem cells.
83. The assay of paragraph 81, wherein the high-throughput assay assesses a plurality of different induced pluripotent stem cells from a subject.
84. The assay of paragraph 83, wherein the subject is a mammal.
85. The assay of paragraph 83, wherein the subject is a human subject.
86. The assay of any of paragraphs 70-85, wherein DNA methylation genes are selected from the group consisting of cancer genes, oncogenes, tumor suppressor genes, developmental genes, lineage marker genes, and any combinations thereof.
87. The method of any of paragraphs 70-86, wherein DNA methylation genes are selected from the group consisting of BMP4, CAT, CD14, CXCL5, DAZL, DNMT3B, GATA6, GAPDH, LEFTY2, MEG3, PAX6, S100A6, SOX2, SNAIL, TF, and any combinations thereof.
88. The assay of any of paragraphs 70-86, wherein the gene expression assay determines the expression of genes selected from any combination of genes listed in Table 7 or Tables 13A or Table 14.
89. The assay of any of paragraphs 70-88, wherein the DNA methylation assay determines the DNA methylation levels of any combination of a plurality of target genes selected from the group listed in Table 12A or Tables 13A or Table 14.
90. The assay of any of paragraphs 70-89, wherein the DNA methylation assay determines the DNA methylation levels of any combination of at least 200 genes listed in Table 12A or Tables 13A or Table 14.
91. The assay of any of paragraphs 70-89, wherein the DNA methylation assay determines the DNA methylation levels of any combination of at least 200 genes of genes of Numbers 1-500 listed in Table 12A or Tables 13A or Table 14.
92. The assay of any of paragraphs 70-91, wherein the DNA methylation assay determines the DNA methylation levels of any combination of at least 500 genes listed in Table 12A or Tables 13A or Table 14.
93. The assay of any of paragraphs 70-92, wherein the DNA methylation assay determines the DNA methylation levels of any combination of at least 500 genes of genes of Numbers 1-1000 listed in Table 12A.
94. The assay of any of paragraphs 70-93, wherein the DNA methylation assay determines the DNA methylation levels of any combination of at least 1000 genes listed in Table 12A or Tables 13A or Table 14.
95. The assay of any of paragraphs 70-92, wherein the DNA methylation assay determines the DNA methylation levels of any combination of at least 1000 genes of genes of Numbers 1-2000 listed in Table 12A or Tables 13A or Table 14.
96. The assay of any of paragraphs 70-95, wherein the gene expression assay determines the gene expression level of any combination of a plurality of target genes selected from the group listed in Table 12B.
97. The assay of any of paragraphs 70-96, wherein the gene expression assay determines the gene expression level of any combination of at least 200 genes listed in Table 12B or Tables 13A or Table 14.
98. The assay of any of paragraphs 70-97, wherein the gene expression assay determines the gene expression level of any combination of at least 200 genes of genes of Numbers 1-500 listed in Table 12B or Tables 13A or Table 14.
99. The assay of any of paragraphs 70-96, wherein the gene expression assay determines the gene expression level of any combination of at least 500 genes listed in Table 12B or Tables 13A or Table 14.
100. The assay of any of paragraphs 70-97, wherein the gene expression assay determines the gene expression level of any combination of at least 500 genes of genes of Numbers 1-1000 listed in Table 12B or Tables 13A or Table 14.
101. The assay of any of paragraphs 70-96, wherein the gene expression assay determines the gene expression level of any combination of at least 1000 genes listed in Table 12B or Tables 13A or Table 14.
102. The assay of any of paragraphs 70-97, wherein the gene expression assay determines the gene expression level of any combination of at least 1000 genes of genes of Numbers 1-2000 listed in Table 12B or Tables 13A or Table 14.
103. The use of the assay of any of paragraphs 70-102 to generate a scorecard from at least one or a plurality of pluripotent stem cell lines.
104. A method for generating a pluripotent stem cell scorecard comprising:
   (i) measuring DNA methylation in a first set of target genes in a plurality of pluripotent stem cell lines;
   (ii) measuring gene expression in a second set of target genes in the plurality of pluripotent stem cell lines; and
   (iii) measuring differentiation potential of the plurality of pluripotent stem cell lines.
105. The method of paragraph 104, further comprising:
   (i) calculating an average methylation level for each target gene in the first set of target genes; and
   (ii) calculating an average gene expression level for each target gene in the second set of target genes.
106. The method of any of paragraphs 104-105, wherein the differentiation potential is the ability to differentiate into a lineage selected from the group consisting of mesoderm, endoderm, ectoderm, neuronal, hematopoietic lineages, and any combinations thereof.
107. The method of any of paragraphs 104-106, wherein the plurality of pluripotent stem cell lines is at least 5 pluripotent stem cell lines.
108. The method of any of paragraphs 104-107, wherein the DNA methylation is measured by a bisulfite sequencing assay.
109. The method of any of paragraphs 104-108, wherein the DNA methylation is measured by a whole genome bisulfite sequencing assay.
110. The method of any of paragraphs 104-109, wherein the DNA methylation is measured by any one of the methods selected from the group of: enrichment-based methods (e.g. MeDIP, MBD-seq and MethylCap), bisulfide sequencing and bisulfite-based methods (e.g. RRBS, bisulfite sequencing, Infinium, GoldenGate, COBRA, MSP, MethyLight) and restriction-digestion methods (e.g., MRE-seq).
111. The method of any of paragraphs 104-110 wherein the gene expression is measured by a microarray assay.
112. The assay of any of paragraphs 104-111, wherein the differentiation potential is measured by a quantitative differentiation assay.
113. The method of any of paragraphs 104-112, wherein the ability of the pluripotent cell to differentiate into at least one of the following lineages: mesoderm, endoderm and ectoderm is determined by immunostaining or FAC sorting using an antibody to at least one marker for mesoderm, endoderm and ectoderm lineages.
114. The method of any of paragraphs 104-113, wherein the ability of the pluripotent cell to differentiate into at least one of the following lineages: mesoderm, endoderm and ectoderm is determined by immunostaining the pluripotent stem cell after at least about 7 days in EB.
115. The method of any of paragraphs 104-114, wherein the ability of the pluripotent cell to differentiate along mesoderm lineage is determined by positive immunostaining for VEGF receptor II (KDR) or actin α-2 smooth muscle (ACTA2).
116. The method of any of paragraphs 104-115, wherein the ability of the pluripotent cell to differentiate along ectoderm lineage is determined by positive immunostaining for Nestin or Tubulin 133.
117. The method of any of paragraphs 104-116, wherein the ability of the pluripotent cell to differentiate along endoderm lineage is determined by positive immunostaining for alpha-feto protein (AFP).
118. The method of any of paragraphs 104-117, wherein the first set of genes is selected from the group consisting of cancer genes, oncogenes, tumor suppressor genes, developmental genes, lineage marker genes, and any combinations thereof.
119. The method of any of paragraphs 104-118, wherein the first set of genes comprises at least one gene selected from the group consisting of BMP4, CAT, CD14, CXCL5, DAZL, DNMT3B, GATA6, GAPDH, LEFTY2, MEG3, PAX6, S100A6, SOX2, SNAI1, TF, and any combinations thereof.
120. The method of any of paragraphs 104-119, wherein the first set of DNA methylation genes comprises any combination of a plurality of target genes selected from the group listed in Table 12A or Tables 13A or Table 14.
121. The method of any of paragraphs 104-120, wherein the first set of DNA methylation genes comprises any combination of at least 200 genes listed in Table 12A or Tables 13A or Table 14.
122. The method of any of paragraphs 104-121, wherein the first set of DNA methylation genes comprises any combination of at least 200 genes of genes of Numbers 1-500 listed in Table 12A or Tables 13A or Table 14.
123. The method of any of paragraphs 104-122, wherein the first set of DNA methylation genes comprises any combination of at least 500 genes listed in Table 12A or Tables 13A or Table 14.
124. The method of any of paragraphs 104-123, wherein the first set of DNA methylation genes comprises any combination of at least 500 genes of genes of Numbers 1-1000 listed in Table 12A or Tables 13A or Table 14.
125. The method of any of paragraphs 104-124, wherein the first set of DNA methylation genes comprises any combination of at least 1000 genes listed in Table 12A or Tables 13A or Table 14.
126. The method of any of paragraphs 104-125, wherein the first set of DNA methylation genes comprises any combination of at least 1000 genes of genes of Numbers 1-2000 listed in Table 12A or Tables 13A or Table 14.
127. The method of any of paragraphs 104-126, wherein the second set of gene expression genes comprises any combination of a plurality of target genes selected from the group listed in Table 12B or Tables 13A or Table 14.
128. The method of any of paragraphs 104-127, wherein the second set of gene expression genes comprises any combination of at least 200 genes listed in Table 12B or Tables 13A or Table 14.
129. The method of any of paragraphs 104-128, wherein the second set of gene expression genes comprises any combination of at least 200 genes of genes of Numbers 1-500 listed in Table 12B or Tables 13A or Table 14.
130. The method of any of paragraphs 104-129, wherein the second set of gene expression genes comprises any combination of at least 500 genes listed in Table 12B or Tables 13A or Table 14.
131. The method of any of paragraphs 104-130, wherein the second set of gene expression genes comprises any combination of at least 500 genes of genes of Numbers 1-1000 listed in Table 12B or Tables 13A or Table 14.
132. The method of any of paragraphs 104-131, wherein the second set of gene expression genes comprises any combination of at least 1000 genes listed in Table 12B.
133. The method of any of paragraphs 104-132, wherein the second set of gene expression genes comprises any combination of at least 1000 genes of genes of Numbers 1-2000 listed in Table 12B or Tables 13A or Table 14.
134. A scorecard of the performance parameters of a pluripotent stem cell, the scorecard comprising:
   (i) a first data set comprising the DNA methylation levels for a plurality of DNA methylation target genes from a plurality of pluripotent stem cell lines;
   (ii) a second data set comprising the gene expression levels for a plurality of gene expression target genes from a plurality of pluripotent stem cell lines; and
   (iii) a third data set comprising the differentiation propensity levels for differentiation into ectoderm, mesoderm and endoderm lineages from a plurality of pluripotent stem cell lines.
135. The scorecard of paragraph 134, wherein the plurality of reference DNA methylation genes is at least about 500, at least about 1000, at least about 1500, or at least about 200 reference DNA methylation genes.
136. The scorecard of paragraphs 134 or 135, wherein the plurality of reference DNA methylation genes is selected from any combination of genes listed in Table 12A or Tables 13A or Table 14.
137. The scorecard of paragraphs 134 or 136, wherein the plurality of reference DNA methylation genes is selected from any combination of genes listed in Table 12A or Tables 13A or Table 14.
138. The scorecard of any of paragraphs 134 to 137, the plurality of reference DNA methylation genes is selected from any combination of at least 200 genes listed in Table 12A or Tables 13A or Table 14.
139. The scorecard of any of paragraphs 134 to 138, the plurality of reference DNA methylation genes is selected from any combination of at least 200 genes of genes of Numbers 1-500 listed in Table 12A or Tables 13A or Table 14.
140. The scorecard of any of paragraphs 134 to 139, the plurality of reference DNA methylation genes is selected from any combination of at least 500 genes listed in Table 12A or Tables 13A or Table 14.
141. The scorecard of any of paragraphs 134 to 140, the plurality of reference DNA methylation genes is selected from any combination of at least 500 genes of genes of Numbers 1-1000 listed in Table 12A or Tables 13A or Table 14.
142. The scorecard of any of paragraphs 134 to 141, the plurality of reference DNA methylation genes is selected from any combination of at least 1000 genes listed in Table 12A or Tables 13A or 14.
143. The scorecard of any of paragraphs 134 to 142, the plurality of reference DNA methylation genes is selected from any combination of at least 1000 genes of genes of Numbers 1-2000 listed in Table 12A or Tables 13A or Table 14.
144. The scorecard of any of paragraphs 134 to 143, wherein the plurality of reference DNA methylation genes is the DNA methylation status of the whole genome.
145. The scorecard of any of paragraphs 134 to 144, wherein the plurality of reference DNA methylation genes comprises cancer genes, oncogenes, tumor suppressor genes, development genes and lineage marker genes.
146. The scorecard of any of paragraphs 134 to 145, wherein the plurality of reference DNA methylation genes comprises at least one gene selected from the group consisting of BMP4, CAT, CD14, CXCL5, DAZL, DNMT3B, GATA6, GAPDH, LEFTY2, MEG3, PAX6, S100A6, SOX2, SNAIL, TF, and any combinations thereof.
147. The scorecard of any of paragraphs 134 to 146, wherein at least the first and/or the second data set are connected to a data storage device.
148. The scorecard of any of paragraphs 134 to 147, wherein at least the first and/or second data set are connected to a data storage device, and the data storage device is a database located on a computer device.
149. The scorecard of any of paragraphs 134 to 148, wherein the plurality of stem cell lines is at least 5, at least 10, at least 15, or at least 20 pluripotent stem cell lines.
150. The scorecard of any of paragraphs 134 to 149, wherein the plurality of stem cell lines comprises at least one pluripotent stem cell line selected from the group consisting of HUES64, HUES3, HUES8, HUES53, HUES28, HUES49, HUES9, HUES48, HUES45, HUES1, HUES44, HUES6, H1, HUES62, HUES65, H7, HUES13, HUES63, HUES66, and any combinations thereof.
151. The scorecard of any of paragraphs 134 to 140, wherein the plurality of stem cell lines comprises at least 5 pluripotent stem cell lines independently selected from the group consisting HUES64, HUES3, HUES8, HUES53, HUES28, HUES49, HUES9, HUES48, HUES45, HUES1, HUES44, HUES6, H1, HUES62, HUES65, H7, HUES13, HUES63, HUES66.
152. The scorecard of any of paragraphs 134 to 151, wherein the plurality of pluripotent stem cell lines comprises at least one mammalian pluripotent stem cell line.
153. The score card of any of paragraphs 134 to 152, wherein all the pluripotent stem cell lines of the plurality of pluripotent stem cell lines are mammalian pluripotent stem cell lines.
154. The scorecard of any of paragraphs 134 to 153, wherein the plurality of pluripotent stem cell lines comprises at least human pluripotent stem cell line.
155. The scorecard of any of paragraphs 134 to 154, wherein all the pluripotent stem cell lines of the plurality of pluripotent stem cell lines are human pluripotent stem cell lines.
156. The scorecard of any of paragraphs 134 to 155, wherein the pluripotent stem cell is a mammalian pluripotent stem cell
157. The scorecard of any of paragraphs 134 to 156, wherein the pluripotent stem cell is a human pluripotent stem cell.
158. The scorecard of any of paragraphs 134 to 157, wherein the pluripotent stem cell is an induced pluripotent stem (iPS) cell.
159. The scorecard of any of paragraphs 134 to 158, wherein the pluripotent stem cell is an embryonic stem cell.
160. The scorecard of any of paragraphs 134 to 159, wherein the pluripotent stem cell is an adult stem cell.
161. The scorecard of any of paragraphs 134 to 160, wherein the pluripotent stem cell is an autologous stem cell.
162. A kit comprising a scorecard of any of paragraphs 134-161.
163. The kit of paragraph 162, further comprising instructions of use.
164. The use of the scorecard of any of paragraphs 134-161 to distinguish an induced pluripotent stem cell from an embryonic stem cell line.
165. A kit for carrying out a method of any of paragraphs 1-46, wherein, the kit comprising:
   (i) reagents for measuring DNA methylation status; and
   (ii) reagents for measuring differentiation propensity of a pluripotent stem cell.
166. The kit of paragraph 165, further comprising reagents for measuring gene expression levels of a target gene expression gene.
167. The kit of any of paragraphs 165-166, further comprising instructions of use.
168. The kit of any of paragraphs 165-166, further comprising a scorecard of any of paragraphs 134-161.
169. A computer system for generating a quality assurance scorecard of a pluripotent stem cell, comprising:
   (a) at least one memory containing at least one program comprising the steps of:
      (i) receiving DNA methylation data of a set of DNA methylation target genes in the pluripotent stem cell line and performing a comparison of the DNA methylation data with a reference DNA methylation level of the same target genes;
      (ii) receiving differentiation potential data of the pluripotent stem cell line and comparing the differentiation potential data with a reference differentiation potential data;
      (iii) generating a quality assurance scorecard based on the comparison of the DNA methylation data as compared to reference DNA methylation parameters and comparing the differentiation propensity as compared to reference differentiation data; and
   (b) a processor for running said program.
170. The system of paragraph 169, wherein the program further comprises a step of:
   (i) receiving gene expression data of a second set of target genes in the pluripotent stem cell line and comparing the expression data with a reference gene expression level of the same second set of target genes;
   (ii) generating a quality assurance scorecard based on the comparison of the DNA methylation data as compared to reference DNA methylation parameters, and the comparison of the differentiation propensity as compared to reference differentiation data, and the comparison of the gene expression data as compared to reference gene expression levels.
171. The system of any of paragraphs 169-170, wherein the DNA methylation target genes have variable methylation.
172. The system of any of paragraphs 169-171, wherein the DNA methylation target genes are selected from cancer genes, oncogenes, tumor suppressor genes, development genes, lineage marker genes, and any combinations thereof.
173. The system of any of paragraphs 169-172, wherein the DNA methylation target genes are selected from the group consisting of: BMP4, CAT, CD14, CXCL5, DAZL, DNMT3B, GATA6, GAPDH, LEFTY2, MEG3, PAX6, S100A6, SOX2, SNAIL, TF, and any combinations thereof.
174. The system of any of paragraphs 169-173, wherein the reference DNA methylation level is a high level of methylation for epigenetic silencing of oncogenes, and low level of methylation for active transcription of tumor suppressor genes and developmental genes.
175. The system of any of paragraphs 167-174, wherein the DNA methylation target genes are selected from any combination of genes listed in Table 12A.
176. The system of any of paragraphs 167-175, wherein the DNA methylation target genes are selected from at least 200 genes listed in Table 12A.
177. The system of any of paragraphs 167-176, wherein the DNA methylation target genes are selected from any combination of at least 200 genes of gene numbers 1-500 listed in Table 12A or Tables 13A or 14.
178. The system of any of paragraphs 167-177, wherein the DNA methylation target genes are selected from at least 500 genes listed in Table 12A.
179. The system of any of paragraphs 167-178, wherein the DNA methylation target genes are selected from any combination of at least 500 genes of gene numbers 1-1000 listed in Table 12A or Tables 13A or 14.
180. The system of any of paragraphs 167-179, wherein the DNA methylation target genes are selected from at least 1000 genes listed in Table 12A.
181. The system of any of paragraphs 167-180, wherein the DNA methylation target genes are selected from any combination of at least 1000 genes of gene numbers 1-3000 listed in Table 12A or Tables 13A or 14.
182. The system of any of paragraphs 167-181, further comprising a report generating module which generates a stem cell scorecard report based on quality of the pluripotent stem cell line.
183. The system of any of paragraphs 167-182, wherein the memory further comprises a database.
184. The system of any of paragraphs 167-183, wherein the database arranges the DNA methylation gene set in a hierarchical manner.
185. The system of any of paragraphs 167-184, wherein the database arranges the propensity to differentiation into different lineages in a hierarchical manner.
186. The system of any of paragraphs 167-185, wherein the database arranges the gene expression level data set in a hierarchical manner.
187. The system of any of paragraphs 167-186, wherein the memory is connected to the first computer via a network.
188. The system of paragraph 187, wherein the network comprises a wide area network.
189. The system of any of paragraphs 167-188, wherein the scorecard provides an indication of suitable uses or applications of the pluripotent stem cell.
190. The system of any of paragraphs 167-189, wherein the reference DNA methylation level is range of normal variation of methylation for that DNA methylation target gene.
191. The system of any of paragraphs 167-190, wherein the reference DNA methylation level is an average of DNA methylation for that DNA methylation target gene, wherein the average is calculated from DNA methylation of that target gene in a plurality of pluripotent stem cell lines.
192. The system of any of paragraphs 167-191, wherein the differentiation potential of the pluripotent cell line is determined by a quantitative differentiation assay.
193. The system of any of paragraphs 167-192, wherein the reference differentiation potential is the ability to differentiate into a lineage selected from the group consisting of mesoderm, endoderm, ectoderm, neuronal, hematopoietic lineages, and any combinations thereof.
194. The system of any of paragraphs 167-193, wherein the reference gene expression level is range of normal variation of gene expression for that gene expression target gene.
195. The method of any of paragraphs 111-128, wherein the reference gene expression level is an average level of gene expression for that target gene, wherein the average is calculated from expression level of that target gene in a plurality of pluripotent stem cell lines.
196. The system of any of paragraphs 167-194, wherein the reference DNA methylation, differentiation potential data, and gene expression level data is generated from a plurality of pluripotent stem cell lines.
197. The system of paragraph 196, wherein the plurality of pluripotent stem cell lines is at least 5, at least 10, at least 15, or at least 20 pluripotent stem cell lines.
198. The system of any of paragraphs 167-197, wherein the DNA methylation target genes include at least one or more of the gene expression target genes.
199. The system of any of paragraphs 167-198, wherein the gene expression target genes include at least one or more of the DNA methylation target genes.
200. A computer readable medium comprising instructions for generating a quality assurance scorecard of a pluripotent stem cell line, comprising:
   (i) receiving DNA methylation data of a set of DNA methylation target genes in the pluripotent stem cell line and performing a comparison of the DNA methylation data with a reference DNA methylation level of the same target genes;
   (ii) receiving differentiation potential data of the pluripotent stem cell line and comparing the differentiation potential data with a reference differentiation potential data; and
   (iii) generating a quality assurance scorecard based on the comparison of the DNA methylation data as compared to reference DNA methylation parameters and comparing the differentiation propensity as compared to reference differentiation data.
201. The computer-readable medium of paragraph 200, wherein the medium further comprises instructions for:
   a. receiving gene expression data of a second set of target genes in the pluripotent stem cell line and comparing the expression data with a reference gene expression level of the same second set of target genes; and
   b. generating a quality assurance scorecard based on the comparison of the DNA methylation data as compared to reference DNA methylation parameters, and the comparison of the differentiation propensity as compared to reference differentiation data, and the comparison of the gene expression data as compared to reference gene expression levels.
202. A kit for determining the quality of a pluripotent stem cell line, comprising at least two of the following:
   a. reagents for measuring methylation status of a plurality of DNA methylation genes,
   b. reagents for measuring gene expression levels of a plurality of genes; and
   c. reagents for measuring the differentiation propensity of the pluripotent stem cell into ectoderm, mesoderm and endoderm lineages.
203. The kit of paragraph 202, further comprising instructions of use.
204. The kit of any of paragraphs 202-203, further comprising at least one pluripotent stem cell line.
205. The kit of any of paragraphs 202-204, further comprising a scorecard of any of paragraphs 134-161.
206. A method for producing a scorecard to identify the pluripotency of a stem cell line of interest, the method comprising:
   a. providing a computer with associated memory and a processor for executing one or more programs adapted for carrying out one or more of the following:
      (i) obtaining DNA methylation data of a set of DNA methylation target genes and obtaining gene expression data of a set of gene expression genes in at least one pluripotent stem cell line of interest, and
      (ii) obtaining DNA methylation data of a set of DNA methylation target genes and obtaining gene expression data of a set of gene expression genes in at least one reference pluripotent stem cell line;
      (iii) performing data normalization of the gene expression data obtained in elements (i) and (ii);
      (iv) performing gene mapping of the DNA methylation data and gene expression data obtained in elements (i) and (ii);
      (v) comparing the DNA methylation data and the normalized gene expression data from the pluripotent stem cell line of interest obtained in elements (i) and (iii) with normalized DNA methylation data and the normalized gene expression data from the reference pluripotent stem cell line obtained in elements (ii) and (iii) and identify genes in the pluripotent stem cell line having a DNA methylation level or normalized gene expression level which falls outside by a statistically significant amount of the normal range of the DNA methylation levels or gene expression levels of the reference pluripotent stem cell line;
      (vi) apply a relevance filter of genes identified in elements (v) to identify genes which have a DNA methylation difference of greater than 15% or an gene expression change of greater than 1.5-fold as compared to the reference DNA methylation levels or gene expression level of the reference pluripotent stem cell line;
      (vii) obtain gene sets of DNA methylation target genes and gene expression target genes and lineage markers; and
   b. generating a pluripotent scorecard report comprising the number and/or percentage of number of genes identified in element (vi) which have deviations of DNA methylation and/or gene expression in the pluripotent stem cell line of interest as compared to the at least one reference pluripotent stem cell line.
207. The method of paragraph 206, wherein the genes identified in step (v) have a DNA methylation level or normalized gene expression level which falls outside the center quartile by at least 1.2-times the interquartile range of the normal DNA methylation range or gene expression range of the reference pluripotent stem cell line.
208. The method of paragraph 206, wherein the genes identified in step (vi) have a DNA methylation difference of greater than 20% or an gene expression change of greater than 2-fold as compared to the reference DNA methylation levels or gene expression level of the reference pluripotent stem cell line.
209. The method of paragraph 206, wherein the report scorecard further comprises the name of the affected genes which deviate from the DNA methylation and/or gene expression in the pluripotent stem cell line of interest as compared to the at least one reference pluripotent stem cell line.
210. The method of paragraph 206, wherein the DNA methylation data is obtained by whole genome DNA methylation, or reduced-representation bisulfate sequencing (RRBS).
211. The method of paragraph 206, wherein the gene expression data is obtained by microarray data or quantitative PCR (qPCR).
212. The method of paragraph 206, wherein in the gene sets of DNA methylation target genes, gene expression target genes and lineage markers are listed the tables selected from the group selected from: Table 7, Table 12A, Table 12B, Table 12C, Table 13A, Table 13B or Table 14.
213. The method of any of paragraphs 206 to 212, wherein the method is carried out on a computer.
214. The method of any of paragraphs 206 to 213, wherein the method is a computer system.
215. The method of any of paragraphs 206 to 214, wherein the one or more program is performed by a scorecard software program on computer readable media.
216. A method for producing a lineage scorecard to identify the differentiation propensity of a pluripotent stem cell line of interest, the system comprising:
   a. providing a computer with associated memory and a processor for executing one or more programs adapted for carrying out one or more of the following:
      (i) obtaining DNA methylation data and gene expression data of a set of target lineage marker genes in embryoid bodies (EBs) at least one pluripotent stem cell line of interest, and
      (ii) obtaining DNA methylation data and gene expression data of a set of target lineage marker genes in embryoid bodies (EBs) in at least one reference pluripotent stem cell line;
      (iii) optionally performing assay normalization, by rescaling the DNA methylation data and gene expression data obtained in elements (i) and (ii) with a positive control,
      (iv) optionally performing sample normalization and variance stabilization of the DNA methylation data and gene expression data obtained in elements (i) and (ii) across replicate experiments;
      (v) comparing the DNA methylation data and the gene expression data of the lineage marker genes from the pluripotent stem cell line of interest obtained in elements (i) with DNA methylation data and the gene expression data of the lineage marker genes from the reference pluripotent stem cell line obtained in elements (ii) and identify lineage genes in the pluripotent stem cell line having a DNA methylation level or normalized gene expression level which falls which are increased or decreased by a statistically significant amount as compared to the normal range of the DNA methylation levels or gene expression levels of the reference pluripotent stem cell line, thereby producing a variance values for each individual lineage marker gene;
      (vi) obtain gene sets of lineage marker genes for the characteristic cellular lineage or germ layer of interest;
      (vii) perform enrichment analysis by calculating the mean variation from the individual variation value for each lineage marker (obtained in elements (v)) listed in the lineage marker gene set obtained in element (vi); and
   b. generating a lineage scorecard report comprising the mean variation for all genes in the lineage marker gene set of the pluripotent stem cell line as compared to the at least one reference pluripotent stem cell line.
217. The method of paragraph 216, wherein the pluripotent stem cell line has been characterized by the scorecard of paragraph 206.
218. The method of any of paragraphs 216 to 217, wherein in the sets of target lineage gene markers for DNA methylation data and gene expression data are listed the tables selected from the group selected from: Table 7, Table 13A, Table 13B or Table 14.
219. The method of any of paragraphs 216 to 218, wherein the reference comparison in element (v) uses moderated t-test to identify a lineage marker gene with a statistically significant increase or decrease in DNA methylation or gene expression as compared to the DNA methylation or gene expression of the reference pluripotent stem cell line.
220. The method of any of paragraphs 216 to 219, wherein the reference comparison using moderated t-test is performed using Bioconductors Limma package.
221. The method of any of paragraphs 216 to 220, wherein the lineage marker gene sets can be obtained by gene ontology, MolSigDB program or curation.
222. The method of any of paragraphs 216 to 221, wherein the enrichment analysis of element (vii) calculates the mean t-scores from the individial t-scores for each lineage marker.
223. The method of paragraph 216, wherein the sample normalization of element (iv) is performed by Bioconductor VSN package.
224. The method of any of paragraphs 216 to 223, wherein the sets of lineage marker genes in element (vi) are gene sets selected from the group of: ectoderm germ layer, mesoderm germ layer, endoderm germ layer, neural lineage gene sets, hematopoietic lineage gene sets, pluripotent cell signature gene sets, epidermis lineage gene sets, mesenchymal stem cell lineage gene sets, bone lineage gene sets, cartilage lineage gene sets, fat lineage gene sets, muscle lineage gene sets, blood vessel lineage gene sets, heart lineage gene sets, lymphoid cells lineage gene sets, myeloid cells lineage gene sets, liver lineage gene sets, pancreas lineage gene sets, epithelium lineage gene sets, motor neuron lineage gene sets, monocytes-macrophages lineage gene sets, ISCI lineage gene sets, or any selection of genes listed in Table 7 or 13A and 13B and Table 14,
225. The method of any of paragraphs 216 to 224, wherein the method is carried out on a computer.
226. The method of any of paragraphs 216 to 225, wherein the system is a computer system.
227. The method of any of paragraphs 216 to 226, wherein the one or more programs is performed by a scorecard software program on computer readable media.
228. A system for producing a scorecard to identify the pluripotency of a stem cell line of interest, the system comprising at least one or more of the following modules:
   a. a determination module for measuring the DNA methylation levels of DNA methylation target genes and/or gene expression levels of gene expression target genes in a pluripotent stem cell line of interest,
   b. a computer module comprising a processor and associated memory, comprising one or more of the following modules:
      (i) a storage module for storing the DNA methylation levels and gene expression levels measured by the determination module, and storing reference DNA methylation levels of DNA methylation target genes and reference gene expression levels of gene expression target genes of one or more reference pluripotent stem cell lines,
      (ii) a normalization module for normalizing the gene expression levels measured by the determination module,
      (iii) a gene mapping module for matching the DNA methylation levels of DNA methylation target genes measured in the pluripotent stem cell line with the DNA methylation levels of DNA methylation target genes of one or more reference pluripotent stem cell line, and/or matching the gene expression levels of gene expression target genes measured in the pluripotent stem cell line with the gene expression levels of gene expression target genes of one or more reference pluripotent stem cell line,
      (iv) a comparison module for (i) comparing the DNA methylation levels of DNA methylation target genes from the pluripotent stem cell line of interest with the DNA methylation levels of the same DNA methylation target genes from the one or more reference pluripotent stem cell lines, and/or (ii) comparing the gene expression levels of gene expression target genes of the pluripotent stem cell line of interest with the gene expression levels of the same gene expression target genes from the one or more reference pluripotent stem cell lines, and identify genes in the pluripotent stem cell line having a DNA methylation level or normalized gene expression level which falls outside by a statistically significant amount of the normal range of the DNA methylation levels or gene expression levels of the reference pluripotent stem cell line;
      (v) a relevance filter module for selecting genes identified by the comparison module which have a DNA methylation difference of greater than at least 15% or an gene expression change of greater than at least 1.5-fold as compared to the reference DNA methylation level or gene expression level of the reference pluripotent stem cell line;
      (vi) a gene set module for selecting genes identified by the comparison module and/or the relevance filter module of interest,
   c. a display module for displaying a scorecard report comprising the number and/or percentage of number of genes identified by the comparison module and/or the relevance filter module and/or the gene set module which have deviations of DNA methylation and/or gene expression in the pluripotent stem cell line of interest as compared to the at least one reference pluripotent stem cell line.
229. The system of paragraph 228, wherein the determination module can measure the DNA methylation levels of DNA methylation target genes and/or gene expression levels of gene expression genes or lineage marker genes in one or more reference pluripotent stem cell lines.
230. The system of paragraph 228, wherein the storage module can store the measure the DNA methylation levels of DNA methylation target genes and/or gene expression levels of gene expression genes or lineage marker genes in one or more reference pluripotent stem cell lines.
231. The system of paragraph 228, wherein one or more modules can be combined into a single module.
232. A system for producing a lineage scorecard to identify the differentiation propensity of a stem cell line of interest, the system comprising at least one or more of the following modules:
   a. a determination module for measuring the lineage gene expression level of a plurality of lineage marker genes in embroid bodies (EBs) a pluripotent stem cell line of interest,
   b. a computer module comprising a processor and associated memory, comprising one or more of the following modules:
      (i) a storage module for storing the lineage gene expression levels measured by the determination module, and storing reference lineage gene expression levels of lineage marker genes in embroid bodies (EBs) of one or more reference pluripotent stem cell lines,
      (ii) an assay normalization module for normalizing the gene expression levels based on a positive gene expression control,
      (iii) a sample normalization module for normalizing and variance stabilization of the gene expression levels of lineage marker genes across replicate gene expression level measurements of the same lineage marker genes in embroid bodies (EBs) from the same pluripotent stem cell line of interest,
      (iv) a comparison module for comparing the gene expression level of lineage marker genes from embroid bodies (EBs) from the pluripotent stem cell line of interest with the gene expression level of the same lineage marker genes from embroid bodies (EBs) from one or more reference pluripotent stem cell lines, and calculate the statistical difference of the difference in the level of lineage gene expression in the pluripotent stem cell line as compared to the level of lineage gene expression of the reference pluripotent stem cell line(s) for each lineage marker gene;
      (v) a gene set module for selecting a subset of lineage marker genes which are characteristic of a particular cellular lineage of interest;
      (vi) enrichment analysis module for calculating the mean stastistical difference calculated by the comparison module of the genes of the subset of lineage marker genes selected by the gene set module;
   c. a display module for displaying a lineage scorecard report comprising the mean stastistical difference of lineage gene expression for the lineage marker genes in each subset of lineage marker gene set of the pluripotent stem cell line as compared to the at least one reference pluripotent stem cell line.
233. The system of paragraph 232, wherein one or more modules can be combined into a single module.

### EXAMPLES

Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

The developmental potential of human pluripotent stem cells suggests that they can produce disease-relevant cell types for biomedical research. However, substantial variation has been reported among pluripotent cell lines, which could affect their utility and clinical safety. Such cell-line specific differences must be better understood before one can confidently use embryonic stem (ES) or induced pluripotent stem (iPS) cells in translational research. Towards this goal, the inventors have established genome-wide reference maps of DNA methylation and gene expression for 20 previously derived human ES lines and 12 human iPS cell lines, and have measured the in vitro differentiation propensity of these cell lines. This resource enabled the inventors to assess the epigenetic and transcriptional similarity of ES and iPS cells and to predict the differentiation efficiency of individual cell lines. The combination of assays yields a scorecard for quick and comprehensive characterization of pluripotent cell lines.

Pluripotent cell lines are valuable tools for disease modeling, drug screening and regenerative medicine. However, current validation assays for human pluripotent cell lines are cumbersome and not always accurate, which tends to slow down research and has led to some confusion about the potency of human iPS cells. To systematically address these issues, the inventors have established reference maps, herein referred to as “scorecards” of the pluripotent methylome and transcriptome, focusing on 31 low-passage ES and iPS cell lines. Furthermore, the inventors have also developed a quantitative differentiation assay and measured the differentiation propensities of these cell lines. Using this dataset, the inventors quantified the deviation of each ES or iPS cell line from the ES-cell reference, giving rise to a scorecard of cell line quality and utility. The inventors validated this scorecard by showing that (i) it detects DNA methylation defects that prevent differentiation into CD14-positive cells, and that (ii) it accurately predicts cell-line specific differences in the efficiency of making motor neurons. The inventors also compared human ES and iPS cell lines in terms of their DNA methylation, gene expression and differentiation propensities, observing higher variation for iPS cell lines but no single locus or gene sig-nature that could accurately distinguish between ES and iPS cell lines. In summary, the inventors dataset provides a reference for high-throughput characterization of human pluripotent cell lines using genomic assays.

Methods

ES and iPSC Cell Lines and Culture Conditions

A total of 20 human ES cell lines, 13 human iPS cell lines and 6 primary fibroblast cell lines were included in the current study (Table 1). The ES cell lines were obtained from the Human Embryonic Stem Cell Facility of the Harvard Stem Cell Institute (17 ES cell lines) and from WiCell (3 ES cell lines). The iPS cell lines were derived by retroviral transduction of OCT4, SOX2 and KLF4 in dermal fibroblasts. The fibroblasts were derived by skin puncture from the forearm of each respective donor and grown as previously described (Dimos et al., 2009). All pluripotent cell lines have been characterized by conventional methods (Chen et al., 2009; Cowan et al., 2004, Boulting et al., submitted), confirming that they qualify as pluripotent according to established standards (Maherali and Hochedlinger, 2008). The pluripotent stem cells were grown in human ES media consisting of KO-DMEM (Invitrogen), 10% KOSR (Invitrogen), 10% plasmanate (Talecris), 1% glutamax or L-glutamin, non-essential amino acids, penicillin/streptomycin, 0.1% 2-mercaptoethanol and 10-20 ng/mlbFGF. Cultures were grown on a monolayer of irradiated CF1-MEFs (GlobalStem) and passaged using trypsin (0.05%) or dispase (Invitrogen). Before collection of DNA and RNA for analysis, ES and iPS cells were either isolated by trypsin (0.05%) or dispase treatment, or plated on matrigel (BD Biosciences) for one passage and fed with human ES media conditioned in CF1-MEFs for 24 h.

Differentiation Protocols

A total of five ES/iPS cell differentiation protocols were used in the current study:

(i) Non-Directed EB Differentiation.

Undifferentiated cells were harvested using dispase or trypsin and plated in suspension in low-adherence plates in the presence of human ES cell culture media without bFGF and plasmanate. Cell aggregates (EBs) were allowed to grow for a total of 16 days, refreshing media every 48 h.

(ii) Monocyte/Macrophage Differentiation.

Undifferentiated cells were treated with multiple recombinant proteins following a published protocol for hematopoietic differentiation (Grigoriadis et al., 2010). Briefly, feeder depleted pluripotent cells were grown as small aggregates in suspension in 6-well low attachment plates (Corning) in StemPro-34 medium (Invitrogen) containing penicillin/streptomycin, glutamine (2 mM), monothioglycerol (0.0004M), ascorbic acid (50 m/ml) (Sigma-Aldrich) and BMP4 (10 ng/ml) (R&D Systems) for 24 h. To induce primitive steak/mesoderm formation, EBs were washed and cultured further in the StemPro-34 differentiation medium, supplemented with human recombinant bFGF (5 ng/ml) (Millipore) for another 3 days. At day 4, EBs were harvested again and cultured in the differentiation medium described above, additionally containing hVEGF (10 ng/ml) (PeproTech), hbFGF (1 ng/nal), hIL-6 (10 ng/ml) (PeproTech), hIL-3 (40 ng/mL) (PeproTech), hIL-11 (5 ng/mL) (PeproTech), and human recombinant SCF (100 ng/mL) (PeproTech) for another 4 days to induce hematopoietic specification. From day 8 onwards, cells were further cultured in StemPro-34 medium, containing hVEGF (10 ng/ml), human erythropoietin (4 U/ml) (Cell Sciences), human thrombopoietin (50 ng/ml) (Cell Sciences), and human stem cell factor, hIL-6, hIL-11, and hIL-3 to promote hematopoietic cell maturation and expansion.

(iii) Mesoderm Differentiation.

Undifferentiated cells were treated with Activin A and BMP4 according to a published protocol that fosters mesoderm differentiation (Laflamme et al., 2007). Briefly, cells were harvested by incubation with collagenase IV (Invitrogen) and plated onto a Matrigel-coated cell culture dish. To induce mesoderm differentiation, cells were cultured in RPM1-B27 medium (Invitrogen) supplemented with human recombinant Activin A (100 ng/ml) (R&D Systems) for 24 h. Human recombinant BMP4 (10 ng/ml) was added to the medium for four days, after which cells were fed further with supplement-free RBM1-B27 medium.

(iv) Ectoderm Differentiation.

Undifferentiated cells were harvested by incubation with collagenase IV (Invitrogen) and plated onto a Matrigel-coated cell culture dish. Cells were grown in KO-DMEM (Invitrogen) medium, containing knockout serum replacement (Invitrogen), supplemented with Noggin (500 ng/ml) (R&D Systems) and SB431542 (10 μM) (Tocris).

(v) Motor Neuron Differentiation.

Undifferentiated cells were differentiated following a published protocol (DiGiorgio et al., 2008), as described in more detail by Boulting et al. (submitted).

### DNA Methylation Mapping

Reduced representation bisulfite sequencing (RRBS). RRBS (Cowan, C. A. et al., N. Engl. J. Med. 350, 1353 (2004) was performed according to a previously published protocol (Smith, et al., Methods 48, 226 (2009)) with some optimizations for clinical samples and low amounts of input DNA (Gu, H. et al., Nat. Methods 7, 133 (2010)). The main steps were: (i) A total of 50 ng (ES cells) or 1 μg (colon samples) genomic DNA was digested by 5 U to 20 U of MspI (New England Biolabs, NEB) for up to 16 h. (ii) End-repair and adenylation of digested DNA were performed in a 20 μl reaction consisting of 10 U of Klenow fragments (3′→5′ exo-, NEB), 2 μl premixed nucleotide triphosphates (1 mM dGTP, 10 mM dATP, 1 mM 5′ methylated dCTP). The reaction was incubated at 30° C. for 30 min followed by 37° C. for additional 30 min. (iii) Preannealed 5-methylcytosine-containing Illumina adapters were ligated with adenylated DNA fragments in a 20 μl reaction containing of 1 μl concentrated T4 ligase (NEB), 1-2 μl of 15 μM adapters at 16° C. for 16 to 20 hours. (iv) Gel-based selection for fragments with insertion sizes of 40 to 120 basepairs and 120 to 220 basepairs was performed as described previously (Gu, H. et al., Nat. Methods 7, 133 (2010)). (v) Bisulfite treatment with the EpiTect Bisulfite Kit (Qiagen) was conducted following the protocol designated for DNA isolated from formalin-fixed and paraffin-embedded tissues. Two rounds of conversion were performed in order to maximize bisulfite conversion rates. The final bisulfite-converted DNA was eluted with 2×20 μl pre-heated (65° C.) EB buffer. (vi) To determine the minimum number of PCR cycles for final library enrichment, analytical (10 μl) PCR reactions containing 0.5 μl of bisulfite-treated DNA, 0.2 μM each of Illumina PCR primers LPX1.1 and 2.1 and 0.5 U PfuTurbo Cx Hotstart DNA polymerase (Stratagene) were set up. The thermocycler conditions were: 5 min at 95° C., varied cycle numbers (10-20) of 20s at 95° C., 30s at 65° C., 30s at 72° C., followed by 7 min at 72° C. PCR products were visualized by running on a 4-20% polyacrylamide Criterion TBE Gel (Bio-Rad) and stained by SYBR Green. The final libraries were generated by 8 of 25 μl PCR reaction with each one containing 2-3 μl of bisulfite-converted template, 1.25 U PfuTurbo Cx Hotstart polymerase and 0.2 μM each of Illumina LPX1.1 as well as 2.1 PCR primers. The libraries were PCR amplified and sequenced on the Illumina Genome Analyzer II as described previously (Gu, H. et al., Nat. Methods 7, 133 (2010)). The sequencing reads were aligned to the NCBI36 (hg18) assembly of the human genome using a custom alignment software that was developed for RRBS data (Meissner, A. et al., Nature 454, 766 (2008).

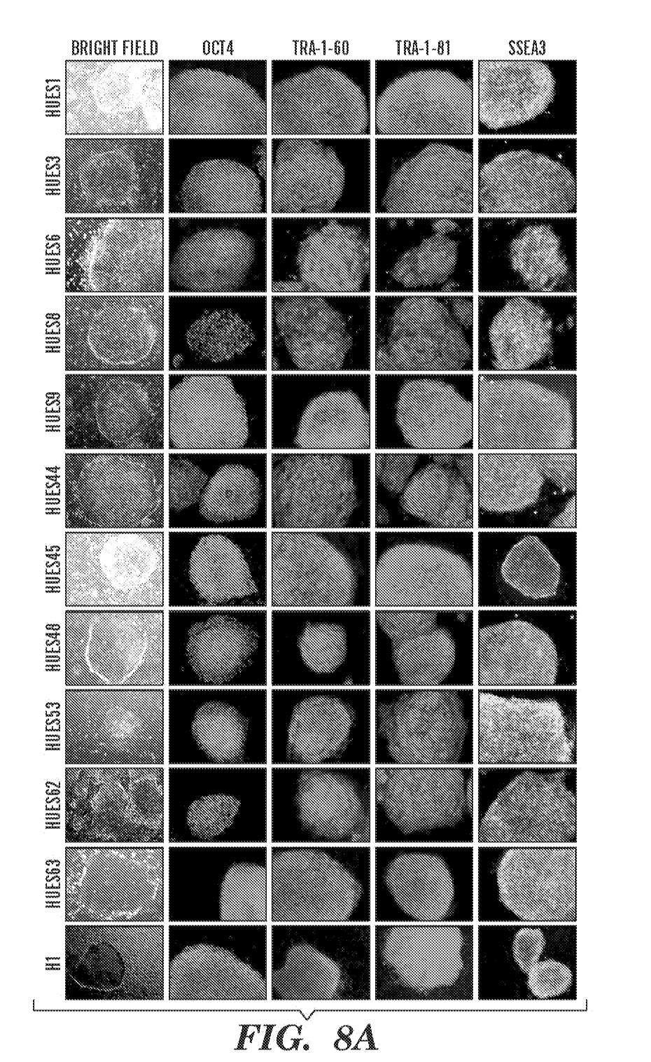

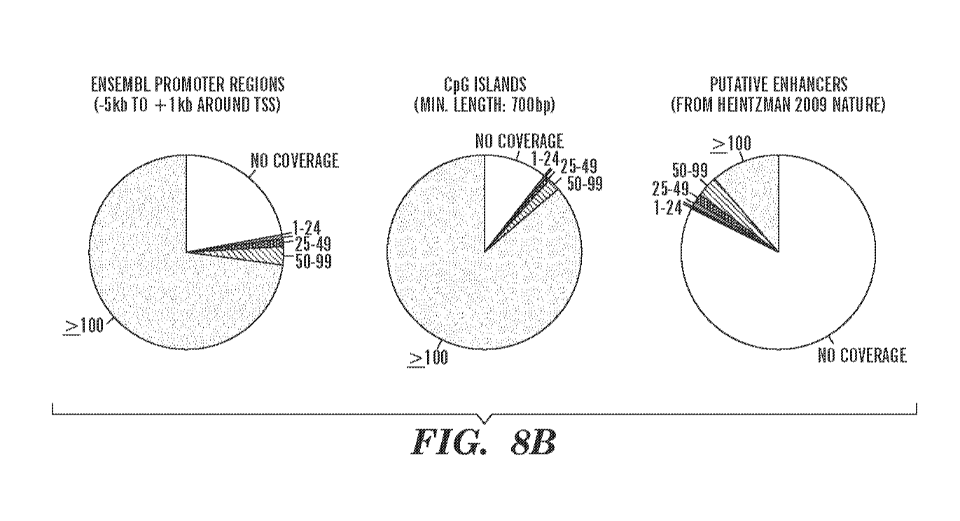

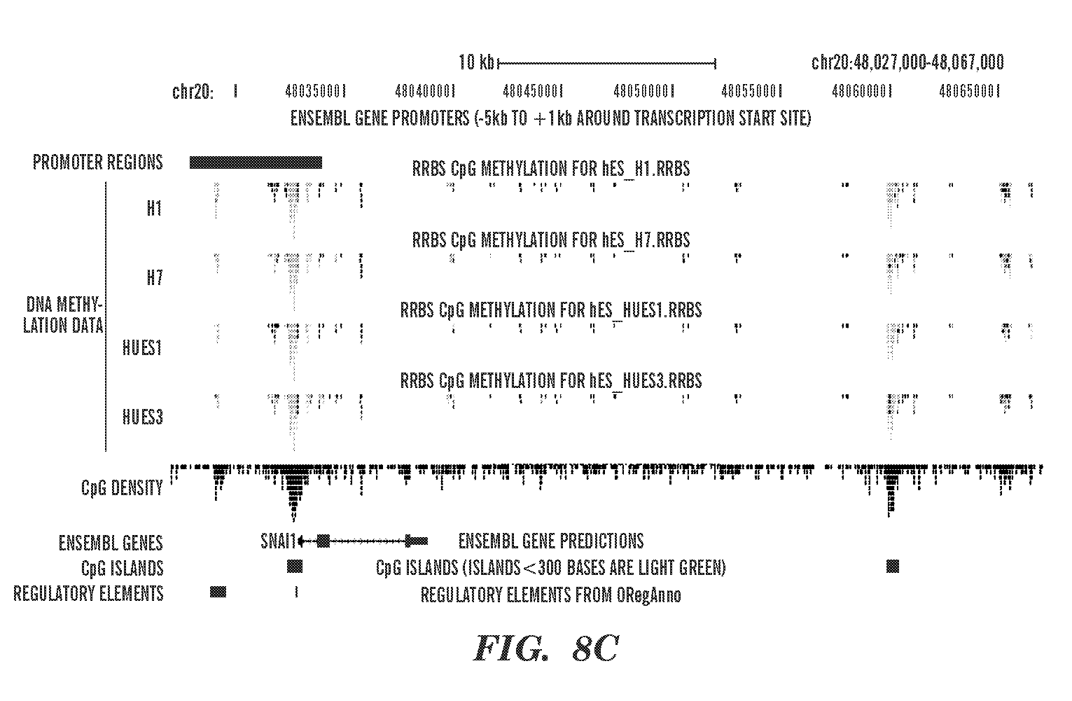

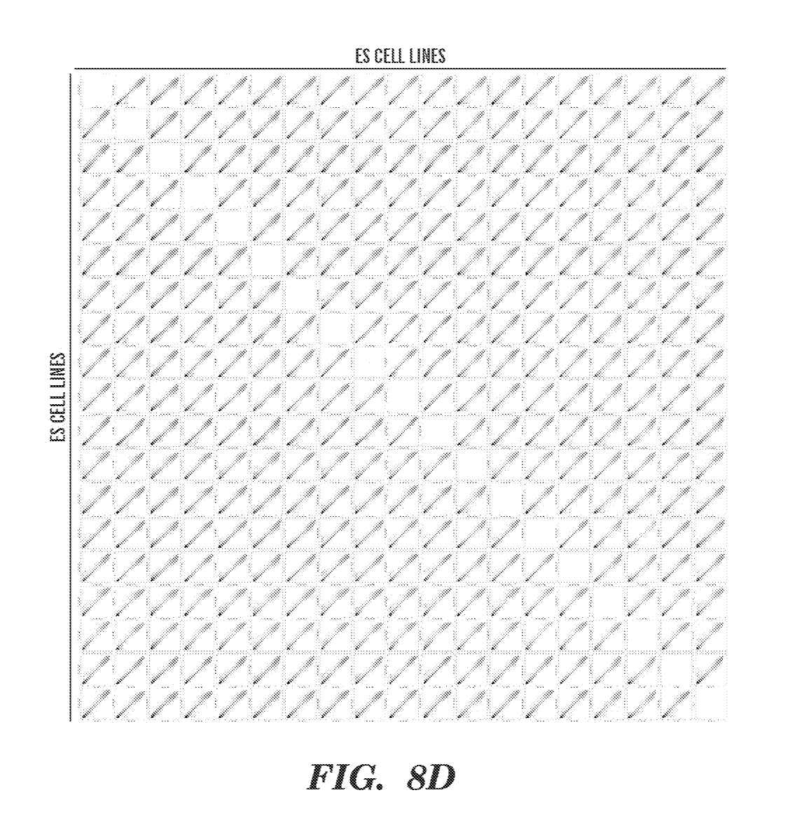

In some embodiments, RRBS was performed according to a previously published protocol (Smith et al., 2009) with some optimizations for small cell numbers (Gu et al., 2010). The raw sequencing reads were aligned using Maq's bisulfite alignment mode (Li et al., 2008) and DNA methylation calling was performed using custom software (Gu et al., 2010). To identify gene promoters in which a given cell line deviates from the reference of all human ES cell lines, the inventors performed weighted t-tests comparing the DNA methylation status of each CpG in a given gene promoter between the cell line of interest and the reference of all human ES cell lines included in the study (but excluding the cell line that is being tested), and then combined the corresponding p-values into a single region-specific p-value using a weighted version of Fisher's combined probability test. Gene promoters were defined as the −5 kb to +1 kb sequence window surrounding the annotated transcription start site of Ensembl-annoted genes (Hubbard et al., 2009). Weighting was performed according to the sequencing coverage at each CpG. Finally, the q-value method was used to account for multiple testing (Storey and Tibshirani, 2003) and called a genomic region differentially methylated if it was statistically significant with a false discovery rate (FDR) of less than 5% and the absolute DNA methylation difference exceeded the commonly used threshold of 20 percentage points (Bibikova et al., 2009), which is also justified in FIG. 8E. Note that differences in the sequencing depth and coverage between samples may influence the statistical power of this test but do not bias the test toward either hypomethylation or hypermethylation. All statistical analyses were performed using the R statistics package (world-wide web at:r-project.org/) and the source code is available on request from the authors.

Clonal Bisulfite Sequencing

Genomic DNA was isolated using PureLink genomic DNA mini kit (Invitrogen), DNA was bisulfite-converted using the EpiTect kit (Qiagen), and 50 ng of bisulfite converted DNA was PCR-amplified. Primer sequences were CD14 forward 5′-AGTTGTGGTTGAGGTTTAGGTT-3′ (SEQ ID NO: 5) and reverse 5′-ACCACAAAACTTACACTTTCCA-3′ (SEQ ID NO: 6). Amplicons were gel-purified and subcloned using TOPO TA cloning kit (Invitrogen). Clones were randomly selected for sequencing, and the sequencing data were processed using the BiQ Analyzer software (Bock et al., 2005).

Other DNA Methylation Mapping Methods:

Methyl-DNA Immunoprecipitation (MeDIP).

MeDIP (Down, et al., Nat. Biotechnol. 26, 779 (2008) was performed using the EZ DNA methylation kit (Zymo Research). A total of 300 ng DNA per sample was sonicated using Bioruptor (Diagenode) with 8 intervals of 10 min (30s on, 30s off), resulting in an average fragment size of 150 basepairs. Sonicated DNA was end-repaired and ligated with sequencing adapters as described previously (Down, et al., Nat. Biotechnol. 26, 779 (2008). Gel-based selection for fragment sizes between 100 and 200 basepairs was followed by methylated DNA immunoprecipitation according to the manufacturer's protocol. A total of 1 μg of monoclonal antibody against 5-methyl-cytosine (included in the EZ DNA methylation kit) was used for immunoprecipitation. The immunoprecipitated DNA was PCR-amplified and the specificity of the enrichment was confirmed by qPCR for selected loci as described previously (Rakyan, V. K et al., Genome Res. 18, 1518 (2008). Two lanes of 36-basepair single-ended sequencing were performed on the Illumina Genome Analyzer II according to the manufacturer's standard protocol. Maq with default parameters was used to align the sequencing reads to the NCBI36 (hg18) assembly of the human genome. (Li, H., Ruan, J., and Durbin, R., Genome Res. 18, 1851 (2008).

Methylated-DNA Capture (MethylCap):

MethylCap (Brinkman, A. B. et al., Methods (2010)) was performed in a robotized procedure using a SX-8G/IP-Star (Diagenode). 2 μg of His6-GST-MBD (Diagenode) was combined with 1 μg of sonicated DNA in 200 μl of binding buffer (BB, 20 mM Tris-HCl pH 8.5, 0.1% Triton X-100) containing 200 mM NaCl. This solution was incubated at 4° C. for 2 hours. Magnetic GST-beads were prepared by washing 35 μl of a well-mixed MagneGST glutathione particle suspension (Promega) with 200 μl of binding buffer plus 200 mM NaCl at 4° C. Washing was repeated once and the supernatant was removed. The GST-MBD-DNA solution was added to the washed and collected beads, and this suspension was rotated for another hour at 4° C. After removal of the supernatant (this is the flow-through) the beads-GST-MBD-DNA complexes were eluted by washing. 200 μl of binding buffer with different concentrations of NaCl was added and the suspension was rotated for 10 min at 4° C. Beads were captured using a magnet, and the supernatant was collected. The elution procedure consisted of 1×300 mM (wash), 2×400 mM (wash), 1×500 mM (“low” eluate), 1×600 mM (“medium” eluate), 1×800 mM NaCl (“high” eluate). The collected eluates were purified using QIAquick PCR purification spin columns (Qiagen), eluted with 100 μl elution buffer and prepared for sequencing as described previously (Brinkman, A. B. et al., Methods (2010)). A single lane of 36-basepair single-ended sequencing on performed on the Illumina Genome Analyzer II was performed for the low, medium and high eluates, respectively. The sequencing reads were aligned to the NCBI36 (hg18) assembly of the human genome using Illumina's analysis pipeline (ELAND) with default parameters. The lanes for each of the three eluates are shown separately in FIG. 2, and were tested to determine whether the accuracy relative to the Infinium assay could be improved by taking this additional information into account. However, a linear model that was based on the separate read counts of the three lanes did not outperform a model that was based on the sum of the three lanes.

Microarray-Based Epigenotyping (Infinium).

Infinium (Bibikova, M. et al., Epigenomics 1, 177 (2009) analysis was performed by the Genetic Analysis Platform at the Broad Institute. A total of 1 μg of genomic DNA per sample was bisulfite-treated according to the manufacturer's protocol and hybridized onto Infinium HumanMethylation bead arrays (Illumina). The inventors have previously observed almost perfect agreement between technical replicates (Pearson's r>0.98), which is why only a single hybridization was performed for each sample.

Data Preparation and Quality Control

For MeDIP and MethylCap, the aligned reads were extended to the mean fragment length obtained during sonication, and from each group of duplicate reads (i.e. reads aligned to the exact same start position on the same chromosome) all but one read were discarded, in order to minimize the impact of PCR bias on downstream analysis. For RRBS, the aligned reads were compared to the reference genome, and the DNA methylation status was determined using a custom software as described previously (Gu, H. et al., Nat. Methods 7, 133 (2010)). Infinium HumanMethylation27 data were processed with Illumina's BeadStudio 3.2 software, using the default background subtraction method for normalization. UCSC Genome Browser tracks were constructed by custom scripts implemented in the Python programming language (http://www.python.org/).

Quantification of Absolute DNA Methylation Level.

The inventors used linear regression models to estimate the absolute DNA methylation levels from the MeDIP and MethylCap read counts. Based on a number of different feature selection experiments, the inventors discovered that the following combination of variables was robustly predictive of DNA methylation levels: (i) the square root of the total number of MeDIP or MethylCap reads within the given region, (ii) the square root of the total number of whole-cell extract (WCE) reads within the region (based on a cross-tissue WCE track that the inventors have routinely used for ChIP-seq data normalization), (iii) the logit of the CpG frequency within the region, (iv) the relative GC content of the region, (v) the ratio of Cs relative to CpGs, and (vi) the relative repeat content of the region as determined by RepeatMasker (http://www.repeatmasker.org). For both MeDIP and MethylCap, the inventors discovered that the read frequencies were strongly positively associated with the absolute methylation level according to Infinium data, while the repeat content was moderately positively associated. In contrast, the logit of the CpG frequency was highly negatively associated with DNA methylation, and all other variables as well as the model's intercept exhibited a moderately negative association. For model fitting and performance evaluation, the current dataset was split into equally sized training and test sets. All model fitting was performed using the R statistics package (http://www.r-project.org/).

Identification of Differentially Methylated Region.

In the inventors experience, classical peak detection (Park, P. J., Nat. Rev. Genet. 10, 669 (2009) and Storey, et al, PNAS 100, 9440 (2003)) is not well-suited for DMR identification because of the high number of spurious hits encountered when borderline peaks are detected in one sample but not in the other (C. Bock, unpublished observation). Instead, the inventors used a statistical test to compare two samples directly with each other. For a given region with RRBS data, the inventors count the number of methylated vs. unmethylated CpGs in both samples and perform Fisher's exact test to obtain a p-value that is indicative of the likelihood of the region being a DMR. Similarly, for MeDIP and MethylCap the inventors counted the numbers of reads that align inside the region for both samples and use Fisher's exact test to contrast these values with the total numbers of reads that align elsewhere in the genome. And for the Infinium assay the inventors used a paired-samples t-test to compare the two samples' β-values of all Infinium probes inside the region. These tests are performed on a large number of genomic regions in parallel (e.g., on all CpG islands), and the p-values are corrected for multiple testing using the q-value method (Storey, et al, PNAS 100, 9440 (2003)). Genomic regions with a q-value of less than 0.1 are flagged as hypermethylated or hypomethylated (depending on the directionality of the difference), but only if the absolute DNA methylation difference exceeds 20% (for RRBS and Infinium) or if there is at least a twofold difference in the read number (for MeDIP and MethylCap). These thresholds were chosen by their practical utility in a number of comparisons between different cell types and have no further justification. The inventors also mark genomic regions with insufficient sequencing coverage, but do not exclude them from DMR analysis. For MeDIP and MethylCap the inventors recommend least ten reads per 10 million total reads for the sample with higher read coverage, and for RRBS the inventors recommended to use a minimum of five CpGs with at least five reads each in both samples.

This statistical approach to DMR identification requires us to define sets of genomic regions on which the analysis is being performed. The inventors pursued a two-way strategy to maximize the chances of finding interesting DMRs. One the one hand, the inventors focused specifically on CpG islands and gene promoters, which are prime candidates for epigenetic regulation. This approach provides increased statistical power for regions with well-known functional roles because the relatively low number of CpG islands and gene promoters reduces the burden of multiple-testing correction compared to the genome-wide case. On the other hand, the inventors used a 1-kilobase tiling of the genome to detect DMRs that are located outside of any candidate regions. And to cast an even wider net, the inventors collected a comprehensive set of 13 types of genomic regions, which includes not only CpG islands and gene promoters, but also CpG island shores³⁰, enhancers⁶⁰, evolutionary conserved regions and other types of genomic regions. DMR data for all of these region sets were calculated using a set of Python and R scripts and are available online (http://meth-benchmark.computational-epigenetics.org/).

Experimental Validation.

Based on the CpG islands that were detected as differentially methylated between two different ES cell lines, the inventors manually selected eight method-specific DMRs for experimental validation. To that end, those CpG islands that were identified as statistically significant DMRs by one method (but not by the other two methods) were visually inspected in the UCSC Genome Browser, and regions were selected for validation only if the data fully supported their classification as method-specific DMRs. In particular, regions were not selected if a second method already picked up a suggestive but insignificant trend in the same direction as the first method, or when the data of the first method already suggested that the DMR was a false-positive hit (e.g., because of contradictory trends in the vicinity of the DMR). Experimental validation was performed by clonal bisulfite sequencing following established protocols⁶¹. Primers were designed using MethPrimer⁶² such that the amplicon overlapped with those CpGs that exhibited the highest levels of differential methylation according to the inventors original data. To prepare for bisulfite sequencing, 1 μg of DNA was bisulfite-converted using the EpiTect kit (Qiagen); 50 ng of bisulfite-converted DNA was PCR-amplified; and purified amplicons were cloned using the TOPO TA cloning kit (Invitrogen). For each region an average of 11 clones were randomly chosen for sequencing. All sequencing data were processed using the BiQ Analyzer software (Bock, C. et al., Bioinformatics 21, 4067 (2005)).

Analysis of Repetitive DNA.

Repeat sequences were obtained from database version 14.07 of RepBase Update (Jurka, J., Trends Genet. 16, 418 (2000)), which is publicly available online (http://www.girinst.org/server/RepBase/index.php). From a total of 11,670 prototypic repeat sequences the inventors selected those 1,267 that were annotated either to human or to its ancestors in the taxonomic tree, and the inventors combined these prototypic repeat sequences into a pseudo-genome file. Maq with default parameters was used to align MeDIP, MethylCap, RRBS, ChIP-seq (H3K4me3) and whole-cell extract (WCE) sequencing reads against this pseudo-genome (Li, H., Ruan, J., and Durbin, R., Genome Res. 18, 1851 (2008)). For RRBS, both the reads and the reference genome were bisulfite-converted in silico prior to the alignment. The epigenetic status of each prototypic repeat sequence was quantified as follows: (i) For MeDIP, MethylCap and ChIP-seq the inventors calculated the odds ratios relative to the WCE data. (ii) For RRBS the inventors computed the number of methylated CpGs, total number of CpG measurements and percentage of DNA methylation based on the comparison of the aligned reads with the prototypic repeat sequence.

The inventors discarded rare repeats with WCE coverage below 100 aligned reads or RRBS coverage below 25 CpG measurements, resulting in 553 prototypic repeat sequences that were used for further analysis. Among these were 97 LINE class sequences (92 of them from the L1 family), 51 SINEs (48 of them from the Alu family), 6 SVAs, 62 DNA repeats, 15 satellite repeats, 315 LTRs, 1 low-complexity repeat and 6 RNA repeats. To quantify differential methylation between a pair of MeDIP and MethylCap samples, the inventors calculated the pairwise odds ratio of the read coverage for each prototypic repeat sequence, while the absolute DNA methylation difference was used in the case of RRBS. The significance of the difference was assessed using Fisher's exact test in the same way as for the non-repetitive genome (described above).

### Gene Expression Profiling

Microarray analysis was performed by the microarray core facility at the Broad Institute. Affymetrix GeneChip HT HG-U133A microarrays were used throughout. The microarray intensity data were normalized using Bioconductor's gcRMA package (Gentleman et al., 2004) and quality-controlled using array Quality Metrics (Kauffmann et al., 2009). To identify gene in which a given cell line deviates from the reference of all human ES cell lines sample, the inventors performed a moderated t-test as implemented in the limma package (Smyth, 2005), comparing the cell line of interest to the reference of all human ES cell lines included in this study (but excluding the cell line that is being tested). The inventors called a gene differentially expressed if the level of expression was statistically significant with an FDR of less than 10% and/or at least twofold or at >1 log-2 fold upregulated or downregulated expression level as compared to the reference gene expression for that gene. All statistical analyses were performed using the R statistics package (world-wide web at: r-project.org/) and the source code is available on request from the authors.

Quantitative RT-PCR Analysis

Total RNA was isolated using RNeasy kit (Qiagen) according to manufacturer's recommendation followed by cDNA synthesis using standard protocols. Briefly, cDNA was synthesized using Superscript II Reverse Transcriptase (Invitrogen) and Random Hexamers (Invitrogen) with 500 ng of total RNA input. SYBR Green PCR master mix (Applied Biosystems) was used for qPCR analysis, which was done on a StepOnePlus real time PCR system (Applied Biosystems). PCR conditions were as follow: 94° C. initial denaturation for 5 min, 94° C. 15s, 60° C. 15s, 72° C. 30s for 40 cycles, and 72° C. for 10 min. Primer sequences were: CD14 forward 5′-ACGCCAGAACCTTGTGAGC-3′ (SEQ ID NO: 7) and reverse 5′-GCATGGATCTCCACCTCTACTG-3′ (SEQ ID NO: 8); CD33 forward 5′-TCTTCTCCTGGTTGTCAGCT-3′ (SEQ ID NO: 9) and reverse 5′-GAGGCAGAGACAAAGAGCG-3′ (SEQ ID NO: 10) (Garnache-Ottou et al., 2005); CD64 forward 5′-GTGTCATGCGTGGAAGGATA-3′ (SEQ ID NO: 11) and reverse 5′-GCACTGGAGCTGGAAATAGC-3′ (SEQ ID NO: 12) (Li et al., 2010); and GAPDH forward 5′-ACCCACTCCTCCACCTTTGAC-3′ (SEQ ID NO: 13) and reverse 5′-ACCCTGTTGCTGTAGCCAAATT-3′ (SEQ ID NO: 14). Relative quantification was calculated using the comparative threshold cycle (delta delta Ct) method.

Quantitative Embryoid Body Assay and Lineage Scorecard

For embryoid body differentiation, ES/iPS cells were treated with dispase or trypsin and plated in suspension in low-adherence plates in the presence of human ES culture media without bFGF and plasmanate. Cell aggregates or embryoid bodies were allowed to grow for a total of 16 days, refreshing media every 48 h. On day 16, cells were lysed and total RNA was extracted using Trizol (Invitrogen), followed by column clean-up using RNeasy kit (Qiagen). Subsequently, 300 to 500 ng of RNA was used for analysis on the NanoString nCounter system according to manufacturer's instructions. The nCounter codeset contained 500 genes that were computationally selected for their ability to monitor cell state, pluripotency and differentiation. Because the nCounter system has been introduced only recently, no best practices exist for normalizing the expression values. The inventors tested several different procedures and found that a combination of spike-in normalization using positive controls and the VSN algorithm (Huber et al., 2002) produced best results. Data analysis was performed in much the same way as for the microarray data. Specifically, the inventors used a moderated t-test to compare the gene expression in the embryoid bodies for the cell line of interest to the reference of all ES-cell derived embryoid bodies included in this study (but excluding the cell line that is being tested). To prepare for gene set testing, the inventors calculated the mean and standard deviation of the t-scores over all genes. Next, the inventors calculated the mean t-score separately for all gene sets that were defined a priori, and the inventors performed a parametric test against the mean over all genes as described previously (Kim 2005). For the lineage scorecard diagram, the inventors plotted the signed difference between the gene test mean and the global mean of the t-scores independent of significance, averaged over all contributing gene sets.

### Immunocytochemistry and FACS Analysis

Immunostaining was performed using the following primary antibodies: AFP (Dako), NESTIN (Chemicon), OCT4 (Santa Cruz Biotechnology), alpha-SMA (Sigma), SSEA3 (Biolegend), SSEA4 (Chemicon), TRA-1-60 (Chemicon), TRA-1-81 (Chemicon), beta III Tubulin (Abcam), VEGFRII (Abcam). For FACS analysis, EBs were trypsin-dissociated to single cells, washed with PBS, fixed overnight with 4% paraformaldehyde and permeabilized with 0.5% PBS-Tween for 20 mins-1 hour. Cells (−500 k) were then blocked in 0.1% PBS-Tween supplemented with 10% donkey serum for 1 hr, and incubated with primary antibody (AFP: 1:300, DakoCtomation) overnight and secondary for 1 hr, washed and re-suspended in 1 ml PBS with 0.1% donkey serum. Samples were analyzed using BD Biosystems LSRII analyzer. For FACS analysis, EBs were trypsin-dissociated to single cells, washed with PBS, fixed overnight with 4% paraformaldehyde and permeabilized with 0.5% PBS-Tween for 20 mins-1 hour. Cells (−500 k) were then blocked in 0.1% PBS-Tween supplemented with 10% donkey serum for 1 hr, and incubated with primary antibody (AFP: 1:300, DakoCtomation) overnight and secondary for 1 hr, washed and re-suspended in 1 ml PBS with 0.1% donkey serum. Samples were analyzed using BD Biosystems LSRII analyzer.

Deviation Scorecard Calculation

The deviation scorecard summarizes which and how many genes in a cell line of interest deviate from the ES cell reference. The reference is being constituted by the 20 low-passage ES cell lines—or by the 19 remaining ES cell lines when calculating the deviation scorecard for a cell line that is normally part of the reference. The algorithm for calculating the deviation scorecard (outlined in FIG. 11A) is the same for DNA methylation and gene expression data, with the only exception that the microarray data require an additional normalization step. From a statistical point of view, the deviation scorecard is based on non-parametric outlier detection using Tukey's outlier filter (Tukey, 1977). All genes for which the DNA methylation or gene expression value of the cell line of interest fall outside of the center quartiles by more than 1.5 times the interquartile range are considered suspected outliers and flagged as such. Next, the magnitude of the change is considered and only genes for which the deviation from the ES cell reference is sufficiently large to be considered biologically meaningful are ultimately reported as outliers. A threshold of at least 20 percentage points for DNA methylation and at least twofold for gene expression was used herein, which is consistent with prior work (Bock et al., 2010) and further justified in FIG. 10C. To account for the fact that deviations may be more or less concerning depending on which genes are affected, two lists of genes were assembled which are recommended to be monitored particularly closely for DNA methylation defects, namely lineage marker genes and cancer genes (e.g., tumor suppressor genes and oncogenes). Deviations at these genes are specifically highlighted in the extended version of the deviation scorecard (Table 6). Finally, the inventors have also evaluated alternative strategies for flagging outliers, including a parametric approach that was based on moderated t-tests. Overall, the Tukey's outlier filter was determined to gave the most relevant results, and it has the additional advantage that it can be intuitively visualized by “reference corridor” boxplots (FIGS. 1C and 4A).

Lineage Scorecard Calculation

The lineage scorecard quantifies the differentiation propensity of a cell line of interest relative to a reference constituted by 19 low-passage ES cell lines. The algorithm for calculating the lineage scorecard (outlined in FIG. 11B) uses a combination of moderated t-tests (Smyth, 2004) and gene set enrichment analysis performed on t-scores (Nam and Kim, 2008; Subramanian et al., 2005). To provide a biological basis for quantifying lineage-specific differentiation propensities, several sets of marker genes for each of the three germ layers (ectoderm, mesoderm, endoderm) as well as for the neural and hematopoietic lineages were collected (Table 7, Table 13A and Table 14). Next, Bioconductor's limma package was used to perform moderated t-tests comparing the gene expression in the EBs obtained for the cell line of interest to the EBs obtained for the ES cell reference, and the mean t-scores were calculated across all genes that contribute to a relevant gene set. High mean t-scores indicate increased expression of the gene set's genes in the tested EBs and are considered indicative of a high differentiation propensity for the corresponding lineage. In contrast, low mean t-scores indicate decreased expression of relevant genes and are considered indicative of a low differentiation propensity for the corresponding lineage. To increase the robustness of the analysis, the mean t-scores were averaged over all gene sets assigned to a given lineage. The lineage scorecard diagrams (FIGS. 5B and D) list these “means of gene-set mean t-scores” as quantitative indicators of cell-line specific differentiation propensities. The lineage scorecard analyses and validations were performed using custom R scripts (available from world-wide web: r-project.org/). Finally, motor neuron differentiation efficiencies that were experimentally derived by Boulting et al. provide a genuine test set of cell lines for determining the predictive power of the lineage scorecard. Addidionally, as the bioinformatic algorithms of the lineage scorecard had already been finalized before the first comparisons between the two datasets, and no aspects of the scorecard were retrospectively optimized to improve the fit.

Bioinformatic Analysis and Data Access

In Addition to Method-Specific Data Normalization and the Calculation of the Scorecard (described above), bioinformatic analyses were conducted as follows:

(i) Hierarchical Clustering (FIGS. 1, 3, 8 and 9).

DNA methylation levels were calculated as the coverage-weighted average over all CpGs in the promoter regions of Ensembl-annotated transcripts; gene expression levels were calculated for each Ensembl gene by averaging over all associated probes on the microarray. Prior to hierarchical clustering the two datasets were separately normalized to zero mean and unit variance in order to give equal weight to both datasets. The heatmaps show a representative selection of 250 genes. Hierarchical clustering was performed in R (available from world-wide web: r-project.org/), using a Euclidean distance function and the average-linkage method.

(ii) Annotation Clustering and Promoter Characteristics (FIG. 2D).

Identification of common characteristics among the most variable genes was performed using DAVID (Huang et al., 2007) and EpiGRAPH (Bock et al., 2009) with default parameters and based on Ensembl gene annotations (promoters were defined as the −5 kb to +1 kb sequence window surrounding the transcription start site).

(iii) Classification of ES vs. iPS Cell Lines (FIG. 3D).

To validate the previously reported iPS gene signatures, the mean DNA methylation or expression level over all genes in a given signature was calculated from the current dataset. Logistic regression was used for selecting the most discriminatory threshold, and the predictiveness of each signature was evaluated by leave-one-out cross-validation. To derive new classifiers, support vector machines were trained on the DNA methylation data, the gene expression data, or the combination of both datasets.

Each classification was based on 7500 randomly selected attributes, which was the maximum number of attributes that were computationally feasible in a single analysis. The predictiveness of all classifiers was evaluated by leave-one-out cross-validation, and the average performance over 100 classifications with random attribute sets are reported in FIG. 3D. Note that none of these classifications used feature selection. It is likely that supervised or unsupervised feature selection could increase the prediction accuracy, but in the absence of a second validation dataset it is unclear whether such an improvement reflects a genuine increase in predictiveness or overfitting to the current dataset. All predictions were performed using the Weka software (Frank et al., 2004)

(iv) Linear Models of Epigenetic Memory.

Two alternative linear models were constructed for both DNA methylation and gene expression. The first model regresses the iPS-cell specific mean DNA methylation (or gene expression) levels of each gene on the ES-cell specific mean DNA methylation (or gene expression) levels. The second model regresses the iPS-cell specific mean DNA methylation (or gene expression) levels of each gene on the ES-cell specific and the fibroblast-specific mean DNA methylation (or gene expression) levels. Both models were compared by an analysis of variance (ANOVA). All calculations were performed in R (available from world-wide web: r-project.org/).

### Example 1

### Variation in DNA Methylation and Transcription Between hES Cell Lines

There are many properties of a given ES cell line that could influence its DNA methylation, transcription or differentiation propensities. These could include the genetic background of a cell line, the way in which a line is cultured, selective pressure applied by extended in vitro growth, or unexplained stochastic noise. Before one can attempt to study the potential underlying causes of the variance in pluripotent stem cell line behavior, it is crucial to first determine both the nature and extent of variation that exists within a substantial cohort of lines.

To study inter-line variation between pluripontent stem cell populations or lines, the inventors obtained 19 human ES cell lines at low passage numbers (p15 to 25), cultured them for several passages under standardized conditions, then collected both DNA for analysis of DNA methylation and RNA for transcriptional profiling (Table 1, FIG. 8A). In order to make comparisons to another cell type, both the RNA and DNA was analyzed from 6 low-passage human dermal fibroblast lines obtained from the upper arm of genetically unrelated donors.

Table 1:

Summary of cell lines used in the high-throughput experiments. *verified by presence/absence of chrY and evidence of X-chromosome inactivation in the RRBS, microarray and/or NanoString data.

TABLE 1

| | | | | Sibling | | | |
|---|---|---|---|---|---|---|---|
| | | | | Pairs | | | |
| | | | | (ES)/ | Passage | Passage No. | Passage No. for |
| | | Donor | Donor | Donor | No. for | for | Lineage |
| Cell Line | Reference | Age | Sex* | (iPS) | RRBS | Microarray | Scorecard |
| HUES1 | Cowan et al. 2004 | NA | female | | 22 | 26 | 26, 26 |
| HUES3 | Cowan et al. 2004 | NA | male | | 27 | 27 | 27, 28 |
| HUES6 | Cowan et al. 2004 | NA | female | | 23 | 23 | 19, 21 |
| HUES8 | Cowan et al. 2004 | NA | male | | 27 | 27 | 25, 26 |
| HUES9 | Cowan et al. 2004 | NA | female | | 21 | 21 | 19, 18 |
| HUES13 | Cowan et al. 2004 | NA | male | | 47 | 47 | NA |
| HUES28 | Chen et al. 2009 | NA | female | | 17 | 17 | 13, 15 |
| HUES44 | Chen et al. 2009 | NA | female | | 18 | 18 | 15, 16 |
| HUES45 | Chen et al. 2009 | NA | female | | 20 | 20 | 17, 19 |
| HUES48 | Chen et al. 2009 | NA | female | | 19 | 19 | 16, 17 |
| HUES49 | Chen et al. 2009 | NA | female | | 17 | 17 | 14, 14 |
| HUES53 | Chen et al. 2009 | NA | male | A | 17 | 18 | 17, 18 |
| HUES62 | Chen et al. 2009 | NA | female | B | 14 | 17 | 15, 16, 16, 16, 18 |
| HUES63 | Chen et al. 2009 | NA | male | B | 19 | 14 | 19, 17 |
| HUES64 | Chen et al. 2009 | NA | male | B | 19 | 19 | 18, 20 |
| HUES65 | Chen et al. 2009 | NA | male | | 19 | 19 | 16, 17 |
| HUES66 | Chen et al. 2009 | NA | female | A | 20 | 20 | 15, 15 |
| H1 | Thomson et al. 1998 | NA | male | | 34 | 34 | 33, 34 |
| H7 | Thomson et al. 1998 | NA | female | | 48 | 48 | NA |
| H9 | Thomson et al. 1998 | NA | female | | NA | 58 | 57, 58 |
| hiPS 11a | Boulting et al. | 36 | male | 11 | 22 | 22 | 14, 18, 27, 29 |
| hiPS 11b | Boulting et al. | 36 | male | 11 | 13 | 13 | 15, 18, 25, 31 |
| hiPS 15b | Boulting et al. | 48 | female | 15 | 27 | 16 | 29, 30, 41, 44 |
| hiPS 17a | Boulting et al. | 71 | female | 17 | 14 | 12 | 10, 16, 17, 19 |
| hiPS 17b | Boulting et al. | 71 | female | 17 | 32 | 32 | 18, 20, 38 |
| hiPS 18a | Boulting et al. | 48 | female | 18 | 30 | 30 | 31, 32, 46 |
| hiPS 18b | Boulting et al. | 48 | female | 18 | 27 | 27 | 20, 37 |
| hiPS 18c | Boulting et al. | 48 | female | 18 | 36 | 27 | 30, 32 |
| hiPS 20b | Boulting et al. | 55 | male | 20 | 43 | 43 | 26, 31, 46, 50 |
| hiPS 27b | Boulting et al. | 29 | female | 27 | 31 | 31 | 27, 28 |
| hiPS 27e | Boulting et al. | 29 | female | 27 | 32 | 30 | 30, 31, 32, 32, 35 |
| hiPS 29d | Boulting et al. | 82 | female | 29 | NA | NA | 14, 15 |
| hiPS 29e | Boulting et al. | 82 | female | 29 | NA | NA | 25, 27 |
| hFib_11 | Boulting et al. | 36 | male | 11 | 8 | 8 | 7, 8 |
| hFib_15 | Boulting et al. | 48 | female | 15 | 7 | 7 | 6, 7 |
| hFib_17 | Boulting et al. | 71 | female | 17 | 7 | 7 | 6, 7 |
| hFib_18 | Boulting et al. | 48 | female | 18 | 7 | 7 | 6,7 |
| hFib_20 | Boulting et al. | 55 | male | 20 | 7 | 7 | 6, 7 |
| hFib_27 | Boulting et al. | 29 | female | 27 | 7 | 7 | 6, 7 |

*verified by presence/absence of chrY and evidence of X-chromosome inactivation in the RRBS, microarray and/or NanoString data

The inventors chose to study DNA methylation in ES cells rather than other chromatin modifications for several reasons. Methylation of CpG dinucleotides in promoter regions is associated with long-term, mitotically heritable gene silencing (Bird, 2002; Reik, 2007). Differential DNA methylation between cell lines might therefore result in variable gene expression during differentiation, potentially influencing developmental potency. Another rationale for studying DNA methylation is that it can be measured by a highly quantitative assay: bisulfite modification of DNA followed by DNA sequencing (Laird, 2010). Following a systematic comparison of established methods for determining genome-wide levels of DNA methylation (Bock et al. submitted), the inventors selected reduced-representation bisulfite sequencing (RRBS) for use in this study (Gu et al., 2010; Meissner et al., 2008).

Using RRBS, the inventors quantified the methylation status of more than four million individual CpG dinucleotides for each cell line. This genome-scale coverage allowed us to determine methylation levels at three quarters of all gene promoters, the majority of CpG islands and many other genomic elements (FIGS. 8B and 8C; and data not shown). The inventors determined that the average of 15-20 DNA methylation measurements in each cell line at the around 4 million CpGs enabled the detection of small quantitative differences in DNA methylation between cell lines.

As is common practice for studies of this scale (Adewumi et al., 2007; ENCODE Project Consortium, 2007; Meissner et al., 2008; Miller et al., 2008; Narva et al., 2010), the inventors analyzed only a single replicate of most cell lines. However, for a subset of cell lines (n=4) the inventors performed additional replicates to assess the consistency of the measurements. The inventors demonstrated excellent technical reproducibility (Pearson's r>0.99) for both RRBS and microarray profiling. Biological reproducibility was also high (Pearson's r>0.95), and biological replicates collected from the same cell line two to seven passages apart were also more similar to each other than to other ES cell lines. Although the inventors demonstrated a strong correlation (Pearson's r>0.95) when they compared high (passage >45) and low-passage (passage <30) cells from the same lines, these samples were no longer more similar to each other than they were to those taken from distinct ES cell lines (data not shown). Because prolonged culture induced additional variation in DNA methylation and transcription, the inventors focused the subsequent analysis only on the 19 low-passage samples (see Table 1).

To determine whether combined global patterns of transcription and DNA methylation would be sufficient to segregate ES cell lines into subclasses that might have different functional properties, the inventors performed joint hierarchical clustering on the datasets (FIG. 1A). As a control, the inventors included similar data sets from 6 non-pluripotent fibroblast cell lines in the analysis. As would be expected, two well-separated clusters of cell lines emerged. One cluster included all of the ES cell lines and the other included all the fibroblast control cell lines. Importantly, within the cluster of human ES cell lines, there was little or no evidence of further sub-clustering. This lack of sub-clustering suggests that there were no outlier ES cell lines with global methylation and transcriptional signatures that could skew subsequent analyses. Additionally, the absence of distinct ES cell sub-classes reassuringly suggested that all 19 ES cell lines had a similar overall pattern of transcription and DNA methylation.

While global patterns of methylation and transcription were well conserved in each ES cell line a number of loci exhibited variance between the lines (FIG. 1A). Based on their gene expression and DNA methylation patterns, the inventors determined that most loci can be classified into one of four different categories. FIG. 1B shows representative examples of each class. Many essential genes, such as SOX2, exhibited no variation between lines in either DNA methylation or transcription. In contrast, some genes, such as CD14, had variable methylation between lines, while other genes, such as GATA6, showed distinct levels of transcription, but no variance in DNA methylation. Finally an additional small class of genes, which included S100A6, displayed variation in both transcription and methylation (FIG. 1B).

To determine if the variation in DNA methylation or transcription between lines is in part responsible for differences in cell line behavior, the inventors then identified each of the genes with variable properties, and then determined the magnitude of that variance to be able to predict the differentiation propensities of any given line. The inventors therefore calculated the average levels of methylation and transcription for each locus in the 19 ES cell lines, as well as the amount of variance in these measurements (Tables 3-5). These results encompass as “reference corridor” or “reference DNA methylation levels” or “reference Gene expression levels” to provide a range of values of the expected levels and range of DNA methylation or transcription levels respectively in ES cells for any gene, e.g., target DNA methylation genes, and target Gene expression genes. This is illustrated in FIG. 1C, displaying the concept of a “reference corridor” using boxplots to display the average levels and range of DNA methylation or transcription for several selected genes (FIG. 1C). These plots impose upper and lower thresholds on the DNA methylation and expression levels for each locus that are considered “within the range of the ES cell reference”. The inventors also assigned a significance-of-deviation score to all measurements from the 19 lines that fell outside the “corridor” (FIGS. 8D and 8E illustrate the DNA methylation data and the thresholds used for identifying significant differences between cell lines). With this reference in hand, one of ordinary skill in the art is able to determine the number and identity of deviations from the corridor in any pluripotent cell line by performing stringent statistical tests. Additionally, using this “reference map” for variation between cell lines, the inventors could investigate both the nature and potential sources of this variation and can determine how the gene expression and/or DNA methylation affects stem cell behavior.

### Example 2

### Causes and Consequences of Epigenetic and Transcriptional Variation Among Human ES Cell Lines

To begin to understand the causes and consequences of variation in transcription and methylation between the ES cell lines, the inventors used a “reference map” to quantify the level of variance in these measures for each locus (Tables 4 and 5). This quantification allowed the inventors to determine the proportion of genes that varied and the identity of genes with either minimal or substantial variance. The resulting distributions were highly skewed, with only 16% of all genes accounting for 50% of DNA methylation variation, and only 28% of all genes accounting for 50% of gene expression variation (FIG. 2A). Thus, most variation between cell lines is restricted to only a subset of loci and suggests that the identities of genes in these two classes might provide insight into why they vary and whether their variance would have any bearing on the properties of given lines.

The inventors next proceeded to note the identity of both highly variant and invariant loci within the cohort of cell lines (FIG. 2A, Tables 4 and 5). As expected housekeeping genes such as GAPDH were among the least variable genes between stem cell lines. Similarly, the inventors demonstrated observed only low to moderate variation among genes such as SOX2 and DNMT3B, whose functions are associated with the pluripotent state (FIG. 2A). In contrast, the inventors surprisingly discovered that moderate to high levels of epigenetic or transcriptional variation for several genes that regulate embryonic development, including GATA6, LEFTY2 and PAX6. Finally, there were a small number of loci that displayed highly variant levels of DNA methylation between lines. For these genomic elements, the levels in DNA methylation varied between nearly 0% methylation in some cell lines to almost 100% methylation in other cell lines. These rare, but highly variant, genes included the transferrin-encoding gene TF, the catalase-encoding gene CAT and the macrophage/granulocyte specific marker gene CD14.

The inventors next assessed whether the identity of variant genes could provide insight into why their properties varied between cell lines. The inventors initially focused on genes with the highest levels of epigenetic and transcriptional variation, respectively. Surprisingly, the inventors demonstrated that a substantial percentage of the most variable genes were located on the sex chromosomes (FIG. 2B). This discovery is likely the result of the inclusion of both male and female cell lines. Y-linked methylation and transcription would be expected to vary between cell lines as that chromosome is absent in female lines. Substantial variance in X-chromosome inactivation has also been reported for distinct female ES cell lines, providing a potential explanation for the high degree of methylation and transcriptional variance in X-linked genes (FIG. 2B) (Hanna et al., 2010; Lengner et al., 2010). As sex-chromosome linked genes were such a significant source of variation, the inventors were concerned that they might limit the ability to identify gene features that might more subtly influence their transcriptional or epigenetic variability. Therefore in subsequent analyses the inventors excluded loci linked to the X and Y chromosomes.

When the inventors focused exclusively on autosomal loci, the inventors demonstrated that there was a clear and significant overlap between the sets of genes that showed the greatest epigenetic and transcriptional variability, respectively (p<10⁻¹¹, Fisher's exact test, FIG. 2C). This correlation demonstrates that DNA methylation may be a regulatory mechanism for a subset of the most transcriptionally variable genes. Analysis of gene function and promoter characteristics highlighted relevant differences between the varying and non-varying genes (FIG. 2D). The inventors demonstrated that loci with variable transcription were highly enriched for Gene Ontology categories related to cellular signaling and the response to external stimuli.

In contrast, genes with variable methylation levels showed little evidence of enrichment for any particular function. Instead, the inventors demonstrated that the promoters of these genes shared common structural characteristics. Most notably, these promoters were relatively depleted in CpG dinucleotides, a known characteristic of genomic regions that are susceptible to variation in DNA methylation (Bock et al., 2006; Keshet et al., 2006; Meissner et al., 2008).

To study the functional consequences of variation among human ES cell lines, the inventors next investigated in more detail genes that exhibited highly variable DNA methylation levels among ES cell lines, but which were invariably silent in ES cells (FIG. 1B). The inventors assessed if epigenetic defects at these genes may have a delayed effect on transcription, impairing differentiation along trajectories for which the affected genes are relevant. To demonstrate this, the inventors performed unbiased embryoid body (EB) differentiation of two ES cell lines with strong DNA methylation differences (HUES6 and HUES8), and then measured DNA methylation as well as gene expression in 16-day EBs (FIG. 2D). The data demonstrated that the majority of DNA methylation differences between the two cell lines were retained in 16-day EBs (p<10⁻¹⁶, Fisher's exact test) and that these DNA methylation differences were often associated with differential gene expression between the two cell lines (p<10⁻⁵, Fisher's exact test). CD14 is an example of a gene that is silent in both ES cell lines but hypermethylated only in HUES8. During EB differentiation CD14 is upregulated only in HUES6; its hypermethylated gene promoter in HUES8 correlates with its failure to activate in that ES cell line upon differentiation. Given CD14's role as a canonical surface marker of macrophages and neutrophil granulocytes, the inventors determined that those who wish to generate large numbers of these cells by directed differentiation should avoid this particular line of HUES8. More generally it highlights the relevance of monitoring DNA methylation as a marker for predicting limitations or possible biases in differentiation that are not detectable at the transcriptional level in undifferentiated ES cells.

### Example 3

### Global Patterns of DNA Methylation and Transcription are Similar Between hES Cells and hiPS Cells

The inventors “reference maps” of human ES cell line variation have enabled the inventors to determine the number and identity of genes that deviate from the norm in any new cell line through statistical comparisons with the ES-cell “reference corridor”. With the use of defined factor reprogramming to produce human iPS cell lines for various applications (Park et al., 2008b; Takahashi et al., 2007; Yu et al., 2007), there is an increasing need to determine how to select the most appropriate iPS cell lines for a given purpose. Mapping the variance in DNA methylation and transcription across iPS cell lines could allow one of ordinary skill in the art to determine whether there are loci that are systematically different between reprogrammed cells and their ES cell counterparts. This would furthermore help guide selection of high quality iPS cell lines similar to what is described herein for ES cells.

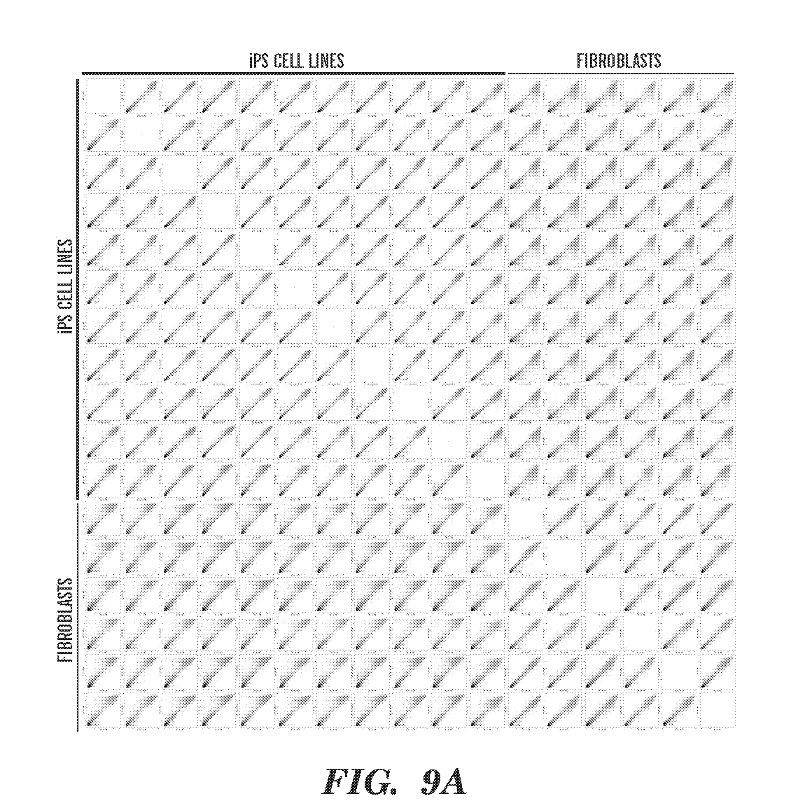

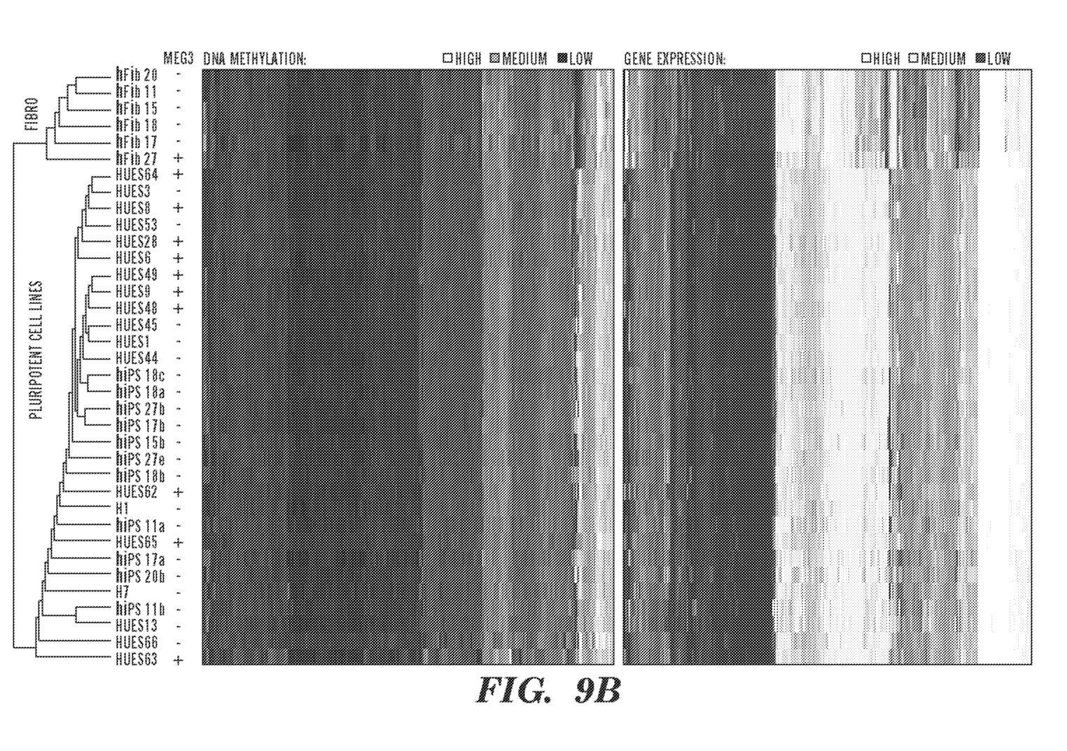

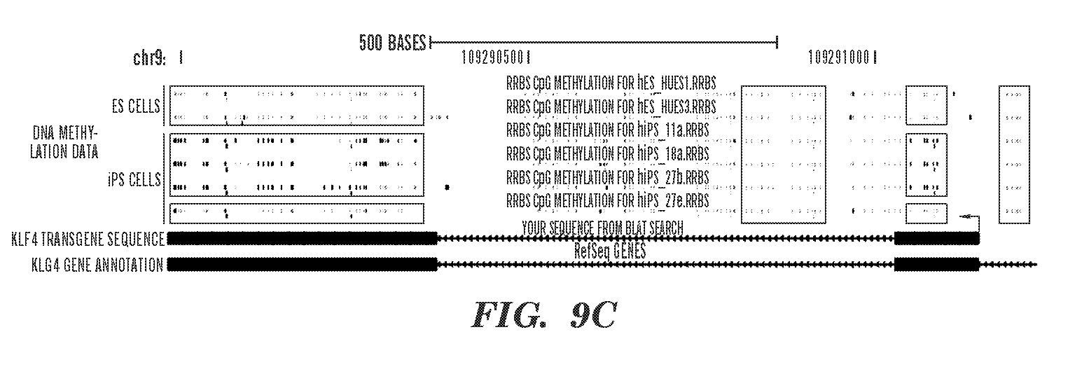

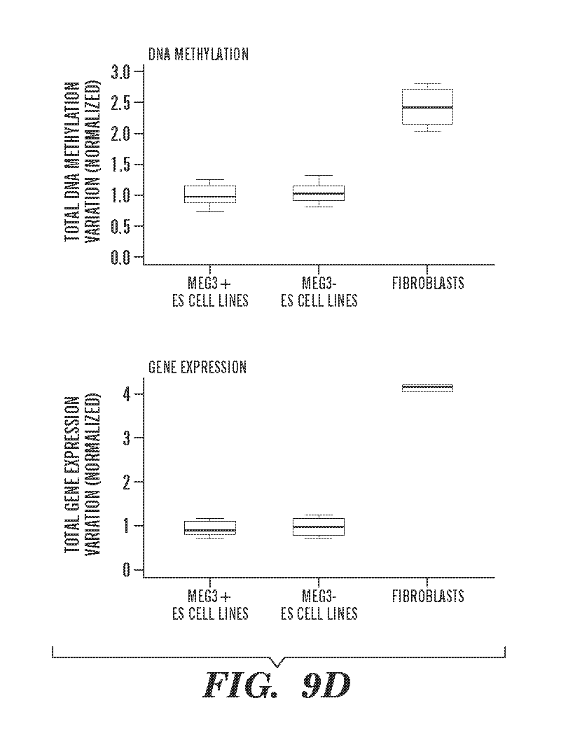

The inventors therefore mapped DNA methylation and gene expression in 11 iPS cell lines (see Table 1) derived from six distinct donors by retroviral transduction of OCT4, SOX2 and KLF4. These iPS cell lines have been characterized extensively (Boulting et al., co-submitted) and were maintained under culture conditions similar to the 19 reference ES cell lines and harvested for DNA and RNA at comparable passage numbers. DNA methylation and transcriptional profiling of these iPS cell lines were performed as for the ES cell lines and again yielded highly reproducible data (FIG. 9A).

The inventors initially asked whether the iPS cell lines had global patterns of transcription and DNA methylation that were distinct from ES cells. The inventors performed joint hierarchical clustering using the full data sets from the 19 ES cell lines and 11 iPS cell lines. As a control, the inventors also included datasets from the 6 fibroblast lines used for clustering analysis (FIG. 1A). As in the previous analysis, two well-separated clusters emerged. One cluster contained the fibroblast cell lines and the other contained all the ES and iPS cell lines (FIG. 3A and FIG. 9B). Importantly, the inventors did not identify subclustering among the pluripotent cell lines, demonstrating that if there were any systematic differences between ES and iPS cells, they were not strong enough to register in this form of analysis.

To produce a more quantitative comparison between these two pluripotent cell types, the inventors began with data from all 30 cell lines and calculated the average degree of deviation from the ES-cell “reference corridor” for each gene in the dataset (Tables 4 and 5). The observed concordance between the variation of the 19 ES cell lines from the reference and the variation of the 11 iPS cell lines from the reference was high, with a Pearson's correlation coefficient of r=0.89 for both DNA methylation and gene expression, indicating that most genes displaying deviation in iPS cells were also hypervariable among the ES cell lines (FIG. 3B). For example, genes such as TF, CAT and CD14, which displayed the most variable levels of DNA methylation between ES cell lines, also showed the greatest variation between iPS cell lines. Similarly as expected, GAPDH did not vary between ES or iPS cell lines (FIG. 3B). Although the correlation between the nature of the variant genes in ES and iPS cells was high, the quantitative degree of epigenetic and transcriptional deviation from the ES-cell reference for these genes was slightly higher for iPS cell lines (FIG. 3C). In conclusion, the lists of genes with invariant and variant levels of methylation and transcription overlap almost entirely in the sampling of ES and iPS cells herein.

### Example 4

### Differential Methylation or Transcription of Individual Genes Cannot Accurately Distinguish ES and iPS Cells

Despite the overall similarity, the inventors demonstrated that a small number of genes that exhibited substantially increased deviation from the “reference” levels of methylation and transcription in iPS cell lines. Some genes were hypermethylated in subsets of iPS lines, such as the protease HTRA4 (9 out of 11 iPS cell lines), the neuron-specific RNA-binding protein NOVA1 (2 out of 11 iPS cell lines) and the relaxin hormones RLN1/2 (RLN1: 8 out of 11 iPS cell lines, RLN2: 5 out of 11 iPS cell lines). Others were transcribed at higher levels in iPS cell lines, such as the lysophospholipase CLC (3 out of 11 iPS cell lines) and the crystallin CRYBB1 (3 out of 11 iPS cell lines) (FIG. 3B).

The promoter region of HTRA4 is hypermethylated in 9 out of 11 iPS cell lines and 6 out of 6 fibroblast cell lines but is unmethylated in all ES cell lines (n=19). Such a deviation in DNA methylation patterns between ES cells and iPS cells could be construed as evidence for incomplete reprogramming and epigenetic “memory” of the differentiated state. Such “memory” would be predicted to result in the mirroring of DNA methylation levels between iPS cells and somatic cells at certain loci. To directly and quantitatively test whether there was significant memory of the somatic epigenetic state in iPS cells, the inventors constructed a statistical model that tests for the predictiveness of gene-specific somatic cell memory while controlling for the confounding effect of variability among ES cell lines. Specifically, the inventors derived linear models predicting the direction and magnitude of iPS cell deviation from the ES cell reference based on either mean and variation of the ES cell reference or mean and variation of the ES cell reference as well as the direction and magnitude in which fibroblasts deviate from the ES cell reference. When the inventors statistically compared these two models, the inventors demonstrated that the latter model, which took into account “epigenetic memory” explained the levels of epigenetic deviation in iPS cell lines only marginally better than the former (0.5% additional variance explained). While there may be other confounding factors that the inventors did not control for that could have modestly reduced the variance explained by epigenetic memory, the inventors data clearly demonstrate that epigenetic memory is not a significant determinant of variation in DNA methylation levels between human ES cells and iPS cells.

Another gene of note, MEG3, is reportedly expressed differentially in mouse ES and iPS cells that fail to generate mice by tetraploid embryo complementation (Liu et al., 2010; Stadtfeld et al., 2010b). MEG3 is an imprinted gene found in the imprinted DLK1/DIO3 domain on human chromosome 12 and displays developmentally regulated expression patterns across various tissues. The expression of MEG3 was highly variable in 10 of the 19 human ES cell lines and silent in the remaining 9. In contrast to its variable expression among ES cell lines, MEG3 transcription was not detected in any of the iPS cell lines and was modestly expressed in only one of the 6 fibroblast cell lines from which the iPS cell lines were derived (FIG. 9B).

The inventors discovery that silencing of MEG3 should not be considered an iPS-specific phenomenon. The inventors demonstrated that MEG3 is also silent in many dermal fibroblast cell lines, implying that some form of improper silencing during reprogramming is not required to arrive at the low levels of MEG3 observed in human iPS cell lines. Additionally, many human ES cell lines did not express MEG3, demonstrating that its expression is not required for human pluripotency. However, it is likely that the subtle effects caused by differential MEG3 expression could be difficult to detect in the context of human pluripotent cell lines given that the effects could only be observed in the mouse by tetraploid embryo complementation (Stadtfeld et al., 2010b). From a more practical perspective, it is reassuring that both cell lines that do and do not express MEG3 have been widely and productively used. As a final possibility, the inventors assessed whether variation in MEG3 expression might serve as a useful marker and indicator of the overall level of epigenetic and/or transcriptional variation in an ES cell or iPS cell line. However, the inventors did not find this to be the case (FIG. 9D).

### Example 5

### Statistical Modeling of Variation in DNA Methylation and Transcription has Limited Power to Discern Between iPS Cells and ES Cells

The inventors approaches for investigating differences between iPS cells and ES cells had utilized either hierarchical clustering, and a very global approach, or systematic benchmarking of individual, hand-picked candidates such as HTRA4 and MEG3. Neither of these approaches can accurately describe the overall distinction between ES and iPS cell lines. Another approach is to use transcriptional signatures relying on multiple genes to distinguish between ES and iPS cell lines (Chin et al., 2009). Moreover, levels of DNA methylation at multiple genomic regions taken together are predictive of whether a cell is an ES cell or an iPS cell (Doi et al., 2009). Accordingly, the inventors assessed both the transcriptional and DNA methylation signatures in the dataset, re-optimizing the threshold that classifies cell lines as either ES or iPS but not the gene sets themselves. For the gene expression signature the inventors demonstrated an accuracy of 67%, which was better than expected by chance alone. However, the previously reported DNA methylation signature (Doi et al., 2009) failed to correctly identify any of the iPS cell lines in the inventors study (FIG. 3D).

The inventors next investigated the methylation or transcription signatures from the dataset (Table 2). Using a previously reported gene expression signature (Chin et al., 2009), the inventors determined a robust 3.4-fold enrichment of classifying (ES vs. iPS) genes showing the same directionality of effect in both studies, although only five genes passed stringent statistical testing. The difference between the average gene expression profiles of ES and iPS cell lines is therefore conserved between the present study and the previous one (Chin et al., 2009), but this difference is too weak to accurately identify a cell line as either ES or iPS.

For the DNA methylation signature, a third of the iPS-specific differentially methylated regions (Doi et al., 2009) with sufficient data were also differentially methylated in the dataset, but seven out of 12 regions exhibited an opposite tendency to that previously reported. Importantly, 98% of the differences between fibroblasts and iPS cells from the same study could be confirmed with the same directionality in the study, indicating that the lack of agreement for the iPS-specific differentially methylated regions is not a side effect of the different methods used for DNA methylation mapping (Doi et al., 2009). The inventors therefore determined that the previous study by Doi et al. likely picked up highly variable genomic regions that were differentially methylated by chance, rather than true iPS-specific DNA methylation defects.

Table 2. Validation of previously reported iPS-specific DNA methylation and gene expression. DNA methylation data. Validation of previously published genes/genomic regions distinguishing ES cells from iPS cells. Tables 11A-11C are DNA methylation data (based on Doi et al. 2009 Nature Genetics, http://www.ncbi.nlm.nih.gov/pubmed/19881528). Tables 11D-11F are Gene expression data (based on Chin et al. 2009 Cell Stem Cell, at world-wide web site: “ncbi.nlm.nih.gov/pubmed/19570518”).

TABLE 2A
DNA methylation data

| Significant changes (FDR < 0.1) | | Doi et al. | |
|---|---|---|---|
| | | Up in ES cells | Up in iPS cells |
| Current | Up in ES cells | 0 | 0 |
| dataset | Up in iPS cells | 7 | 5 |
| | p-value | 1.00 | |
| | odds ratio | 0.00 | |
| Marginal changes (p-val < 1) | | Doi et al. | |
| | | Up in ES cells | Up in iPS cells |
| Current | Up in ES cells | 6 | 5 |
| dataset | Up in iPS cells | 13 | 11 |
| | p-value | 1.00 | |
| | odds ratio | 1.02 | |
| Fibrablasts (FDR < 0.1) | | Doi et A | |
| | | Up in | Up in iPS cells |
| | | fibroblasts | |
| Current | Up in fibroblasts | 572 | 1 |
| dataset | Up in iPS cells | 20 | 300 |
| | p-value | <2.2e−16 | |
| | odds ratio | 7792.74 | |

TABLE 2B
Gene expression data
Table 2B: Gene Expression data

| | | Chin et al. | |
|---|---|---|---|
| | | Up in ES cells | Up in iPS cells |
| Significant changes | | | |
| (FDR <0.1) | | | |
| Current | Up in ES cells | 3 | 1 |
| dataset | Up in iPS cells | 1 | 2 |
| | p-value | 0.486 | |
| | odds ratio | 4.45 | |
| Marginal changes | | | |
| (p-val <1) | | | |
| Current | Up in ES cells | 122 | 92 |
| dataset | Up in iPS cells | 45 | 114 |
| | p-value | 3.61E−08 | |
| | odds ratio | 3.35 | |

Finally, the inventor assessed whether one could use the dataset of 19 ES cell lines and 11 iPS cell lines to develop a novel and more accurate method for distinguishing ES and iPS cell lines based on their DNA methylation and/or gene expression profiles. To minimize the risk of over-fitting the training data, or over-estimating the prediction accuracy of the classifier, the inventors employed a stringent statistical learning approach (Hastie et al., 2001). The inventors abstained from any manual parameter optimization or supervised feature selection (these are notorious for bloating prediction accuracies if used incorrectly). Specifically, the inventors trained logistic regression models as well as support vector machines on (i) the DNA methylation data, (ii) the gene expression data and (iii) the combination of both, and then assessed the performance of the trained classifiers on test cases that were not included in the training data set. Although the support vector machine achieved an accuracy of 90% (which is substantially higher than the randomly expected 50% or 63.3%), none of the classifiers could perfectly discriminate between ES and iPS cell lines (FIG. 3D).

### Example 6

### A Scorecard for Quality Assessment of Human Pluripotent Cell Lines

The inventors results thus far indicate that variance in DNA methylation and transcription exists between human ES and iPS cell lines (FIG. 1), that this variation is limited to a subset of genes and that knowledge concerning the variance of loci in a given cell line are in part predictive of its behavior (FIG. 2). However, there do not seem to be gene signatures that can robustly distinguish between human ES cells and iPS cells (FIG. 3). One conclusion from these data is that iPS cell lines collectively mirror ES cell lines at the population level, and that iPS cells are therefore characteristic of human pluripotent stem cells to a similar degree overall. Nevertheless, at the level of the individual investigator working with a limited number of ES and/or iPS cell lines, it is important to determine to what degree the undoubted genetic variation within either of these groups will affect experimental outcomes.

To develop a simple and efficient approach to select cell lines for a given application, the inventors used statistical tests to distil the epigenetic and transcriptional deviations in specific cell lines into a “scorecard” that would predict its behavior (FIGS. 4A, 4B and Table 6). To do this, the inventors focused on the characteristics of a cell line that distinguish it from the norm. These selection criteria can also be used as criteria for exclusion of certain lines.

An exemplary example would be that the “scorecard” would help those interested in macrophage differentiation avoid cell lines in which the CD14 promoter is hypermethylated (FIG. 2E). However, there may be many characteristics of a cell line that cannot be predicted from variation of transcription and methylation from the “reference” data set. These might include the individual genetic makeup of each cell line, epigenetic variation that cannot be accounted for by monitoring DNA methylation, or other factors that the inventors might not yet appreciate. To overcome these limitations, the inventors sought to add measurements to the “scorecard” that might provide a means for selecting cell lines based on their likelihood to perform well in a given differentiation paradigm.

TABLE 6
Summary of deviations from the ES-cell reference map for each ES/iPS cell line.
Table 6A is the DNA methylation derivation data for each ES/iPS cell line. Table 6B is the Gene Expression derivation
data for each ES/iPS cell line. The explanations for each column abbreviation is at the end of the Table 6B.

| Cell line | TABLE 6A: DNA methylation | | | | | | |
|---|---|---|---|---|---|---|---|
| sample name | variation | #incr | #decr | #lineage | #cancer | lineage markers | cancer genes |
| hES_HUES1 | 108.0% | 289 | 19 | 6 | 13 | CHRDL1+, | ARHGEF6+, FGF13+, |
| | | | | | | CHRDL1+, | FOXO4+, FOXO4+, FOXO4+, |
| | | | | | | CHRDL1+, EDA+, | LCK+, LCK+, LCK+, PAK3+, |
| | | | | | | EDA+, ZIC3+ | PAK3+, PIM2+, RUNX1T1+, |
| | | | | | | | STK3+ |
| hES_HUES3 | 92.2% | 50 | 27 | 3 | 1 | CD14+, CD14+, | BCL2L10+ |
| | | | | | | CDX4− | |
| hES_HUES6 | 124.3% | 66 | 65 | 1 | 2 | SP7− | ERN2+, RARB− |
| hES_HUES8 | 73.0% | 23 | 19 | 2 | 0 | CD14+, CD14+ | <none> |
| hES_HUES9 | 73.6% | 62 | 21 | 1 | 1 | ERAS+ | ERAS+ |
| hES_HUES13 | 117.1% | 212 | 168 | 9 | 12 | AMN+, CAMK2A−, | BCL2L10+, CAMK2A−, |
| | | | | | | CAMK2A−, CD14+, | CAMK2A−, CFLAR+, |
| | | | | | | CD14+, CDX4+, | CFLAR+, GNA14+, MX1+, |
| | | | | | | POU5F1+, | NCR1−, POU5F1+, PRKCZ−, |
| | | | | | | WNT16+, ZFP42+ | WNT16+, ZNF266+ |
| hES_HUES28 | 96.0% | 47 | 146 | 2 | 1 | CD14−, GCNT2− | ALOX15B+ |
| hES_HUES44 | 90.7% | 318 | 2 | 10 | 7 | AMN+, CD14+, | ERAS+, FAM123B+, FGF13+, |
| | | | | | | CD14+, ERAS+, | MAOA+, PAK3+, PAK3+, |
| | | | | | | RENBP+, RENBP+, | STK3+ |
| | | | | | | RENBP+, SYP+, | |
| | | | | | | SYP+, ZIC3+ | |
| hES_HUES45 | 80.3% | 49 | 20 | 3 | 1 | CD14+, CD14+, | ERAS+ |
| | | | | | | ERAS+ | |
| hES_HUES48 | 88.4% | 48 | 3 | 2 | 0 | CD14−, DDX3X+ | <none> |
| hES_HUES49 | 98.5% | 248 | 4 | 13 | 10 | CITED1+, | AR+, ARAF+, ARAF+, |
| | | | | | | CITED1+, EDA+ | FAM123B+, FAM123B+, |
| | | | | | | EDA+, HTATSF1+, | PIM2+, SEPT6+, SEPT6+, |
| | | | | | | MTM1+, MTM1+, | SEPT6+, SFN+ |
| | | | | | | RENBP+, RENBP+ | |
| | | | | | | RENBP+, SYN1+, | |
| | | | | | | SYP+, SYP+ | |
| hES_HUES53 | 104.4% | 41 | 176 | 6 | 2 | ANGPTL2+, | ERAS−, FGF17+ |
| | | | | | | ANGPTL2+, | |
| | | | | | | CDX4−, DPPA3−, | |
| | | | | | | ERAS−, SP7− | |
| hES_HUES62 | 114.1% | 327 | 44 | 12 | 20 | ABCB7+, ABCB7+, | ALOX15B+, CAMK2A−, |
| | | | | | | CAMK2A−, CD14− | CD40+, CD40+, CD74+, |
| | | | | | | CD40+, CD40+, | CD74+, CFLAR+, CFLAR+, |
| | | | | | | DES+, ERAS+, | ELK1+, ELK1+, ERAS+, |
| | | | | | | LAMP2+, RBPJ+, | ERN2+, SEPT9−, SRC+, SRC+, |
| | | | | | | SYN1+, ZFP42+ | TCL1A+, TNFRSF25+, |
| | | | | | | | XIAP+, XIAP+, XIAP+ |
| hES_HUES63 | 98.3% | 59 | 21 | 0 | 0 | <none> | <none> |
| hES_HUES64 | 87.3% | 126 | 13 | 3 | 3 | DES+, ERAS+, | ALOX15B+, ERAS+, SRC+ |
| | | | | | | RBPJ+ | |
| hES_HUES65 | 114.3% | 18 | 293 | 6 | 7 | ANGPTL2+, | ALOX15B−, ERAS−, FGF17+, |
| | | | | | | ANGPTL2+, | PSEN1−, TCL1A−, WHSC1−, |
| | | | | | | CDX4−, CSF1R−, | ZFP37− |
| | | | | | | DES−, ERAS− | |
| hES_HUES66 | 112.3% | 32 | 278 | 2 | 5 | CD14−, DPPA3− | BCL2L10−, ELN−, ELN−, ELN−, |
| | | | | | | | ZFP37− |
| hES_H1 | 95.2% | 138 | 69 | 13 | 10 | CD14+, CD14+, | ALOX15B−, BCL2L10+, |
| | | | | | | CD14+, CDX4−, | CEACAM5+, CEACAM5+, |
| | | | | | | CEACAM5+, | ERAS−, ERN2−, LGALS1+, |
| | | | | | | CEACAM5+, DES−, | SEPT9+, TCL1A−, ZFP37− |
| | | | | | | ERAS−, GRM1+, | |
| | | | | | | GRM1+, ITGB2−, | |
| | | | | | | ITGB2−, ITGB2− | |
| hES_H7 | 132.1% | 428 | 144 | 10 | 29 | ALX1+, AMN+, | ALOX15B−, BCL2L10+, |
| | | | | | | CDX4+, ERAS+ | CACNA1B+, CACNA1B+, |
| | | | | | | GCNT2+, GRM1+, | CACNA1B+, CACNA1B+, |
| | | | | | | GRM1+, LAMP2+, | CACNA1B+, CASC5−, |
| | | | | | | PCSK9+, ZFP42+ | CASC5−, CFLAR+, CFLAR+, |
| | | | | | | | DCTN1+, DCTN1+, ERAS+, |
| | | | | | | | ERN2+, GOPC+, LGALS1−, |
| | | | | | | | NOS3−, PCSK9+, PIK3R5−, |
| | | | | | | | RAC2−, RAC2−, RAC2−, |
| | | | | | | | RAC2−, SEPT9−, SFN−, |
| | | | | | | | SRD5A2+, SRD5A2+, |
| | | | | | | | ZNF443+ |
| hiPS_11a | 119.9% | 128 | 40 | 10 | 2 | AMN+, | KLF4+, POU5F1+ |
| | | | | | | ANGPTL2+, | |
| | | | | | | ANGPTL2+, | |
| | | | | | | CD14+, CD14+, | |
| | | | | | | CD14+, CD8A+, | |
| | | | | | | CD8A+, KLF4+, | |
| | | | | | | POU5F1+ | |
| hiPS_11b | 104.2% | 56 | 106 | 10 | 10 | CAMK2A−, | CAMK2A−, CAMK2A−, CD74−, |
| | | | | | | CAMK2A−, CD72+, | CD74−, ERAS−, GLI2−, |
| | | | | | | CD72+, CD93−, | POU5F1+, TCL1A−, ZFP37+, |
| | | | | | | ELAVL4−, ERAS−, | ZNF471+ |
| | | | | | | IGHD−, POU5F1+, | |
| | | | | | | SOX2+ | |
| hiPS_15b | 92.5% | 75 | 52 | 10 | 6 | ANGPTL2+, | ERAS−, KLF4+, POU2AF1−, |
| | | | | | | ANGPTL2+, ERAS−, | POU5F1+ TCL1A−, ZNF471+ |
| | | | | | | KLF4+, POU5F1+, | |
| | | | | | | RENBP+, RENBP+, | |
| | | | | | | RENBP+, SOX2+, | |
| | | | | | | SP7− | |
| hiPS_17a | 144.4% | 472 | 7 | 17 | 19 | ARX+, CD14+, | BCL2L10+, CFLAR+, |
| | | | | | | CD14+, CD72+, | CFLAR+, ELF4+, ELF4+, |
| | | | | | | CD72+, CDX4+, | ERAS+, ERN2+, GNA14+, |
| | | | | | | DES+, ERAS+, | PDE4DIP+, PDE4DIP+, |
| | | | | | | POU5F1+, | PDE4DIP+, PDE4DIP+, |
| | | | | | | RENBP+, RENBP+ | PDE4DIP+, POU5F1+, |
| | | | | | | RENBP+, SIPA1+ | RPL31+, SRC+, STK3+, |
| | | | | | | SOX2+, WNT16+, | WNT16+, ZNF471+ |
| | | | | | | ZFP42+, ZIC3+ | |
| hiPS_17b | 120.7% | 511 | 3 | 19 | 20 | CD14+, CD14+, | ALOX15B+, BCL2L10+, |
| | | | | | | CD14+, CD8A+, | CFLAR+, CFLAR+, DCTN1+, |
| | | | | | | CD8A+, CD8A+, | ERAS+, ERN2+, GNA14+, |
| | | | | | | CDX4+, DES+, | GOPC+, MAOA+, PCSK9+, |
| | | | | | | ERAS+, GCNT2+, | PLAGL1−, POU5F1+, |
| | | | | | | HFE2+, LAMP2+, | RUNX1T1+, SFN+, SRC+, |
| | | | | | | PCSK9+, PLAGL1−, | TNFRSF25+, TNFRSF25+, |
| | | | | | | POU5F1+, RBPJ+, | WNT16+, ZNF471+ |
| | | | | | | SIPA1+, WNT16+, | |
| | | | | | | ZFP42+ | |
| hiPS_18a | 95.5% | 168 | 44 | 5 | 8 | CDX4+, DES+, | CFLAR+, CFLAR+, FGF13+, |
| | | | | | | KLF4+, POU5F1+, | KLF4+, POU5F1+, RPS4X+, |
| | | | | | | SOX2+ | RPS4X+, STK3+ |
| hiPS_18b | 107.7% | 287 | 49 | 16 | 12 | ABCB7+, ABCB7+, | CD40+, CD40+, CFLAR+, |
| | | | | | | CD40+, CD40+, | CFLAR+, ERAS+, LSM5+, |
| | | | | | | CDX4+, CHRDL1+, | MAOA+, PAK3+, PAK3+, |
| | | | | | | CHRDL1+, | POU5F1+, RPS4X+, RPS4X+ |
| | | | | | | CHRDL1+, ERAS+ | |
| | | | | | | FOXG1+, FOXG1+, | |
| | | | | | | LAMP2+, | |
| | | | | | | POU5F1+, SIPA1+, | |
| | | | | | | SOX2+, ZFP42+ | |
| hiPS_18c | 93.4% | 377 | 23 | 19 | 18 | CDX4+, CITED1+, | ARHGEF6+, ARHGEF6+, |
| | | | | | | CITED1+, | ELF4+, ELF4+, ELK1+, |
| | | | | | | DDX3X+, EDA+, | ELK1+, FGF13+, GPC3+, |
| | | | | | | EDA+, GPC3+, | GPC3+, GPC3+, KLF4+, |
| | | | | | | GPC3+, GPC3+, | POU5F1+, RPS4X+, RPS4X+, |
| | | | | | | HTATSF1+, | STK3+, XIAP+, XIAP+, |
| | | | | | | KLF4+, MTM1+, | XIAP+ |
| | | | | | | MTM1+, OSR1+, | |
| | | | | | | POU5F1+, SIPA1+, | |
| | | | | | | SOX2+, SYN1+, | |
| | | | | | | ZIC3+ | |
| hiPS_20b | 119.7% | 432 | 26 | 11 | 17 | CDX4+, CFDP1+ | ALOX15B+, DCTN1+, |
| | | | | | | DES+, ERAS+, | ERAS+, ERN2+, GNA14+, |
| | | | | | | GCNT2+, ID2+, | GNAS+, GNAS+, GNAS+, |
| | | | | | | PCSK9+, | GOPC+, PCSK9+, POU5F1+, |
| | | | | | | POU5F1+, RBPJ+, | SEPT9−, SRC+, TFCP2+, |
| | | | | | | WNT16+, ZFP42+ | TNFRSF25+, WNT16+, |
| | | | | | | | ZNF471+ |
| hiPS_27b | 107.5% | 291 | 32 | 10 | 16 | CDX4+, DES+, | CFLAR+, CFLAR+, ERAS+, |
| | | | | | | ERAS+, HFE2+, | ERN2+, FGF13+, GOPC+, |
| | | | | | | ID2+, PAX4+, | PDE4DIP+, PDE4DIP+, |
| | | | | | | PAX4+, POU5F1+ | PDE4DIP+, PDE4DIP+, |
| | | | | | | TNFRSF8+, | PDE4DIP+, POU5F1+, |
| | | | | | | ZFP42+ | RPS4X+, RPS4X+, STK3+, |
| | | | | | | | TNFRSF8+ |
| hiPS_27e | 169.1% | 59 | 504 | 16 | 12 | ANGPTL2+, | ALOX15B−, CD74−, CD74−, |
| | | | | | | ANGPTL2+, CD14−, | ERAS−, ERN2−, FZD10+, |
| | | | | | | CDX4−, CSF3−, | LGALS1−, PLAGL1−, |
| | | | | | | CSF3−, CSF3−, | POU2AF1−, POU5F1+, |
| | | | | | | ELAVL4−, ERAS−, | TCL1A−, TPM4− |
| | | | | | | GCNT2−, ITGB2−, | |
| | | | | | | ITGB2−, ITGB2−, | |
| | | | | | | PLAGL1−, | |
| | | | | | | POU5F1+, TNNI3− | |
| hES_min | 73.0% | 18 | 2 | 0 | 0 | N/A | N/A |
| hES_quartile1 | 89.6% | 48 | 19 | 2 | 1 | N/A | N/A |
| hES_mean | 100.0% | 136 | 81 | 5 | 7 | N/A | N/A |
| hES_quartile3 | 113.2% | 230 | 145 | 10 | 10 | N/A | N/A |
| hES_max | 132.1% | 428 | 293 | 13 | 29 | N/A | N/A |
| hiPS_min | 92.5% | 56 | 3 | 5 | 2 | N/A | N/A |
| hiPS_quartile1 | 99.8% | 102 | 25 | 10 | 9 | N/A | N/A |
| hiPS_mean | 115.9% | 260 | 81 | 13 | 13 | N/A | N/A |
| hiPS_quartile3 | 120.3% | 405 | 51 | 17 | 18 | N/A | N/A |
| hiPS_max | 169.1% | 511 | 504 | 19 | 20 | N/A | N/A |
| Cell line | TABLE 6B Gene expression: | | | | | | |
| sample name | variation | #incr | #decr | #lineage | #cancer | lineage markers | cancer genes |
| hES_HUES1 | 74.6% | 7 | 1 | 1 | 0 | LHX2+ | <none> |
| hES_HUES3 | 81.6% | 5 | 2 | 1 | 0 | CD151− | <none> |
| hES_HUES6 | 88.5% | 18 | 2 | 1 | 0 | HLA−DRA+ | <none> |
| hES_HUES8 | 82.7% | 6 | 1 | 0 | 1 | <none> | MSN+ |
| hES_HUES9 | 72.0% | 5 | 0 | 0 | 0 | <none> | <none> |
| hES_HUES13 | 215.3% | 847 | 500 | 100 | 131 | ABCB1+, | ABCB1+, AGR2+, AKT3+, |
| | | | | | | ACTA2+, AGR2+, | ALB+, ALPL−, ARNT2+, |
| | | | | | | ALB+, | ASXL1+, BCL11A−, BCL2+, |
| | | | | | | ALDH1A1+, | BCL7A+, BIK−, BMI1+, |
| | | | | | | ALPL−, ARID3B−, | BNIP3L+, BOP1−, BRAF−, |
| | | | | | | ASCL1+, BGN+, | CANT1+, CAPN2+, CARD8+, |
| | | | | | | BMI1+, BMPR2+, | CASP9−, CCL2+, CCND2+, |
| | | | | | | BSG−, CAPN1−, | CCNE1−, CDCP1−, CDH1−, |
| | | | | | | CD55−, CD9−, | CDH11+, CDKN2D+, CHEK2−, |
| | | | | | | CDCP1−, CDH1−, | COL1A1+, COL4A1+, |
| | | | | | | CDH3−, | COL4A2+, COL4A6+, |
| | | | | | | CEACAM6+, | COPZ2+, CRTC3 |
| | | | | | | CLDN6−, | |
| | | | | | | COL1A1+, | |
| | | | | | | COL1A2+, | |
| | | | | | | COL2A1+, | |
| | | | | | | COL3A1+, | |
| | | | | | | COL4A2+, | |
| | | | | | | CSPG5+, CST3+, | |
| | | | | | | CTNND2+, DCN+, | |
| | | | | | | DCX+, DPPA4−, | |
| | | | | | | DZIP1+, ELAVL4+ | |
| hES_HUES28 | 112.8% | 34 | 17 | 1 | 3 | UTF1+ | CHN1−, HRK+, MLH1− |
| hES_HUES44 | 92.0% | 5 | 2 | 0 | 2 | <none> | CREB5+, DPF1+ |
| hES_HUES45 | 72.0% | 1 | 0 | 0 | 1 | <none> | LMO1+ |
| hES_HUES48 | 104.6% | 15 | 4 | 0 | 0 | <none> | <none> |
| hES_HUES49 | 75.6% | 5 | 0 | 0 | 0 | <none> | <none> |
| hES_HUES53 | 80.6% | 20 | 0 | 2 | 0 | CGB+, FABP1+ | <none> |
| hES_HUES62 | 117.8% | 40 | 7 | 2 | 6 | CITED1+, | ARC+, FGF3+, HOXA2+, |
| | | | | | | PPARGC1A+ | NAIP+, VLDLR−, WNT4+ |
| hES_HUES63 | 92.3% | 6 | 1 | 0 | 1 | <none> | BCL6+ |
| hES_HUES64 | 84.0% | 0 | 2 | 0 | 0 | <none> | <none> |
| hES_HUES65 | 110.6% | 43 | 2 | 7 | 6 | DPP4+, FOXA2+, | GATA4+, IL6+, LAMC3+, |
| | | | | | | GATA4+, LHX1+, | LIFR+, SST+, TBX3+ |
| | | | | | | SST+, TBX3+, | |
| | | | | | | Unannotated+ | |
| hES_HUES66 | 108.8% | 21 | 21 | 2 | 6 | BST2−, FGF8+ | EIF4A3+, FGF8+, GGPS1−, |
| | | | | | | | GRB2+, HRAS+, PHB+ |
| hES_H1 | 126.5% | 58 | 55 | 5 | 9 | BMP4−, ETV1+, | BMP4−, CCND2+, DHCR7+, |
| | | | | | | FAM65B+, | EIFSB−, ETV1+, FANCF+, |
| | | | | | | GABRA1+, NEFH− | LAMB1−, PSMC3−, RHOH+ |
| hES_H7 | 107.5% | 28 | 8 | 2 | 2 | LLGL1+, NGFR+ | NGFR+, SEPT9+ |
| hiPS_11a | 154.1% | 161 | 255 | 10 | 29 | CLDN6+, CST3+, | CCNA1+, CD74+, CDK2−, |
| | | | | | | IFNGR1−, ITGA6−, | CHEK2−, CHN1−, CREB1−, |
| | | | | | | PUM2−, ROCK1−, | CRK−, DHX9−, DPF1+, |
| | | | | | | SOX12+, TNNT2+, | EIF4EBP1+, EML4−, ERC1−, |
| | | | | | | UTF1+, ZMYM2− | FOXO4+, HRAS+, ITGA6−, |
| | | | | | | | MSH6−, NONO−, |
| | | | | | | | PAFAH1B2+, PIK3CA−, |
| | | | | | | | PMS1−, PSEN1−, PTK2−, |
| | | | | | | | PTPN11−, SFRS1−, TFCP2−, |
| | | | | | | | TNFAIP8−, TOP2A−, TSC1−, |
| | | | | | | | ZMYM2− |
| hiPS_11b | 195.3% | 390 | 129 | 38 | 40 | AGR2+, ALB+, | AGR2+, ALB+, ASXL1+, |
| | | | | | | ALDH1A1+, | BAX−, BCL11B+, BMI1+, |
| | | | | | | BMI1+, BMPR2+, | BNIP3L+, BTG1+, CCNE1−, |
| | | | | | | COL2A1+, DCN+, | COL4A6+, COPZ2+, CTBP1+, |
| | | | | | | DLX2+, DPPA4−, | DAP+, DDB2−, EGLN1+, |
| | | | | | | ELAVL4+, | FGF9+, FZD1+, GDF10+, |
| | | | | | | EPYC+, GDF10+, | GLT25D2+, HTATIP2−, |
| | | | | | | GREM2+, | LEF1+, LMO2+, MITF+, |
| | | | | | | HOXA5+, | MLLT3+, NR2F1+, PDGFC+, |
| | | | | | | HOXC4+, ISL1+, | PDGFD+, PGF+, PIK3CD−, |
| | | | | | | LEF1+, LHX2+, | PIK3R1+, PLAGL1+, |
| | | | | | | LMO2+, LPL+, | PRRX1+, RALGDS |
| | | | | | | MAP2+, MEF2C+, | |
| | | | | | | MEIS1+, | |
| | | | | | | MEOX1+, MSX1+, | |
| | | | | | | NEFL+, NEFM+, | |
| | | | | | | NR2F1+, PDGFC+, | |
| | | | | | | PLAGL1+, | |
| | | | | | | SLC2A1+, SOX9+, | |
| | | | | | | SST+, TACSTD | |
| hiPS_15b | 122.8% | 43 | 39 | 4 | 4 | CD46−, DGCR6+, | CCNL1−, ORM2+, RNF7+, |
| | | | | | | IFITM3−, ZMYM2− | ZMYM2− |
| hiPS_17a | 146.9% | 132 | 208 | 15 | 25 | CD81−, COL1A1− | ACSL3−, BAX−, BCL6−, BID+, |
| | | | | | | COL1A2−, | COL1A1−, COL4A1−, |
| | | | | | | COL4A2−, | COL4A2−, CRADD+, JUP−, |
| | | | | | | DGCR6+, IFITM3−, | LAMA5−, LASP1−, LMO1+, |
| | | | | | | ITGAE+, | LSM5+, MEN1−, MYH9−, |
| | | | | | | LAMP1−, LXN+ | NOTCH1−, NOTCH2−, |
| | | | | | | MKI67−, NCSTN− | NR3C1+, RNF7+, SMARCA4−, |
| | | | | | | NES−, NOTCH1− | SOCS2+, TPR−, TRAF6+, |
| | | | | | | NOTCH2−, | TSC2−, VLDLR− |
| | | | | | | SMARCA4− | |
| hiPS_17b | 83.4% | 0 | 3 | 0 | 0 | <none> | <none> |
| hiPS_18a | 85.0% | 3 | 2 | 0 | 0 | <none> | <none> |
| hiPS_18b | 102.3% | 32 | 3 | 0 | 5 | <none> | CREB5+, DDB2+, FOXL2+, |
| | | | | | | | IL1A+, LAMC2+ |
| hiPS_18c | 121.3% | 57 | 103 | 2 | 11 | CD46−, LHX1+ | AXIN1+, BCL6−, ELP4−, |
| | | | | | | | EML4−, FANCG−, NUDT2−, |
| | | | | | | | PALB2−, PJA2−, SS18L1−, |
| | | | | | | | TNFAIP8−, TRAF5− |
| hiPS_20b | 172.2% | 338 | 361 | 16 | 55 | AHCTF1−, BST2−, | ACSL3−, ARHGEF6−, ATM−, |
| | | | | | | CD46−, CNN1+, | BAK1+, BID+, BRCA2−, |
| | | | | | | CNN2+, CSPGS+, | C16orf5+, CASP6+, CCNL1−, |
| | | | | | | DGCR6+, ITGA6−, | CHIC2+, CIAPIN1+, CLTC−, |
| | | | | | | ITGAE+, KLF6−, | DDB2+, DEK−, DICER1−, |
| | | | | | | MKI67−, ROCK1−, | EIF4EBP1+, EIF5B−, ERC1−, |
| | | | | | | SDC1+, TCF4−, | FUS−, GNA14+, GPX1+, |
| | | | | | | TNNT2+, | HRAS+, HSP90B1−, IL1A+, |
| | | | | | | ZMYM2− | ITGA6−, KLF6−, KTN1−, |
| | | | | | | | LAMB1−, MLL−, NRAS+, |
| | | | | | | | OPA1−, PCM1−, PEA15+, P |
| hiPS_27b | 97.5% | 21 | 0 | 1 | 5 | FZD9+ | ARC+, CEP110+, FZD9+, |
| | | | | | | | JUNB+, PROC+ |
| hiPS_27e | 101.9% | 27 | 1 | 1 | 5 | PPP1R13B+ | EIF2S2+, ELF4+, MX1+, |
| | | | | | | | PPP1R13B+, TFE3+ |
| hES_min | 72.0% | 0 | 0 | 0 | 0 | N/A | N/A |
| hES_quartile1 | 81.1% | 5 | 1 | 0 | 0 | N/A | N/A |
| hES_mean | 100.0% | 61 | 33 | 7 | 9 | N/A | N/A |
| hES_quartile3 | 109.7% | 31 | 8 | 2 | 5 | N/A | N/A |
| hES_max | 215.3% | 847 | 500 | 100 | 131 | N/A | N/A |
| hiPS_min | 83.4% | 0 | 0 | 0 | 0 | N/A | N/A |
| hiPS_quartile1 | 99.7% | 24 | 3 | 1 | 5 | N/A | N/A |
| hiPS_mean | 125.7% | 109 | 100 | 8 | 16 | N/A | N/A |
| hiPS_quartile3 | 150.5% | 147 | 169 | 13 | 27 | N/A | N/A |
| hiPS_max | 195.3% | 390 | 361 | 38 | 55 | N/A | N/A |

Explanation for TABLE 6A and 6B

| | |
|---|---|
| variation | Mean variation (DNA methylation or gene expression) across all genes, |
| | normalized to a percentage value relative to all ES cell lines. |
| | Example: 100% −> same amount of variation as an average ES cell line |
| #incr | Number of genes with significantly increased DNA methylation / |
| | gene expression levels relative to the reference of all ES cells |
| #decr | Number of genes with significantly decreased DNA methylation / |
| | gene expression levels relative to the reference of all ES cells |
| #lineage | Number of lineage marker genes with significant increase or decrease |
| #cancer | Number of lineage marker genes with significant increase or decrease |
| lineage markers | Lineage marker genes with significantly increased (+) or decreased (−) |
| | DNA methylation / gene expression levels (*) |
| cancer genes | Cancer genes with significantly increased (+) or decreased (−) |
| | DNA methylation / gene expression levels (*) |

(*) duplicates are due to alternative promoters of the same gene

Any appropriate method for positive selection of cell lines should be simple to perform in a short period of time, be inexpensive and be predictive for applications in differentiation down as many distinct lineages as possible. The inventors assessed if the differentiation of a given cell-line was initiated in a relatively unbiased manner, then its natural differentiation propensities might be predictive of its performance in directed differentiation protocols. In other words, the inventors assessed if a cell line that had a natural propensity to form ectoderm or cells of the neural lineage would also perform optimally in for example motor neuron directed differentiation. To assess this, the inventors designed a simple, rapid, and inexpensive assay for pluripotent cell line differentiation propensities and then determined whether it could predict cell line behavior under directed differentiation (FIG. 5A).

To measure differentiation propensities, the inventors first initiated differentiation by enzymatically passaging ES or iPS cell lines and then placing them in suspension culture in the presence of human ES culture media without bFGF and plasmanate. EBs were cultured in this environment for a total of 16 days then were collected for isolation of total RNA. RNA was analyzed using the Nanostring nCounter system using a signature gene set designed to include 500 lineage specific genes representing the three embryonic germ layers as well as specific somatic lineages such as the neural and hematopoietic lineage (Table 7). An advantage of the nCounter system over standard microarrays is its high sensitivity, large dynamic range of measurement (Geiss et al., 2008) and easy, rapid handling together with low cost per sample. After data collection the inventors statistically compared the gene expression profiles of the two biological replicates to those of a set of “reference” measurements from control EBs (Table 10). Finally, the inventors performed a gene set enrichment analysis (Nam and Kim, 2008; Subramanian et al., 2005) on the differential expression t-scores in order to quantify cell-line specific differentiation propensities relative to the control “reference” EBs.

TABLE 7
Gene set annotations used for construction of the lineage scorecard.

Neural lineage

| | |
|---|---|
| | NCAM1, EN1, FGFR2, GATA2, GATA3, HAND1, MNX1, NEFL, NES, |
| List1_Ectoderm | NOG, OTX2, PAX3, PAX6, PAX7, SNAI2, SOX10, SOX9, TDGF1 |
| | APOE, PDGFRA, MCAM, FUT4, NGFR, ITGB1, CD44, ITGA4, ITGA6, |
| List2_Ectoderm | ICAM1, NCAM1, THY1, FAS, ABCG2, CRABP2, MAP2, CDH2, NES, |
| | NEUROG3, NOG, NOTCH1, SOX2, SYP, MAPT, TH |
| List3_Neural_stem_cells | ABCG2, BMP2, CAMK2A, DLX5, EOMES, FGF2, FGFR3, FOXD3, ISL1, |
| | ITGA4, LMX1A, MAP2, MNX1, MSI1, NES, NEUROG1, NGFR, NOTCH1, |
| | NR2E1, OLIG2, PAX3, SHH, SNAI2, SOX1, SOX4, SOX9, TCF3, TCF4 |
| List4_Neuronal_markers | CAMK2A, CD34, CEACAM1, CEACAM5, DLX5, EOMES, EPHB4, ISL1, |
| | ITGAM, ITGB1, MAP2, MNX1, MSI1, NCAM1, NEFL, NES, NEUROG1, |
| | NR2E1, OLIG2, PAX6, POU5F1, SDC1, SNAI2, SOX10, SOX2, SOX4, |
| | THY1, TWIST1 |
| List5_Neural_stem_cells | ABCG2, BMP2,CAMK2A, DLL1, DLX5, EOMES, FGF2, FGFR3, FOXD3, |
| | ISL1, ITGA4, LMX1A, MAP2, MNX1, MSI1, NES, NEUROG1, NGFR, |
| | NOTCH1, NR2E1, OLIG2,PAX3, SHH, SNAI2, SOX1, SOX4, SOX9, TCF3, |
| | TCF4 |
| List6_Neural_stem_cells | MCAM, FUT4, NGFR, ITGB1, ITGA6, ICAM1, FAS, ABCG2, NES, NOG, |
| | NOTCH1, SOX2 |
| List7_Neuronal_cells | APOE, NGFR, NCAM1, THY1, MAP2, CDH2, NES, SYP, MAPT, TH |

Hennatopuietir lineage

| | |
|---|---|
| List1_Mesoderm | CD34, DLL1, HHEX, INHBA, LEF1, SRF, T, TWIST1 |
| List2_Mesoderm | CD34, HHEX, INHBA, LEF1, SRF, T, TWIST1 |
| List3_Mesoderm | ADIPOQ, MME, KIT, ITGAL, ITGAM, ITGAX, TNFRSF1A, ANPEP, |
| | SDC1, CDH5, MCAM, FUT4, NGFR, ITGB1, PECAM1, CDH1, CDH2, |
| | CD34, CD36, CD4, CD44, ITGA4, ITGA6, ITGAV, ICAM1, NCAM1, |
| | ITGB3, CEACAM1, THY1, ABCG2, KDR, GATA3, GATA4, MYOD1, |
| | MYOG, NES, NOTCH1, SPI1, STAT3 |
| List4_Hematopoietic_ | ABCG2, ANPEP, BMI1, BMPR1A, CD22, CD28, CD34, CD36, CD3E, CD4, |
| progenitor | CD40, CD44, CDH2, CEACAM1, DLL1, EBF1, EPHB4, ERG, ETV2, FAS, |
| | FASLG, FUT4, GATA1, ICAM1, IFNGR1, ITGA6, ITGAL, ITGAM, |
| | ITGAV, ITGAX, ITGB3, JMJD6, KDR, KIT, MME, MPL, NCAM1, |
| | NOTCH1, PECAM1, PODXL, RUNX1, SDC1, SPEN, T, TALI, THY1, |
| | ZBTB16, ZFX |
| List5_Blood | ANPEP, CD36, ITGAV, PECAM1, THPO |
| List6_Adaptive_immunity | CD22, CD28, NCAM1, CD3E, CD4, CD40, CEACAM1, CEACAM5, |
| | FASLG, GATA3, ICAM1, MME, THY1 |
| List7_Innate_immunity | FAS, FASLG, IFNGR1, IRF6, JMJD6, TNFRSF1A |
| List8_Hematopoietic_ | ABCG2, ANPEP, BMI1, BMPR1A, CD22, CD28, CD34, CD36 , CD3E, |
| progenitors | CD4, CD40, CD44, CDH2, CEACAM1, CEACAM5, DLL1, EBF1, EPHB4, |
| | ERG, ETV2, FAS, FASLG, FUT4, GATA1, ICAM1, IFNGR1, ITGA6, |
| | ITGAL, ITGAM, ITGAV, ITGAX, ITGB3, JMJD6, KDR, KIT, MME, MPL |
| | NCAM1, NOTCH1, PECAM1, PODXL, RUNX1, SDC1, SPEN, |
| | T, TAL1, THY1, ZBTB16, ZFX |

Ectoderm germ layer

| | |
|---|---|
| List1_Ectoderm | NCAM1, EN1, FGFR2, GATA2, GATA3, HAND1, MNX1, NEFL, NES, |
| | NOG, OTX2, PAX3, PAX6, PAX7, SNAI2, SOX10, SOX9, TDGF1 |
| List2_Ectoderm | APOE, PDGFRA, MCAM, FUT4, NGFR, ITGB1, CD44, ITGA4, ITGA6, |
| | ICAM1, NCAM1, THY1, FAS, ABCG2, CRABP2, MAP2, CDH2, NES, |
| | NEUROG3, NOG, NOTCH1, SOX2, SYP, MAPT, TH |

Mesoderm germ layer

| | |
|---|---|
| Lis1_Mesoderm | CD34, DLL1, HHEX, INHBA, LEF1, SRF, T, TWIST1 |
| List2_Mesoderm | CD34, HHEX, INHBA, LEF1, SRF, T, TWIST1 |
| List3_Mesoderm | ADIPOQ, MME, KIT, ITGAL, ITGAM, ITGAX, TNFRSF1A, ANPEP, |
| | SDC1, CDH5, MCAM, FUT4, NGFR, ITGB1, PECAM1, CDH1, CDH2, |
| | CD34, CD36, CD4, CD44, ITGA4, ITGA6, ITGAV, ICAM1, NCAM1, |
| | ITGB3, CEACAM1, THY1, ABCG2, KDR, GATA3, GATA4, MYOD1, |
| | MYOG, NES, NOTCH1, SPI1, STAT3 |

Endoderm germ layer

| | |
|---|---|
| List1_Endoderm | APOE, CDX2, FOXA2, GATA4, GATA6, GCG, ISL1, NKX2-5, PAX6, |
| | PDX1, SLC2A2, SST |
| List2_Endoderm | APOE, ITGB1, CD44, ITGA6, THY1, CDX2, GATA4 , HNF1A, HNF1B, |
| | CDH2, NEUROG3, CTNNB1, SYP |

To assess and calibrate this new positive component of the “scorecard” for pluripotent cells, the inventors initially used the scorecard to monitor gene expression in the 19 low-passage ES cell lines used for other analyses in this report (FIG. 5B, FIG. 10B and Table 8). The results of this experiment demonstrated that each cell line displayed quantitative differences in its propensity for differentiation down each of the three germ layers. For example, HUES8 showed the greatest propensity for endoderm differentiation, corroborating previous reports that this cell line performs well in directed endoderm differentiation (Osafune et al., 2008). This result also demonstrates why HUES8 is a frequently used cell line for those engaged in directed endoderm differentiation (Borowiak et al., 2009).

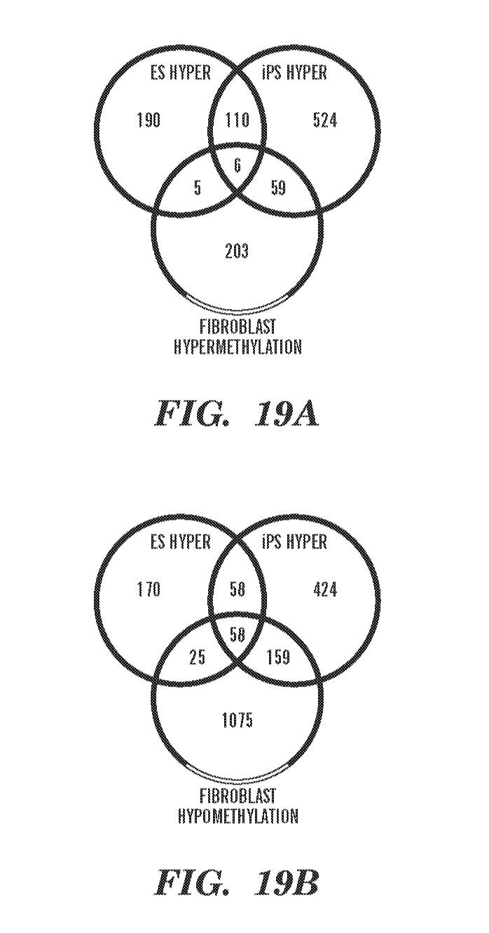

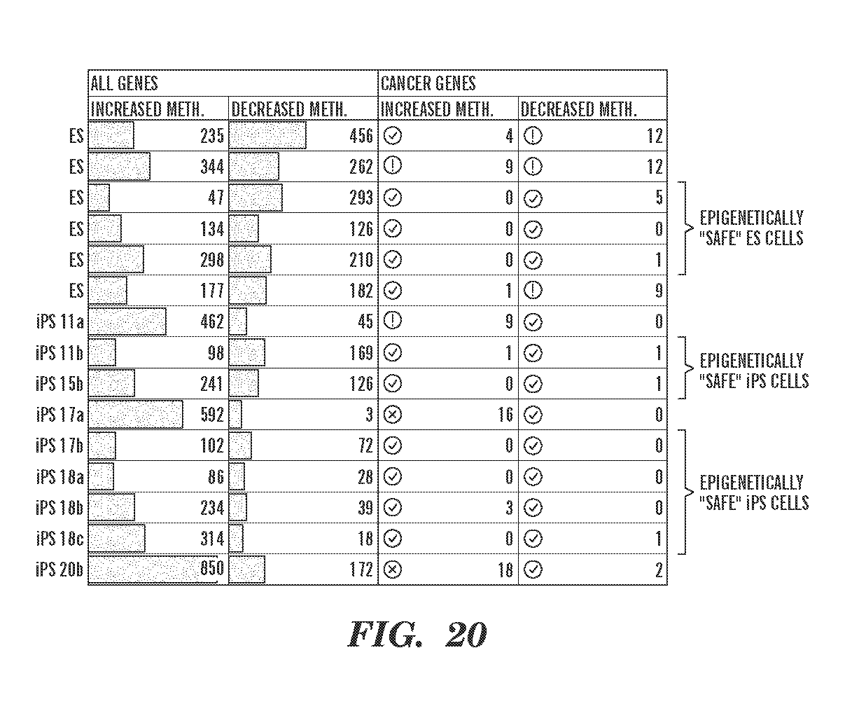

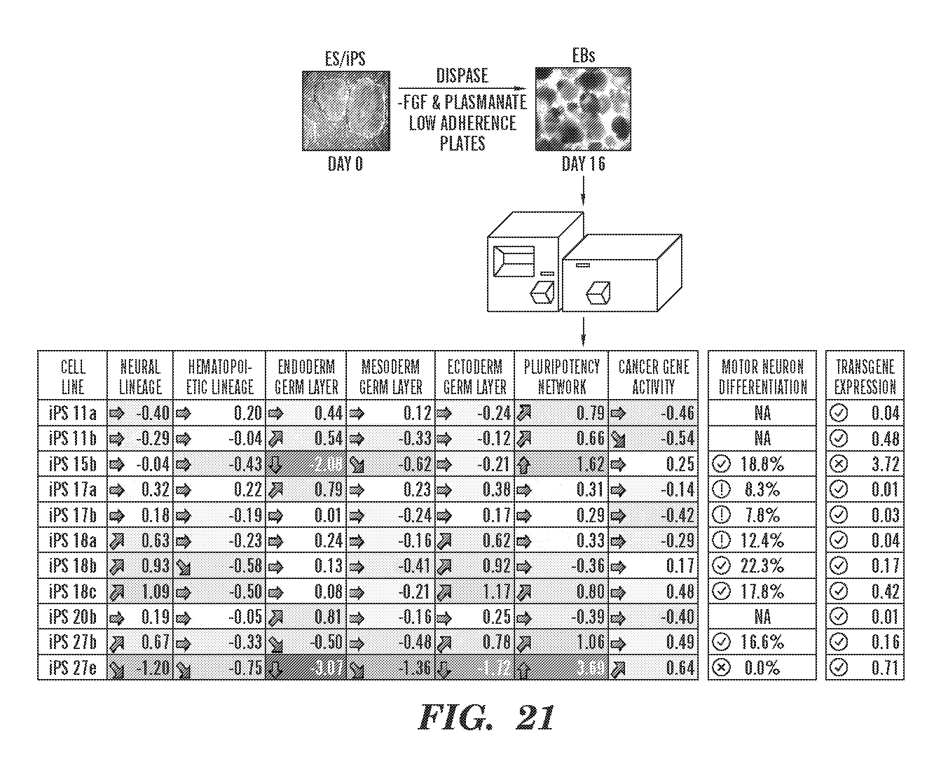

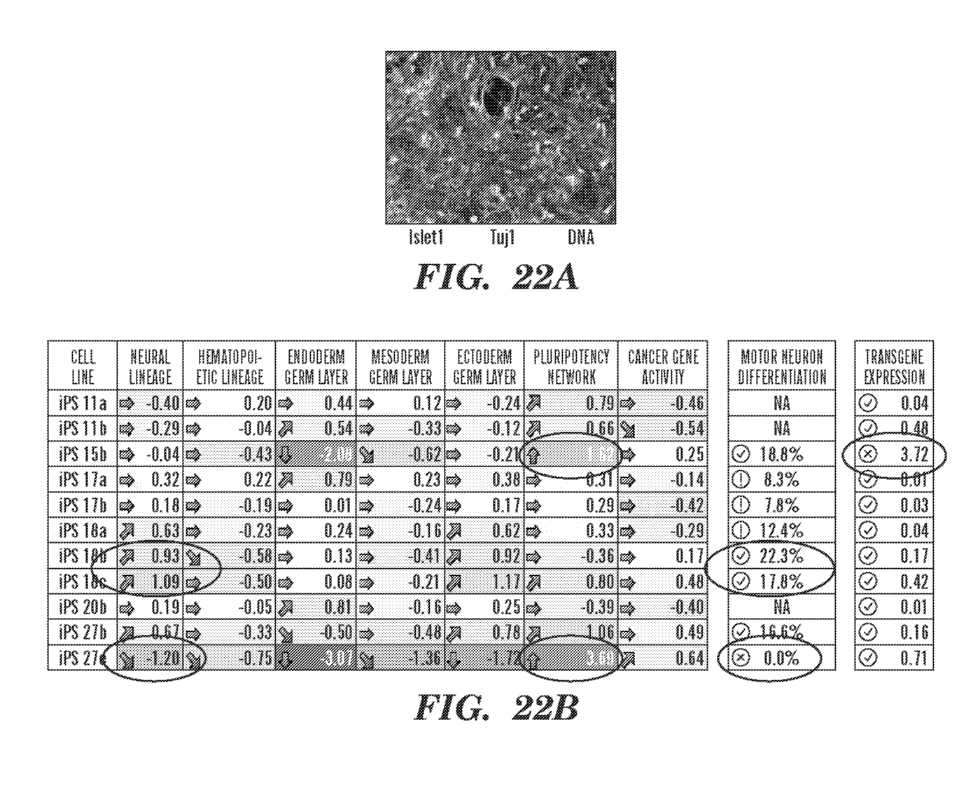

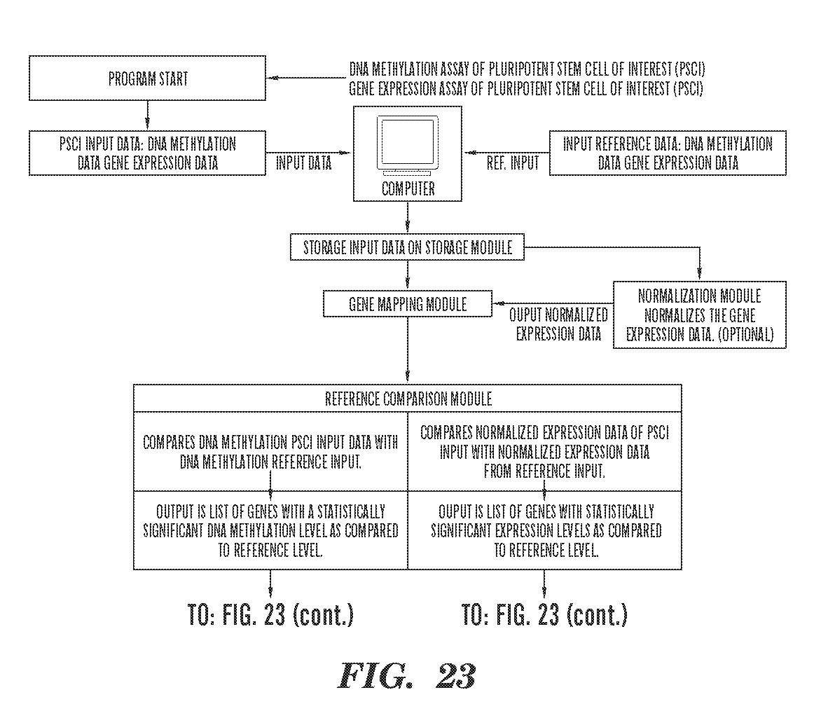

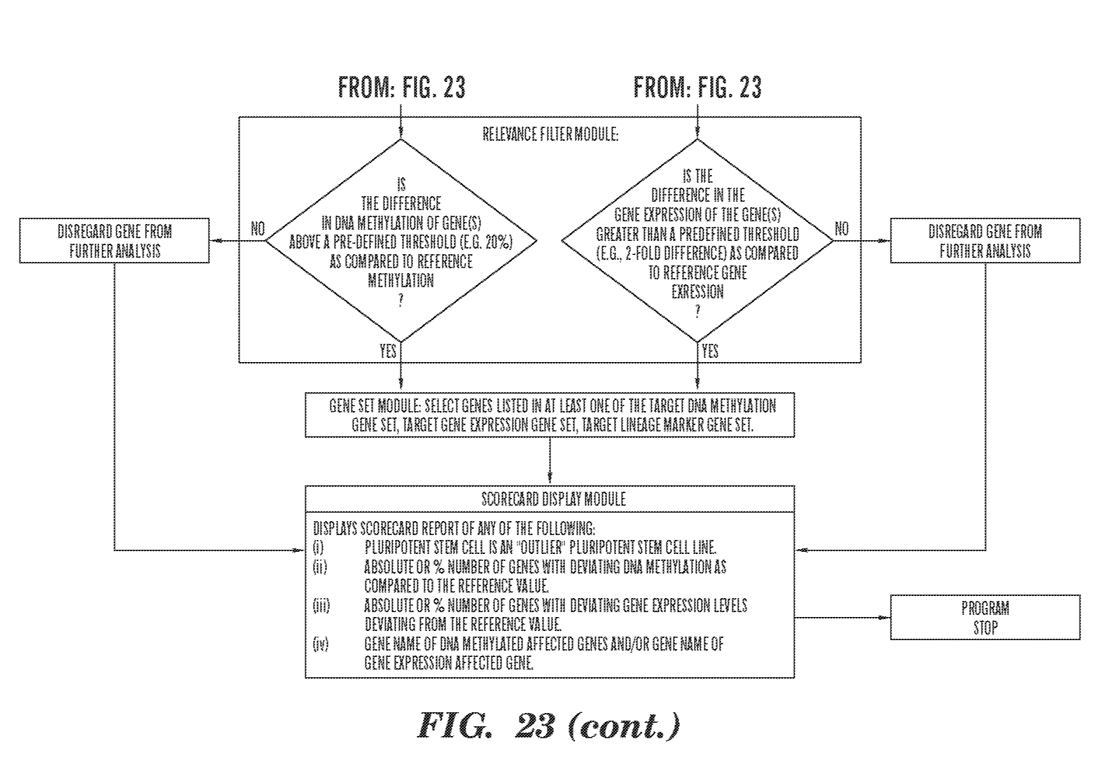

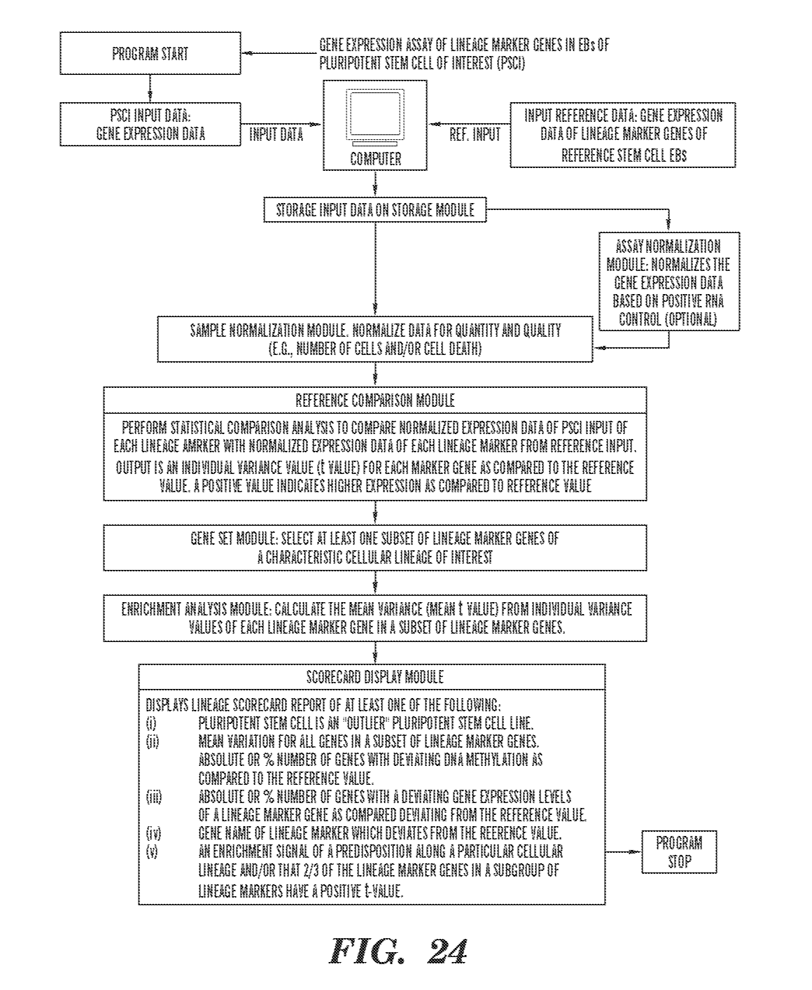

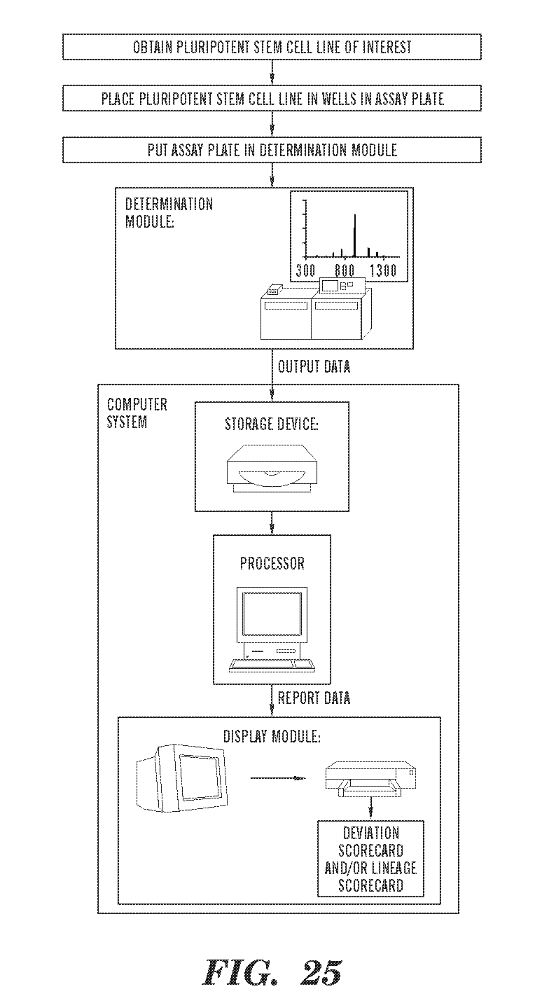

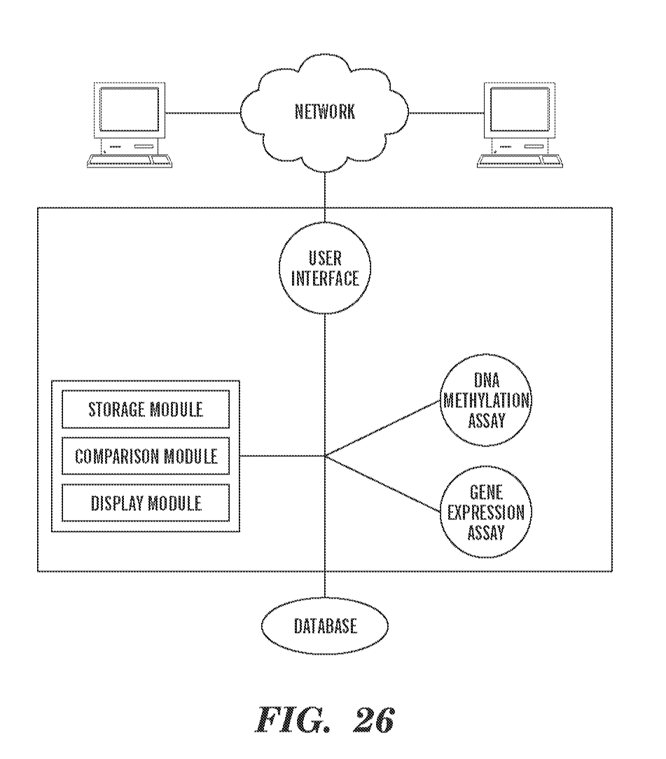

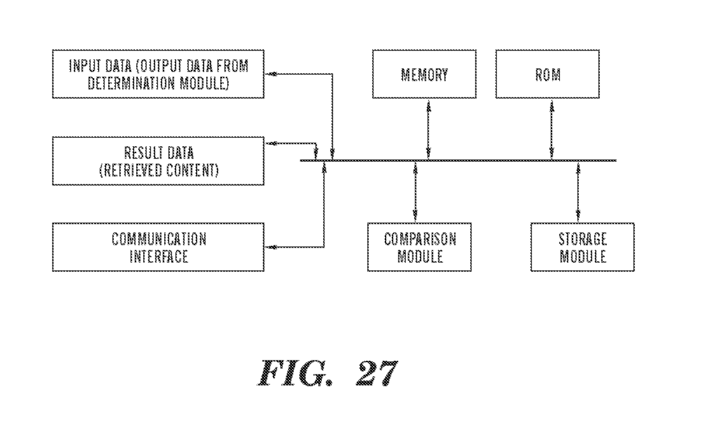

In contrast, H1 and H9 received high “scores” for neural lineage differentiation (FIG. 5B demonstrating that they might be excellent choices for applications in the study or treatment of neural degeneration. Indeed it has been previously reported that these cell lines performed well in a motor neuron-directed differentiation assay (Hu et al., 2010). Although, the inventors initial use of the scorecard as disclosed herein was effective at predicting past utility, the inventors further validated the reproducibility of the lineage scorecard. To this end, the inventors selected lines based on the “scorecard” that performed relatively well or relatively poorly in the production of particular lineages and then assessed whether these propensities were reproducible and whether they could be validated by an independent assay. When the inventors performed an additional, independent round of EB differentiation for several cell lines, and then measured the mRNA levels of 5 genes (NES, TUBB3, KDR, ACTA2, AFP) that are expressed only in discrete lineages, the inventors observed good agreement between the RNA levels for each gene and differentiation propensities predicted by the “scorecard” as disclosed herein (FIG. 11B). Additionally, a more qualitative assessment of these differentiation experiments was carried out by plating EBs under adherent conditions and then immuno-staining with antibodies specific to various differentiated cell types representing all three germ-layers. Again, the inventors scorecard provided a good prediction for the differentiation behaviors of a given cell line (FIGS. 19 and 20).

The inventors initial results demonstrated that a simple transcriptional assay can predict the reproducible behavior of a given ES cell line. The inventors next assessed whether this same lineage “scorecard” could be used to predict the behavior of iPS cells. To this end, the inventors selected several well characterized iPS cell lines (Boulting et al; co-submitted), performed standard EB differentiation, collected RNAs, analyzed them using the Nanostring and normalized the resulting data to the “reference” ES cell-derived EBs. The result was a lineage “scorecard” for the behavior of the selected iPS cell lines (FIGS. 5C and 5D, and FIG. 10C). Table 9 demonstrates a lineage scorecard for predicting the reproducible behaviour of a given pluripotent stem cell line, e.g., ES cell line or iPS cell line.

TABLE 9
Lineage scorecard prediction (Table 9A) and differentiation efficacy into motor neurons (Table 9B).
TABLE 9A: Lineage scorecard prediction

| | hiPS | hiPS | hiPS | hiPS | hiPS | hiPS | hiPS | hiPS | hiPS | hiPS | hiPS | hiPS | hiPS |
|---|---|---|---|---|---|---|---|---|---|---|---|---|---|
| Cell line | 11a | 11b | 15b | 17a | 17b | 18a | 18b | 18c | 20b | 27b | 27e | 29d | 29e |
| No. of replicates | 4 | 4 | 4 | 5 | 3 | 3 | 2 | 2 | 4 | 2 | 5 | 2 | 2 [symbol] |
| Neural lineage | −0.41 | −0.73 | 0.34 | 0.14 | 0.02 | 0.24 | 0.74 | 0.84 | −0.12 | 0.49 | −1.11 | 0.10 | −0.96 [symbol] |
| (mean) | | | | | | | | | | | | | |
| Hematopoietic | −0.12 | −0.43 | −0.56 | −0.11 | −0.39 | −0.44 | −0.54 | −0.55 | −0.39 | −0.49 | −0.81 | 0.20 | −0.76 [symbol] |
| lineage (mean) | | | | | | | | | | | | | |
| Ectoderm germ | −0.28 | −0.68 | −0.50 | 0.17 | 0.01 | 0.21 | 0.75 | 0.89 | −0.13 | 0.56 | −1.50 | 0.03 | −1.19 [symbol] |
| layer (mean) | | | | | | | | | | | | | |
| Mesoderm germ | −0.43 | −1.01 | −0.84 | −0.18 | −0.65 | −0.57 | −0.46 | −0.35 | −0.83 | −0.63 | −1.35 | −0.33 | −1.31 [symbol] |
| layer (mean) | | | | | | | | | | | | | |
| Endoderm germ | 0.23 | −0.05 | −1.90 | 0.41 | −0.11 | −0.11 | −0.08 | −0.08 | 0.06 | −0.57 | −2.20 | 0.45 | −1.31 [symbol] |
| layer (mean) | | | | | | | | | | | | | |
| Neural lineage | 0.25 | 0.61 | 0.63 | 0.31 | 0.40 | 0.45 | 0.01 | 0.08 | 0.38 | 0.13 | 0.11 | 0.20 | 0.55 [symbol] |
| (stdev) | | | | | | | | | | | | | |
| Hematopoietic | 0.10 | 0.52 | 0.29 | 0.17 | 0.19 | 0.22 | 0.01 | 0.12 | 0.19 | 0.20 | 0.19 | 0.17 | 0.06 [symbol] |
| lineage (stdev) | | | | | | | | | | | | | |
| Ectoderm germ | 0.16 | 0.75 | 0.83 | 0.29 | 0.44 | 0.50 | 0.06 | 0.02 | 0.44 | 0.23 | 0.18 | 0.21 | 0.58 [symbol] |
| layer (stdev) | | | | | | | | | | | | | |
| Mesoderm germ | 0.18 | 0.82 | 0.71 | 0.28 | 0.52 | 0.50 | 0.30 | 0.49 | 0.53 | 0.08 | 0.44 | 0.21 | 0.22 [symbol] |
| layer (stdev) | | | | | | | | | | | | | |
| Endoderm germ | 0.19 | 0.89 | 0.80 | 0.33 | 0.21 | 0.45 | 0.30 | 0.09 | 0.69 | 0.08 | 0.21 | 0.15 | 0.22 [symbol] |
| layer (stdev) | | | | | | | | | | | | | |
| Neural lineage | 0.12 | 0.30 | 0.31 | 0.14 | 0.23 | 0.26 | 0.01 | 0.06 | 0.19 | 0.09 | 0.05 | 0.14 | 0.39 [symbol] |
| (std.err) | | | | | | | | | | | | | |
| Hematopoietic | 0.05 | 0.26 | 0.14 | 0.08 | 0.11 | 0.13 | 0.01 | 0.09 | 0.10 | 0.14 | 0.09 | 0.12 | 0.05 [symbol] |
| lineage (std.err) | | | | | | | | | | | | | |
| Ectoderm germ | 0.08 | 0.38 | 0.41 | 0.13 | 0.25 | 0.29 | 0.04 | 0.02 | 0.22 | 0.17 | 0.08 | 0.15 | 0.41 [symbol] |
| layer (std.err) | | | | | | | | | | | | | |
| Mesoderm germ | 0.09 | 0.41 | 0.36 | 0.12 | 0.30 | 0.29 | 0.22 | 0.35 | 0.26 | 0.06 | 0.20 | 0.15 | 0.16 [symbol] |
| layer (std.err) | | | | | | | | | | | | | |
| Endoderm germ | 0.09 | 0.45 | 0.40 | 0.15 | 0.12 | 0.26 | 0.22 | 0.07 | 0.34 | 0.06 | 0.09 | 0.11 | 0.16 [symbol] |
| layer (std.err) | | | | | | | | | | | | | |
| Neural lineage | 10 | 11 | 9 | 5 | 6 | 4 | 2 | 1 | 8 | 3 | 13 | 7 | 12 [symbol] |
| (rank) | | | | | | | | | | | | | |
| Hematopoietic | 2 | 6 | 11 | 1 | 5 | 7 | 9 | 10 | 4 | 8 | 13 | 3 | 12 [symbol] |
| lineage (rank) | | | | | | | | | | | | | |
| Ectoderm germ | 9 | 11 | 10 | 5 | 7 | 4 | 2 | 1 | 8 | 3 | 13 | 6 | 12 [symbol] |
| layer (rank) | | | | | | | | | | | | | |
| Mesoderm germ | 4 | 11 | 10 | 1 | 8 | 6 | 5 | 3 | 9 | 7 | 13 | 2 | 12 [symbol] |
| layer (rank) | | | | | | | | | | | | | |
| Endoderm germ | 3 | 5 | 12 | 2 | 8 | 9 | 6 | 7 | 4 | 10 | 13 | 1 | 11 [symbol] |
| layer (rank) | | | | | | | | | | | | | |

TABLE 9B: Differentiation efficiency into motor neurons (percentage of ISL1-positive cells)

| | hiPS | hiPS | hiPS | hiPS | hiPS | hiPS | hiPS | hiPS | hiPS | hiPS | hiPS | hiPS | hiPS |
|---|---|---|---|---|---|---|---|---|---|---|---|---|---|
| cell line | 11a | 11b | 15b | 17a | 17b | 18a | 18b | 18c | 20b | 27b | 27e | 29d | 29e |
| No. of | 5 | 4 | 1 | 1 | 2 | 6 | 5 | 6 | 1 | 5 | 6 | 6 | 3 [symbol] |
| experiments (3 | | | | | | | | | | | | | |
| replicates each) | | | | | | | | | | | | | |
| efficiency (mean) | 6.23 | 0.00 | 13.29 | 8.32 | 7.17 | 10.73 | 13.04 | 15.26 | 7.61 | 11.27 | 0.00 | 9.87 | 0.00 [symbol] |
| efficiency (stdev) | 1.67 | 0.00 | 2.63 | 2.63 | 0.29 | 3.02 | 3.86 | 4.34 | 2.63 | 5.03 | 0.00 | 4.37 | 0.00 [symbol] |
| efficiency (std.err) | 0.75 | 0.00 | 2.63 | 2.63 | 0.21 | 1.23 | 1.73 | 1.77 | 2.63 | 2.25 | 0.00 | 1.78 | 0.00 [symbol] |
| efficiency (rank) | 10 | 11 | 2 | 7 | 9 | 5 | 3 | 1 | 8 | 4 | 11 | 6 | 11 [symbol] |

[symbol]

indicates data missing or illegible when filed

To independently validate the differentiation “scorecard” by another assay, the inventors repeated the differentiation of several iPS cell lines and then used flow cytometry to analyze the percentage of cells that expressed a gene specific to the endoderm (AFP) (FIG. 10D). Again, the scorecard could accurately predict the lines that had a propensity for endoderm differentiation (FIG. 10D).

To further confirm the robustness and reproducibility of the scorecard for predicting the behavior of iPS cell lines, the inventors differentiated each iPS cell line up to five independent times and then analyzed harvested RNA using a simple transcriptional assay (Table 11A, and Table 11B). Importantly, the inventors observed excellent overall correlation between the scorecard predictions generated by each replicate from a given cell-line (Pearson's r=0.82).

TABLE 11
Consistency and reproducibility of the lineage scorecard assay
TABLE 11A: Consistency and reproducibility of the lineage scorecard assay

| | | | | | | Correlation |
|---|---|---|---|---|---|---|
| | | | | | | between |
| | Neural | Hematopoietic- | Ectoderm | Mesoderm | Endoderm | biological |
| Biological replicate | lineage | ietic lineage | germ layer | germ layer | germ layer | replicates |
| hEB16d_11a_p14 | −0.68 | −0.24 | −0.44 | −0.33 | 0.36 | 0.81 |
| hEB16d_11a_p18 | −0.13 | −0.03 | −0.16 | −0.24 | 0.12 | 0.91 |
| hEB16d_11a_p27 | −0.53 | −0.04 | −0.39 | −0.56 | 0.03 | 0.81 |
| hEB16d_11a_p29 | −0.28 | −0.16 | −0.12 | −0.60 | 0.42 | |
| hEB16d_11b_p18 | −1.56 | −1.14 | −1.72 | −2.09 | −1.38 | 0.73 |
| hEB16d_11b_p25 | −0.50 | −0.41 | −0.49 | −1.12 | 0.21 | 0.76 |
| hEB16d_11b_p15 | −0.13 | −0.27 | 0.08 | −0.19 | 0.48 | 0.55 |
| hEB16d_11b_p31 | −0.73 | 0.11 | −0.58 | −0.62 | 0.48 | |
| hEB16d_15b_p29 | 0.57 | −0.17 | 0.71 | 0.22 | −0.72 | 0.72 |
| hEB16d_15b_p30 | −0.66 | −0.62 | −1.01 | −1.06 | −2.48 | 0.97 |
| hEB16d_15b_p41 | −0.44 | −0.57 | −0.67 | −1.19 | −2.27 | 1.00 |
| hEB16d_15b_p44 | −0.83 | −0.87 | −1.04 | −1.31 | −2.13 | |
| hEB16d_17a_p17 | −0.16 | 0.04 | −0.02 | −0.12 | 0.91 | 0.81 |
| hEB16d_17a_p10 | −0.16 | −0.32 | −0.17 | −0.57 | 0.21 | 0.90 |
| hEB16d_17a_p19 | 0.26 | −0.15 | 0.36 | −0.23 | 0.48 | 0.69 |
| hEB16d_17a_p16 | 0.56 | −0.20 | 0.56 | −0.17 | 0.05 | 0.69 |
| hEB16d_17a_p12 | 0.18 | 0.09 | 0.10 | 0.20 | 0.38 | |
| hEB16d_17b_p18 | 0.49 | −0.17 | 0.51 | −0.11 | 0.03 | 0.81 |
| hEB16d_17b_p20 | −0.23 | −0.49 | −0.27 | −0.71 | −0.35 | 0.92 |
| hEB16d_17b_p38 | −0.19 | −0.52 | −0.22 | −1.14 | 0.00 | 0.66 |
| hEB16d_18a_p31 | 0.36 | −0.54 | 0.33 | −0.65 | −0.28 | 0.93 |
| hEB16d_18a_p32 | 0.61 | −0.18 | 0.63 | −0.03 | 0.40 | 0.78 |
| hEB16d_18a_p46 | −0.26 | −0.59 | −0.34 | −1.02 | −0.45 | |
| hEB16d_18b_p20 | 0.73 | −0.54 | 0.79 | −0.24 | 0.14 | 0.95 |
| hEB16d_18b_p37 | 0.74 | −0.53 | 0.71 | −0.67 | −0.29 | 1.00 |
| hEB16d_18c_p30 | 0.89 | −0.63 | 0.90 | −0.69 | −0.14 | 0.94 |
| hEB16d_18c_p32 | 0.78 | −0.46 | 0.87 | 0.00 | −0.01 | |
| hEB16d_20b_p31 | −0.02 | −0.21 | 0.04 | −0.43 | 0.40 | 0.96 |
| hEB16d_20b_p26 | 0.36 | −0.27 | 0.39 | −0.33 | 0.79 | 0.72 |
| hEB16d_20b_p50 | −0.50 | −0.46 | −0.59 | −1.24 | −0.18 | 0.66 |
| hEB16d_20b_p46 | −0.32 | −0.63 | −0.37 | −1.33 | −0.78 | 0.78 |
| hEB16d_27b_p27 | 0.58 | −0.63 | 0.72 | −0.69 | −0.62 | 0.99 |
| hEB16d_27b_p28 | 0.40 | −0.35 | 0.39 | −0.57 | −0.51 | |
| hEB16d_27e_p30 | −1.01 | −0.51 | −1.28 | −0.70 | −1.85 | 0.99 |
| hEB16d_27e_p32 | −1.26 | −0.79 | −1.73 | −1.13 | −2.33 | 0.92 |
| hEB16d_27e_p31 | −1.00 | −0.83 | −1.51 | −1.47 | −2.36 | 0.97 |
| hEB16d_27e_p32 | −1.11 | −0.90 | −1.39 | −1.72 | −2.28 | 0.99 |
| hEB16d_27e_p35 | −1.17 | −1.03 | −1.60 | −1.74 | −2.20 | |
| hEB16d_29d_p15 | 0.04 | −0.32 | 0.17 | −0.47 | 0.34 | 0.61 |
| hEB16d_29d_p14 | −0.24 | −0.08 | −0.12 | −0.18 | 0.55 | |
| hEB16d_29e_p25 | −1.35 | −0.80 | −1.60 | −1.46 | −1.46 | 0.40 |
| hEB16d_29e_p27 | −0.57 | −0.71 | −0.78 | −1.15 | −1.15 | |
| hFib_11_p7 | −1.35 | 0.14 | −1.03 | −0.51 | −2.16 | 0.89 |
| hFib_11_p8 | −1.58 | 0.36 | −1.51 | −0.81 | −1.65 | |
| hFib_15_p6 | −1.85 | 0.26 | −1.87 | −0.64 | −2.08 | 0.95 |
| hFib_15_p7 | −2.15 | 0.10 | −2.11 | −0.92 | −1.63 | |
| hFib_17_p6 | −1.60 | 0.17 | −1.56 | −0.71 | −2.46 | 0.83 |
| hFib_17_p7 | −1.74 | 0.30 | −1.76 | −0.51 | −1.28 | |
| hFib_18_p6 | −1.61 | 0.60 | −1.58 | −0.25 | −2.37 | 0.96 |
| hFib_18_p7 | −1.32 | 0.39 | −1.25 | −0.86 | −2.04 | |
| hFib_20_p6 | −2.12 | 0.22 | −2.17 | −0.74 | −2.30 | 0.98 |
| hFib_20_p7 | −1.95 | 0.16 | −1.94 | −0.82 | −1.68 | |
| hFib_27_p6 | −1.75 | 0.88 | −1.81 | 0.70 | −2.57 | 1.00 |
| hFib_27_p7 | −1.74 | 0.95 | −1.87 | 0.59 | −2.68 | |
| hMN_11a_p21 | −0.95 | −0.49 | −1.29 | −1.45 | −1.58 | |
| hMN_15b_p27 | −0.60 | −0.84 | −1.34 | −1.93 | −1.36 | |
| hMN_17a_p9 | −0.92 | −0.49 | −1.48 | −1.33 | −1.80 | |
| hMN_17b_p31 | −0.92 | −0.82 | −1.42 | −1.90 | −1.53 | |
| hMN_18a_p28 | −0.30 | −0.78 | −0.55 | −1.42 | −1.50 | |
| hMN_18b_p25 | −0.51 | −0.71 | −0.94 | −1.48 | −1.39 | |
| hMN_18c_p34 | −0.07 | −0.57 | −0.37 | −1.27 | −1.28 | |
| hMN_20b_p33 | 0.08 | −0.56 | −0.36 | −0.28 | −1.28 | |
| hMN_27b_p34 | −0.92 | −0.72 | −1.03 | −2.16 | −1.05 | |
| hES_HUES1_p26 | −0.15 | −0.31 | −0.53 | −0.26 | −1.59 | 1.00 |
| hES_HUES1_p26 | −0.10 | −0.25 | −0.49 | −0.27 | −1.51 | |
| hES_HUES3_p27 | −0.69 | −0.42 | −1.25 | −0.59 | −1.80 | 0.91 |
| hES_HUES3_p28 | −0.70 | −0.44 | −1.33 | −0.72 | −1.26 | |
| hES_HUES6_p19 | −0.80 | −0.46 | −1.27 | −0.83 | −1.43 | 0.97 |
| hES_HUES6_p21 | −0.58 | −0.14 | −1.20 | −0.52 | −1.84 | |
| hES_HUES8_p25 | −0.50 | 0.02 | −1.14 | −0.22 | −0.69 | 0.88 |
| hES_HUES8_p26 | −0.61 | 0.29 | −1.25 | 0.19 | −1.51 | |
| hES_HUES9_p19 | −0.94 | −0.11 | −1.66 | −0.38 | −1.95 | 0.93 |
| hES_HUES9_p18 | −0.64 | −0.47 | −1.22 | −0.71 | −1.19 | |
| hES_HUES28_p13 | −0.69 | −0.30 | −1.49 | −0.17 | −1.64 | 0.98 |
| hES_HUES28_p15 | −0.53 | −0.23 | −1.21 | −0.13 | −1.67 | |
| hES_HUES44_p15 | −0.67 | −0.34 | −1.36 | −0.66 | −1.41 | 1.00 |
| hES_HUES44_p16 | −0.60 | −0.23 | −1.31 | −0.57 | −1.25 | |
| hES_HUES45_p17 | −0.06 | −0.20 | −0.49 | −0.24 | −0.82 | 0.99 |
| hES_HUES45_p19 | −0.06 | −0.28 | −0.51 | −0.31 | −0.83 | |
| hES_HUES48_p16 | −0.11 | 0.56 | −0.69 | 0.42 | −1.04 | 0.99 |
| hES_HUES48_p17 | −0.11 | 0.45 | −0.64 | 0.36 | −1.27 | |
| hES_HUES49_p14 | −0.67 | −0.12 | −1.36 | −0.37 | −1.46 | 1.00 |
| hES_HUES49_p14 | −0.72 | −0.17 | −1.40 | −0.51 | −1.43 | |
| hES_HUES53_p17 | −0.80 | −0.35 | −1.20 | −0.43 | −0.87 | 0.97 |
| hES_HUES53_p18 | −0.57 | −0.35 | −0.92 | −0.35 | −0.78 | |
| hES_HUES62_p16 | −0.08 | 0.45 | −0.54 | 0.39 | −0.62 | 0.92 |
| hES_HUES62_p15 | −0.57 | −0.37 | −1.21 | −0.58 | −1.59 | 0.66 |
| hES_HUES62_p16 | 0.72 | 0.03 | 0.42 | 0.28 | −1.03 | 1.00 |
| hES_HUES62_p16 | 0.78 | 0.03 | 0.50 | 0.28 | −0.96 | 1.00 |
| hES_HUES62_p18 | 0.70 | 0.01 | 0.41 | 0.28 | −0.91 | |
| hES_HUES63_p19 | −0.51 | −0.15 | −1.24 | −0.43 | −1.54 | 0.97 |
| hES_HUES63_p17 | −0.67 | −0.26 | −1.43 | −0.20 | −1.65 | |
| hES_HUES64_p18 | −0.09 | 0.41 | −0.56 | 0.37 | −0.61 | 0.98 |
| hES_HUES64_p20 | −0.15 | 0.54 | −0.73 | 0.38 | −1.15 | |
| hES_HUES65_p16 | −0.21 | 0.09 | −0.67 | 0.25 | −0.56 | 0.27 |
| hES_HUES65_p17 | 0.71 | −0.02 | 0.46 | 0.30 | −1.04 | |
| hES_HUES66_p15 | −0.84 | −0.32 | −1.56 | −0.68 | −1.58 | 0.97 |
| hES_HUES66_p15 | −0.49 | −0.13 | −1.21 | −0.41 | −1.58 | |
| hES_H1_p33 | −0.43 | −0.22 | −0.92 | −0.30 | −2.29 | 1.00 |
| hES_H1_p34 | −0.57 | −0.39 | −1.07 | −0.52 | −2.76 | |
| hES_H9_p57 | 0.33 | −0.01 | −0.05 | 0.45 | −1.07 | 0.99 |
| hES_H9_p58 | 0.30 | 0.06 | 0.00 | 0.59 | −0.98 | |
| hiPS_11a_p14 | −0.89 | 0.32 | −1.27 | 0.41 | −2.10 | 0.77 |
| hiPS_11a_p18 | −1.11 | −0.24 | −1.68 | −0.77 | −1.25 | |
| hiPS_11b_p15 | −0.73 | 0.16 | −1.19 | −0.33 | −0.99 | 0.83 |
| hiPS_11b_p18 | −0.92 | −0.22 | −1.38 | −0.66 | −2.16 | |
| hiPS_15b_p29 | −1.33 | −0.55 | −1.83 | −1.17 | −2.89 | 0.99 |
| hiPS_15b_p30 | −1.40 | −0.55 | −1.92 | −1.11 | −2.57 | |
| hiPS_17a_p16 | −0.65 | −0.28 | −1.07 | −0.27 | −1.68 | 0.74 |
| hiPS_17a_p16 | −0.37 | 0.07 | −0.84 | 0.34 | −0.48 | |
| hiPS_17b_p18 | −0.78 | −0.18 | −1.15 | −0.20 | −1.57 | 0.92 |
| hiPS_17b_p20 | −0.55 | −0.42 | −0.96 | −0.40 | −1.85 | 0.77 |
| hiPS_17b_p38 | −0.80 | −0.20 | −1.37 | −0.44 | −1.27 | |
| hiPS_18a_p31 | −0.40 | −0.23 | −0.72 | −0.35 | −1.85 | 0.29 |
| hiPS_18a_p32 | −1.02 | −0.49 | −1.45 | −0.44 | −0.89 | |
| hiPS_18b_p20 | −1.12 | −0.54 | −1.56 | −0.78 | −1.97 | 0.86 |
| hiPS_18b_p37 | −0.17 | −0.18 | −0.44 | 0.17 | −1.51 | |
| hiPS_18c_p30 | −0.18 | −0.28 | −0.30 | −0.28 | −1.79 | 0.78 |
| hiPS_18c_p32 | −0.68 | −0.04 | −1.04 | −0.03 | −1.70 | |
| hiPS_20b_p31 | −0.37 | −0.33 | −0.62 | −0.25 | −1.05 | 0.32 |
| hiPS_20b_p26 | −1.19 | −0.60 | −1.65 | −0.69 | −0.97 | |
| hiPS_27b_p27 | −0.66 | −0.16 | −1.10 | −0.29 | −1.62 | 1.00 |
| hiPS_27b_p28 | −0.93 | −0.32 | −1.35 | −0.47 | −1.96 | |
| hiPS_27e_p30 | −1.04 | −0.33 | −1.73 | −0.51 | −2.21 | 0.98 |
| hiPS_27e_p32 | −1.48 | −0.46 | −2.03 | −1.08 | −2.71 | |
| hiPS_29d_p15 | −0.49 | −0.28 | −0.75 | −0.40 | −1.12 | 0.70 |
| hiPS_29d_p14 | −0.58 | −0.15 | −1.06 | −0.45 | −0.73 | |
| hiPS_29e_p25 | −1.57 | −0.90 | −2.13 | −1.59 | −1.74 | 0.91 |
| hiPS_29e_p27 | −1.55 | −0.92 | −2.08 | −1.46 | −1.31 | |

TABLE 11B

| Sample | | Mean correlation |
|---|---|---|
| type | Description | between replicates |
| hEB16d | 16-day embryoid bodies | 0.82 |
| hFib | Human fibroblasts | 0.93 |
| hES | Human ES cell lines | 0.92 |
| hiPS | Human iPS cell lines | 0.78 |

The utility of the inventors “scorecard” for pluripotent cell differentiation propensity would be substantially increased if it could predict how a given cell line will perform in a directed differentiation assay. The inventors assessed if a cell line with a natural propensity for differentiation towards a given lineage would also perform well in directed differentiation strategies aimed at producing particular cell-types from that lineage. The inventors assessed this to determine if the “scorecard” as disclosed herein would have broad utility in cell line selection for any application in which human ES or iPS cells were used for directed differentiation. To assess this, the inventors assessed if the scorecard could predict the efficiency by which each line from a large cohort of iPS cell lines produced motor neurons when subjected to a robust directed differentiation protocol (Wichterle et al., 2002) (Di Giorgio et al., 2008) (Boulting et al., co-submitted).

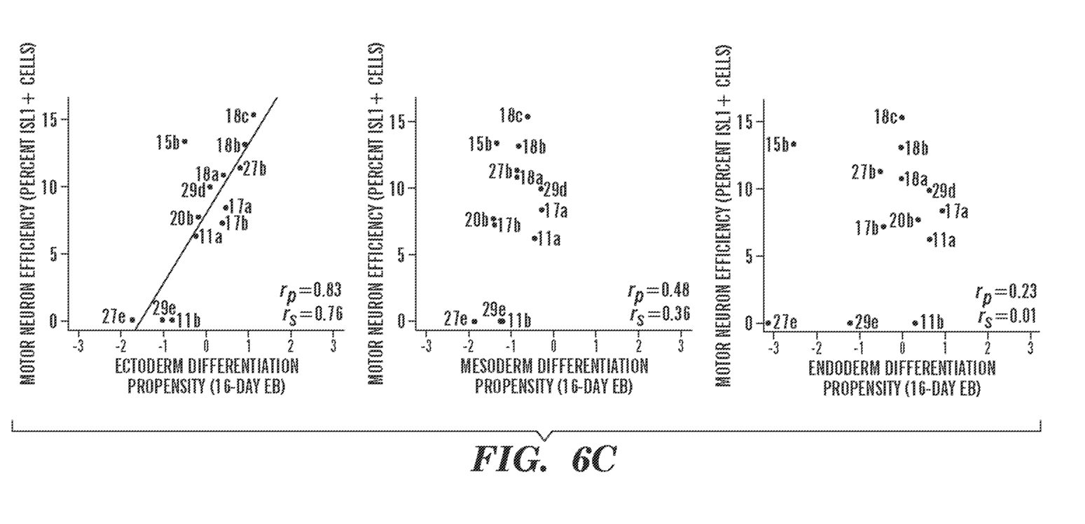

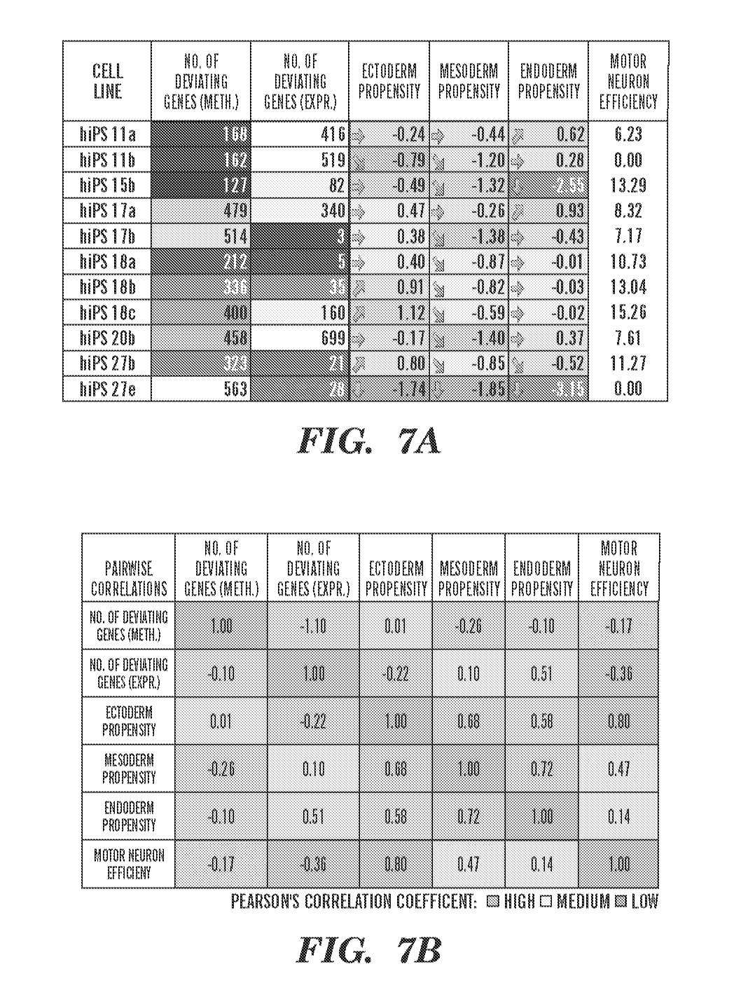

In brief, each iPS cell line was subjected to motor neuron directed differentiation and the efficiency of motor neuron production was monitored by automated quantification of cells that were immuno-reactive for the motor neuron specific transcription factors ISL1/2 and HB9 (FIG. 6A in Boulting et al., co-submission). These directed differentiation data provided a genuine test-set for determining the predictive power of the “scorecard” in this context. The identity of genes whose expression was monitored by a simple transcriptional assay had already been finalized before the first comparisons between the two datasets were made, and no parameters of the “scorecard” were retrospectively optimized to improve the fit. When the inventors compared the estimate for the neural lineage differentiation propensity of a given cell line that was made by the “scorecard” with the actual efficiency by which each cell line produced motor neurons, the inventors observed a remarkably high correlation (FIG. 6B) (Pearson's 7=0.85 for ISL1, r=0.86 for HB9). This initial result demonstrates that measuring the differentiation propensity of a given cell line can be used to predict the pluripotent stem cell's behavior in a directed differentiation protocol. However, if the “scorecard” is only useful in predicting the overall recalcitrance or amenability of a cell line towards differentiation into any sort of cell it can be determined by the efficiency by which that line generates motor neurons.

To determine the specificity of scorecard predictions for a given lineage, the inventors correlated the efficiency of motor neuron differentiation with scorecard predictions for propensity of differentiation down each of the three embryonic germ layers (FIG. 6C and FIG. 11A). The inventors demonstrated an excellent correlation between the estimation for ectoderm differentiation propensity and motor neuron production (Pearson's r=0.83 for ISL1, r=0.82 for HB9). In contrast, there was a much poorer correlation between the efficiency by which a cell line produces motor neurons and its predicted propensities for mesoderm differentiation (Pearson's r=0.48 for ISL1, r=0.44 for HB9) or endoderm differentiation (Pearson's r=0.23 for ISL1, r=0.26 for HB9). In summary, the inventors have clearly demonstrated a rapid assay that can be performed by any lab by one of ordinary skill in the art in order to optimally select iPS or ES cell lines for a given application.

### Example 7

### Toward High-Throughput Evaluation of Pluripotent Cell Quality and Utility

The inventors have described three genomic assays that can be used for quality assessment of human ES and iPS cell lines and have calibrated these assays by establishing a “reference map” of variation that exists in each measure among low-passage human ES cell lines. The Inventors have demonstrated use of the assays as disclosed herein to design an initial “scorecard” that they demonstrate can predict the differentiation propensities of any pluripotent cell line. The scorecard output as shown in FIG. 7A, which summarizes the number and identity of epigenetic and transcriptional deviations in any new ES or iPS cell line and also provides a systematic estimate of a cell line's differentiation propensities. To increase the utility and put the characterization of pluripotent stem cell lines within the reach of any investigator of ordinary skill in the art, the inventors revisited key components of the initial scorecard and attempted to identify opportunities to simplify the assays and to further reduce cost.

First, the inventors assessed whether all three assays were strictly required or whether DNA methylation, gene expression or the quantitative differentiation assay could be omitted without compromising the accuracy of the score-card. The inventors data clearly point toward the importance of the three assays: No single assay was redundant in the sense that its ranking of the different iPS cell lines was perfectly correlated with the results of another assay (FIG. 7B). Nevertheless, it seems possible to reduce the cost and complexity of DNA methylation assays by exploiting the bias of DNA methylation defects toward a small number of highly susceptible genes (FIG. 2A). Based on the inventor's dataset, the inventors would detect 80% of the DNA methylation deviations in iPS cell lines by monitoring only the 10% most variable genes in ES cells (FIG. 7C). Focusing on the ˜3,000 most variable genes (plus another ˜1,000 manually selected genes that should be monitored even for rare defects) brings the number of promoter regions well within the range commercial epigenotyping assays (Bibikova et al., 2009), which are widely available through microarray core facilities.

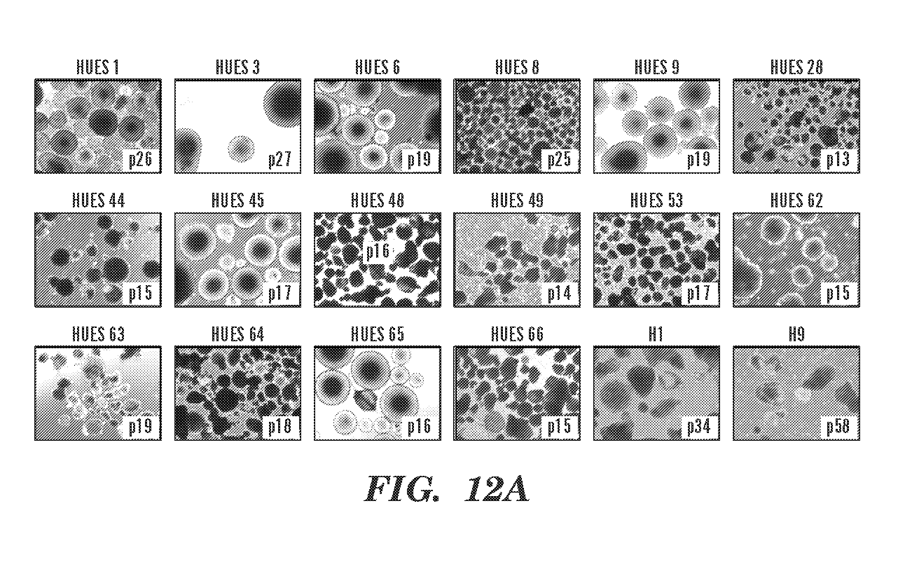

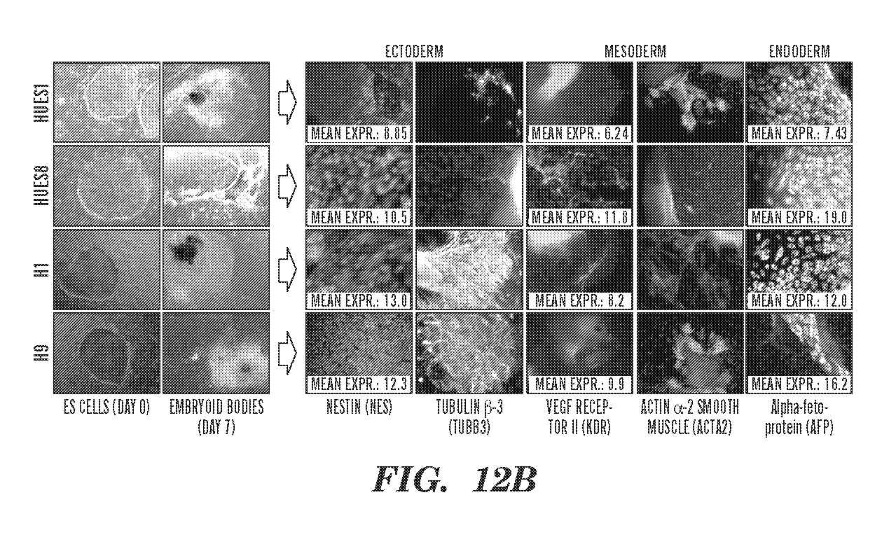

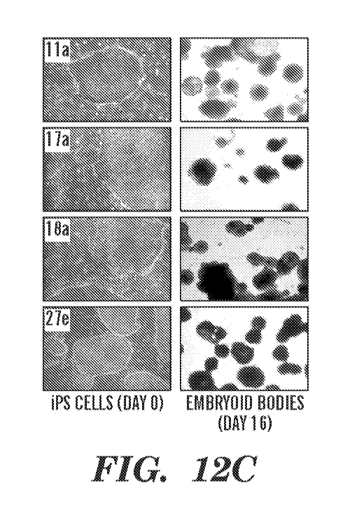

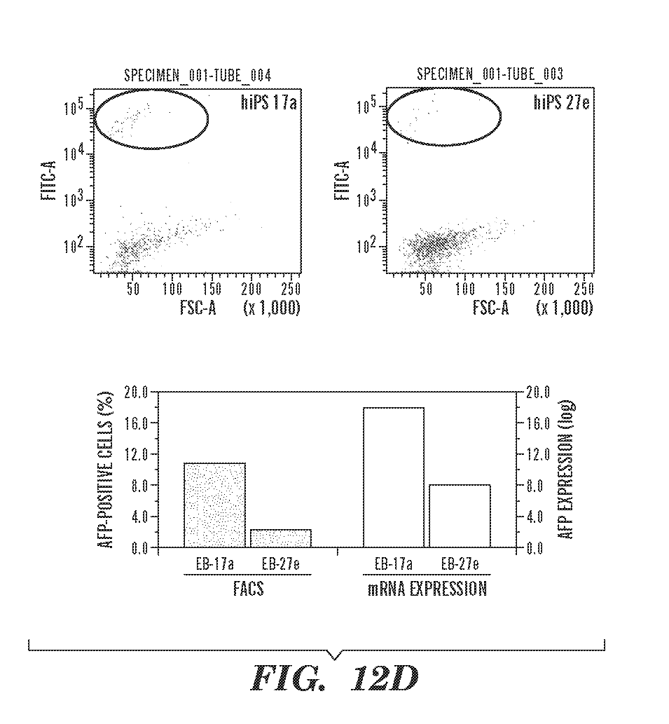

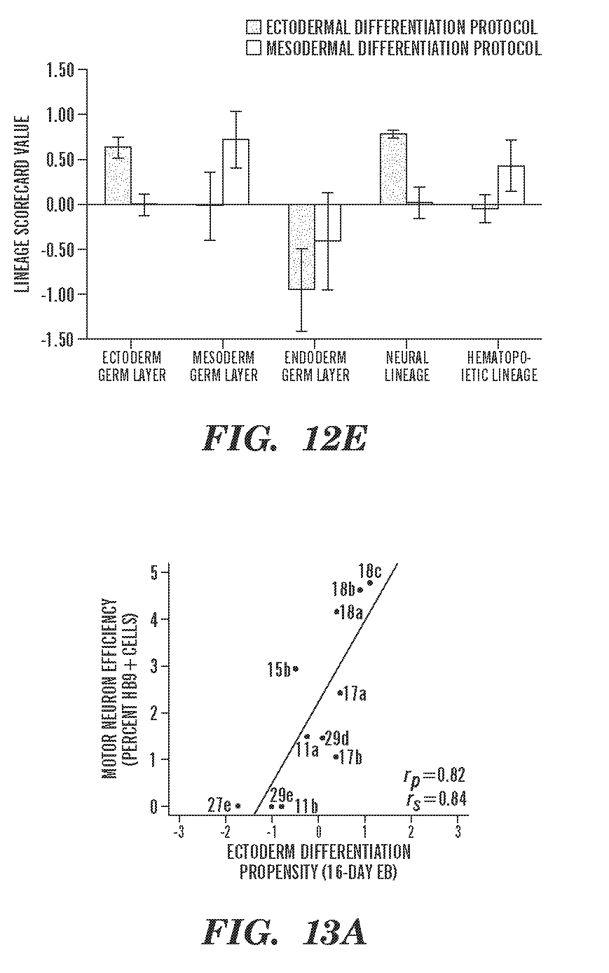

In contrast, for gene expression it is not possible to focus on a small number of ES-cell variable genes while still capturing a complete range of the iPS-specific deviations (FIG. 12). However, the inventors have demonstrated that is not a practical limitation. Commercially available microarrays for monitoring transcription are widely available, easy-to-use and relatively cost-efficient for one of ordinary skill in the art.

As an additional measure, the inventors aimed to reduce the total length of time it took to perform the quantitative differentiation assay. Accordingly, shortening the duration of the assay is advantageous as it decreases the time-to-results and also minimizes the logistical costs in terms of incubator space and need for media changes. The inventors optimized the quantitative differentiation assay so it is sensitive enough to estimate differentiation propensities using RNA isolated directly from the undifferentiated pluripotent cell lines, most likely by detecting low levels of cellular differentiation in otherwise self-renewing cultures.

To assess the effect of shortening the duration of the quantitative differentiation assay, the inventors purified total RNA from each ES and iPS cell lines under self-renewing conditions, performed transcriptional analysis using the Nanostring and constructed a new “score-card” for these ES and iPS cell lines (FIG. 7D). Interestingly, there was some limited correlation between this new ES/iPS scorecard and the original EB scorecard (“r” ranged between 0.59 and 0.82) (FIG. 7D), demonstrating that some reasonable predictions can be made using RNA expressed from the pluripotent cell lines themselves. Surprisingly, the dynamic range of the predictions made with the undifferentiated cells was substantially lower than that of the scorecard generated using RNA from EBs subjected to 16 days of differentiation. Therefore, although analyzing RNA from a pluripotent stem cell line can be performed, it is likely to reduce the robustness of the assay. As an alternative, the inventors assessed whether the duration of the EB assay could be reduced from 16 days to 7 days. In this case, the inventors demonstrated an excellent agreement between the two assays on four representative iPS cell lines (Pearson's r>0.9), demonstrating that it is possible to reduce the duration of the differentiation assay without jeopardizing its accuracy.

### Example 8

The inventors also investigated how robust and reproducible the results from the “scorecard” remained when the inventors compared the same pluripotent stem lines across several passages and between independent labs. Because the inventors methods for analyzing DNA methylation and transcription have been shown to be reproducible (Gu et al., 2010; Irizarry et al., 2005) and because the inventors have already investigated how these measures change with passage (data not shown), the inventors focused on the reproducibility of the quantitative differentiation assay. Because differentiation of ES cells in EBs is likely to be sensitive to differences in such parameters as physical handling, media renewal and plasticware, the inventors assessed how predictive the results from the differentiation assay would be of cell line behavior in another lab and with a distinct investigator.

The inventors therefore performed a systematic comparison in which one cell line (hiPS 17b) was cultured for two passages by two different investigators in two different labs, who also performed the EB assay separately and independently. The correlation between the lineage scorecard predictions was lower than the r=0.82 observed above when the assay was carried out in the same lab by the same investigator. However, the inventors demonstrated a correlation that is considered reproducible (r=0.59). Therefore, for optimal cell line selection, the inventors recommend that each lab should use the combined assays which are described here to generate a scorecard for their own lines, under their own culture conditions. To maintain accurate estimates of differentiation propensity, the inventors recommend repeating the scorecard assay when a line is newly sub-cloned or subjected to substantial passage as it is common practice with karyotypic analysis.

### Example 9

In the study herein the inventors utilized several genomic assays to investigate the variation observed among a large cohort of pluripotent cell lines and developed a scorecard that can be applied to classify existing or newly derived lines (ES and iPS cells) and predict their differentiation propensities. The inventors “reference levels” of commonly observed variation and the development of the “scorecard” as disclosed herein is particularly relevant due to several developments in the human stem cell field.

Until recently, only a few human pluripotent cell lines were widely available for biomedical research. For this reason, researchers have mostly relied on these readily accessible and well characterized cell lines (Cowan et al., 2004; Mitalipova et al., 2003; Thomson et al., 1998). Funding restrictions placed on human ES cell research in the United States further limited the selection of cell lines available. As a result, investigators simply used any lines they could for their application of interest with little need for a diagnostic that could predict how well a given cell line would behave in a given assay.

However, the continued derivation of human ES cell lines by many labs (Chen et al., 2009) and the lifting of funding restrictions in the US, has substantially increased the number of ES cell lines that investigators may choose from. Additionally, it has become clear that not all human ES cell lines are equally suited for every purpose (Osafune et al., 2008). This suggests that any new research project should perform a deliberate and informed selection of the cell lines that are most qualified for an application of interest.

The discovery of factors that reprogram somatic cells from patients into iPS cells has lead to a further inflection in the number of pluripotent cell lines available to, and needed by, the research community. As investigators gather together existing cell lines, or derive new ones for their application of interest, there is little information or guidance concerning how to select cell lines that are most appropriate. The inventors herein provide a clear path to guide investigators to proceed from patient samples, to fully reprogrammed iPS cells, to a selected and manageable set of lines that can be used at a reasonable scale for disease modeling.

Here, the inventors demonstrate methods to accurately predict the propensities of human pluripotent cell lines, thereby allowing investigators to select lines that would perform optimally in their given application. Importantly, the use of the “scorecard” as disclosed herein for pluripotent cell line quality and utility, can be readily scaled for the characterization of any number of pluripotent cell lines, e.g., as few as about 5 pluripotent stem cell lines to 10's and 100's of pluripotent stem cell lines.

In aggregate, the scorecard as disclosed herein reports many different characteristics of a given pluripotent cell line's state and behaviors that an investigator would wish to understand before investing significant time and resources into its use in any particular application. For instance, the scorecard as disclosed herein incorporates gene expression profiles for the pluripotent cell lines, allowing investigators to be confident that cell lines they select transcribe the appropriate level of genes that are normally expressed in pluripotent cells (FIG. 1). In some embodiments, these gene expression profiles can also be used to measure somatic gene expression signatures to ensure that a cell line of interest has not been mishandled and some cells have differentiated to become a mixed population of both pluripotent and differentiated cells.

For those interested in developing cell therapies, it may be critical to demonstrate that a pluripotent cell line being put forward for clinical development fits to “standard” criteria from preparation to preparation and does not express aberrant levels of either tumor suppressor or oncogenes. Accordingly, the inventors production and use of the “scorecard” as disclosed herein is useful for these important safety measures before administering a pluripotent stem cell or their progeny to a subject in therapeutic use.

In some embodiments, the inventors “scorecard” also includes profiling of DNA methylation levels in order to detect epigenetic variation between lines that is not reflected in the transcriptional profiles of the undifferentiated cells (FIGS. 1 and 2). Here, the inventors have demonstrated that an understanding of this variation in general, coupled to a specific measurement of DNA methylation in a given line of interest, can be used to avoid, or negatively select out, cell lines whose epigenetic profile could impede their differentiation down a lineage of interest (FIG. 2E), or would indicate that a pluripotent stem cell lines does not express aberrant levels of either tumor suppressor or oncogenes.

One of the assays that contributes information on a pluripotent cell line propensities into the scorecard is a novel and quantitative differentiation assay. This quantitative differentiation assay uses transcriptional measures of genes expressed in specific lineages as a counting device to quantify the prevalence of cell types from each lineage in heterogeneous EBs.

In order to comprehensively calibrate and validate the “scorecard” for use with both human iPS and ES cell lines, the inventors established “reference maps” for the genome wide levels of transcription and DNA methylation of at least 19 ES cell lines and 11 iPS cell lines. In order to ensure that a single “scorecard” could be relevant to both human ES and iPS cells, the inventors performed comprehensive statistical comparisons of both measures in these two pluripotent cell types. The results of these comparisons confirm that the inventors “scorecard” is highly relevant to both cell types. Importantly, these statistical results were also functionally confirmed by the implementation of the “scorecard” to predict the past behavior of a number of human ES cell lines in a directed endoderm differentiation assay as well as to predict with high accuracy the efficiency by which 11 of the iPS cell lines could be differentiated into motor neurons (FIGS. 6 and 7).

As an aside, the inventors datasets and the statistical comparisons which were made between cell lines also enabled the inventors to assess whether ES cells and iPS cell lines are distinct from one another. Unlike previous reports (Doi et al., 2009; Stadtfeld et al., 2010b), the 30 cell lines the inventors analyzed herein provided a data set with sufficient “power of numbers” to come to a statistically informed answer to this question. Using a robust statistical learning approach the inventors evaluated previously published iPS-specific signatures and derived a classifier that could distinguish between the ES and iPS cell lines used in this study at higher-than-random accuracy (FIG. 3D). It was clear from the inventors analyses that no single locus or gene signature could accurately distinguish between all ES and all iPS cell lines. In other words, epigenetic and transcriptional differences can distinguish the average ES cell line from the average iPS cell line, but these differences are insufficient to draw conclusions about the characteristics of any single ES or iPS cell line under consideration. In other words, the inventors determined that some ES cell lines are more suited for a given application than others, and the same is true of iPS cells. As a result of these studies, the inventors have determined that that current methods of reprogramming are surprisingly robust.

The inventors also determined that rather than trying to find the optimal ES cell line or the perfect reprogramming protocol for all needs and applications, what seems to be required is a rapid assay that can match suitable cell lines to a given application. Accordingly, the methods, systems and the “scorecard” as disclosed herein are useful to determine and predict the propensities of human pluripotent cell lines, such that an appropriate pluripotent stem cell with desired propensities could be matched and selected for use in specific downstream applications.

While the inventors demonstrate here “scorecard” for pluripotent cells, the inventors also have demonstrated “reference maps” of the pluripotent epigenome and transcriptome which provide a valuable source of biological insights into the epigenetic and transcriptional regulation of pluripotent stem cells. For example, the inventors demonstrated that epigenetic variation among ES cell lines is highly correlated with DNA sequence motifs that have previously been shown to render genomic regions susceptible to DNA methylation (Bock et al., 2006; Keshet et al., 2006; Meissner et al., 2008).

Surprisingly, the inventors also demonstrated a striking enrichment of gene expression of genes that function in cell signaling in the class of the most transcription-variable gene. This demonstrated that each pluripotent cell line may have adapted in different ways to the selective pressures of in vitro culture. Accordingly, based on this data, ES cell lines are also useful to provide a model system for investigating the ramifications of cellular competition and epigenetic adaption to growth conditions. Finally, the inventors also demonstrated some pluripotent stem cell lines had variable levels of methylation at the CD14 promoter, demonstrating that promoter hypermethylation is a means of silencing key genes in a developmental pathway occurs in pluripotent stem cell lines and will be useful to developmental research to determine additional insights into the epigenetic regulation of “gatekeeper genes” (Hemberger et al., 2009) during human embryonic development.

In summary, the inventors have analyzed and measured DNA methylation, transcription and differentiation propensities in many human pluripotent cell lines and lead to the development of simple systems, methods and assays that any investigator of ordinary skill in the art can utilize to generate a “scorecard” to predict the behavior of any new, or existing, pluripotent cell line (FIG. 7E). Presently, without the current invention, after obtaining an existing pluripotent stem cell line, or generating a new one, an investigator would perform a number of time-consuming, laborious and expensive assays including immunostaining for specific antigens and teratoma generation. While these assays may provide some confidence that a given cell line is pluripotent, they are unable to predict whether a pluripotent cell line is well suited to a given application. In contrast, the present methods, kits, systems, assays and scorecards as disclosed herein are useful to predict the behavior of the pluripotent stem cell in a quick, efficient and effective manner, which is not time or labor intensive and relatively inexpensive.

Accordingly, using the methods, kits, systems, assays and scorecards as disclosed herein, a researcher interested in disease modeling of, for example, amyotrophic lateral sclerosis (ALS), could analyze their pluripotent stem cells of interest and perform the quantitative differentiation assay as disclosed herein (FIG. 5D). The researcher can then select those pluripotent stem cell lines exhibiting normal to high differentiation propensity for the neural lineage for further studies. Next, the selected pluripotent cell lines can then be subjected to DNA methylation analysis and/or transcriptional profiling. Accordingly, using the methods, systems and scorecards as disclosed herein, an investigator can inspect cell lines for variation in the parameters that would best predict the utility of the pluripotent stem cell line in their particular desired application (FIG. 7E).

The inventors methods, assays, scorecards and kits as disclosed herein enable an investigator to delay the most time-consuming and expensive assay, teratoma formation, to be started on a particular pluripotent stem cell line only at a time when the “scorecard” has predicted that the selected pluripotent cell line is likely to differentiate into motor neurons, or other cells of interest at a high efficiency and did not exhibit other serious limitations (e.g., expression of oncogenes or repression of tumor suppressor genes etc). Over time, the use of the methods, assays, scorecards and kits as disclosed herein may enable one to eliminate the teratoma generation assay completely if the methods, assays, scorecards as disclosed herein are used to accurately predict pluripotent stem cell lines with the potential to form teratomas.

In conclusion, the discovery of human pluripotent cells and the reprogramming methods to produce human iPS cells from selected patient populations has revolutionized how researchers think about studying and treating human disease. However, if use of human pluripotent stem cells and iPS cells are to efficiently and effectively used in research as well as cell therapy and therapeutic use to improve the lives of patients, it is imperative to establish a quality assessment and validation method such as the methods, assays, systems and “scorecard” as disclosed herein to streamline, standardize and optimize the selection of pluripotent cell lines for studying, for drug development and toxicity assays as well as for a particular therapeutic implication, or for treating a given indication or disease.

### REFERENCES

The references are incorporated herein in their entirety by reference.
Adewumi, O., Aflatoonian, B., Ahrlund-Richter, L., Amit, M., Andrews, P. W., Beighton, G., Bello, P. A., Benvenisty, N., Berry, L. S., Bevan, S., et al. (2007). Characterization of human embryonic stem cell lines by the International Stem Cell Initiative. Nat. Biotechnol 25, 803-816
Allison, D. B., Cui, X., Page, G. P., and Sabripour, M. (2006). Microarray data analysis: from disarray to consolidation and consensus. Nat Rev Genet. 7, 55-65.
Bibikova, M., Le, J., Barnes, B., Saedinia-Melnyk, S., Zhou, L., Shen, R., and Gunderson, K. L. (2009). Genome-wide DNA methylation profiling using Infinium assay. Epigenomics 1, 177-200.
Bird, A. (2002). DNA methylation patterns and epigenetic memory. Genes Dev 16, 6-21.
Bock, C., Halachev, K., Büch, J., and Lengauer, T. (2009). EpiGRAPH: User-friendly software for statistical analysis and prediction of (epi-) genomic data. Genome Biol 10, R14.
Bock, C., Paulsen, M., Tierling, S., Mikeska, T., Lengauer, T., and Walter, J. (2006). CpG island methylation in human lymphocytes is highly correlated with DNA sequence, repeats, and predicted DNA structure. PLoS Genet. 2, e26.
Borowiak, M., Maehr, R., Chen, S., Chen, A. E., Tang, W., Fox, J. L., Schreiber, S. L., and Melton, D. A. (2009). Small molecules efficiently direct endodermal differentiation of mouse and human embryonic stem cells. Cell Stem Cell 4, 348-358.
Carvajal-Vergara, X., Sevilla, A., D'Souza, S. L., Ang, Y. S., Schaniel, C., Lee, D. F., Yang, L., Kaplan, A. D., Adler, E. D., Rozov, R., et al. (2010). Patient-specific induced pluripotent stem-cell-derived models of LEOPARD syndrome. Nature 465, 808-812.
Chen, A. E., Egli, D., Niakan, K., Deng, J., Akutsu, H., Yamaki, M., Cowan, C., Fitz-Gerald, C., Zhang, K., Melton, D. A., et al. (2009). Optimal timing of inner cell mass isolation increases the efficiency of human embryonic stem cell derivation and allows generation of sibling cell lines. Cell stem cell 4, 103-106.
Chin, M. H., Mason, M. J., Xie, W., Volinia, S., Singer, M., Peterson, C., Ambartsumyan, G., Aimiuwu, 0., Richter, L., Zhang, J., et al. (2009). Induced pluripotent stem cells and embryonic stem cells are distinguished by gene expression signatures. Cell Stem Cell 5, 111-123.
Colman, A., and Dreesen, O. (2009). Pluripotent stem cells and disease modeling. Cell Stem Cell 5, 244-247. Cowan, C. A., Klimanskaya, I., McMahon, J., Atienza, J., Witmyer, J., Zucker, J. P., Wang, S., Morton, C. C., McMahon, A. P., Powers, D., et al. (2004). Derivation of embryonic stem-cell lines from human blastocysts. N Engl J Med 350, 1353-1356.
Daley, G. (2010). Straight talk with . . . George Daley. Interview by Elie Dolgin. Nat. Med 16, 624.
Di Giorgio, F. P., Boulting, G. L., Bobrowicz, S., and Eggan, K. C. (2008). Human embryonic stem cell-derived motor neurons are sensitive to the toxic effect of glial cells carrying an ALS-causing mutation. Cell Stem Cell 3, 637-648.
Dimos, J. T., Rodolfa, K. T., Niakan, K. K., Weisenthal, L. M., Mitsumoto, H., Chung, W., Croft, G. F., Saphier, G., Leibel, R., Goland, R., et al. (2008). Induced pluripotent stem cells generated from patients with ALS can be differentiated into motor neurons. Science 321, 1218-1221.
Doi, A., Park, I. H., Wen, B., Murakami, P., Aryee, M. J., Irizarry, R., Herb, B., Ladd-Acosta, C., Rho, J., Loewer, S., et al. (2009). Differential methylation of tissue- and cancer-specific CpG island shores distinguishes human induced pluripotent stem cells, embryonic stem cells and fibroblasts. Nat. Genet.
Ebert, A. D., Yu, J., Rose, F. F., Jr., Mattis, V. B., Lorson, C. L., Thomson, J. A., and Svendsen, C. N. (2009). Induced pluripotent stem cells from a spinal muscular atrophy patient. Nature 457, 277-280.
Eiges, R., Urbach, A., Malcov, M., Frumkin, T., Schwartz, T., Amit, A., Yaron, Y., Eden, A., Yanuka, O., Benvenisty, N., et al. (2007). Developmental study of fragile X syndrome using human embryonic stem cells derived from preimplantation genetically diagnosed embryos. Cell Stem Cell 1, 568-577.
ENCODE Project Consortium (2007). Identification and analysis of functional elements in 1% of the human genome by the ENCODE pilot project. Nature 447, 799-816.
Geiss, G. K., Bumgarner, R. E., Birditt, B., Dahl, T., Dowidar, N., Dunaway, D. L., Fell, H. P., Ferree, S., George, R. D., Grogan, T., et al. (2008). Direct multiplexed measurement of gene expression with color-coded probe pairs. Nature Biotechnology 26, 317-325.
Gentleman, R. C., Carey, V. J., Bates, D. M., Bolstad, B., Dettling, M., Dudoit, S., Ellis, B., Gautier, L., Ge, Y., Gentry, J., et al. (2004). Bioconductor: open software development for computational biology and bioinformatics. Genome Biol 5, R80.
Gu, H., Bock, C., Mikkelsen, T. S., Jager, N., Smith, Z. D., Tomazou, E., Gnirke, A., Lander, E. S., and Meissner, (2010). Genome-scale DNA methylation mapping of clinical samples at single-nucleotide resolution. Nat Methods 7, 133-136.
Hanna, J., Cheng, A. W., Saha, K., Kim, J., Lengner, C. J., Soldner, F., Cassady, J. P., Muffat, J., Carey, B. W., and Jaenisch, R. (2010). Human embryonic stem cells with biological and epigenetic characteristics similar to those of mouse ESCs. Proc Natl Acad Sci USA 107, 9222-9227.
Hastie, T., Tibshirani, R., and Friedman, J. H. (2001). The elements of statistical learning: data mining, inference, and prediction (New York, Springer).
Hawkins, R. D., Hon, G. C., Lee, L. K., Ngo, Q., Lister, R., Pelizzola, M., Edsall, L. E., Kuan, S., Luu, Y., Klugman, S., et al. (2010). Distinct epigenomic landscapes of pluripotent and lineage-committed human cells. Cell Stem Cell 6, 479-491.
Hemberger, M., Dean, W., and Reik, W. (2009). Epigenetic dynamics of stem cells and cell lineage commitment: digging Waddington's canal. Nature Reviews Molecular Cell Biology 10, 526-537.
Hu, B. Y., Weick, J. P., Yu, J., Ma, L. X., Zhang, X. Q., Thomson, J. A., and Zhang, S. C. (2010). Neural differentiation of human induced pluripotent stem cells follows developmental principles but with variable potency. Proc Natl Acad Sci USA 107, 4335-4340.
Huang, D. W., Sherman, B. T., Tan, Q., Kir, J., Liu, D., Bryant, D., Guo, Y., Stephens, R., Baseler, M. W., Lane, H. C., et al. (2007). DAVID Bioinformatics Resources: expanded annotation database and novel algorithms to better extract biology from large gene lists. Nucleic Acids Res 35, W169-175.
Hubbard, T. J., Aken, B. L., Ayling, S., Ballester, B., Beal, K., Bragin, E., Brent, S., Chen, Y., Clapham, P., Clarke, L., et al. (2009). Ensembl 2009. Nucleic Acids Res 37, D690-697.
Huber, W., von Heydebreck, A., Sultmann, H., Poustka, A., and Vingron, M. (2002). Variance stabilization applied to microarray data calibration and to the quantification of differential expression. Bioinformatics 18 Suppl 1, S96-104.
Irizarry, R. A., Warren, D., Spencer, F., Kim, I. F., Biswal, S., Frank, B. C., Gabrielson, E., Garcia, J. G., Geoghegan, J., Germino, G., et al. (2005). Multiple-laboratory comparison of microarray platforms. Nature Methods 2, 345-350.
Kauffmann, A., Gentleman, R., and Huber, W. (2009). arrayQualityMetrics—a bioconductor package for quality assessment of microarray data. Bioinformatics 25, 415-416.
Keshet, I., Schlesinger, Y., Farkash, S., Rand, E., Hecht, M., Segal, E., Pikarski, E., Young, R. A., Niveleau, A., Cedar, H., et al. (2006). Evidence for an instructive mechanism of de novo methylation in cancer cells. Nat Genet. 38, 149-153.
Laird, P. W. (2010). Principles and challenges of genome-wide DNA methylation analysis. Nat Rev Genet. 11, 191-203.
Lee, G., Papapetrou, E. P., Kim, H., Chambers, S. M., Tomishima, M. J., Fasano, C. A., Ganat, Y. M., Menon, J., Shimizu, F., Viale, A., et al. (2009). Modelling pathogenesis and treatment of familial dysautonomia using patient-specific iPSCs. Nature. Lengner, C. J., Gimelbrant, A. A., Erwin, J. A., Cheng, A. W., Guenther, M. G., Welstead, G. G., Alagappan, R., Frampton, G. M., Xu, P., Muffat, J., et al. (2010). Derivation of pre-X inactivation human embryonic stem cells under physiological oxygen concentrations. Cell 141, 872-883.
Li, H., Ruan, J., and Durbin, R. (2008). Mapping short DNA sequencing reads and calling variants using mapping quality scores. Genome Res 18, 1851-1858.
Lister, R., Pelizzola, M., Dowen, R. H., Hawkins, R. D., Hon, G., Tonti-Filippini, J., Nery, J. R., Lee, L., Ye, Z., Ngo, Q. M., et al. (2009). Human DNA methylomes at base resolution show widespread epigenomic differences. Nature 462, 315-322.
Liu, L., Luo, G. Z., Yang, W., Zhao, X., Zheng, Q., Lv, Z., Li, W., Wu, H. J., Wang, L., Wang, X. J., et al. (2010). Activation of the imprinted Dlk1-Dio3 region correlates with pluripotency levels of mouse stem cells. J Biol Chem 285, 19483-19490.
Lu, R., Markowetz, F., Unwin, R. D., Leek, J. T., Airoldi, E. M., MacArthur, B. D., Lachmann, A., Rozov, R., Ma'ayan, A., Boyer, L. A., et al. (2009). Systems-level dynamic analyses of fate change in murine embryonic stem cells. Nature 462, 358-362.
Maherali, N., and Hochedlinger, K. (2008). Guidelines and techniques for the generation of induced pluripotent stem cells. Cell Stem Cell 3, 595-605.
Meissner, A., Mikkelsen, T. S., Gu, H., Wernig, M., Hanna, J., Sivachenko, A., Zhang, X., Bernstein, B. E., Nusbaum, C., Jaffe, D. B., et al. (2008). Genome-scale DNA methylation maps of pluripotent and differentiated cells. Nature 454, 766-770.
Mikkelsen, T. S., Hanna, J., Zhang, X., Ku, M., Wernig, M., Schorderet, P., Bernstein, B. E., Jaenisch, R., Lander, E. S., and Meissner, A. (2008). Dissecting direct reprogramming through integrative genomic analysis. Nature 454, 49-55.
Mikkelsen, T. S., Ku, M., Jaffe, D. B., Issac, B., Lieberman, E., Giannoukos, G., Alvarez, P., Brockman, W., Kim, T. K., Koche, R. P., et al. (2007). Genome-wide maps of chromatin state in pluripotent and lineage-committed cells. Nature 448, 553-560.
Mitalipova, M., Calhoun, J., Shin, S., Wininger, D., Schulz, T., Noggle, S., Venable, A., Lyons, I., Robins, A., and Stice, S. (2003). Human embryonic stem cell lines derived from discarded embryos. Stem Cells 21, 521-526.
Milller, F. J., Laurent, L. C., Kostka, D., Ulitsky, I., Williams, R., Lu, C., Park, I. H., Rao, M. S., Shamir, R., Schwartz, P. H., et al. (2008). Regulatory networks define phenotypic classes of human stem cell lines. Nature 455, 401-405.
Nam, D., and Kim, S. Y. (2008). Gene-set approach for expression pattern analysis. Briefings in Bioinformatics 9, 189-197.
Narva, E., Autio, R., Rahkonen, N., Kong, L., Harrison, N., Kitsberg, D., Borghese, L., Itskovitz-Eldor, J., Rasool, O., Dvorak, P., et al. (2010). High-resolution DNA analysis of human embryonic stem cell lines reveals culture-induced copy number changes and loss of heterozygosity. Nat. Biotechnol.
Osafune, K., Caron, L., Borowiak, M., Martinez, R. J., Fitz-Gerald, C. S., Sato, Y., Cowan, C. A., Chien, K. R., and Melton, D. A. (2008). Marked differences in differentiation propensity among human embryonic stem cell lines. Nat Biotechnol 26, 313-315.
Park, I. H., Arora, N., Huo, H., Maherali, N., Ahfeldt, T., Shimamura, A., Lensch, M. W., Cowan, C., Hochedlinger, K., and Daley, G. Q. (2008a). Disease-specific induced pluripotent stem cells. Cell 134, 877-886.
Park, I. H., Zhao, R., West, J. A., Yabuuchi, A., Huo, H., Ince, T. A., Lerou, P. H., Lensch, M. W., and Daley, G. Q. (2008b). Reprogramming of human somatic cells to pluripotency with defined factors. Nature 451, 141-146.
Reik, W. (2007). Stability and flexibility of epigenetic gene regulation in mammalian development. Nature 447, 425-432.
Rossant, J. (2008). Stem cells and early lineage development. Cell 132, 527-531.
Smith, Z. D., Gu, H., Bock, C., Gnirke, A., and Meissner, A. (2009). High-throughput bisulfite sequencing in mammalian genomes. Methods 48, 226-232.
Smyth, G. K. (2005). Limma: linear models for microarray data. In Bioinformatics and Computational Biology Solutions using R and Bioconductor, R. Gentleman, V. Carey, S. Dudoit, R. Irizarry, and W. Huber, eds. (New York, Springer), pp. 397-420.
Stadtfeld, M., Apostolou, E., Akutsu, H., Fukuda, A., Follett, P., Natesan, S., Kono, T., Shioda, T., and Hochedlinger, K. (2010a). Aberrant silencing of imprinted genes on chromosome 12qF1 in mouse induced pluripotent stem cells. Nature.
Stadtfeld, M., Apostolou, E., Akutsu, H., Fukuda, A., Follett, P., Natesan, S., Kono, T., Shioda, T., and Hochedlinger, K. (2010b). Aberrant silencing of imprinted genes on chromosome 12qF1 in mouse induced pluripotent stem cells. Nature 465, 175-181.
Storey, J. D., and Tibshirani, R. (2003). Statistical significance for genomewide studies. Proc Natl Acad Sci USA 100, 9440-9445.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proceedings of the National Academy of Sciences of the United States of America 102, 15545-15550.
Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131, 861-872.
Takahashi, K., and Yamanaka, S. (2006). Induction of pluripotent stem cells from mouse embryonic and adult fibroblast cultures by defined factors. Cell 126, 663-676.
Thomson, J. A., Itskovitz-Eldor, J., Shapiro, S. S., Waknitz, M. A., Swiergiel, J. J., Marshall, V. S., and Jones, J. M. (1998). Embryonic stem cell lines derived from human blastocysts. Science 282, 1145-1147.
Wichterle, H., Lieberam, I., Porter, J. A., and Jessell, T. M. (2002). Directed differentiation of embryonic stem cells into motor neurons. Cell 110, 385-397.
Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., et al. (2007). Induced pluripotent stem cell lines derived from human somatic cells. Science 318, 1917-1920.

### LENGTHY TABLES

The patent application contains eleven (11) lengthy Tables; Tables 3, Table 4, Table 5, Table 8, Table 10, Table 12A, Table 12B, Table 12C, Table 13A, Table 13B and Table 14. A copy of the Tables (Tables 3, Table 4, Table 5, Table 8, Table 10, Table 12A, Table 12B, Table 12C, Table 13A, Table 13B and Table 14) are available in electronic form from the USPTO web site. An electronic copy of the table will also be available from the USPTO upon request and payment of the fee set forth in 37 CFR 1.19(b)(3).

1. A method for selecting a pluripotent stem cell line, comprising
a. measuring DNA methylation of a set of target genes in the pluripotent stem cell line, and performing a comparison of the DNA methylation data with a reference DNA methylation data of the same target genes;
b. measuring differentiation potential of the pluripotent stem cell line by undirected or directed differentiation of the pluripotent stem cell by measuring the gene expression and/or DNA methylation of a plurality of lineage marker genes; and comparing the gene expression and/or DNA methylation differentiation with a reference gene expression and/or DNA methylation differentiation of the same lineage marker genes; and
c. selecting a pluripotent stem cell line which does not differ by a statistically significant amount in the DNA methylation of the target genes as compared to the reference DNA methylation level, and does not differ by a statistically significant amount in the propensity to differentiate along mesoderm, ectoderm and endoderm lineages as compared to a reference differentiation potential; or discarding a pluripotent stem cell line which differs by a statistically significant amount in the in the DNA methylation of the target genes as compared to the reference DNA methylation level, and differs by a statistically significant amount in the propensity to differentiate along mesoderm, ectoderm and endoderm lineages as compared to a reference differentiation potential.

2. (canceled)

3. (canceled)

4. (canceled)

5. The method of claim 1, further comprising:
a. measuring the gene expression of a second set of target genes in the pluripotent stem cell line and performing a comparison of the gene expression data with a reference gene expression level of the same target genes; and
b. selecting a pluripotent stem cell line which does not differ by a statistically significant amount in the level of gene expression of the target genes as compared to the reference gene expression level; or discarding a pluripotent stem cell line which differs by a statistically significant amount in the expression level of the target genes as compared to the reference gene expression level.

6. (canceled)

7. (canceled)

8. (canceled)

9. The method of claim 1, wherein DNA methylation for the pluripotent cell line and/or the reference is determined by a DNA methylation assay is selected from the group consisting of: enrichment-based methods (e.g. MeDIP, MBD-seq and MethylCap), bisulfide sequencing, whole-genome bisulfite assay, reduced-representation bisulfite sequencing (RBBS), and bisulfite-based methods (e.g., Infinium, GoldenGate, COBRA, MSP, MethyLight) and restriction-digestion methods (e.g., MRE-seq).

10. (canceled)

11. (canceled)

12. (canceled)

13. (canceled)

14. (canceled)

15. The method of claim 5, wherein the gene expression of the pluripotent cell line and/or reference is determined by a microarray assay or a quantitative differentiation assay.

16. (canceled)

17. The method of claim 1, wherein the reference differentiation potential is the ability to differentiate into a lineage selected from the group consisting of mesoderm, endoderm, ectoderm, neuronal, hematopoietic lineages, and any combinations thereof.

18. (canceled)

19. (canceled)

20. (canceled)

21. The method of claim 1, wherein the pluripotent cell line DNA methylation target genes and/or the reference DNA methylation target genes are selected from the group listed in Table 12A or Table 13A or Table 14, and any combinations thereof.

22. (canceled)

23. (canceled)

24. The method of claim 21, wherein DNA methylation target genes and/or the reference DNA methylation target genes are developmental genes are selected from any combination of genes listed in Table 7 or Table 13A or Table 14.

25. (canceled)

26. (canceled)

27. (canceled)

28. The method of claim 1, wherein the pluripotent cell line gene expression target genes and/or the reference gene expression target genes are selected from the group listed in Table 12B or Table 13A or Table 14, and any combinations thereof.

29. The method of claim 1, wherein the DNA methylation of least about 200 target genes selected from any combination of genes in the list in Table 12A or Table 13A or Table 14 are measured in the pluripotent cell line, and compared to the reference DNA methylation level of the same set of at least 200 target genes.

30. (canceled)

31. (canceled)

32. (canceled)

33. (canceled)

34. (canceled)

35. (canceled)

36. (canceled)

37. The method of claim 1, wherein the gene expression of least about 200 target genes selected from any combination of genes in the list in Table 12B or Table 13A or Table 14 are measured in the pluripotent cell line, and compared to the reference gene expression level of the same set of at least 200 target genes.

38.-44. (canceled)

45. The method of claim 1, wherein the pluripotent stem cell is a mammalian pluripotent stem cell or a human pluripotent stem cells or a human induced pluripotent stem cells (iPSC).

46-69. (canceled)

70-72. (canceled)

73. The assay of claim 70, wherein DNA methylation assay is selected from the group consisting of: enrichment-based methods (e.g. MeDIP, MBD-seq and MethylCap), bisulfide sequencing and whole genome bisulfite sequencing, and bisulfite-based methods (e.g. RRBS, bisulfite sequencing, Infinium, GoldenGate, COBRA, MSP, MethyLight) and restriction-digestion methods (e.g., MRE-seq).

74. The assay of claim 70, wherein the gene expression assay is a microarray assay.

75. (canceled)

76. The assay of claim 70, wherein the differentiation assay assess the ability of the pluripotent cell to differentiate into at least one of the following lineages: mesoderm, endoderm, ectoderm, neuronal, or hematopoietic lineages.

77-81. (canceled)

82. The assay of claim 70, wherein the assay is a high-throughput assay for assaying a plurality of different pluripotent stem cells or induced pluripotent stem cells (iPSCs) from a subject.

83-87. (canceled)

88. The assay of claim 70, wherein the gene expression assay determines the expression of genes selected from any combination of genes listed in Table 7 or Tables 13A or Table 14.

89. The assay of claim 70, wherein the DNA methylation assay determines the DNA methylation levels of any combination of a plurality of target genes selected from the group listed in Table 12A or Tables 13A or Table 14.

90.-95. (canceled)

96. The assay of claim 70, wherein the gene expression assay determines the gene expression level of any combination of a plurality of target genes selected from the group listed in Table 12B or Tables 13A or Table 14.

97.-133. (canceled)

134. A scorecard of the performance parameters of a pluripotent stem cell, the scorecard comprising:
(i) a first data set comprising the DNA methylation levels for a plurality of DNA methylation target genes from a plurality of pluripotent stem cell lines;
(ii) a second data set comprising the gene expression levels for a plurality of gene expression target genes from a plurality of pluripotent stem cell lines; and
(iii) a third data set comprising the differentiation propensity levels for differentiation into ectoderm, mesoderm and endoderm lineages from a plurality of pluripotent stem cell lines.

135. (canceled)

136. The scorecard of claim 134, wherein the plurality of reference DNA methylation genes is selected from any combination of genes listed in Table 12A or Tables 13A or Table 14.

137-147. (canceled)

148. The scorecard of claim 134, wherein at least the first and/or second data set are connected to a data storage device, and the data storage device is a database located on a computer device.

149.-233. (canceled)


SEQUENCE LISTING (15 sequences)

SEQ ID NO: 1 - DNA, 21 bp, Artificial Sequence
  source: /note=“Description of Artificial Sequence Synthetic primer”
atggtgtttgtggaaggggaa

SEQ ID NO: 2 - DNA, 27 bp, Artificial Sequence
  source: /note=“Description of Artificial Sequence Synthetic primer”
tccaaacaactaaaatatacaaaacct

SEQ ID NO: 3 - DNA, 28 bp, Artificial Sequence
  source: /note=“Description of Artificial Sequence Synthetic primer”
taatatgaggtaattagtttagtttagt

SEQ ID NO: 4 - DNA, 27 bp, Artificial Sequence
  source: /note=“Description of Artificial Sequence Synthetic primer”
taatttcaaactctaacttcaaataat

SEQ ID NO: 5 - DNA, 22 bp, Artificial Sequence
  source: /note=“Description of Artificial Sequence Synthetic primer”
agttgtggttgaggtttaggtt

SEQ ID NO: 6 - DNA, 22 bp, Artificial Sequence
  source: /note=“Description of Artificial Sequence Synthetic primer”
accacaaaacttacactttcca

SEQ ID NO: 7 - DNA, 19 bp, Artificial Sequence
  source: /note=“Description of Artificial Sequence Synthetic primer”
acgccagaaccttgtgagc

SEQ ID NO: 8 - DNA, 22 bp, Artificial Sequence
  source: /note=“Description of Artificial Sequence Synthetic primer”
gcatggatctccacctctactg

SEQ ID NO: 9 - DNA, 20 bp, Artificial Sequence
  source: /note=“Description of Artificial Sequence Synthetic primer”
tcttctcctggttgtcagct

SEQ ID NO: 10 - DNA, 19 bp, Artificial Sequence
  source: /note=“Description of Artificial Sequence Synthetic primer”
gaggcagagacaaagagcg

SEQ ID NO: 11 - DNA, 20 bp, Artificial Sequence
  source: /note=“Description of Artificial Sequence Synthetic primer”
gtgtcatgcgtggaaggata

SEQ ID NO: 12 - DNA, 20 bp, Artificial Sequence
  source: /note=“Description of Artificial Sequence Synthetic primer”
gcactggagctggaaatagc

SEQ ID NO: 13 - DNA, 21 bp, Artificial Sequence
  source: /note=“Description of Artificial Sequence Synthetic primer”
acccactcctccacctttgac

SEQ ID NO: 14 - DNA, 22 bp, Artificial Sequence
  source: /note=“Description of Artificial Sequence Synthetic primer”
accctgttgctgtagccaaatt

SEQ ID NO: 15 - protein, 6 aa, Artificial Sequence
  source: /note=“Description of Artificial Sequence Synthetic 6xHis tag”
HHHHHH